(19) United States
(12) Patent Application Publication
(10) Pub. No.: US 2026/0317771 A1
(43) Pub. Date: Oct. 15, 2026

(54) RNAI AGENTS FOR INHIBITING EXPRESSION OF DM1 PROTEIN KINASE (DMPK) COMPOSITIONS THEREOF, AND METHODS OF USE

(71) Applicant: ARROWHEAD PHARMACEUTICALS, INC., Pasadena, CA (US)

(72) Inventors: Jonathan VAN DYKE, Madison, WI (US); Teng AI, San Diego, CA (US); Xiaokai LI, San Diego, CA (US); Zhi-Ming DING, Madison, WI (US); Tao PEI, Madison, WI (US); Casi SCHIENEBECK, Madison, WI (US)

(73) Assignee: ARROWHEAD PHARMACEUTICALS, INC., Pasadena, CA (US)

(21) Appl. No.: 19/685,709

(22) Filed: May 22, 2026

Related U.S. Application Data
(63) Continuation of application No. 19/120,297, filed on Oct. 18, 2023.
(60) Provisional application No. 63/380,171, filed on Oct. 19, 2022.
(60) Provisional application No. 63/584,283, filed on Sep. 21, 2023.

(51) Int. Cl. C12N 15/113; A61P 21/00

(52) U.S. Cl. CPC C12N 15/1137; A61P 21/00; C12Y 207/11001; C12N 2310/14; C12N 2310/315; C12N 2310/321; C12N 2310/322; C12N 2310/333; C12N 2310/334; C12N 2310/335; C12N 2310/336; C12N 2310/3513

(57) ABSTRACT

Described are RNAi agents, compositions that include RNAi agents, and methods for inhibition of DM1 protein kinase (DMPK) gene. The DMPK RNAi agents and RNAi agent conjugates disclosed herein inhibit the expression of a DMPK gene. Pharmaceutical compositions that include one or more DMPK RNAi agents, optionally with one or more additional therapeutics, are also described. Delivery of the described DMPK RNAi agents to skeletal muscle cells in vivo, provides for inhibition of DMPK gene expression and a reduction in DMPK protein levels, and more specifically a reduction in mutant DMPK-CUG protein levels, which can provide a therapeutic benefit to subjects, including human subjects, suffering from certain skeletal muscle-related diseases or disorders including myotonic dystrophy type 1.

### CROSS REFERENCE TO RELATED APPLICATIONS

This application is a continuation of U.S. application Ser. No. 19/120,297, which is a 35 U.S.C. § 371 National Phase Application of International Application No. PCT/US2023/077179, filed on Oct. 18, 2023, which claims the benefit of priority of U.S. Provisional Patent Application Ser. No. 63/380,171, filed on Oct. 19, 2022, and U.S. Provisional Patent Application Ser. No. 63/584,283, filed on Sep. 21, 2023, the contents of each of which are incorporated herein by reference in their entirety.

### SEQUENCE LISTING

This application contains a Sequence Listing which has been submitted in XML format and is hereby incorporated by reference in its entirety. The XML copy is named 4140_1180003_SequenceListing_ST26.xml, was created on May 19, 2026, and is 9,222,356 bytes in size.

### FIELD OF THE INVENTION

The present disclosure relates to RNA interference (RNAi) agents, e.g., double stranded RNAi agents, for inhibition of DM1 protein kinase (DMPK) gene expression, compositions that include DMPK RNAi agents, and methods of use thereof.

### BACKGROUND

Myotonic dystrophy protein kinase (DMPK), or DM1 protein kinase, is expressed primarily in muscle. Myotonic dystrophy type 1 (DM1) is an autosomal dominant multisystemic disorder caused by a >50 CTG repeat expansion in the 3′ untranslated region (3′ UTR) of the DMPK gene (Legare et al., Neurology Genetics 2019: 5 (3)). The expanded CTG repeats of the 3′UTR, when transcribed, result in an mRNA transcript with an expanded CUG repeat sequence in the 3′ UTR (DMPK-CUG) that renders the transcript unable to transit the nuclear pore into the cytoplasm and a resultant nuclear accumulation. The accumulation of the mutant DMPK-CUG leads to the misregulation of several RNA-binding proteins, including CUGBP1 and MBNL1, known to be responsible for proper RNA processing of a multitude of gene products prior to translation. It is this misregulation of RNA processing that leads to expression of incorrect protein isoforms, mislocalization of proteins, or disruption in synthesis of critical proteins eventually leading to loss of cell function and viability.

DM1 affects all systems including muscular, cardiac, respiratory, endocrine, and central nervous system (CNS). The hallmark feature of DM1 is the presence of myotonia, including progressive skeletal muscle impairment with a distal to proximal pattern that can also affect muscles responsible for respiration due to misregulation of RNA processing of gene products responsible for normal muscle function.

Currently, no therapy exists to treat the root cause of DM1. The degradation of accumulated DMPK-CUG is thought to be a powerful approach for the alleviation of DM1 pathology. By reducing the accumulated transcript in myonuclei, proper mRNA splicing regulation can be reestablished and normal cell function returned. RNA interference agents such as those described in this document are effective for selectively reducing mRNA targets and are expected to reduce accumulated DMPK-CUG in DM1 patients and return normal function of affected skeletal muscle.

### SUMMARY

There is a need for novel RNA interference (RNAi) agents (also herein referred to as RNAi agent, RNAi trigger, or trigger), e.g., double stranded RNAi agents such as small interfering RNAs (siRNAs), that are able to selectively and efficiently inhibit the expression of a DM1 protein kinase (DMPK) gene, particularly in vivo. Further, there exists a need for compositions of novel DMPK-specific RNAi agents for the treatment of diseases or disorders such as myotonic dystrophy type 1 that can be ameliorated at least in part by a reduction in mutant DMPK-CUG protein levels.

In general, the present disclosure features DMPK RNAi agents, compositions that include such RNAi agents, and methods for inhibiting expression of a DMPK gene in vitro and/or in vivo using the RNAi agents and compositions that include the RNAi agents described herein. The DMPK RNAi agents described herein are able to selectively and efficiently decrease, inhibit, or silence expression of a DMPK gene.

The described DMPK RNAi agents can be used in methods for therapeutic treatment (including preventative, intervention, or prophylactic treatment) of symptoms and diseases such as myotonic dystrophy type 1. The methods disclosed herein include the administration of one or more DMPK RNAi agents to a subject, e.g., a human or animal subject, using any suitable methods known in the art, such as for example, subcutaneous (SQ) injection, intramuscular injection, or intravenous (IV) administration.

In one aspect, the disclosure features RNAi agents for inhibiting expression of a DMPK gene, wherein the RNAi agent includes a sense strand (also referred to as a passenger strand) and an antisense strand (also referred to as a guide strand). The sense strand and the antisense strand can be partially, substantially, or fully complementary to each other. The length of the RNAi agent sense strands described herein each can be 15 to 49 nucleotides in length. The length of the RNAi agent antisense strands described herein each can be 17 to 49 nucleotides in length. In some embodiments, the sense and antisense strands are independently 17 to 26 nucleotides in length. The sense and antisense strands can be either the same length or different lengths. In some embodiments, the sense and antisense strands are independently 21 to 26 nucleotides in length. In some embodiments, the sense and antisense strands are independently 21 to 24 nucleotides in length. In some embodiments, both the sense strand and the antisense strand are 21 nucleotides in length. In some embodiments, the antisense strands are independently 17, 18, 19, 20, 21, 22, 23, 24, 25, 26, 27, 28, 29, or 30 nucleotides in length. In some embodiments, the sense strands are independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The RNAi agents described herein, upon delivery to a cell expressing DMPK such as a skeletal muscle cell (a skeletal myofiber), inhibit the expression of one or more DMPK gene transcripts in vivo and/or in vitro.

The DMPK RNAi agents disclosed herein target a DM1 protein kinase (DMPK) gene (see, e.g., SEQ ID NO: 1, Homo sapiens transcript variant 1). In some embodiments, the RNAi agents disclosed herein target a portion of a DMPK gene having the sequence of any of the sequences disclosed in Table 1.

In another aspect, the disclosure features pharmaceutical compositions that include one or more of the disclosed DMPK RNAi agents that are able to selectively and efficiently decrease expression of a DMPK gene. The pharmaceutical compositions that include one or more DMPK RNAi agents described herein can be administered to a subject, such as a human or animal subject, for the treatment (including intervention or prophylactic treatment or inhibition) of symptoms and diseases that can be ameliorated at least in part by a reduction in DMPK protein levels, and more specifically a reduction in mutant DMPK-CUG protein levels. The pharmaceutical compositions described herein include an RNAi agent capable of inhibiting the expression of a DMPK gene and at least one pharmaceutically acceptable excipient.

Examples of DMPK RNAi agent sense strands and antisense strands that can be used in a DMPK RNAi agent are provided in Tables 3 and Tables 4.1, 4.2, 4.3, 4.4, 4.5, 4.6, 5.4, and 5.5. Examples of DMPK RNAi agent duplexes are provided in Tables 5.1, 5.2, 5.3, 5.4, 5.6, and 5.7. Examples of 19-nucleotide core stretch sequences that may consist of or may be included in the sense strands and antisense strands of certain DMPK RNAi agents disclosed herein, are provided in Table 2.

One aspect described herein is an RNAi agent for inhibiting expression of a DMPK gene comprising:
(i) an antisense strand comprising at least 17 contiguous nucleotides differing by 0 or 1 nucleotides from any one of the sequences provided in Table 3 or Table 5.4; and
(ii) a sense strand comprising a nucleotide sequence that is at least partially complementary to the antisense strand.

In another aspect described herein is an RNAi agent for inhibiting expression of a DMPK gene comprising:
(i) an antisense strand comprising at least 17 contiguous nucleotides differing by 0 or 1 nucleotides from any one of the sequences provided in Table 3 or Table 5.4;
(ii) a sense strand comprising a nucleotide sequence that is at least partially complementary to the antisense strand;
(iii) a targeting ligand linked to the sense strand that has affinity for skeletal muscle cells and/or a receptor present on skeletal muscle cells; and
(iv) a PK/PD modulator linked to the sense strand.

In yet a further aspect described herein is an RNAi agent for inhibiting expression of a DMPK gene comprising:
(i) an antisense strand comprising at least 17 contiguous nucleotides differing by 0 or 1 nucleotides from any one of the sequences provided in Table 3 or Table 5.4;
(ii) a sense strand comprising a nucleotide sequence that is at least partially complementary to the antisense strand;
(iii) a targeting ligand linked to the sense strand that has affinity for skeletal muscle cells and/or a receptor present on skeletal muscle cells wherein the targeting ligand is linked to the 5′ terminal end of the sense strand; and
(iv) a PK/PD modulator linked to the 3′ terminal end of the sense strand.

In another aspect described herein is an RNAi agent for inhibiting expression of a DMPK gene comprising:
(i) an antisense strand comprising at least 17 contiguous nucleotides differing by 0 or 1 nucleotides from any one of the sequences provided in Table 3 or Table 5.4;
(ii) a sense strand comprising a nucleotide sequence that is at least partially complementary to the antisense strand;
(iii) a targeting ligand that comprises a chemical structure represented in Table 6.2 or 6.3 herein, wherein the targeting ligand is linked to the 5′ terminal end of the sense strand; and
(iv) a PK/PD modulator that comprises a chemical structure represented in Table 6.5 or 6.7 herein, wherein the PK/PD modulator is linked to the 3′ terminal end of the sense strand.

In another aspect, the disclosure features methods for delivering DMPK RNAi agents to skeletal muscle cells in a subject, such as a mammal, e.g., a human subject, in vivo. Also described herein are compositions for use in such methods.

The one or more DMPK RNAi agents can be delivered to target cells or tissues using any oligonucleotide delivery technology known in the art. In some embodiments, a DMPK RNAi agent is delivered to cells or tissues by covalently linking the RNAi agent to a targeting group. In some embodiments, the targeting group can include a cell receptor ligand. A targeting group can be linked to the 3′ or 5′ end of a sense strand or an antisense strand of a DMPK RNAi agent, or can be linked via one or more internal nucleotides of the sense strand or the antisense strand. In some embodiments, a targeting group is linked to the 3′ or 5′ end of the sense strand. In some embodiments, a targeting group is linked to the 5′ end of the sense strand. In some embodiments, a targeting group is linked internally to a nucleotide on the sense strand and/or the antisense strand of the RNAi agent. In some embodiments, a targeting group is linked to the RNAi agent via a linker. Example targeting ligands suitable for use that have affinity for skeletal muscle cells and/or receptors present on skeletal muscle cells (e.g., integrin alpha-v-beta-6 (αvβ6)), are shown in Table 6.2 and 6.3 herein. The synthesis and conjugation of certain targeting ligands suitable for use with the DMPK RNAi agents disclosed herein are described in Example 1.

In some embodiments, the DMPK RNAi agents disclosed herein that are conjugated to targeting groups or targeting ligands that direct the RNAi agent to skeletal muscle cells, whereby the RNAi agents can be selectively internalized either through receptor-mediated endocytosis or by other means.

In another aspect, the disclosure features methods for inhibiting DMPK gene expression in a subject, the methods including administering to the subject an amount of a DMPK RNAi agent capable of inhibiting the expression of a DMPK gene, wherein the DMPK RNAi agent comprises a sense strand and an antisense strand, and wherein the antisense strand includes the sequence of any one of the antisense strand nucleotide sequences in Table 2, Table 3, or Table 5.4. In a further aspect, the disclosure features methods of treatment (including prophylactic, intervention, or preventative treatment) of diseases or symptoms that can be ameliorated at least in part by a reduction in DMPK protein levels (and more specifically a reduction in mutant DMPK-CUG protein levels), the methods comprising administering to a subject in need thereof a DMPK RNAi agent that includes an antisense strand comprising the sequence of any of the sequences in Table 2, Table 3, or Table 5.4. Pharmaceutical compositions for use in such methods are also described.

In some embodiments, a DMPK RNAi agent is linked to one or more linking groups or other non-nucleotide groups or compounds, such as pharmacokinetic/pharmacodynamic (PK/PD) modulators. PK/PD modulators can increase circulation time of the conjugated drug and/or increase the activity of the RNAi agent through improved cell receptor binding, improved cellular uptake, and/or other means. Examples of PK/PD modulators suitable for use with the DMPK RNAi agents disclosed herein can be found in Table 6.5 and 6.7, herein.

In some embodiments, a DMPK RNAi agent is conjugated to a targeting group, a linking group, a PK/PD modulator, and/or another non-nucleotide group. In some embodiments, a DMPK RNAi agent is conjugated to a targeting group and a PK/PD modulator.

The use of DMPK RNAi agents provides methods for therapeutic (including prophylactic or intervention) treatment of diseases or disorders that can be ameliorated at least in part by a reduction in DMPK protein levels, specifically including a reduction in mutant DMPK-CUG protein levels. Described herein are compositions for delivery of DMPK RNAi agents to skeletal muscle cells to a subject. In some embodiments, the DMPK RNAi agents disclosed herein are able to reduce DMPK gene expression in paraspinal, facial, torso, abdominal, and limb muscle tissues of the subject, for example, in the triceps, biceps, quadriceps, pectoralis, gastrocnemius, soleus, masseter, EDL (extensor digitorum longus), TA (Tibialis anterior), trapezius, and/or diaphragm, of the subject.

In some embodiments, methods for the treatment (including prophylactic or intervention treatment) of a pathological state mediated at least in part by DMPK expression, such as myotonic dystrophy type 1, are disclosed herein, wherein the methods include administering to a subject a therapeutically effective amount of an RNAi agent that includes a sense strand comprising the sequence of any of the sequences in Table 2, Table 4.1, Table 4.2, Table 4.3, Table 4.4, Table 4.5, Table 4.6, Table 5.4, or Table 5.5.

In some embodiments, methods for the treatment (including prophylactic or intervention treatment) of a pathological state mediated at least in part by DMPK expression are disclosed herein, wherein the methods include administering to a subject a therapeutically effective amount of a DMPK RNAi agent that includes a sense strand comprising the sequence of any of the sequences in Table 4.1, 4.2, 4.3, 4.4, 4.5, 4.6, 5.4, or 5.5 herein, and an antisense strand comprising the sequence of any of the sequences in Table 3 or Table 5.4.

In some embodiments, methods of inhibiting expression of a DMPK gene are disclosed herein, wherein the methods include administering to a subject a DMPK RNAi agent that includes a sense strand consisting of the nucleobase sequence of any of the sequences in Table 4.1, 4.2, 4.3, 4.4, 4.5, 4.6, 5.4, or 5.5 herein, and the antisense strand consisting of the nucleobase sequence of any of the sequences in Table 3 or Table 5.4. In other embodiments, disclosed herein are methods of inhibiting expression of a DMPK gene, wherein the methods include administering to a subject a DMPK RNAi agent that includes a sense strand consisting of the modified sequence of any of the modified sequences in Table 4.1, 4.2, 4.3, 4.4, 4.5, 4.6, 5.4, 5.5 herein, and the antisense strand consisting of the modified sequence of any of the modified sequences in Table 3 or Table 5.4.

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: short (or small) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted (i.e., a DMPK mRNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, the term “nucleotide” has the same meaning as commonly understood in the art, and thus refers to a glycoside comprising a sugar moiety, a base moiety and a covalently linked group (linkage group), such as a phosphate or phosphorothioate or phosphorodithioate internucleoside linkage group, and covers both naturally occurring nucleotides, such as DNA or RNA, and non-naturally occurring nucleotides comprising modified sugar and/or base moieties, which are also referred to as nucleotide analogs or modified nucleotides herein. Herein, a single nucleotide can be referred to as a monomer or unit.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of a DMPK mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “individual”, “patient” and “subject”, are used interchangeably to refer to a member of any animal species including, but not limited to, birds, humans and other primates, and other mammals including commercially relevant mammals or animal models such as mice, rats, monkeys, cattle, pigs, horses, sheep, cats, and dogs. Preferably, the subject is a human.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic or intervention treatment, and/or inhibition or reduction of the number, severity, and/or frequency of one or more symptoms of a disease in a subject.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

Unless stated otherwise, use of the symbol

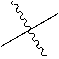

as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention. A typical pharmaceutically acceptable salt of the disclosed DMPK RNAi agents is in the form of a sodium salt.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Where a value is explicitly recited, it is to be understood that values which are about the same quantity or amount as the recited value are also within the scope of the disclosure.

Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Other objects, features, aspects, and advantages of the invention will be apparent from the following detailed description, accompanying figures, and from the claims.

### BRIEF DESCRIPTION OF THE DRAWINGS

FIG. 1. shows normal splicing conditions and the forward and reverse primer sets designed against exons flanking exons known to be excluded or included under normal transcript splicing conditions and transcripts known to be mis-spliced in the accumulated presence of mutant DMPK-CUG transcript in the nucleus, as described in Example 17.

FIG. 2. Shows the expression of hDMPK transcript in mice under various dosing conditions according to the description in Example 18.

FIG. 3. Shows the relative missplicing of mCacna1 in mice under various dosing conditions according to the description in Example 18.

FIG. 4. Shows the relative missplicing of mLdb3 in mice under various dosing conditions according to the description in Example 18.

FIG. 5. Shows the relative missplicing of mMbnl1 in mice under various dosing conditions according to the description in Example 18.

FIG. 6. Shows the relative missplicing of mAtp2a1 in mice under various dosing conditions according to the description in Example 18.

### DETAILED DESCRIPTION

Described herein are RNAi agents for inhibiting expression of a DMPK gene (referred to herein as DMPK RNAi agents or DMPK RNAi triggers). Each DMPK RNAi agent comprises a sense strand and an antisense strand. The sense strand can be 15 to 49 nucleotides in length. The antisense strand each can be 17 to 49 nucleotides in length. The sense and antisense strands can be either the same length or they can be different lengths. In some embodiments, the sense and antisense strands are each independently 17 to 27 nucleotides in length. In some embodiments, the sense and antisense strands are each independently 19-21 nucleotides in length. In some embodiments, both the sense and antisense strands are each 21-26 nucleotides in length. In some embodiments, the sense and antisense strands are each 21-24 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments, both the sense and antisense strands are each 21 nucleotides in length. In some embodiments, the RNAi agent sense strands are 15, 16, 17, 18, 19, 20, 21, 22, 23, 24, 25, 26, 27, 28, 29, 30, 31, 32, 33, 34, 35, 36, 36, 38, 39, 40, 41, 42, 43, 44, 45, 46, 47, 48, or 49 nucleotides in length. In some embodiments, the RNAi agent antisense strands are 17, 18, 19, 20, 21, 22, 23, 24, 25, 26, 27, 28, 29, or 30 nucleotides in length. In some embodiments, a double-stranded RNAi agent has a duplex length of about 15, 16, 17, 18, 19, 20, 21, 22, 23 or 24 nucleotides.

Examples of nucleotide sequences used in forming DMPK RNAi agents are provided in Tables 2, 3, 4.1, 4.2, 4.3, 4.4, 4.5, 4.6, 5.4, and 5.5. Examples of RNAi agent duplexes, that include the sense strand and antisense strand sequences in Tables 2, 3, 4.1, 4.2, 4.3, 4.4, 4.5, 4.6, are shown in Tables 5.1, 5.2, 5.3, 5.4, 5.6, and 5.7.

In some embodiments, the region of perfect, substantial, or partial complementarity between the sense strand and the antisense strand (sometimes referred to the “duplex region”) is 12-26 (e.g., 12, 13, 14, 15, 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementary).

A sense strand of the DMPK RNAi agents described herein includes at least 12 consecutive nucleotides that have at least 85% identity to a core stretch sequence (also referred to herein as a “core stretch” or “core sequence”) of the same number of nucleotides in a DMPK mRNA. In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the DMPK mRNA target. In some embodiments, this sense strand core stretch is 12, 13, 14, 15, 16, 17, 18, 19, 20, 21, 22, or 23 nucleotides in length. In some embodiments, this sense strand core stretch is 17 nucleotides in length. In some embodiments, this sense strand core stretch is 19 nucleotides in length. In some embodiments, this sense strand core stretch is 21 nucleotides in length.

An antisense strand of a DMPK RNAi agent described herein includes at least 17 consecutive nucleotides that have at least 85% complementarity to a core stretch of the same number of nucleotides in a DMPK mRNA, and in some embodiments, to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in the DMPK mRNA target. In some embodiments, this antisense strand core stretch is 17, 18, 19, 20, 21, 22, or 23 nucleotides in length. In some embodiments, this antisense strand core stretch is 19 nucleotides in length. In some embodiments, this antisense strand core stretch is 17 nucleotides in length. In some embodiments, this antisense strand core stretch is 21 nucleotides in length. In some embodiments, this antisense strand core stretch is 23 nucleotides in length. A sense strand core stretch sequence can be the same length as a corresponding antisense core sequence or it can be a different length.

The DMPK RNAi agent sense and antisense strands anneal to form a duplex. A sense strand and an antisense strand of a DMPK RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 16, at least 17, at least 18, at least 19, at least 20, at least 21, at least 22, or at least 23 nucleotides that is at least 85% or 100% complementary to a corresponding 16, 17, 18, 19, 20, 21, 22, or 23 nucleotide sequence of the antisense strand core stretch sequence (i.e., the sense and antisense core stretch sequences of a DMPK RNAi agent have a region of at least 16, at least 17, at least 18, at least 19, at least 20, at least 21, at least 22, or at least 23 nucleotides that is at least 85% base paired or 100% base paired.)

In some embodiments, the antisense strand of a DMPK RNAi agent disclosed herein differs by 0, 1, 2, or 3 nucleotides from any of the antisense strand sequences in Table 2, Table 3, or Table 5.4.

In some embodiments, the sense strand of a DMPK RNAi agent disclosed herein differs by 0, 1, 2, or 3 nucleotides from any of the sense strand sequences in Table 2 or Table 4.1, or Table 4.2, or Table 4.3, or Table 4.4, or Table 4.5, Table 4.6, Table 5.4, or Table 5.5.

In some embodiments, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in the DMPK mRNA. The sense strand additional nucleotides, if present, may or may not be identical to the corresponding sequence in the DMPK mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, a DMPK RNAi agent has an antisense strand having a 3′ extension and a sense strand having a 5′ extension. In some embodiments, the extension nucleotide(s) are unpaired and form an overhang. As used herein, an “overhang” refers to a stretch of one or more unpaired nucleotides located at a terminal end of either the sense strand or the antisense strand that does not form part of the hybridized or duplexed portion of an RNAi agent disclosed herein.

In some embodiments, a DMPK RNAi agent comprises an antisense strand having a 3′ extension of 1, 2, 3, 4, 5, or 6 nucleotides in length. In other embodiments, a DMPK RNAi agent comprises an antisense strand having a 3′ extension of 1, 2, or 3 nucleotides in length. In some embodiments, one or more of the antisense strand extension nucleotides comprise nucleotides that are complementary to the corresponding DMPK mRNA sequence. In some embodiments, one or more of the antisense strand extension nucleotides comprise nucleotides that are not complementary to the corresponding DMPK mRNA sequence.

In some embodiments, a DMPK RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in the DMPK mRNA sequence. In some embodiments, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some embodiments, a DMPK RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in the DMPK mRNA sequence. In some embodiments, the sense strand 5′ extension is one of the following sequences, but is not limited to: CA, AUAGGC, AUAGG, AUAG, AUA, A, AA, AC, GCA, GGCA, GGC, UAUCA, UAUC, UCA, UAU, U, UU (each listed 5′ to 3′).

Examples of sequences used in forming DMPK RNAi agents are provided in Tables 2, 3, and 4.1, 4.2, 4.3, 4.4, 4.5, 4.6, and 5.4. In some embodiments, a DMPK RNAi agent antisense strand includes a sequence of any of the sequences in Tables 2 or 3. In certain embodiments, a DMPK RNAi agent antisense strand comprises or consists of any one of the modified sequences in Table 3 or Table 5.4. In some embodiments, a DMPK RNAi agent antisense strand includes the sequence of nucleotides (from 5′ end→3′ end) 1-17, 2-15, 2-17, 1-18, 2-18, 1-19, 2-19, 1-20, 2-20, 1-21, or 2-21, of any of the sequences in Tables 2, 3, or 5.4. In some embodiments, a DMPK RNAi agent sense strand includes the sequence of any of the sequences in Tables 2, 4.1, 4.2, 4.3, 4.4, 4.5, 4.6, or 5.4. In some embodiments, a DMPK RNAi agent sense strand includes the sequence of nucleotides (from 5′ end→3′ end) 1-18, 1-19, 1-20, 1-21, 2-19, 2-20, 2-21, 3-20, 3-21, or 4-21 of any of the sequences in Tables 2, 4.1, 4.2, 4.3, 4.4, 4.5, 4.6, or 5.4. In certain embodiments, a DMPK RNAi agent sense strand comprises or consists of a modified sequence of any one of the modified sequences in Table 4.1, Table 4.2, Table 4.3, Table 4.4, Table 4.5, Table 4.6, Table 5.4, or Table 5.5.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands from a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The DMPK RNAi agents disclosed herein may also be comprised of one or more modified nucleotides. In some embodiments, substantially all of the nucleotides of the sense strand and substantially all of the nucleotides of the antisense strand of the DMPK RNAi agent are modified nucleotides. The DMPK RNAi agents disclosed herein may further be comprised of one or more modified internucleoside linkages, e.g., one or more phosphorothioate or phosphorodithioates linkages. In some embodiments, a DMPK RNAi agent contains one or more modified nucleotides and one or more modified internucleoside linkages. In some embodiments, a 2′-modified nucleotide is combined with modified internucleoside linkage.

In some embodiments, a DMPK RNAi agent is prepared or provided as a salt, mixed salt, or a free-acid. In some embodiments, a DMPK RNAi agent is prepared as a sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administering of the oligonucleotide construct.

In some embodiments, a DMPK RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Methyl, 2′-fluoro nucleotides, morpholino nucleotides, vinyl phosphonate-containing nucleotides, and cyclopropyl phosphonate-containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to as 2′-methoxy nucleotides), 2′-fluoro nucleotides (also referred to herein as 2′-deoxy-2′-fluoro nucleotides), 2′-deoxy nucleotides, 2′-methoxyethyl(2′-O-(2-methoxylethyl)) nucleotides (also referred to as 2′-MOE), 2′-amino nucleotides, and 2′-alkyl nucleotides. It is not necessary for all positions in a given compound to be uniformly modified. Conversely, more than one modification can be incorporated in a single DMPK RNAi agent or even in a single nucleotide thereof. The DMPK RNAi agent sense strands and antisense strands can be synthesized and/or modified by methods known in the art. Modification at one nucleotide is independent of modification at another nucleotide. Various modified nucleotides are well known and described in the art.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine (hypoxanthine), xanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense sense strand wherein substantially all of the nucleotides present are modified nucleotides is an antisense strand having two or fewer (i.e., 0, 1, or 2) nucleotides in the sense strand being unmodified ribonucleotides. In some embodiments, one or more nucleotides of an RNAi agent is an unmodified ribonucleotide. Chemical structures for certain modified nucleotides are set forth in Table 6.1 herein.

### Modified Internucleoside Linkages

In some embodiments, one or more nucleotides of a DMPK RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, diphosphorothioates, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of a DMPK RNAi agent can contain 1, 2, 3, 4, 5, or 6 phosphorothioate linkages, an antisense strand of a DMPK RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of a DMPK RNAi agent can contain 1, 2, 3, or 4 phosphorothioate linkages, an antisense strand of a DMPK RNAi agent can contain 1, 2, 3, or 4 phosphorothioate linkages, or both the sense strand and the antisense strand independently can contain 1, 2, 3, or 4 phosphorothioate linkages.

In some embodiments, a DMPK RNAi agent sense strand contains at least two phosphorothioate internucleoside linkages. In some embodiments, the phosphorothioate internucleoside linkages are between the nucleotides at positions 1-3 from the 3′ end of the sense strand. In some embodiments, one phosphorothioate internucleoside linkage is at the 5′ end of the sense strand nucleotide sequence, and another phosphorothioate linkage is at the 3′ end of the sense strand nucleotide sequence. In some embodiments, two phosphorothioate internucleoside linkage are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, a DMPK RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1~4 from the 5′ end of the antisense strand, and a fourth phosphorothioate internucleoside linkage is located between positions 20-21 from the 5′ end of the antisense strand. In some embodiments, a DMPK RNAi agent contains at least three or four phosphorothioate internucleoside linkages in the antisense strand.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table 6.1). (See, e.g., F. Czauderna, Nucleic Acids Res., 2003, 31 (11), 2705-16; U.S. Pat. No. 5,998,203). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, one or more inverted abasic residues or inverted abasic sites are inserted between the PK/PD modulator and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb) s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. Chemical structures for inverted abasic deoxyribose residues are shown in Table 6.1 below.

### DMPK RNAi Agents

The DMPK RNAi agent embodiments disclosed herein were designed to target specific positions on a DMPK gene (i.e., specific positions on a DMPK gene transcript). As defined herein, an antisense strand sequence is designed to target a DMPK gene at a specific position on the gene when the 5′ terminal nucleobase of the antisense strand is aligned with a position that is 21 nucleotides downstream (towards the 3′ end) from the position on the gene when base pairing to the gene. For example, as illustrated in Tables 1 and 2 herein, an antisense strand sequence designed to target a DMPK gene at position 820 requires that when base pairing to the gene, the 5′ terminal nucleobase of the antisense strand is aligned with position 840 of the DMPK gene.

As provided herein, for the specific embodiments disclosed herein, a DMPK RNAi agent does not require that the nucleobase at position 1 (5′→3′) of the antisense strand be complementary to the gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 16 consecutive nucleotides. For example, for a DMPK RNAi agent disclosed herein that is designed to target position 820 of a DMPK gene, the 5′ terminal nucleobase of the antisense strand of the of the DMPK RNAi agent must be aligned with position 840 of the gene; however, the 5′ terminal nucleobase of the antisense strand may be, but is not required to be, complementary to position 840 of a DMPK gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 16 consecutive nucleotides. As shown by, among other things, the various examples disclosed herein, the specific site of binding of the gene by the antisense strand of the DMPK RNAi agent (e.g., whether the DMPK RNAi agent is designed to target a DMPK gene at position 820, at position 865, or at some other position) is important to the level of inhibition achieved by the DMPK RNAi agent.

In some embodiments, the DMPK RNAi agents disclosed herein target a DMPK gene at or near the positions of the DMPK sequence shown in Table 1. In some embodiments, the antisense strand of a DMPK RNAi agent disclosed herein includes a core stretch sequence that is fully, substantially, or at least partially complementary to a target DMPK 19-mer sequence disclosed in Table 1.

TABLE 1
DMPK 19-mer mRNA Target Sequences (taken from homo sapiens DM1 protein
kinase (DMPK), transcript variant 1, GenBank NM_001081563.2)

| | DMPK 19-mer | Corresponding | Targeted Gene |
|---|---|---|---|
| SEQ ID | Target Sequences | Positions of Sequence | Position (as |
| No. | (5′ → 3′) | on SEQ ID NO: 1 | referred to herein) |
| 2 | GCGAGGUAGCGGUAGUGAA | 822-840 | 820 |
| 3 | CCAUGAAGAUCAUGAACAA | 867-885 | 865 |
| 4 | AUGAAGAUCAUGAACAAGU | 869-887 | 867 |
| 5 | UGAAGAUCAUGAACAAGUG | 870-888 | 868 |
| 6 | AAGUGGGACAUGCUGAAGA | 884-902 | 882 |
| 7 | AGUGGGACAUGCUGAAGAG | 885-903 | 883 |
| 8 | GGGCGAGGUGUCGUGCUUC | 904-922 | 902 |
| 9 | GGGACGUGUUGGUGAAUGG | 933-951 | 931 |
| 10 | GCACUUCGCCUUCCAGGAU | 976-994 | 974 |
| 11 | UCCAGGAUGAGAACUACCU | 987-1005 | 985 |
| 12 | CAGGAUGAGAACUACCUGU | 989-1007 | 987 |
| 13 | GAUGAGAACUACCUGUACC | 992-1010 | 990 |
| 14 | AUGAGAACUACCUGUACCU | 993-1011 | 991 |
| 15 | ACCUGUACCUGGUCAUGGA | 1002-1020 | 1000 |
| 16 | UGGUCAUGGAGUAUUACGU | 1011-1029 | 1009 |
| 17 | CUGACACUGCUGAGCAAGU | 1043-1061 | 1041 |
| 18 | ACACUGCUGAGCAAGUUUG | 1046-1064 | 1044 |
| 19 | ACUGCUGAGCAAGUUUGGG | 1048-1066 | 1046 |
| 20 | ACAUCAAACCCGACAACAU | 1158-1176 | 1156 |
| 21 | AUCAAACCCGACAACAUCC | 1160-1178 | 1158 |
| 22 | AAACCCGACAACAUCCUGC | 1163-1181 | 1161 |
| 23 | CAGACGCCCUUCUACGCGG | 1397-1415 | 1395 |
| 24 | GCAAGAUCGUCCACUACAA | 1437-1455 | 1435 |
| 25 | AGAUCGUCCACUACAAGGA | 1440-1458 | 1438 |
| 26 | ACAAGGAGCACCUCUCUCU | 1452-1470 | 1450 |
| 27 | GACACAUCCCUUCUUCUUU | 1576-1594 | 1574 |
| 28 | CACAUCCCUUCUUCUUUGG | 1578-1596 | 1576 |
| 29 | CCUUUACACCGGAUUUCGA | 1632-1650 | 1630 |
| 30 | UUUACACCGGAUUUCGAAG | 1634-1652 | 1632 |
| 31 | CACCGGAUUUCGAAGGUGC | 1638-1656 | 1636 |
| 32 | CCGACACAUGCAACUUCGA | 1659-1677 | 1657 |
| 33 | GACACAUGCAACUUCGACU | 1661-1679 | 1659 |
| 34 | ACACAUGCAACUUCGACUU | 1662-1680 | 1660 |
| 35 | AUGCAACUUCGACUUGGUG | 1666-1684 | 1664 |
| 36 | AUGAAACAGCUGAAGUGGC | 1911-1929 | 1909 |
| 37 | CCUGAACCCUAGAACUGUC | 2460-2478 | 2458 |
| 38 | AACCCUAGAACUGUCUUCG | 2464-2482 | 2462 |
| 39 | ACCCUAGAACUGUCUUCGA | 2465-2483 | 2463 |
| 40 | CCCUAGAACUGUCUUCGAC | 2466-2484 | 2464 |
| 41 | CCUAGAACUGUCUUCGACU | 2467-2485 | 2465 |
| 42 | CUAGAACUGUCUUCGACUC | 2468-2486 | 2466 |
| 43 | GCUUACUCGGGAAAUUUGC | 2949-2967 | 2947 |
| 44 | CUUACUCGGGAAAUUUGCU | 2950-2968 | 2948 |
| 45 | UCGGGAAAUUUGCUUUUGC | 2955-2973 | 2953 |
| 46 | GGAAAUUUGCUUUUGCCAA | 2958-2976 | 2956 |
| 47 | AAAUUUGCUUUUGCCAAAC | 2960-2978 | 2958 |
| 48 | GGCGGUUUGGAUAUUUAUU | 3054-3072 | 3052 |
| 49 | GCGGUUUGGAUAUUUAUUG | 3055-3073 | 3053 |
| 50 | GGUUUGGAUAUUUAUUGAC | 3057-3075 | 3055 |
| 51 | CCCCAAUCCACGUUUUGGA | 3118-3136 | 3116 |
| 52 | CCCAAUCCACGUUUUGGAU | 3119-3137 | 3117 |
| 53 | CAAUCCACGUUUUGGAUGC | 3121-3139 | 3119 |
| 54 | UCCACGUUUUGGAUGCACU | 3124-3142 | 3122 |
| 55 | GUUUUGGAUGCACUGAGAC | 3129-3147 | 3127 |
| 56 | UUUUGGAUGCACUGAGACC | 3130-3148 | 3128 |
| 57 | CCGACAUUCCUCGGUAUUU | 3149-3167 | 3147 |
| 58 | AUUCCUCGGUAUUUAUUGU | 3154-3172 | 3152 |
| 59 | UCCUCGGUAUUUAUUGUCU | 3156-3174 | 3154 |
| 60 | CCUCGGUAUUUAUUGUCUG | 3157-3175 | 3155 |
| 61 | UCGGUAUUUAUUGUCUGUC | 3159-3177 | 3157 |
| 62 | CGGUAUUUAUUGUCUGUCC | 3160-3178 | 3158 |
| 63 | UUGUCUGUCCCCACCUAGG | 3169-3187 | 3167 |
| 64 | CCCUCGCGAAUAAAAGGCC | 3202-3220 | 3200 |

Homo sapiens DM1 protein kinase (DMPK), transcript variant 1, GenBank NM_001081563.2, gene transcript (3243 bases):

| | |
|---|---|
| 1 | gccacaagcc tccaccccag ctggtccccc acccaggctg cccagtttaa cattcctagt |
| 61 | cataggacct tgacttctga gaggcctgat tgtcatctgt aaataagggg taggactaaa |
| 121 | gcactcctcc tggaggactg agagatgggc tggaccggag cacttgagtc tgggatatgt |
| 181 | gaccatgcta cctttgtctc cctgtcctgt tccttccccc agccccaaat ccagggtttt |
| 241 | ccaaagtgtg gttcaagaac cacctgcatc tgaatctaga ggtactggat acaaccccac |
| 301 | gtctgggccg ttacccagga cattctacat gagaacgtgg gggtggggcc ctggctgcac |
| 361 | ctgaactgtc acctggagtc agggtggaag gtggaagaac tgggtcttat ttccttctcc |
| 421 | ccttgttctt tagggtctgt ccttctgcag actccgttac cccaccctaa ccatcctgca |
| 481 | cacccttgga gccctctggg ccaatgccct gtcccgcaaa gggcttctca ggcatctcac |
| 541 | ctctatggga gggcattttt ggcccccaga accttacacg gtgtttatgt ggggaagccc |
| 601 | ctgggaagca gacagtccta gggtgaagct gagaggcaga gagaagggga gacagacaga |
| 661 | gggtggggct ttcccccttg tctccagtgc cctttctggt gaccctcggt tcttttcccc |
| 721 | caccaccccc ccagcggagc ccatcgtggt gaggcttaag gaggtccgac tgcagaggga |
| 781 | cgacttcgag attctgaagg tgatcggacg cggggcgttc agcgaggtag cggtagtgaa |
| 841 | gatgaagcag acgggccagg tgtatgccat gaagatcatg aacaagtggg acatgctgaa |
| 901 | gaggggcgag gtgtcgtgct tccgtgagga gagggacgtg ttggtgaatg gggaccggcg |
| 961 | gtggatcacg cagctgcact tcgccttcca ggatgagaac tacctgtacc tggtcatgga |
| 1021 | gtattacgtg ggcggggacc tgctgacact gctgagcaag tttggggagc ggattccggc |
| 1081 | cgagatggcg cgcttctacc tggcggagat tgtcatggcc atagactcgg tgcaccggct |
| 1141 | tggctacgtg cacagggaca tcaaacccga caacatcctg ctggaccgct gtggccacat |
| 1201 | ccgcctggcc gacttcggct cttgcctcaa gctgcgggca gatggaacgg tgcggtcgct |
| 1261 | ggtggctgtg ggcaccccag actacctgtc ccccgagatc ctgcaggctg tgggcggtgg |
| 1321 | gcctgggaca ggcagctacg ggcccgagtg tgactggtgg gcgctgggtg tattcgccta |
| 1381 | tgaaatgttc tatgggcaga cgcccttcta cgcggattcc acggcggaga cctatggcaa |
| 1441 | gatcgtccac tacaaggagc acctctctct gccgctggtg gacgaagggg tccctgagga |
| 1501 | ggctcgagac ttcattcagc ggttgctgtg tcccccggag acacggctgg gccggggtgg |
| 1561 | agcaggcgac ttccggacac atcccttctt ctttggcctc gactgggatg gtctccggga |
| 1621 | cagcgtgccc ccctttacac cggatttcga aggtgccacc gacacatgca acttcgactt |
| 1681 | ggtggaggac gggctcactg ccatggtgag cgggggcggg gagacactgt cggacattcg |
| 1741 | ggaaggtgcg ccgctagggg tccacctgcc ttttgtgggc tactcctact cctgcatggc |
| 1801 | cctcagggac agtgaggtcc caggccccac acccatggaa ctggaggccg agcagctgct |
| 1861 | tgagccacac gtgcaagcgc ccagcctgga gccctcggtg tccccacagg atgaaacagc |
| 1921 | tgaagtggca gttccagcgg ctgtccctgc ggcagaggct gaggccgagg tgacgctgcg |
| 1981 | ggagctccag gaagccctgg aggaggaggt gctcacccgg cagagcctga gccgggagat |
| 2041 | ggaggccatc cgcacggaca accagaactt cgccagtcaa ctacgcgagg cagaggctcg |
| 2101 | gaaccgggac ctagaggcac acgtccggca gttgcaggag cggatggagt tgctgcaggc |
| 2161 | agagggagcc acagctgtca cgggggtccc cagtccccgg gccacggatc caccttccca |
| 2221 | tctagatggc cccccggccg tggctgtggg ccagtgcccg ctggtggggc caggccccat |
| 2281 | gcaccgccgc cacctgctgc tccctgccag ggtccctagg cctggcctat cggaggcgct |
| 2341 | ttccctgctc ctgttcgccg ttgttctgtc tcgtgccgcc gccctgggct gcattgggtt |
| 2401 | ggtggcccac gccggccaac tcaccgcagt ctggcgccgc ccaggagccg cccgcgctcc |
| 2461 | ctgaacccta gaactgtctt cgactccggg gccccgttgg aagactgagt gcccggggca |
| 2521 | cggcacagaa gccgcgccca ccgcctgcca gttcacaacc gctccgagcg tgggtctccg |
| 2581 | cccagctcca gtcctgtgat ccgggcccgc cccctagcgg ccggggaggg aggggccggg |
| 2641 | tccgcggccg gcgaacgggg ctcgaagggt ccttgtagcc gggaatgctg ctgctgctgc |
| 2701 | tgctgctgct gctgctgctg ctgctgctgc tgctgctgct gctgctgggg ggatcacaga |
| 2761 | ccatttcttt ctttcggcca ggctgaggcc ctgacgtgga tgggcaaact gcaggcctgg |
| 2821 | gaaggcagca agccgggccg tccgtgttcc atcctccacg cacccccacc tatcgttggt |
| 2881 | tcgcaaagtg caaagctttc ttgtgcatga cgccctgctc tggggagcgt ctggcgcgat |
| 2941 | ctctgcctgc ttactcggga aatttgcttt tgccaaaccc gctttttcgg ggatcccgcg |
| 3001 | cccccctcct cacttgcgct gctctcggag ccccagccgg ctccgcccgc ttcggcggtt |
| 3061 | tggatattta ttgacctcgt cctccgactc gctgacaggc tacaggaccc ccaacaaccc |
| 3121 | caatccacgt tttggatgca ctgagacccc gacattcctc ggtatttatt gtctgtcccc |
| 3181 | acctaggacc cccacccccg accctcgcga ataaaaggcc ctccatctgc ccaaagctct |
| 3241 | gga |

In some embodiments, a DMPK RNAi agent includes an antisense strand wherein position 19 of the antisense strand (5′→3′) is capable of forming a base pair with position 1 of a 19-mer target sequence disclosed in Table 1. In some embodiments, a DMPK RNAi agent includes an antisense strand wherein position 1 of the antisense strand (5′→3′) is capable of forming a base pair with position 19 of a 19-mer target sequence disclosed in Table 1.

In some embodiments, a DMPK RNAi agent includes an antisense strand wherein position 2 of the antisense strand (5′→3′) is capable of forming a base pair with position 18 of a 19-mer target sequence disclosed in Table 1. In some embodiments, a DMPK RNAi agent includes an antisense strand wherein positions 2 through 18 of the antisense strand (5′→3′) are capable of forming base pairs with each of the respective complementary bases located at positions 18 through 2 of the 19-mer target sequence disclosed in Table 1.

For the RNAi agents disclosed herein, the nucleotide at position 1 of the antisense strand (from 5′ end→3′ end) can be perfectly complementary to the DMPK gene, or can be non-complementary to the DMPK gene. In some embodiments, the nucleotide at position 1 of the antisense strand (from 5′ end→3′ end) is a U, A, or dT. In some embodiments, the nucleotide at position 1 of the antisense strand (from 5′ end→3′ end) forms an A: U or U: A base pair with the sense strand.

In some embodiments, a DMPK RNAi agent antisense strand comprises the sequence of nucleotides (from 5′ end→3′ end) 2-18 or 2-19 of any of the antisense strand sequences in Table 2, Table 3, or Table 5.4. In some embodiments, a DMPK RNAi sense strand comprises the sequence of nucleotides (from 5′ end→3′ end) 1-17, 1-18, or 2-18 of any of the sense strand sequences in Table 2 or Tables 4.1, 4.2, 4.3, 4.4, 4.5, 4.6, 5.4, or 5.5.

In some embodiments, a DMPK RNAi agent is comprised of (i) an antisense strand comprising the sequence of nucleotides (from 5′ end→3′ end) 2-18 or 2-19 of any of the antisense strand sequences in Table 2 or Table 3, and (ii) a sense strand comprising the sequence of nucleotides (from 5′ end→3′ end) 1-17 or 1-18 of any of the sense strand sequences in Table 2 or Tables 4.1, 4.2, 4.3, 4.4, 4.5, 4.6, 5.4, or 5.5.

In some embodiments, the DMPK RNAi agents include core 19-mer nucleotide sequences shown in the following Table 2.

TABLE 2
DMPK RNAi Agent Antisense Strand and Sense Strand Core Stretch Base Sequences
(N = any nucleobase)

| | Antisense Strand Base | | Sense Strand Base | Corresponding | |
|---|---|---|---|---|---|
| | Sequence | | Sequence | Positions of | |
| | (5′ → 3′) | | (5′ → 3′) | Identified | Targeted |
| SEQ ID | (Shown as an Unmodified | SEQ ID | (Shown as an Unmodified | Sequence on | Gene |
| NO:. | Nucleotide Sequence) | NO:. | Nucleotide Sequence) | SEQ ID NO: 1 | Position |
| 65 | UUCACUACCGCUACCUCGC | 349 | GCGAGGUAGCGGUAGUGAA | 822-840 | 820 |
| 66 | AUCACUACCGCUACCUCGC | 350 | GCGAGGUAGCGGUAGUGAU | 822-840 | 820 |
| 67 | NUCACUACCGCUACCUCGC | 351 | GCGAGGUAGCGGUAGUGAN | 822-840 | 820 |
| 68 | NUCACUACCGCUACCUCGN | 352 | NCGAGGUAGCGGUAGUGAN | 822-840 | 820 |
| 69 | UUGUUCAUGAUCUUCAUGG | 353 | CCAUGAAGAUCAUGAACAA | 867-885 | 865 |
| 70 | AUGUUCAUGAUCUUCAUGG | 354 | CCAUGAAGAUCAUGAACAU | 867-885 | 865 |
| 71 | NUGUUCAUGAUCUUCAUGG | 355 | CCAUGAAGAUCAUGAACAN | 867-885 | 865 |
| 72 | NUGUUCAUGAUCUUCAUGN | 356 | NCAUGAAGAUCAUGAACAN | 867-885 | 865 |
| 73 | ACUUGUUCAUGAUCUUCAU | 357 | AUGAAGAUCAUGAACAAGU | 869-887 | 867 |
| 74 | UCUUGUUCAUGAUCUUCAU | 358 | AUGAAGAUCAUGAACAAGA | 869-887 | 867 |
| 75 | NCUUGUUCAUGAUCUUCAU | 359 | AUGAAGAUCAUGAACAAGN | 869-887 | 867 |
| 76 | NCUUGUUCAUGAUCUUCAN | 360 | NUGAAGAUCAUGAACAAGN | 869-887 | 867 |
| 77 | CACUUGUUCAUGAUCUUCA | 361 | UGAAGAUCAUGAACAAGUG | 870-888 | 868 |
| 78 | UACUUGUUCAUGAUCUUCA | 362 | UGAAGAUCAUGAACAAGUA | 870-888 | 868 |
| 79 | AACUUGUUCAUGAUCUUCA | 363 | UGAAGAUCAUGAACAAGUU | 870-888 | 868 |
| 80 | NACUUGUUCAUGAUCUUCA | 364 | UGAAGAUCAUGAACAAGUN | 870-888 | 868 |
| 81 | NACUUGUUCAUGAUCUUCN | 365 | NGAAGAUCAUGAACAAGUN | 870-888 | 868 |
| 82 | UCUUCAGCAUGUCCCACUU | 366 | AAGUGGGACAUGCUGAAGA | 884-902 | 882 |
| 83 | ACUUCAGCAUGUCCCACUU | 367 | AAGUGGGACAUGCUGAAGU | 884-902 | 882 |
| 84 | NCUUCAGCAUGUCCCACUU | 368 | AAGUGGGACAUGCUGAAGN | 884-902 | 882 |
| 85 | NCUUCAGCAUGUCCCACUN | 369 | NAGUGGGACAUGCUGAAGN | 884-902 | 882 |
| 86 | CUCUUCAGCAUGUCCCACU | 370 | AGUGGGACAUGCUGAAGAG | 885-903 | 883 |
| 87 | UUCUUCAGCAUGUCCCACU | 371 | AGUGGGACAUGCUGAAGAG | 885-903 | 883 |
| 88 | AUCUUCAGCAUGUCCCACU | 372 | AGUGGGACAUGCUGAAGAG | 885-903 | 883 |
| 89 | NUCUUCAGCAUGUCCCACU | 373 | AGUGGGACAUGCUGAAGAG | 885-903 | 883 |
| 90 | NUCUUCAGCAUGUCCCACN | 374 | AGUGGGACAUGCUGAAGAG | 885-903 | 883 |
| 91 | GAAGCACGACACCUCGCCC | 375 | GGGCGAGGUGUCGUGCUUC | 904-922 | 902 |
| 92 | UAAGCACGACACCUCGCCC | 376 | GGGCGAGGUGUCGUGCUUA | 904-922 | 902 |
| 93 | AAAGCACGACACCUCGCCC | 377 | GGGCGAGGUGUCGUGCUUU | 904-922 | 902 |
| 94 | NAAGCACGACACCUCGCCC | 378 | GGGCGAGGUGUCGUGCUUN | 904-922 | 902 |
| 95 | NAAGCACGACACCUCGCCN | 379 | NGGCGAGGUGUCGUGCUUN | 904-922 | 902 |
| 96 | CCAUUCACCAACACGUCCC | 380 | GGGACGUGUUGGUGAAUGG | 933-951 | 931 |
| 97 | UCAUUCACCAACACGUCCC | 381 | GGGACGUGUUGGUGAAUGA | 933-951 | 931 |
| 98 | ACAUUCACCAACACGUCCC | 382 | GGGACGUGUUGGUGAAUGU | 933-951 | 931 |
| 99 | NCAUUCACCAACACGUCCC | 383 | GGGACGUGUUGGUGAAUGN | 933-951 | 931 |
| 100 | NCAUUCACCAACACGUCCN | 384 | NGGACGUGUUGGUGAAUGN | 933-951 | 931 |
| 101 | AUCCUGGAAGGCGAAGUGC | 385 | GCACUUCGCCUUCCAGGAU | 976-994 | 974 |
| 102 | UUCCUGGAAGGCGAAGUGC | 386 | GCACUUCGCCUUCCAGGAA | 976-994 | 974 |
| 103 | NUCCUGGAAGGCGAAGUGC | 387 | GCACUUCGCCUUCCAGGAN | 976-994 | 974 |
| 104 | NUCCUGGAAGGCGAAGUGN | 388 | NCACUUCGCCUUCCAGGAN | 976-994 | 974 |
| 105 | AGGUAGUUCUCAUCCUGGA | 389 | UCCAGGAUGAGAACUACCU | 987-1005 | 985 |
| 106 | UGGUAGUUCUCAUCCUGGA | 390 | UCCAGGAUGAGAACUACCA | 987-1005 | 985 |
| 107 | NGGUAGUUCUCAUCCUGGA | 391 | UCCAGGAUGAGAACUACCN | 987-1005 | 985 |
| 108 | NGGUAGUUCUCAUCCUGGN | 392 | NCCAGGAUGAGAACUACCN | 987-1005 | 985 |
| 109 | ACAGGUAGUUCUCAUCCUG | 393 | CAGGAUGAGAACUACCUGU | 989-1007 | 987 |
| 110 | UCAGGUAGUUCUCAUCCUG | 394 | CAGGAUGAGAACUACCUGA | 989-1007 | 987 |
| 111 | NCAGGUAGUUCUCAUCCUG | 395 | CAGGAUGAGAACUACCUGN | 989-1007 | 987 |
| 112 | NCAGGUAGUUCUCAUCCUN | 396 | NAGGAUGAGAACUACCUGN | 989-1007 | 987 |
| 113 | GGUACAGGUAGUUCUCAUC | 397 | GAUGAGAACUACCUGUACC | 992-1010 | 990 |
| 114 | UGUACAGGUAGUUCUCAUC | 398 | GAUGAGAACUACCUGUACA | 992-1010 | 990 |
| 115 | AGUACAGGUAGUUCUCAUC | 399 | GAUGAGAACUACCUGUACU | 992-1010 | 990 |
| 116 | NGUACAGGUAGUUCUCAUC | 400 | GAUGAGAACUACCUGUACN | 992-1010 | 990 |
| 117 | NGUACAGGUAGUUCUCAUN | 401 | NAUGAGAACUACCUGUACN | 992-1010 | 990 |
| 118 | AGGUACAGGUAGUUCUCAU | 402 | AUGAGAACUACCUGUACCU | 993-1011 | 991 |
| 119 | UGGUACAGGUAGUUCUCAU | 403 | AUGAGAACUACCUGUACCA | 993-1011 | 991 |
| 120 | NGGUACAGGUAGUUCUCAU | 404 | AUGAGAACUACCUGUACCN | 993-1011 | 991 |
| 121 | NGGUACAGGUAGUUCUCAN | 405 | NUGAGAACUACCUGUACCN | 993-1011 | 991 |
| 122 | UCCAUGACCAGGUACAGGU | 406 | ACCUGUACCUGGUCAUGGA | 1002-1020 | 1000 |
| 123 | ACCAUGACCAGGUACAGGU | 407 | ACCUGUACCUGGUCAUGGU | 1002-1020 | 1000 |
| 124 | NCCAUGACCAGGUACAGGU | 408 | ACCUGUACCUGGUCAUGGN | 1002-1020 | 1000 |
| 125 | NCCAUGACCAGGUACAGGN | 409 | NCCUGUACCUGGUCAUGGN | 1002-1020 | 1000 |
| 126 | ACGUAAUACUCCAUGACCA | 410 | UGGUCAUGGAGUAUUACGU | 1011-2019 | 1009 |
| 127 | UCGUAAUACUCCAUGACCA | 411 | UGGUCAUGGAGUAUUACGA | 1011-2019 | 1009 |
| 128 | NCGUAAUACUCCAUGACCA | 412 | UGGUCAUGGAGUAUUACGN | 1011-2019 | 1009 |
| 129 | NCGUAAUACUCCAUGACCN | 413 | NGGUCAUGGAGUAUUACGN | 1011-2019 | 1009 |
| 130 | ACUUGCUCAGCAGUGUCAG | 414 | CUGACACUGCUGAGCAAGU | 1041-1059 | 1041 |
| 131 | UCUUGCUCAGCAGUGUCAG | 415 | CUGACACUGCUGAGCAAGA | 1041-1059 | 1041 |
| 132 | NCUUGCUCAGCAGUGUCAG | 416 | CUGACACUGCUGAGCAAGN | 1041-1059 | 1041 |
| 133 | NCUUGCUCAGCAGUGUCAN | 417 | NUGACACUGCUGAGCAAGN | 1041-1059 | 1041 |
| 134 | CAAACUUGCUCAGCAGUGU | 418 | ACACUGCUGAGCAAGUUUG | 1046-1064 | 1044 |
| 135 | UAAACUUGCUCAGCAGUGU | 419 | ACACUGCUGAGCAAGUUUA | 1046-1064 | 1044 |
| 136 | AAAACUUGCUCAGCAGUGU | 420 | ACACUGCUGAGCAAGUUUU | 1046-1064 | 1044 |
| 137 | NAAACUUGCUCAGCAGUGU | 421 | ACACUGCUGAGCAAGUUUN | 1046-1064 | 1044 |
| 138 | NAAACUUGCUCAGCAGUGN | 422 | ACACUGCUGAGCAAGUUUN | 1046-1064 | 1044 |
| 139 | CCCAAACUUGCUCAGCAGU | 423 | ACUGCUGAGCAAGUUUGGG | 1048-1066 | 1046 |
| 140 | UCCAAACUUGCUCAGCAGU | 424 | ACUGCUGAGCAAGUUUGGA | 1048-1066 | 1046 |
| 141 | ACCAAACUUGCUCAGCAGU | 425 | ACUGCUGAGCAAGUUUGGG | 1048-1066 | 1046 |
| 142 | NCCAAACUUGCUCAGCAGU | 426 | ACUGCUGAGCAAGUUUGGN | 1048-1066 | 1046 |
| 143 | NCCAAACUUGCUCAGCAGN | 427 | ACUGCUGAGCAAGUUUGGN | 1048-1066 | 1046 |
| 144 | AUGUUGUCGGGUUUGAUGU | 428 | ACAUCAAACCCGACAACAU | 1158-1176 | 1156 |
| 145 | UUGUUGUCGGGUUUGAUGU | 429 | ACAUCAAACCCGACAACAA | 1158-1176 | 1156 |
| 146 | NUGUUGUCGGGUUUGAUGU | 430 | ACAUCAAACCCGACAACAN | 1158-1176 | 1156 |
| 147 | NUGUUGUCGGGUUUGAUGN | 431 | NCAUCAAACCCGACAACAN | 1158-1176 | 1156 |
| 148 | GGAUGUUGUCGGGUUUGAU | 432 | AUCAAACCCGACAACAUCC | 1160-1178 | 1158 |
| 149 | UGAUGUUGUCGGGUUUGAU | 433 | AUCAAACCCGACAACAUCA | 1160-1178 | 1158 |
| 150 | AGAUGUUGUCGGGUUUGAU | 434 | AUCAAACCCGACAACAUCU | 1160-1178 | 1158 |
| 151 | NGAUGUUGUCGGGUUUGAU | 435 | AUCAAACCCGACAACAUCN | 1160-1178 | 1158 |
| 152 | NGAUGUUGUCGGGUUUGAN | 436 | NUCAAACCCGACAACAUCN | 1160-1178 | 1158 |
| 153 | GCAGGAUGUUGUCGGGUUU | 437 | AAACCCGACAACAUCCUGC | 1163-1181 | 1161 |
| 154 | UCAGGAUGUUGUCGGGUUU | 438 | AAACCCGACAACAUCCUGA | 1163-1181 | 1161 |
| 155 | ACAGGAUGUUGUCGGGUUU | 439 | AAACCCGACAACAUCCUGU | 1163-1181 | 1161 |
| 156 | NCAGGAUGUUGUCGGGUUU | 440 | AAACCCGACAACAUCCUGN | 1163-1181 | 1161 |
| 157 | NCAGGAUGUUGUCGGGUUN | 441 | NAACCCGACAACAUCCUGN | 1163-1181 | 1161 |
| 158 | CCGCGUAGAAGGGCGUCUG | 442 | CAGACGCCCUUCUACGCGG | 1397-1415 | 1395 |
| 159 | UCGCGUAGAAGGGCGUCUG | 443 | CAGACGCCCUUCUACGCGA | 1397-1415 | 1395 |
| 160 | ACGCGUAGAAGGGCGUCUG | 444 | CAGACGCCCUUCUACGCGU | 1397-1415 | 1395 |
| 161 | NCGCGUAGAAGGGCGUCUG | 445 | CAGACGCCCUUCUACGCGN | 1397-1415 | 1395 |
| 162 | NCGCGUAGAAGGGCGUCUN | 446 | NAGACGCCCUUCUACGCGN | 1397-1415 | 1395 |
| 163 | UUGUAGUGGACGAUCUUGC | 447 | GCAAGAUCGUCCACUACAA | 1437-1455 | 1435 |
| 164 | AUGUAGUGGACGAUCUUGC | 448 | GCAAGAUCGUCCACUACAU | 1437-1455 | 1435 |
| 165 | NUGUAGUGGACGAUCUUGC | 449 | GCAAGAUCGUCCACUACAN | 1437-1455 | 1435 |
| 166 | NUGUAGUGGACGAUCUUGN | 450 | NCAAGAUCGUCCACUACAN | 1437-1455 | 1435 |
| 167 | UCCUUGUAGUGGACGAUCU | 451 | AGAUCGUCCACUACAAGGA | 1440-1458 | 1438 |
| 168 | ACCUUGUAGUGGACGAUCU | 452 | AGAUCGUCCACUACAAGGU | 1440-1458 | 1438 |
| 169 | NCCUUGUAGUGGACGAUCU | 453 | AGAUCGUCCACUACAAGGN | 1440-1458 | 1438 |
| 170 | NCCUUGUAGUGGACGAUCN | 454 | NGAUCGUCCACUACAAGGN | 1440-1458 | 1438 |
| 171 | AGAGAGAGGUGCUCCUUGU | 455 | ACAAGGAGCACCUCUCUCU | 1452-1470 | 1450 |
| 172 | UGAGAGAGGUGCUCCUUGU | 456 | ACAAGGAGCACCUCUCUCA | 1452-1470 | 1450 |
| 173 | NGAGAGAGGUGCUCCUUGU | 457 | ACAAGGAGCACCUCUCUCN | 1452-1470 | 1450 |
| 174 | NGAGAGAGGUGCUCCUUGN | 458 | NCAAGGAGCACCUCUCUCN | 1452-1470 | 1450 |
| 175 | AAAGAAGAAGGGAUGUGUC | 459 | GACACAUCCCUUCUUCUUU | 1576-1594 | 1574 |
| 176 | UAAGAAGAAGGGAUGUGUC | 460 | GACACAUCCCUUCUUCUUA | 1576-1594 | 1574 |
| 177 | NAAGAAGAAGGGAUGUGUC | 461 | GACACAUCCCUUCUUCUUN | 1576-1594 | 1574 |
| 178 | NAAGAAGAAGGGAUGUGUN | 462 | NACACAUCCCUUCUUCUUN | 1576-1594 | 1574 |
| 179 | CCAAAGAAGAAGGGAUGUG | 463 | CACAUCCCUUCUUCUUUGG | 1578-1596 | 1576 |
| 180 | UCAAAGAAGAAGGGAUGUG | 464 | CACAUCCCUUCUUCUUUGA | 1578-1596 | 1576 |
| 181 | ACAAAGAAGAAGGGAUGUG | 465 | CACAUCCCUUCUUCUUUGU | 1578-1596 | 1576 |
| 182 | NCAAAGAAGAAGGGAUGUG | 466 | CACAUCCCUUCUUCUUUGN | 1578-1596 | 1576 |
| 183 | NCAAAGAAGAAGGGAUGUN | 467 | NACAUCCCUUCUUCUUUGN | 1578-1596 | 1576 |
| 184 | UCGAAAUCCGGUGUAAAGG | 468 | CCUUUACACCGGAUUUCGA | 1632-1650 | 1630 |
| 185 | ACGAAAUCCGGUGUAAAGG | 469 | CCUUUACACCGGAUUUCGU | 1632-1650 | 1630 |
| 186 | NCGAAAUCCGGUGUAAAGG | 470 | CCUUUACACCGGAUUUCGN | 1632-1650 | 1630 |
| 187 | NCGAAAUCCGGUGUAAAGN | 471 | NCUUUACACCGGAUUUCGN | 1632-1650 | 1630 |
| 188 | CUUCGAAAUCCGGUGUAAA | 472 | UUUACACCGGAUUUCGAAG | 1634-1652 | 1632 |
| 189 | UUUCGAAAUCCGGUGUAAA | 473 | UUUACACCGGAUUUCGAAA | 1634-1652 | 1632 |
| 190 | AUUCGAAAUCCGGUGUAAA | 474 | UUUACACCGGAUUUCGAAU | 1634-1652 | 1632 |
| 191 | NUUCGAAAUCCGGUGUAAA | 475 | UUUACACCGGAUUUCGAAN | 1634-1652 | 1632 |
| 192 | NUUCGAAAUCCGGUGUAAN | 476 | NUUACACCGGAUUUCGAAN | 1634-1652 | 1632 |
| 193 | CCACCUUCGAAAUCCGGUG | 477 | CACCGGAUUUCGAAGGUGC | 1638-1656 | 1636 |
| 194 | UCACCUUCGAAAUCCGGUG | 478 | CACCGGAUUUCGAAGGUGA | 1638-1656 | 1636 |
| 195 | ACACCUUCGAAAUCCGGUG | 479 | CACCGGAUUUCGAAGGUGU | 1638-1656 | 1636 |
| 196 | NCACCUUCGAAAUCCGGUG | 480 | CACCGGAUUUCGAAGGUGN | 1638-1656 | 1636 |
| 197 | NCACCUUCGAAAUCCGGUN | 481 | NACCGGAUUUCGAAGGUGN | 1638-1656 | 1636 |
| 198 | UCGAAGUUGCAUGUGUCGG | 482 | CCGACACAUGCAACUUCGA | 1659-1677 | 1657 |
| 199 | ACGAAGUUGCAUGUGUCGG | 483 | CCGACACAUGCAACUUCGU | 1659-1677 | 1657 |
| 200 | NCGAAGUUGCAUGUGUCGG | 484 | CCGACACAUGCAACUUCGN | 1659-1677 | 1657 |
| 201 | NCGAAGUUGCAUGUGUCGN | 485 | NCGACACAUGCAACUUCGN | 1659-1677 | 1657 |
| 202 | AGUCGAAGUUGCAUGUGUC | 486 | GACACAUGCAACUUCGACU | 1661-1679 | 1659 |
| 203 | UGUCGAAGUUGCAUGUGUC | 487 | GACACAUGCAACUUCGACA | 1661-1679 | 1659 |
| 204 | NGUCGAAGUUGCAUGUGUC | 488 | GACACAUGCAACUUCGACN | 1661-1679 | 1659 |
| 205 | NGUCGAAGUUGCAUGUGUN | 489 | NACACAUGCAACUUCGACN | 1661-1679 | 1659 |
| 206 | AAGUCGAAGUUGCAUGUGU | 490 | ACACAUGCAACUUCGACUU | 1662-1680 | 1660 |
| 207 | UAGUCGAAGUUGCAUGUGU | 491 | ACACAUGCAACUUCGACUA | 1662-1680 | 1660 |
| 208 | NAGUCGAAGUUGCAUGUGU | 492 | ACACAUGCAACUUCGACUN | 1662-1680 | 1660 |
| 209 | NAGUCGAAGUUGCAUGUGN | 493 | NCACAUGCAACUUCGACUN | 1662-1680 | 1660 |
| 210 | CACCAAGUCGAAGUUGCAU | 494 | AUGCAACUUCGACUUGGUG | 1666-1684 | 1664 |
| 211 | UACCAAGUCGAAGUUGCAU | 495 | AUGCAACUUCGACUUGGUA | 1666-1684 | 1664 |
| 212 | AACCAAGUCGAAGUUGCAU | 496 | AUGCAACUUCGACUUGGUU | 1666-1684 | 1664 |
| 213 | NACCAAGUCGAAGUUGCAU | 497 | AUGCAACUUCGACUUGGUN | 1666-1684 | 1664 |
| 214 | NACCAAGUCGAAGUUGCAN | 498 | NUGCAACUUCGACUUGGUN | 1666-1684 | 1664 |
| 215 | GCCACUUCAGCUGUUUCAU | 499 | AUGAAACAGCUGAAGUGGC | 1911-1929 | 1909 |
| 216 | UCCACUUCAGCUGUUUCAU | 500 | AUGAAACAGCUGAAGUGGA | 1911-1929 | 1909 |
| 217 | ACCACUUCAGCUGUUUCAU | 501 | AUGAAACAGCUGAAGUGGU | 1911-1929 | 1909 |
| 218 | NCCACUUCAGCUGUUUCAU | 502 | AUGAAACAGCUGAAGUGGN | 1911-1929 | 1909 |
| 219 | NCCACUUCAGCUGUUUCAN | 503 | NUGAAACAGCUGAAGUGGN | 1911-1929 | 1909 |
| 220 | GACAGUUCUAGGGUUCAGG | 504 | CCUGAACCCUAGAACUGUC | 2460-2478 | 2458 |
| 221 | UACAGUUCUAGGGUUCAGG | 505 | CCUGAACCCUAGAACUGUA | 2460-2478 | 2458 |
| 222 | AACAGUUCUAGGGUUCAGG | 506 | CCUGAACCCUAGAACUGUU | 2460-2478 | 2458 |
| 223 | NACAGUUCUAGGGUUCAGG | 507 | CCUGAACCCUAGAACUGUN | 2460-2478 | 2458 |
| 224 | NACAGUUCUAGGGUUCAGN | 508 | NCUGAACCCUAGAACUGUN | 2460-2478 | 2458 |
| 225 | CGAAGACAGUUCUAGGGUU | 509 | AACCCUAGAACUGUCUUCG | 2464-2482 | 2462 |
| 226 | UGAAGACAGUUCUAGGGUU | 510 | AACCCUAGAACUGUCUUCA | 2464-2482 | 2462 |
| 227 | AGAAGACAGUUCUAGGGUU | 511 | AACCCUAGAACUGUCUUCU | 2464-2482 | 2462 |
| 228 | NGAAGACAGUUCUAGGGUU | 512 | AACCCUAGAACUGUCUUCN | 2464-2482 | 2462 |
| 229 | NGAAGACAGUUCUAGGGUN | 513 | NACCCUAGAACUGUCUUCN | 2464-2482 | 2462 |
| 230 | UCGAAGACAGUUCUAGGGU | 514 | ACCCUAGAACUGUCUUCGA | 2465-2483 | 2463 |
| 231 | ACGAAGACAGUUCUAGGGU | 515 | ACCCUAGAACUGUCUUCGU | 2465-2483 | 2463 |
| 232 | NCGAAGACAGUUCUAGGGU | 516 | ACCCUAGAACUGUCUUCGN | 2465-2483 | 2463 |
| 233 | NCGAAGACAGUUCUAGGGN | 517 | NCCCUAGAACUGUCUUCGN | 2465-2483 | 2463 |
| 234 | GUCGAAGACAGUUCUAGGG | 518 | CCCUAGAACUGUCUUCGAC | 2466-2484 | 2464 |
| 235 | UUCGAAGACAGUUCUAGGG | 519 | CCCUAGAACUGUCUUCGAA | 2466-2484 | 2464 |
| 236 | AUCGAAGACAGUUCUAGGG | 520 | CCCUAGAACUGUCUUCGAU | 2466-2484 | 2464 |
| 237 | NUCGAAGACAGUUCUAGGG | 521 | CCCUAGAACUGUCUUCGAN | 2466-2484 | 2464 |
| 238 | NUCGAAGACAGUUCUAGGN | 522 | NCCUAGAACUGUCUUCGAN | 2466-2484 | 2464 |
| 239 | AGUCGAAGACAGUUCUAGG | 523 | CCUAGAACUGUCUUCGACU | 2467-2485 | 2465 |
| 240 | UGUCGAAGACAGUUCUAGG | 524 | CCUAGAACUGUCUUCGACA | 2467-2485 | 2465 |
| 241 | NGUCGAAGACAGUUCUAGG | 525 | CCUAGAACUGUCUUCGACN | 2467-2485 | 2465 |
| 242 | NGUCGAAGACAGUUCUAGN | 526 | NCUAGAACUGUCUUCGACN | 2467-2485 | 2465 |
| 243 | GAGUCGAAGACAGUUCUAG | 527 | CUAGAACUGUCUUCGACUC | 2468-2486 | 2466 |
| 244 | UAGUCGAAGACAGUUCUAG | 528 | CUAGAACUGUCUUCGACUA | 2468-2486 | 2466 |
| 245 | AAGUCGAAGACAGUUCUAG | 529 | CUAGAACUGUCUUCGACUU | 2468-2486 | 2466 |
| 246 | NAGUCGAAGACAGUUCUAG | 530 | CUAGAACUGUCUUCGACUN | 2468-2486 | 2466 |
| 247 | NAGUCGAAGACAGUUCUAN | 531 | NUAGAACUGUCUUCGACUN | 2468-2486 | 2466 |
| 248 | GCAAAUUUCCCGAGUAAGC | 532 | GCUUACUCGGGAAAUUUGC | 2949-2967 | 2947 |
| 249 | UCAAAUUUCCCGAGUAAGC | 533 | GCUUACUCGGGAAAUUUGA | 2949-2967 | 2947 |
| 250 | ACAAAUUUCCCGAGUAAGC | 534 | GCUUACUCGGGAAAUUUGU | 2949-2967 | 2947 |
| 251 | NCAAAUUUCCCGAGUAAGC | 535 | GCUUACUCGGGAAAUUUGN | 2949-2967 | 2947 |
| 252 | NCAAAUUUCCCGAGUAAGN | 536 | NCUUACUCGGGAAAUUUGN | 2949-2967 | 2947 |
| 253 | AGCAAAUUUCCCGAGUAAG | 537 | CUUACUCGGGAAAUUUGCU | 2950-2968 | 2948 |
| 254 | UGCAAAUUUCCCGAGUAAG | 538 | CUUACUCGGGAAAUUUGCA | 2950-2968 | 2948 |
| 255 | NGCAAAUUUCCCGAGUAAG | 539 | CUUACUCGGGAAAUUUGCN | 2950-2968 | 2948 |
| 256 | NGCAAAUUUCCCGAGUAAN | 540 | NUUACUCGGGAAAUUUGCN | 2950-2968 | 2948 |
| 257 | GCAAAAGCAAAUUUCCCGA | 541 | UCGGGAAAUUUGCUUUUGC | 2955-2973 | 2953 |
| 258 | UCAAAAGCAAAUUUCCCGA | 542 | UCGGGAAAUUUGCUUUUGA | 2955-2973 | 2953 |
| 259 | ACAAAAGCAAAUUUCCCGA | 543 | UCGGGAAAUUUGCUUUUGU | 2955-2973 | 2953 |
| 260 | NCAAAAGCAAAUUUCCCGA | 544 | UCGGGAAAUUUGCUUUUGN | 2955-2973 | 2953 |
| 261 | NCAAAAGCAAAUUUCCCGN | 545 | NCGGGAAAUUUGCUUUUGN | 2955-2973 | 2953 |
| 262 | UUGGCAAAAGCAAAUUUCC | 546 | GGAAAUUUGCUUUUGCCAA | 2958-2976 | 2956 |
| 263 | AUGGCAAAAGCAAAUUUCC | 547 | GGAAAUUUGCUUUUGCCAU | 2958-2976 | 2956 |
| 264 | NUGGCAAAAGCAAAUUUCC | 548 | GGAAAUUUGCUUUUGCCAN | 2958-2976 | 2956 |
| 265 | NUGGCAAAAGCAAAUUUCN | 549 | NGAAAUUUGCUUUUGCCAN | 2958-2976 | 2956 |
| 266 | GUUUGGCAAAAGCAAAUUU | 550 | AAAUUUGCUUUUGCCAAAC | 2960-2978 | 2958 |
| 267 | UUUUGGCAAAAGCAAAUUU | 551 | AAAUUUGCUUUUGCCAAAA | 2960-2978 | 2958 |
| 268 | AUUUGGCAAAAGCAAAUUU | 552 | AAAUUUGCUUUUGCCAAAU | 2960-2978 | 2958 |
| 269 | NUUUGGCAAAAGCAAAUUU | 553 | AAAUUUGCUUUUGCCAAAN | 2960-2978 | 2958 |
| 270 | NUUUGGCAAAAGCAAAUUN | 554 | NAAUUUGCUUUUGCCAAAN | 2960-2978 | 2958 |
| 271 | AAUAAAUAUCCAAACCGCC | 555 | GGCGGUUUGGAUAUUUAUU | 3054-3072 | 3052 |
| 272 | UAUAAAUAUCCAAACCGCC | 556 | GGCGGUUUGGAUAUUUAUA | 3054-3072 | 3052 |
| 273 | NAUAAAUAUCCAAACCGCC | 557 | GGCGGUUUGGAUAUUUAUN | 3054-3072 | 3052 |
| 274 | NAUAAAUAUCCAAACCGCN | 558 | NGCGGUUUGGAUAUUUAUN | 3054-3072 | 3052 |
| 275 | CAAUAAAUAUCCAAACCGC | 559 | GCGGUUUGGAUAUUUAUUG | 3055-3073 | 3053 |
| 276 | UAAUAAAUAUCCAAACCGC | 560 | GCGGUUUGGAUAUUUAUUA | 3055-3073 | 3053 |
| 277 | AAAUAAAUAUCCAAACCGC | 561 | GCGGUUUGGAUAUUUAUUU | 3055-3073 | 3053 |
| 278 | NAAUAAAUAUCCAAACCGC | 562 | GCGGUUUGGAUAUUUAUUN | 3055-3073 | 3053 |
| 279 | NAAUAAAUAUCCAAACCGN | 563 | NCGGUUUGGAUAUUUAUUN | 3055-3073 | 3053 |
| 280 | GUCAAUAAAUAUCCAAACC | 564 | GGUUUGGAUAUUUAUUGAC | 3057-3075 | 3055 |
| 281 | UUCAAUAAAUAUCCAAACC | 565 | GGUUUGGAUAUUUAUUGAA | 3057-3075 | 3055 |
| 282 | AUCAAUAAAUAUCCAAACC | 566 | GGUUUGGAUAUUUAUUGAU | 3057-3075 | 3055 |
| 283 | NUCAAUAAAUAUCCAAACC | 567 | GGUUUGGAUAUUUAUUGAN | 3057-3075 | 3055 |
| 284 | NUCAAUAAAUAUCCAAACN | 568 | NGUUUGGAUAUUUAUUGAN | 3057-3075 | 3055 |
| 285 | UCCAAAACGUGGAUUGGGG | 569 | CCCCAAUCCACGUUUUGGA | 3118-3136 | 3116 |
| 286 | ACCAAAACGUGGAUUGGGG | 570 | CCCCAAUCCACGUUUUGGU | 3118-3136 | 3116 |
| 287 | NCCAAAACGUGGAUUGGGG | 571 | CCCCAAUCCACGUUUUGGN | 3118-3136 | 3116 |
| 288 | NCCAAAACGUGGAUUGGGN | 572 | NCCCAAUCCACGUUUUGGN | 3118-3136 | 3116 |
| 289 | AUCCAAAACGUGGAUUGGG | 573 | CCCAAUCCACGUUUUGGAU | 3119-3137 | 3117 |
| 290 | UUCCAAAACGUGGAUUGGG | 574 | CCCAAUCCACGUUUUGGAA | 3119-3137 | 3117 |
| 291 | NUCCAAAACGUGGAUUGGG | 575 | CCCAAUCCACGUUUUGGAN | 3119-3137 | 3117 |
| 292 | NUCCAAAACGUGGAUUGGN | 576 | NCCAAUCCACGUUUUGGAN | 3119-3137 | 3117 |
| 293 | GCAUCCAAAACGUGGAUUG | 577 | CAAUCCACGUUUUGGAUGC | 3121-3139 | 3119 |
| 294 | UCAUCCAAAACGUGGAUUG | 578 | CAAUCCACGUUUUGGAUGA | 3121-3139 | 3119 |
| 295 | ACAUCCAAAACGUGGAUUG | 579 | CAAUCCACGUUUUGGAUGU | 3121-3139 | 3119 |
| 296 | NCAUCCAAAACGUGGAUUG | 580 | CAAUCCACGUUUUGGAUGN | 3121-3139 | 3119 |
| 297 | NCAUCCAAAACGUGGAUUN | 581 | NAAUCCACGUUUUGGAUGN | 3121-3139 | 3119 |
| 298 | AGUGCAUCCAAAACGUGGA | 582 | UCCACGUUUUGGAUGCACU | 3124-3142 | 3122 |
| 299 | UGUGCAUCCAAAACGUGGA | 583 | UCCACGUUUUGGAUGCACA | 3124-3142 | 3122 |
| 300 | NGUGCAUCCAAAACGUGGA | 584 | UCCACGUUUUGGAUGCACN | 3124-3142 | 3122 |
| 301 | NGUGCAUCCAAAACGUGGN | 585 | NCCACGUUUUGGAUGCACN | 3124-3142 | 3122 |
| 302 | GUCUCAGUGCAUCCAAAAC | 586 | GUUUUGGAUGCACUGAGAC | 3129-3147 | 3127 |
| 303 | UUCUCAGUGCAUCCAAAAC | 587 | GUUUUGGAUGCACUGAGAA | 3129-3147 | 3127 |
| 304 | AUCUCAGUGCAUCCAAAAC | 588 | GUUUUGGAUGCACUGAGAU | 3129-3147 | 3127 |
| 305 | NUCUCAGUGCAUCCAAAAC | 589 | GUUUUGGAUGCACUGAGAN | 3129-3147 | 3127 |
| 306 | NUCUCAGUGCAUCCAAAAN | 590 | NUUUUGGAUGCACUGAGAN | 3129-3147 | 3127 |
| 307 | GGUCUCAGUGCAUCCAAAA | 591 | UUUUGGAUGCACUGAGACC | 3130-3148 | 3128 |
| 308 | UGUCUCAGUGCAUCCAAAA | 592 | UUUUGGAUGCACUGAGACA | 3130-3148 | 3128 |
| 309 | AGUCUCAGUGCAUCCAAAA | 593 | UUUUGGAUGCACUGAGACU | 3130-3148 | 3128 |
| 310 | NGUCUCAGUGCAUCCAAAA | 594 | UUUUGGAUGCACUGAGACN | 3130-3148 | 3128 |
| 311 | NGUCUCAGUGCAUCCAAAA | 595 | NUUUGGAUGCACUGAGACN | 3130-3148 | 3128 |
| 312 | UAAUACCGAGGAAUGUCGG | 596 | CCGACAUUCCUCGGUAUUA | 3149-3167 | 3147 |
| 313 | AAAUACCGAGGAAUGUCGG | 597 | CCGACAUUCCUCGGUAUUU | 3149-3167 | 3147 |
| 314 | NAAUACCGAGGAAUGUCGG | 598 | CCGACAUUCCUCGGUAUUN | 3149-3167 | 3147 |
| 315 | NAAUACCGAGGAAUGUCGN | 599 | NCGACAUUCCUCGGUAUUN | 3149-3167 | 3147 |
| 316 | ACAAUAAAUACCGAGGAAU | 600 | AUUCCUCGGUAUUUAUUGU | 3154-3172 | 3152 |
| 317 | UCAAUAAAUACCGAGGAAU | 601 | AUUCCUCGGUAUUUAUUGA | 3154-3172 | 3152 |
| 318 | NCAAUAAAUACCGAGGAAU | 602 | AUUCCUCGGUAUUUAUUGN | 3154-3172 | 3152 |
| 319 | NCAAUAAAUACCGAGGAAN | 603 | NUUCCUCGGUAUUUAUUGN | 3154-3172 | 3152 |
| 320 | AGACAAUAAAUACCGAGGA | 604 | UCCUCGGUAUUUAUUGUCU | 3156-3174 | 3154 |
| 321 | UGACAAUAAAUACCGAGGA | 605 | UCCUCGGUAUUUAUUGUCA | 3156-3174 | 3154 |
| 322 | NGACAAUAAAUACCGAGGA | 606 | UCCUCGGUAUUUAUUGUCN | 3156-3174 | 3154 |
| 323 | NGACAAUAAAUACCGAGGN | 607 | NCCUCGGUAUUUAUUGUCN | 3156-3174 | 3154 |
| 324 | CAGACAAUAAAUACCGAGG | 608 | CCUCGGUAUUUAUUGUCUG | 3157-3175 | 3155 |
| 325 | UAGACAAUAAAUACCGAGG | 609 | CCUCGGUAUUUAUUGUCUA | 3157-3175 | 3155 |
| 326 | AAGACAAUAAAUACCGAGG | 610 | CCUCGGUAUUUAUUGUCUU | 3157-3175 | 3155 |
| 327 | NAGACAAUAAAUACCGAGG | 611 | CCUCGGUAUUUAUUGUCUN | 3157-3175 | 3155 |
| 328 | NAGACAAUAAAUACCGAGN | 612 | NCUCGGUAUUUAUUGUCUN | 3157-3175 | 3155 |
| 329 | GACAGACAAUAAAUACCGA | 613 | UCGGUAUUUAUUGUCUGUC | 3159-3177 | 3157 |
| 330 | UACAGACAAUAAAUACCGA | 614 | UCGGUAUUUAUUGUCUGUA | 3159-3177 | 3157 |
| 331 | AACAGACAAUAAAUACCGA | 615 | UCGGUAUUUAUUGUCUGUU | 3159-3177 | 3157 |
| 332 | NACAGACAAUAAAUACCGA | 616 | UCGGUAUUUAUUGUCUGUN | 3159-3177 | 3157 |
| 333 | NACAGACAAUAAAUACCGN | 617 | NCGGUAUUUAUUGUCUGUN | 3159-3177 | 3157 |
| 334 | GGACAGACAAUAAAUACCG | 618 | CGGUAUUUAUUGUCUGUCC | 3160-3178 | 3158 |
| 335 | UGACAGACAAUAAAUACCG | 619 | CGGUAUUUAUUGUCUGUCA | 3160-3178 | 3158 |
| 336 | AGACAGACAAUAAAUACCG | 620 | CGGUAUUUAUUGUCUGUCU | 3160-3178 | 3158 |
| 337 | NGACAGACAAUAAAUACCG | 621 | CGGUAUUUAUUGUCUGUCN | 3160-3178 | 3158 |
| 338 | NGACAGACAAUAAAUACCN | 622 | NGGUAUUUAUUGUCUGUCN | 3160-3178 | 3158 |
| 339 | CCUAGGUGGGGACAGACAA | 623 | UUGUCUGUCCCCACCUAGG | 3169-3187 | 3167 |
| 340 | UCUAGGUGGGGACAGACAA | 624 | UUGUCUGUCCCCACCUAGA | 3169-3187 | 3167 |
| 341 | ACUAGGUGGGGACAGACAA | 625 | UUGUCUGUCCCCACCUAGU | 3169-3187 | 3167 |
| 342 | NCUAGGUGGGGACAGACAA | 626 | UUGUCUGUCCCCACCUAGN | 3169-3187 | 3167 |
| 343 | NCUAGGUGGGGACAGACAN | 627 | NUGUCUGUCCCCACCUAGN | 3169-3187 | 3167 |
| 344 | GGCCUUUUAUUCGCGAGGG | 628 | CCCUCGCGAAUAAAAGGCC | 3202-3220 | 3200 |
| 345 | UGCCUUUUAUUCGCGAGGG | 629 | CCCUCGCGAAUAAAAGGCA | 3202-3220 | 3200 |
| 346 | AGCCUUUUAUUCGCGAGGG | 630 | CCCUCGCGAAUAAAAGGCU | 3202-3220 | 3200 |
| 347 | NGCCUUUUAUUCGCGAGGG | 631 | CCCUCGCGAAUAAAAGGCN | 3202-3220 | 3200 |
| 348 | NGCCUUUUAUUCGCGAGGN | 632 | NCCUCGCGAAUAAAAGGCN | 3202-3220 | 3200 |

The DMPK RNAi agent sense strands and antisense strands that comprise or consist of the nucleotide sequences in Table 2 can be modified nucleotides or unmodified nucleotides. In some embodiments, the DMPK RNAi agents having the sense and antisense strand sequences that comprise or consist of any of the nucleotide sequences in Table 2 are all or substantially all modified nucleotides.

In some embodiments, the antisense strand of a DMPK RNAi agent disclosed herein differs by 0, 1, 2, or 3 nucleotides from any of the antisense strand sequences in Table 2. In some embodiments, the sense strand of a DMPK RNAi agent disclosed herein differs by 0, 1, 2, or 3 nucleotides from any of the sense strand sequences in Table 2.

As used herein, each N listed in a sequence disclosed in Table 2 may be independently selected from any and all nucleobases (including those found on both modified and unmodified nucleotides). In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments, an N nucleotide listed in a sequence disclosed in Table 2 has a nucleobase that is different from the N nucleotide at the corresponding position on the other strand.

Certain modified DMPK RNAi agent sense and antisense strands are provided in Table 3 and Tables 4.1, 4.2, 4.3, 4.4, 4.5, 4.6, 5.4, and 5.5. Modified DMPK RNAi agent antisense strands, as well as their underlying unmodified nucleobase sequences, are provided in Table 3. Modified DMPK RNAi agent sense strands, as well as their underlying unmodified nucleobase sequences, are provided in Tables 4.1, 4.2, 4.3, 4.4, 4.5, 4.6, 5.4, and 5.5. In forming DMPK RNAi agents, each of the nucleotides in each of the underlying base sequences listed in Tables 3 and Tables 4.1, 4.2, 4.3, 4.4, 4.5, 4.6, 5.4, and 5.5, as well as in Table 2, above, can be a modified nucleotide.

The DMPK RNAi agents described herein are formed by annealing an antisense strand with a sense strand. A sense strand containing a sequence listed in Table 2 or Tables 4.1, 4.2, 4.3, 4.4, 4.5, 4.6, 5.4, or 5.5 can be hybridized to any antisense strand containing a sequence listed in Table 2, Table 3, or Table 5.4 provided the two sequences have a region of at least 85% complementarity over a contiguous 16, 17, 18, 19, 20, or 21 nucleotide sequence.

In some embodiments, a DMPK RNAi agent antisense strand comprises a nucleotide sequence of any of the sequences in Table 2, Table 3, or Table 5.4.

In some embodiments, a DMPK RNAi agent comprises or consists of a duplex having the nucleobase sequences of the sense strand and the antisense strand of any of the sequences in Table 2, Table 3, or Tables 4.1, 4.2, 4.3, 4.4, 4.5, 4.6, 5.4, or 5.5.

Examples of antisense strands containing modified nucleotides are provided in Table 3. Examples of sense strands containing modified nucleotides are provided in Tables 4.1, 4.2, 4.3, 4.4, 4.5, 4.6, 5.4, and 5.5.

As used in Tables 3 and Tables 4.1, 4.2, 4.3, 4.4, 4.5, 4.6, 5.4, and 5.5 the following notations are used to indicate modified nucleotides, targeting groups, and linking groups:

| | |
|---|---|
| A = | adenosine-3′-phosphate |
| C = | cytidine-3′-phosphate |
| G = | guanosine-3′-phosphate |
| U = | uridine-3′-phosphate |
| I = | inosine-3′-phosphate |
| a = | 2′-O-methyladenosine-3′-phosphate |
| as = | 2′-O-methyladenosine-3′-phosphorothioate |
| c = | 2′-O-methylcytidine-3′-phosphate |
| cs = | 2′-O-methylcytidine-3′-phosphorothioate |
| g = | 2′-O-methylguanosine-3′-phosphate |
| gs = | 2′-O-methylguanosine-3′-phosphorothioate |
| i = | 2′-O-methylinosine-3′-phosphate |
| is = | 2′-O-methylinosine-3′-phosphorothioate |
| t = | 2′-O-methyl-5-methyluridine-3′-phosphate |
| ts = | 2′-O-methyl-5-methyluridine-3′-phosphorothioate |
| u = | 2′-O-methyluridine-3′-phosphate |
| us = | 2′-O-methyluridine-3′-phosphorothioate |
| Af = | 2′-fluoroadenosine-3′-phosphate |
| Afs = | 2′-fluoroadenosine-3′-phosporothioate |
| Cf = | 2′-fluorocytidine-3′-phosphate |
| Cfs = | 2′-fluorocytidine-3′-phosphorothioate |
| Gf = | 2′-fluoroguanosine-3′-phosphate |
| Gfs = | 2′-fluoroguanosine-3′-phosphorothioate |
| Tf = | 2′-fluoro-5′-methyluridine-3′-phosphate |
| Tfs = | 2′-fluoro-5′-methyluridine-3′-phosphorothioate |
| Uf = | 2′-fluorouridine-3′-phosphate |
| Ufs = | 2′-fluorouridine-3′-phosphorothioate |
| dT = | 2′-deoxythymidine-3′-phosphate |
| A_{UNA} = | 2′,3′-seco-adenosine-3′-phosphate |
| A_{UNA}s = | 2′,3′-seco-adenosine-3′-phosphorothioate |
| C_{UNA} = | 2′,3′-seco-cytidine-3′-phosphate |
| C_{UNA}s = | 2′,3′-seco-cytidine-3′-phosphorothioate |
| G_{UNA} = | 2′,3′-seco-guanosine-3′-phosphate |
| G_{UNA}s = | 2′,3′-seco-guanosine-3′-phosphorothioate |
| U_{UNA} = | 2′,3′-seco-uridine-3′-phosphate |
| U_{UNA}s = | 2′,3′-seco-uridine-3′-phosphorothioate |
| a_2N = | 2′-O-methyl-2-aminoadenosine-3′-phosphate, see Table 6.1 |
| a_2Ns = | 2′-O-methyl-2-aminoadenosine-3′-phosphorothioate, see Table 6.1 |
| (invAb) = | inverted abasic deoxyribonucleotide-5′- |
| | phosphate, see Table 6.1 |
| (invAb)s = | inverted abasic deoxyribonucleotide-5′- |
| | phosphorothioate, see Table 6.1 |
| s = | phosphorothioate linkage |
| ss = | phosphorodithioate linkage |
| p = | terminal phosphate (as synthesized) |
| vpdN = | vinyl phosphonate deoxyribonucleotide |
| cPrpa = | 5′-cyclopropyl phosphonate-2′-O-methyladenosine-3′-phosphate |
| | (see Table 6.1) |
| cPrpas = | 5′-cyclopropyl phosphonate-2′-O-methyladenosine-3′- |
| | phosphorothioate (see Table 6.1) |
| cPrpu = | 5′-cyclopropyl phosphonate-2′-O-methyluridine-3′-phosphate (see |
| | Table 6.1) |
| cPrpus = | 5′-cyclopropyl phosphonate-2′-O-methyluridine-3′- |
| | phosphorothioate (see Table 6.1) |
| aAlk = | 2′-O-propargyladenosine-3′-phosphate, see Table 6.1 |
| aAlks = | 2′-O-propargyladenosine-3′-phosphorothioate, see Table 6.1 |
| cAlk = | 2′-O-propargylcytidine-3′-phosphate, see Table 6.1 |
| cAlks = | 2′-O-propargylcytidine-3′-phosphorothioate, see Table 6.1 |
| gAlk = | 2′-O-propargylguanosine-3′-phosphate, see Table 6.1 |
| gAlks = | 2′-O-propargylguanosine-3′-phosphorothioate, see Table 6.1 |
| tAlk = | 2′-O-propargyl-5-methyluridine-3′-phosphate, see Table 6.1 |
| tAlks = | 2′-O-propargyl-5-methyluridine-3′-phosphorothioate, see Table 6.1 |
| uAlk = | 2′-O-propargyluridine-3′-phosphate, see Table 6.1 |
| uAlks = | 2′-O-propargyluridine-3′-phosphorothioate, see Table 6.1 |
| (Alk-SS-C6) = | see Table 6.1 |
| (C6-SS-Alk) = | see Table 6.1 |
| (C6-SS-C6) = | see Table 6.1 |
| (6-SS-6) = | see Table 6.1 |
| (C6-SS-Alk-Me) = | see Table 6.1 |
| (NH2-C6) = | see Table 6.1 |
| (Alk-cyHex) = | see Table 6.1 |
| (Alk-cyHex)s = | see Table 6.1 |
| avb6-pep1 = | αvβ6 Peptide 1, see Table 6.3 |

As the person of ordinary skill in the art would readily understand, unless otherwise indicated by the sequence (such as, for example, by a phosphorothioate linkage “s” or by a phosphorodithioate linkage “ss”), when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the DMPK RNAi agents and compositions of DMPK RNAi agents disclosed herein.

Certain examples of targeting groups and linking groups used with the DMPK RNAi agents disclosed herein are included in the chemical structures provided below in Table 6.1, 6.2 and 6.3. Each sense strand and/or antisense strand can have any targeting groups or linking groups listed herein, as well as other targeting or linking groups, conjugated to the 5′ and/or 3′ end of the sequence.

TABLE 3
DMPK RNAi Agent Antisense Strand Sequences

| | | | Underlying Base | |
|---|---|---|---|---|
| | | | Sequence (5′ → 3′) | |
| Antisense | | Seq ID | (Shown as an Unmodified | Seq |
| Strand ID | Modified Antisense Strand (5′ → 3′) | No. | Nucleotide Sequence) | ID No. |
| AM12527-AS | usCfscsAfcUfuCfaGfcUfgUfuUfcAfuCfsc | 633 | UCCACUUCAGCUGUUUCAUCC | 1443 |
| AM12529-AS | usAfscsAfgUfuCfuAfgGfgUfuCfaGfgGfsa | 634 | UACAGUUCUAGGGUUCAGGGA | 1444 |
| AM12531-AS | usCfsgsAfaGfaCfaGfuUfcUfaGfgGfuUfsc | 635 | UCGAAGACAGUUCUAGGGUUC | 1445 |
| AM12533-AS | usUfscsGfaAfgAfcAfgUfuCfuAfgGfgUfsc | 636 | UUCGAAGACAGUUCUAGGGUC | 1446 |
| AM12535-AS | asGfsusCfgAfaGfaCfaGfuUfcUfaGfgGfsu | 637 | AGUCGAAGACAGUUCUAGGGU | 1447 |
| AM12537-AS | usAfsgsUfcGfaAfgAfcAfgUfuCfuAfgGfsg | 638 | UAGUCGAAGACAGUUCUAGGG | 1448 |
| AM12539-AS | asGfscsAfaAfuUfuCfcCfgAfgUfaAfgCfsa | 639 | AGCAAAUUUCCCGAGUAAGCA | 1449 |
| AM12541-AS | usUfsgsGfcAfaAfaGfcAfaAfuUfuCfcCfsg | 640 | UUGGCAAAAGCAAAUUUCCCG | 1450 |
| AM12543-AS | asUfscsCfaAfaAfcGfuGfgAfuUfgGfgGfsu | 641 | AUCCAAAACGUGGAUUGGGGU | 1451 |
| AM12545-AS | usCfsasUfcCfaAfaAfcGfuGfgAfuUfgGfsg | 642 | UCAUCCAAAACGUGGAUUGGG | 1452 |
| AM13806-AS | usCfsasUfuCfaCfcAfaCfaCfgUfcCfcUfsc | 643 | UCAUUCACCAACACGUCCCUC | 1453 |
| AM13808-AS | asGfsgsUfaCfaGfgUfaGfuUfcUfcAfuCfsc | 644 | AGGUACAGGUAGUUCUCAUCC | 1454 |
| AM13810-AS | asCfsgsUfaAfuAfcUfcCfaUfgAfcCfaGfsg | 645 | ACGUAAUACUCCAUGACCAGG | 1455 |
| AM13812-AS | asUfsgsUfuGfuCfgGfgUfuUfgAfuGfuCfsc | 646 | AUGUUGUCGGGUUUGAUGUCC | 1456 |
| AM13815-AS | usUfsgsUfaGfuGfgAfcGfaUfcUfuGfcCfsa | 647 | UUGUAGUGGACGAUCUUGCCA | 1457 |
| AM13818-AS | usCfsgsAfaAfuCfcGfgUfgUfaAfaGfgGfsg | 648 | UCGAAAUCCGGUGUAAAGGGG | 1458 |
| AM13820-AS | asAfsgsUfcGfaAfgUfuGfcAfuGfuGfuCfsg | 649 | AAGUCGAAGUUGCAUGUGUCG | 1459 |
| AM13822-AS | usGfsasAfgAfcAfgUfuCfuAfgGfgUfuCfsc | 650 | UGAAGACAGUUCUAGGGUUCC | 1460 |
| AM13825-AS | usCfsasAfaUfuUfcCfcGfaGfuAfaGfcAfsg | 651 | UCAAAUUUCCCGAGUAAGCAG | 1461 |
| AM13827-AS | usUfscsAfaUfaAfaUfaUfcCfaAfaCfcGfsc | 652 | UUCAAUAAAUAUCCAAACCGC | 1462 |
| AM13853-AS | asCfsasAfuAfaAfuAfcCfgAfgGfaAfuGfsc | 653 | ACAAUAAAUACCGAGGAAUGC | 1463 |
| AM13856-AS | usGfscsCfuUfuUfaUfuCfgCfgAfgGfgUfsc | 654 | UGCCUUUUAUUCGCGAGGGUC | 1464 |
| AM14133-AS | cPrpusAfsasGfcAfcGfaCfaCfcUfcGfcCfcCfsu | 655 | UAAGCACGACACCUCGCCCCU | 1465 |
| AM14135-AS | cPrpusCfsgsCfgUfaGfaAfgGfgCfgUfcUfgCfsc | 656 | UCGCGUAGAAGGGCGUCUGCC | 1466 |
| AM14139-AS | cPrpusAfsasUfaCfcGfaGfgAfaUfgUfcGfgGfsg | 657 | UAAUACCGAGGAAUGUCGGGG | 1467 |
| AM14141-AS | cPrpusAfsgsAfcAfaUfaAfaUfaCfcGfaGfgAfsa | 658 | UAGACAAUAAAUACCGAGGAA | 1468 |
| AM14143-AS | usUfscsAfcUfaCfcGfcUfaCfcUfcGfcusg | 659 | UUCACUACCGCUACCUCGCUG | 1469 |
| AM14145-AS | asUfscsCfuGfgAfaGfgCfgAfaGfuGfcasg | 660 | AUCCUGGAAGGCGAAGUGCAG | 1470 |
| AM14148-AS | usUfscsAfcUfaCfcGfcUfaCfcUfcGfcUfsg | 661 | UUCACUACCGCUACCUCGCUG | 1469 |
| AM14150-AS | asUfscsCfuGfgAfaGfgCfgAfaGfuGfcAfsg | 662 | AUCCUGGAAGGCGAAGUGCAG | 1470 |
| AM14152-AS | usAfsasGfcAfcGfaCfaCfcUfcGfcCfcCfsu | 663 | UAAGCACGACACCUCGCCCCU | 1465 |
| AM14154-AS | usCfsgsCfgUfaGfaAfgGfgCfgUfcUfgCfsc | 664 | UCGCGUAGAAGGGCGUCUGCC | 1466 |
| AM14156-AS | usAfsasUfaCfcGfaGfgAfaUfgUfcGfgGfsg | 665 | UAAUACCGAGGAAUGUCGGGG | 1467 |
| AM14158-AS | usAfsgsAfcAfaUfaAfaUfaCfcGfaGfgAfsa | 666 | UAGACAAUAAAUACCGAGGAA | 1468 |
| AM14439-AS | usCfsasAfaAfgCfaAfaUfuUfcCfcGfaGfsu | 667 | UCAAAAGCAAAUUUCCCGAGU | 1471 |
| AM14441-AS | usUfsusUfgGfcAfaAfaGfcAfaAfuUfuCfsc | 668 | UUUUGGCAAAAGCAAAUUUCC | 1472 |
| AM14443-AS | asAfsusAfaAfuAfuCfcAfaAfcCfgCfcGfsa | 669 | AAUAAAUAUCCAAACCGCCGA | 1473 |
| AM14445-AS | usAfsasUfaAfaUfaUfcCfaAfaCfcGfcCfsg | 670 | UAAUAAAUAUCCAAACCGCCG | 1474 |
| AM14447-AS | usCfscsAfaAfaCfgUfgGfaUfuGfgGfgUfsu | 671 | UCCAAAACGUGGAUUGGGGUU | 1475 |
| AM14449-AS | asGfsusGfcAfuCfcAfaAfaCfgUfgGfaUfsc | 672 | AGUGCAUCCAAAACGUGGAUC | 1476 |
| AM14451-AS | usUfscsUfcAfgUfgCfaUfcCfaAfaAfcGfsu | 673 | UUCUCAGUGCAUCCAAAACGU | 1477 |
| AM14453-AS | usGfsusCfuCfaGfuGfcAfuCfcAfaAfaCfsg | 674 | UGUCUCAGUGCAUCCAAAACG | 1478 |
| AM14455-AS | asGfsasCfaAfuAfaAfuAfcCfgAfgGfaAfsu | 675 | AGACAAUAAAUACCGAGGAAU | 1479 |
| AM14457-AS | usAfscsAfgAfcAfaUfaAfaUfaCfcGfaGfsg | 676 | UACAGACAAUAAAUACCGAGG | 1480 |
| AM14459-AS | usGfsasCfaGfaCfaAfuAfaAfuAfcCfgAfsg | 677 | UGACAGACAAUAAAUACCGAG | 1481 |
| AM14461-AS | usCfsusAfgGfuGfgGfgAfcAfgAfcAfaUfsc | 678 | UCUAGGUGGGGACAGACAAUC | 1482 |
| AM14548-AS | cPrpasCfsasAfuAfaAfuAfcCfgAfgGfaAfuGfsc | 679 | ACAAUAAAUACCGAGGAAUGC | 1463 |
| AM14550-AS | cPrpasCfsgsUfaAfuAfcUfcCfaUfgAfcCfaGfsg | 680 | ACGUAAUACUCCAUGACCAGG | 1455 |
| AM14612-AS | cPrpusAfsgsaCfaauaaaUfaCfcGfaggasa | 681 | UAGACAAUAAAUACCGAGGAA | 1468 |
| AM14613-AS | cPrpusAfsgsacAfauaaaUfaCfcGfaggasa | 682 | UAGACAAUAAAUACCGAGGAA | 1468 |
| AM14614-AS | cPrpusAfsgsacaaUfaaaUfaCfcGfaggasa | 683 | UAGACAAUAAAUACCGAGGAA | 1468 |
| AM14615-AS | cPrpusAfsgsacaauAfaaUfaCfcGfaggasa | 684 | UAGACAAUAAAUACCGAGGAA | 1468 |
| AM14617-AS | cPrpusAfsgsaCfaauaaaUfaCfcGfaggasc | 685 | UAGACAAUAAAUACCGAGGAC | 1483 |
| AM14620-AS | cPrpasCfsgsUfaauacucCfaUfgAfccagsg | 686 | ACGUAAUACUCCAUGACCAGG | 1455 |
| AM14622-AS | cPrpasCfsGfsuaauacucCfaUfgAfccagsg | 687 | ACGUAAUACUCCAUGACCAGG | 1455 |
| AM14623-AS | cPrpasCfsgsuAfauacucCfaUfgAfccagsg | 688 | ACGUAAUACUCCAUGACCAGG | 1455 |
| AM14624-AS | cPrpasCfsgsuaAfuacucCfaUfgAfccagsg | 689 | ACGUAAUACUCCAUGACCAGG | 1455 |
| AM15037-AS | usUfsGfsuaguggacGfaUfcUfugccsa | 690 | UUGUAGUGGACGAUCUUGCCA | 1457 |
| AM15038-AS | usUfsgsUfaguggacGfaUfcUfugccsa | 691 | UUGUAGUGGACGAUCUUGCCA | 1457 |
| AM15039-AS | usUfsgsuAfguggacGfaUfcUfugccsa | 692 | UUGUAGUGGACGAUCUUGCCA | 1457 |
| AM15041-AS | cPrpusUfsgsUfaGfuGfgAfcGfaUfcUfuGfcCfsa | 693 | UUGUAGUGGACGAUCUUGCCA | 1457 |
| AM15042-AS | usUfsgUfaGfuGfgAfcGfaUfcUfuGfcsCfsa | 694 | UUGUAGUGGACGAUCUUGCCA | 1457 |
| AM15277-AS | cPrpusAfsgsAfcAfaUfaAfaUfaCfcGfaGfgAfsc | 695 | UAGACAAUAAAUACCGAGGAC | 1483 |
| AM15278-AS | cPrpusAfsgAfcAfaUfaAfaUfaCfcGfaGfgsAfsa | 696 | UAGACAAUAAAUACCGAGGAA | 1468 |
| AM15279-AS | cPrpusAfsgAfcAfaUfaAfaUfaCfcGfaGfgAfsa | 697 | UAGACAAUAAAUACCGAGGAA | 1468 |
| AM15280-AS | cPrpusAfsgsacaAfuaaaUfaCfcGfaggasa | 698 | UAGACAAUAAAUACCGAGGAA | 1468 |
| AM15281-AS | cPrpusAfsgsacaauaAfaUfaCfcGfaggasa | 699 | UAGACAAUAAAUACCGAGGAA | 1468 |
| AM15282-AS | cPrpusAfsgsacaAfuAfAfaUfaCfcGfaggasa | 700 | UAGACAAUAAAUACCGAGGAA | 1468 |
| AM15477-AS | usCfsusUfcAfgCfaUfgUfcCfcAfcUfuGfsu | 701 | UCUUCAGCAUGUCCCACUUGU | 1484 |
| AM15479-AS | usUfscsUfuCfaGfcAfuGfuCfcCfaCfuUfsg | 702 | UUCUUCAGCAUGUCCCACUUG | 1485 |
| AM15481-AS | asGfsgsUfaGfuUfcUfcAfuCfcUfgGfaAfsg | 703 | AGGUAGUUCUCAUCCUGGAAG | 1486 |
| AM15484-AS | asCfsasGfgUfaGfuUfcUfcAfuCfcUfgGfsa | 704 | ACAGGUAGUUCUCAUCCUGGA | 1487 |
| AM15487-AS | usGfsusAfcAfgGfuAfgUfuCfuCfaUfcCfsu | 705 | UGUACAGGUAGUUCUCAUCCU | 1488 |
| AM15489-AS | usCfscsAfuGfaCfcAfgGfuAfcAfgGfuAfsg | 706 | UCCAUGACCAGGUACAGGUAG | 1489 |
| AM15492-AS | usCfscsAfaAfcUfuGfcUfcAfgCfaGfuGfsu | 707 | UCCAAACUUGCUCAGCAGUGU | 1490 |
| AM15494-AS | usUfsgsUfuCfaUfgAfuCfuUfcAfuGfgCfsa | 708 | UUGUUCAUGAUCUUCAUGGCA | 1491 |
| AM15496-AS | asCfsusUfgUfuCfaUfgAfuCfuUfcAfuGfsg | 709 | ACUUGUUCAUGAUCUUCAUGG | 1492 |
| AM15498-AS | usAfscsUfuGfuUfcAfuGfaUfcUfuCfaUfsg | 710 | UACUUGUUCAUGAUCUUCAUG | 1493 |
| AM15500-AS | asCfsusUfgCfuCfaGfcAfgUfgUfcAfgCfsa | 711 | ACUUGCUCAGCAGUGUCAGCA | 1494 |
| AM15502-AS | usAfsasAfcUfuGfcUfcAfgCfaGfuGfuCfsa | 712 | UAAACUUGCUCAGCAGUGUCA | 1495 |
| AM15504-AS | usGfsasUfgUfuGfuCfgGfgUfuUfgAfuGfsc | 713 | UGAUGUUGUCGGGUUUGAUGC | 1496 |
| AM15506-AS | usCfsasGfgAfuGfuUfgUfcGfgGfuUfuGfsc | 714 | UCAGGAUGUUGUCGGGUUUGC | 1497 |
| AM15509-AS | usCfscsUfuGfuAfgUfgGfaCfgAfuCfuUfsg | 715 | UCCUUGUAGUGGACGAUCUUG | 1498 |
| AM15511-AS | asGfsasGfaGfaGfgUfgCfuCfcUfuGfuAfsg | 716 | AGAGAGAGGUGCUCCUUGUAG | 1499 |
| AM15514-AS | asAfsasGfaAfgAfaGfgGfaUfgUfgUfcCfsg | 717 | AAAGAAGAAGGGAUGUGUCCG | 1500 |
| AM15516-AS | usCfsasAfaGfaAfgAfaGfgGfaUfgUfgUfsc | 718 | UCAAAGAAGAAGGGAUGUGUC | 1501 |
| AM15518-AS | usUfsusCfgAfaAfuCfcGfgUfgUfaAfaGfsg | 719 | UUUCGAAAUCCGGUGUAAAGG | 1502 |
| AM15520-AS | usCfsasCfcUfuCfgAfaAfuCfcGfgUfgUfsa | 720 | UCACCUUCGAAAUCCGGUGUA | 1503 |
| AM15522-AS | usCfsgsAfaGfuUfgCfaUfgUfgUfcGfgUfsg | 721 | UCGAAGUUGCAUGUGUCGGUG | 1504 |
| AM15524-AS | asGfsusCfgAfaGfuUfgCfaUfgUfgUfcGfsg | 722 | AGUCGAAGUUGCAUGUGUCGG | 1505 |
| AM15526-AS | usAfscsCfaAfgUfcGfaAfgUfuGfcAfuGfsu | 723 | UACCAAGUCGAAGUUGCAUGU | 1506 |
| AM15780-AS | cPrpusAfsgacaaua AfaUfaCfcGfaggasa | 724 | UAGACAAUAAAUACCGAGGAA | 1468 |
| AM15781-AS | usAfsgsacaauaAfaUfaCfcGfaggasa | 725 | UAGACAAUAAAUACCGAGGAA | 1468 |
| AM15833-AS | asCfsGfsUfAfauacucCfaUfgAfccagsg | 726 | ACGUAAUACUCCAUGACCAGG | 1455 |
| AM15834-AS | asCfsGfsuaAfuaCfucCfaUfgAfccagsg | 727 | ACGUAAUACUCCAUGACCAGG | 1455 |
| AM15835-AS | asCfsgsUfaAfuaCfucCfaUfgAfccagsg | 728 | ACGUAAUACUCCAUGACCAGG | 1455 |
| AM15837-AS | asCfsGfsuaauacucCfaUfgAfccagsg | 729 | ACGUAAUACUCCAUGACCAGG | 1455 |
| AM15838-AS | asCfsgsUfaauacucCfaUfgAfccagsg | 730 | ACGUAAUACUCCAUGACCAGG | 1455 |
| AM16316-AS | usCfsgAfaGfuUfgCfaUfgUfgUfcGfgsUfsg | 731 | UCGAAGUUGCAUGUGUCGGUG | 1504 |
| AM16317-AS | usCfsgAfaGfuUfgCfaUfgUfgUfcGfgUfsg | 732 | UCGAAGUUGCAUGUGUCGGUG | 1504 |
| AM16319-AS | usCfsgsAfaguugcaUfgUfgUfcggusg | 733 | UCGAAGUUGCAUGUGUCGGUG | 1504 |
| AM16320-AS | usCfsgsaAfguugcaUfgUfgUfcggusg | 734 | UCGAAGUUGCAUGUGUCGGUG | 1504 |
| AM16321-AS | usCfsgsaagUfugcaUfgUfgUfcggusg | 735 | UCGAAGUUGCAUGUGUCGGUG | 1504 |
| AM16409-AS | cPrpusAfscsAfgAfcAfaUfaAfaUfaCfcGfaGfsg | 736 | UACAGACAAUAAAUACCGAGG | 1480 |
| AM16537-AS | asCfsGfuaAfuaCfucCfaUfgAfccagsg | 738 | ACGUAAUACUCCAUGACCAGG | 1455 |
| AM16538-AS | cPrpasCfsGfuaAfuaCfucCfaUfgAfccagsg | 739 | ACGUAAUACUCCAUGACCAGG | 1455 |
| AM16540-AS | cPrpusCfsGfuaAfuaCfucCfaUfgAfccagsg | 740 | UCGUAAUACUCCAUGACCAGG | 1507 |
| AM16541-AS | asCfsGfsuAfAfuacucCfaUfgAfccagsg | 741 | ACGUAAUACUCCAUGACCAGG | 1455 |
| AM16542-AS | asCfsGfsuAfaUfacucCfaUfgAfccagsg | 742 | ACGUAAUACUCCAUGACCAGG | 1455 |
| AM16543-AS | asCfsGfsuAfauacUfcCfaUfgAfccagsg | 743 | ACGUAAUACUCCAUGACCAGG | 1455 |
| AM16544-AS | asCfsGfsuaAfUfacucCfaUfgAfccagsg | 744 | ACGUAAUACUCCAUGACCAGG | 1455 |
| AM16574-AS | usUfscgaAfgacaguuCfuAfgggsusu | 745 | UUCGAAGACAGUUCUAGGGUU | 1508 |
| AM16575-AS | cPrpusUfscgaAfgacaguuCfuAfgggsusu | 746 | UUCGAAGACAGUUCUAGGGUU | 1508 |
| AM16577-AS | usAfsgacaauaAfaUfaCfcGfaggasa | 747 | UAGACAAUAAAUACCGAGGAA | 1468 |
| AM16702-AS | usCfsGfsaaguugcaUfgUfgUfcggusg | 748 | UCGAAGUUGCAUGUGUCGGUG | 1504 |
| AM16703-AS | usCfsgsaaguuGfcaUfgUfgUfcggusg | 749 | UCGAAGUUGCAUGUGUCGGUG | 1504 |
| AM16704-AS | usCfsGfsaAfgUfugcaUfgUfgUfcggusg | 750 | UCGAAGUUGCAUGUGUCGGUG | 1504 |
| AM16803-AS | cPrpusUfscsGfaAfgAfcAfgUfuCfuAfgGfgUfsc | 751 | UUCGAAGACAGUUCUAGGGUC | 1446 |
| AM16805-AS | cPrpusCfsgsAfaGfaCfaGfuUfcUfaGfgGfuUfsc | 752 | UCGAAGACAGUUCUAGGGUUC | 1445 |
| AM16905-AS | usUfsgsuagUfggacGfaUfcUfugccsa | 753 | UUGUAGUGGACGAUCUUGCCA | 1457 |
| AM16906-AS | usUfsgsuagugGfacGfaUfcUfugccsa | 754 | UUGUAGUGGACGAUCUUGCCA | 1457 |
| AM16909-AS | usUfsguAfguggacGfaUfcUfugcscsa | 755 | UUGUAGUGGACGAUCUUGCCA | 1457 |
| AM16910-AS | usUfsguAfguggacGfaUfcUfugccsa | 756 | UUGUAGUGGACGAUCUUGCCA | 1457 |
| AM16962-AS | cPrpusCfsGfuAfauacUfcCfaUfgAfccagsg | 757 | UCGUAAUACUCCAUGACCAGG | 1507 |
| AM17362-AS | cPrpusCfsgsaagUfugcaUfgUfgUfcggusg | 758 | UCGAAGUUGCAUGUGUCGGUG | 1504 |
| AM17364-AS | cPrpusUfsgsuAfguggacGfaUfcUfugccsa | 759 | UUGUAGUGGACGAUCUUGCCA | 1457 |
| AM17365-AS | usCfsGfuAfauacUfcCfaUfgAfccagsg | 760 | UCGUAAUACUCCAUGACCAGG | 1507 |
| AM17649-AS | cPrpusAfscsagaCfaaua AfaUfaCfcgagsg | 761 | UACAGACAAUAAAUACCGAGG | 1480 |
| AM17652-AS | cPrpusAfscagaCfaauaAfaUfaCfcgagsg | 762 | UACAGACAAUAAAUACCGAGG | 1480 |
| AM17653-AS | cPrpusAfscsaGfacaauaAfaUfaCfcgagsg | 763 | UACAGACAAUAAAUACCGAGG | 1480 |
| AM17654-AS | cPrpusAfscagacaaUfaAfaUfaCfcgagsg | 764 | UACAGACAAUAAAUACCGAGG | 1480 |
| AM18250-AS | dTssAfsgacaauaAfaUfaCfcGfaggasa | 765 | TAGACAAUAAAUACCGAGGAA | 1623 |
| AM18251-AS | UssAfsgacaauaAfaUfaCfcGfaggasa | 766 | UAGACAAUAAAUACCGAGGAA | 1468 |
| AM19163-AS | dTssAfgacaaua AfaUfaCfcGfaggasa | 767 | TAGACAAUAAAUACCGAGGAA | 1623 |
| CA916066 | cPrpusCfsGfuAfauacUfcCfaUfgAfccagg | 768 | UCGUAAUACUCCAUGACCAGG | 1507 |
| CA916067 | cPrpusCfsGfuAfauacUfcCfaUfggAfccagsg | 769 | UCGUAAUACUCCAUGGACCAGG | 1509 |
| CA916068 | CfsGfuAfauacUfcCfaUfgAfccagsg | 770 | CGUAAUACUCCAUGACCAGG | 1510 |
| CA005473 | cPrpusCfsGfuAfauacUfcCfaUfgAfccagssg | 771 | UCGUAAUACUCCAUGACCAGG | 1507 |
| CA005474 | cPrpusCfsGfuaAfuacUfcCfaUfgAfccagsg | 772 | UCGUAAUACUCCAUGACCAGG | 1507 |
| CA005475 | cPrpusCfsGfuaAfuacUfcCfaUfgAfccagssg | 773 | UCGUAAUACUCCAUGACCAGG | 1507 |
| CA005476 | cPrpusCfsguaaUfacucCfaUfgAfccagsg | 774 | UCGUAAUACUCCAUGACCAGG | 1507 |
| CA005477 | cPrpusCfsguaaUfacucCfaUfgAfccagssg | 775 | UCGUAAUACUCCAUGACCAGG | 1507 |

TABLE 4.1
DMPK RNAi Agent Sense Strand Sequences

| | | | Underlying Base | |
|---|---|---|---|---|
| Sense | | Seq | Sequence (5′ → 3′) | Seq |
| Strand | | ID | (Shown as an Unmodified | ID |
| ID | Modified Sense Strand (5′ → 3′) | No. | Nucleotide Sequence) | No. |
| AM12526-SS | (NAG37)s(invAb)sggaugaaaCfAfGfcugaaguigas(invAb) | 776 | GGAUGAAACAGCUGAAGUIGA | 1511 |
| AM12528-SS | (NAG37)s(invAb)succcugaaCfCfCfuagaacuguas(invAb) | 777 | UCCCUGAACCCUAGAACUGUA | 1512 |
| AM12530-SS | (NAG37)s(invAb)sgaacccuaGfAfAfcugucuucias(invAb) | 778 | GAACCCUAGAACUGUCUUCIA | 1513 |
| AM12532-SS | (NAG37)s(invAb)sgacccuagAfAfCfugucuuciaas(invAb) | 779 | GACCCUAGAACUGUCUUCIAA | 1514 |
| AM12534-SS | (NAG37)s(invAb)sacccuagaAfCfUfgucuuciacus(invAb) | 780 | ACCCUAGAACUGUCUUCIACU | 1515 |
| AM12536-SS | (NAG37)s(invAb)scccuagaaCfUfGfucuuciacuas(invAb) | 781 | CCCUAGAACUGUCUUCIACUA | 1516 |
| AM12538-SS | (NAG37)s(invAb)sugcuuacuCfGfGfgaaauuugcus(invAb) | 782 | UGCUUACUCGGGAAAUUUGCU | 1517 |
| AM12540-SS | (NAG37)s(invAb)scgggaaauUfUfGfcuuuuiccaas(invAb) | 783 | CGGGAAAUUUGCUUUUICCAA | 1518 |
| AM12542-SS | (NAG37)s(invAb)saccccaauCfCfAfcguuuuggaus(invAb) | 784 | ACCCCAAUCCACGUUUUGGAU | 1519 |
| AM12544-SS | (NAG37)s(invAb)scccaauccAfCfGfuuuugiaugas(invAb) | 785 | CCCAAUCCACGUUUUGIAUGA | 1520 |
| AM13805-SS | (NAG37)s(invAb)sgagggacgUfGfUfugguiaaugas(invAb) | 786 | GAGGGACGUGUUGGUIAAUGA | 1521 |
| AM13807-SS | (NAG37)s(invAb)sggaugagaAfCfUfaccuiuaccus(invAb) | 787 | GGAUGAGAACUACCUIUACCU | 1522 |
| AM13809-SS | (NAG37)s(invAb)sccuggucaUfGfGfaguauuacgus(invAb) | 788 | CCUGGUCAUGGAGUAUUACGU | 1523 |
| AM13811-SS | (NAG37)s(invAb)sggacaucaAfAfCfccgacaacaus(invAb) | 789 | GGACAUCAAACCCGACAACAU | 1524 |
| AM13814-SS | (NAG37)s(invAb)suggcaagaUfCfGfuccacuacaas(invAb) | 790 | UGGCAAGAUCGUCCACUACAA | 1525 |
| AM13817-SS | (NAG37)s(invAb)sccccuuuaCfAfCfcggauuucgas(invAb) | 791 | CCCCUUUACACCGGAUUUCGA | 1526 |
| AM13819-SS | (NAG37)s(invAb)scgacacauGfCfAfacuuciacuus(invAb) | 792 | CGACACAUGCAACUUCIACUU | 1527 |
| AM13821-SS | (NAG37)s(invAb)sggaacccuAfGfAfacugucuucas(invAb) | 793 | GGAACCCUAGAACUGUCUUCA | 1528 |
| AM13823-SS | (NAG37)s(invAb)sggaacccuAfGfAfacuguuuucas(invAb) | 794 | GGAACCCUAGAACUGUUUUCA | 1529 |
| AM13824-SS | (NAG37)s(invAb)scugcuuacUfCfGfggaaauuugas(invAb) | 795 | CUGCUUACUCGGGAAAUUUGA | 1530 |
| AM13826-SS | (NAG37)s(invAb)sgcgguuugGfa_2NUfaUfuuauugaas(invAb) | 796 | GCGGUUUGG(A^{2N})UAUUUAUUGAA | 1531 |
| AM13852-SS | (NAG37)s(invAb)sgcauuccuCfGfGfuauuuauugus(invAb) | 797 | GCAUUCCUCGGUAUUUAUUGU | 1532 |
| AM13854-SS | (NAG37)s(invAb)sgcauuccuCfGfGfuauuua_2Nuugus(invAb) | 798 | GCAUUCCUCGGUAUUU(A^{2N})UUGU | 1533 |
| AM13855-SS | (NAG37)s(invAb)sgacccucgCfGfAfauaaaagicas(invAb) | 799 | GACCCUCGCGAAUAAAAGICA | 1534 |
| AM14132-SS | (NH2-C6)s(invAb)saggggcgaGfGfUfgucgugcuuas(invAb) | 800 | AGGGGCGAGGUGUCGUGCUUAT | 1535 |
| | (C6-SS-C6)dT | | | |
| AM14134-SS | (NH2-C6)s(invAb)sggcagacgCfCfCfuucuacgcgas(invAb) | 801 | GGCAGACGCCCUUCUACGCGAT | 1536 |
| | (C6-SS-C6)dT | | | |
| AM14136-SS | (NH2-C6)s(invAb)sggcagaccCfCfCfuucuacgcgas(invAb) | 802 | GGCAGACCCCCUUCUACGCGAT | 1537 |
| | (C6-SS-C6)dT | | | |
| AM14138-SS | (NH2-C6)s(invAb)sccccgacaUfUfCfcucgguauuas(invAb) | 803 | CCCCGACAUUCCUCGGUAUUAT | 1538 |
| | (C6-SS-C6)dT | | | |
| AM14140-SS | (NH2-C6)s(invAb)suuccucggUfAfUfuuauugucuas(invAb) | 804 | UUCCUCGGUAUUUAUUGUCUAT | 1539 |
| | (C6-SS-C6)dT | | | |
| AM14142-SS | (NAG37)s(invAb)sgcgaggUfAfGfcgguagugaadTdTs(invAb) | 805 | GCGAGGUAGCGGUAGUGAATT | 1540 |
| AM14151-SS | (NAG37)s(invAb)saggggcgaGfGfUfgucgugcuuas(invAb) | 806 | AGGGGCGAGGUGUCGUGCUUA | 1541 |
| AM14155-SS | (NAG37)s(invAb)sccccgacaUfUfCfcucgguauuas(invAb) | 807 | CCCCGACAUUCCUCGGUAUUA | 1542 |
| AM14157-SS | (NAG37)s(invAb)suuccucggUfAfUfuuauugucuas(invAb) | 808 | UUCCUCGGUAUUUAUUGUCUA | 1543 |
| AM14438-SS | (NAG37)s(invAb)sacucgggaAfAfUfuugcuuuugas(invAb) | 809 | ACUCGGGAAAUUUGCUUUUGA | 1544 |
| AM14440-SS | (NAG37)s(invAb)sggaaauuuGfCfUfuuugucaaaas(invAb) | 810 | GGAAAUUUGCUUUUGUCAAAA | 1545 |
| AM14442-SS | (NAG37)s(invAb)sucggcgguUfUfGfgauauuua_2Nuus(invAb) | 811 | UCGGCGGUUUGGAUAUUU(A^{2N})UU | 1546 |
| AM14444-SS | (NAG37)s(invAb)scggcgguuUfGfGfauauuua_2Nuuas(invAb) | 812 | CGGCGGUUUGGAUAUUU(A^{2N})UUA | 1547 |
| AM14446-SS | (NAG37)s(invAb)saaccccaaUfCfCfacguuuuggas(invAb) | 813 | AACCCCAAUCCACGUUUUGGA | 1548 |
| AM14448-SS | (NAG37)s(invAb)sgauccacgUfUfUfuggauicacus(invAb) | 814 | GAUCCACGUUUUGGAUICACU | 1549 |
| AM14450-SS | (NAG37)s(invAb)sacguuuugGfAfUfgcacuiagaas(invAb) | 815 | ACGUUUUGGAUGCACUIAGAA | 1550 |
| AM14452-SS | (NAG37)s(invAb)scguuuuggAfUfGfcacuiagacas(invAb) | 816 | CGUUUUGGAUGCACUIAGACA | 1551 |
| AM14454-SS | (NAG37)s(invAb)sauuccucgGfUfAfuuuauugucus(invAb) | 817 | AUUCCUCGGUAUUUAUUGUCU | 1552 |
| AM14456-SS | (NAG37)s(invAb)sccucgguaUfUfUfauuguuuguas(invAb) | 818 | CCUCGGUAUUUAUUGUUUGUA | 1553 |
| AM14547-SS | (NH2-C6)s(invAb)sgcauuccuCfGfGfuauuuauugus(invAb) | 819 | GCAUUCCUCGGUAUUUAUUGUT | 1554 |
| | (C6-SS-C6)dT | | | |
| AM14549-SS | (NH2-C6)s(invAb)sccuggucaUfGfGfaguauuacgus(invAb) | 820 | CCUGGUCAUGGAGUAUUACGUT | 1555 |
| | (C6-SS-C6)dT | | | |
| AM14616-SS | (NH2-C6)s(invAb)sguccucggUfAfUfuuauugucuas(invAb) | 821 | GUCCUCGGUAUUUAUUGUCUAT | 1556 |
| | (C6-SS-C6)dT | | | |
| AM14618-SS | (NH2-C6)s(invAb)suuccucggUfaUfuUfauugucuas(invAb) | 822 | UUCCUCGGUAUUUAUUGUCUAT | 1539 |
| | (C6-SS-C6)dT | | | |
| AM14619-SS | (NAG37)s(invAb)sccuggucaUfgGfaGfuauuacgus(invAb) | 823 | CCUGGUCAUGGAGUAUUACGU | 1523 |
| AM14621-SS | (NAG37)s(invAb)sccuggucaUfgGfaguauuacgus(invAb) | 824 | CCUGGUCAUGGAGUAUUACGU | 1523 |
| AM14668-SS | (NH2-C6)s(invAb)sccccgacaUfUfCfcucgguauuas(invAb) | 825 | CCCCGACAUUCCUCGGUAUUA | 1542 |
| AM14669-SS | (NH2-C6)s(invAb)suuccucggUfAfUfuuauugucuas(invAb) | 826 | UUCCUCGGUAUUUAUUGUCUA | 1543 |
| AM15036-SS | (NAG37)s(invAb)suggcaagaUfcGfuccacuacaas(invAb) | 827 | UGGCAAGAUCGUCCACUACAA | 1525 |
| AM15040-SS | (NAG37)s(invAb)suggcaaGfaUfcGfuccacuacaas(invAb) | 828 | UGGCAAGAUCGUCCACUACAA | 1525 |
| AM15276-SS | (NH2-C6)s(invAb)sguccucggUfaUfuUfauugucuas(invAb) | 829 | GUCCUCGGUAUUUAUUGUCUAT | 1556 |
| | (C6-SS-C6)dT | | | |
| AM15283-SS | (NH2-C6)s(invAb)suuccucggUfaUfUfuauugucuas(invAb) | 830 | UUCCUCGGUAUUUAUUGUCUAT | 1539 |
| | (C6-SS-C6)dT | | | |
| AM15476-SS | (NH2-C6)s(invAb)sacaaguggGfAfCfaugcuiaagas(invAb) | 831 | ACAAGUGGGACAUGCUIAAGAT | 1557 |
| | (C6-SS-C6)dT | | | |
| AM15478-SS | (NH2-C6)s(invAb)scaagugggAfCfAfugcuiaagaas(invAb) | 832 | CAAGUGGGACAUGCUIAAGAAT | 1558 |
| | (C6-SS-C6)dT | | | |
| AM15480-SS | (NH2-C6)s(invAb)scuuccaggAfUfGfagaacuacuus(invAb) | 833 | CUUCCAGGAUGAGAACUACUUT | 1559 |
| | (C6-SS-C6)dT | | | |
| AM15482-SS | (NH2-C6)s(invAb)scuuccaggAfUfGfagaacuaucus(invAb) | 834 | CUUCCAGGAUGAGAACUAUCUT | 1560 |
| | (C6-SS-C6)dT | | | |
| AM15483-SS | (NH2-C6)s(invAb)succaggauGfAfGfaacuaccuius(invAb) | 835 | UCCAGGAUGAGAACUACCUIUT | 1561 |
| | (C6-SS-C6)dT | | | |
| AM15485-SS | (NH2-C6)s(invAb)succaggauGfAfGfaacuacuugus(invAb) | 836 | UCCAGGAUGAGAACUACUUGUT | 1562 |
| | (C6-SS-C6)dT | | | |
| AM15486-SS | (NH2-C6)s(invAb)saggaugagAfAfCfuaccuiuacas(invAb) | 837 | AGGAUGAGAACUACCUIUACAT | 1563 |
| | (C6-SS-C6)dT | | | |
| AM15488-SS | (NH2-C6)s(invAb)scuaccuguAfCfCfuggucauigas(invAb) | 838 | CUACCUGUACCUGGUCAUIGAT | 1564 |
| | (C6-SS-C6)dT | | | |
| AM15490-SS | (NH2-C6)s(invAb)scuaccuguAfCfCfugguuauggas(invAb) | 839 | CUACCUGUACCUGGUUAUGGAT | 1565 |
| | (C6-SS-C6)dT | | | |
| AM15491-SS | (NH2-C6)s(invAb)sacacugcuGfAfGfcaaguuuigas(invAb) | 840 | ACACUGCUGAGCAAGUUUIGAT | 1566 |
| | (C6-SS-C6)dT | | | |
| AM15510-SS | (NAG37)s(invAb)scuacaaggAfGfCfaccucuuucus(invAb) | 841 | CUACAAGGAGCACCUCUUUCU | 1567 |
| AM15512-SS | (NAG37)s(invAb)scuacaaggAfGfCfaccuuucucus(invAb) | 842 | CUACAAGGAGCACCUUUCUCU | 1568 |
| AM15513-SS | (NAG37)s(invAb)scggacacaUfCfCfcuucuucuuus(invAb) | 843 | CGGACACAUCCCUUCUUCUUU | 1569 |
| AM15515-SS | (NAG37)s(invAb)sgacacaucCfCfUfucuucuuugas(invAb) | 844 | GACACAUCCCUUCUUCUUUGA | 1570 |
| AM15517-SS | (NAG37)s(invAb)sccuuuacaCfCfGfgauuucgaaas(invAb) | 845 | CCUUUACACCGGAUUUCGAAA | 1571 |
| AM15519-SS | (NAG37)s(invAb)suacaccggAfUfUfucgaagiugas(invAb) | 846 | UACACCGGAUUUCGAAGIUGA | 1572 |
| AM15521-SS | (NAG37)s(invAb)scaccgacaCfAfUfgcaacuucias(invAb) | 847 | CACCGACACAUGCAACUUCIA | 1573 |
| AM15523-SS | (NAG37)s(invAb)sccgacacaUfGfCfaacuuciacus(invAb) | 848 | CCGACACAUGCAACUUCIACU | 1574 |
| AM15525-SS | (NAG37)s(invAb)sacaugcaaCfUfUfcgacuuiguas(invAb) | 849 | ACAUGCAACUUCGACUUIGUA | 1575 |
| AM15836-SS | (NAG37)s(invAb)sccuggucaUfgGfAfguauuacgus(invAb) | 850 | CCUGGUCAUGGAGUAUUACGU | 1523 |
| AM16408-SS | (NH2-C6)s(invAb)sccucgguaUfUfUfauuguuuguas(invAb) | 851 | CCUCGGUAUUUAUUGUUUGUAT | 1576 |
| | (C6-SS-C6)dT | | | |
| AM16410-SS | (NH2-C6)s(invAb)sccucgguaUfUfUfauugucuguas(invAb) | 852 | CCUCGGUAUUUAUUGUCUGUAT | 1577 |
| | (C6-SS-C6)dT | | | |
| AM16411-SS | (NH2-C6)s(invAb)sccucgguaUfUfUfauugucuiuas(invAb) | 853 | CCUCGGUAUUUAUUGUCUIUAT | 1578 |
| | (C6-SS-C6)dT | | | |
| AM16539-SS | (NAG37)s(invAb)sccuggucaUfgGfAfguauuacgas(invAb) | 854 | CCUGGUCAUGGAGUAUUACGA | 1579 |
| AM16573-SS | (NAG37)cscscuagAfAfCfugucuucgsasa | 855 | CCCUAGAACUGUCUUCGAA | 1580 |
| AM16576-SS | (NAG37)s(invAb)suuccucggUfaUfUfuauugucuas(invAb) | 856 | UUCCUCGGUAUUUAUUGUCUA | 1543 |
| AM16701-SS | (NAG37)s(invAb)scaccgacaCfaUfgCfaacuucias(invAb) | 857 | CACCGACACAUGCAACUUCIA | 1573 |
| AM16802-SS | (NH2-C6)s(invAb)sgacccuagAfAfCfugucuuciaas(invAb) | 858 | GACCCUAGAACUGUCUUCIAAT | 1581 |
| | (C6-SS-C6)dT | | | |
| AM16804-SS | (NH2-C6)s(invAb)sgaacccuaGfAfAfcugucuucias(invAb) | 859 | GAACCCUAGAACUGUCUUCIAT | 1582 |
| | (C6-SS-C6)dT | | | |
| AM16907-SS | (NAG37)s(invAb)suggcaagaUfcGfUfccacuacaas(invAb) | 860 | UGGCAAGAUCGUCCACUACAA | 1525 |
| AM16908-SS | (NAG37)s(invAb)suggcaagaUfcGfuCfcacuacaas(invAb) | 861 | UGGCAAGAUCGUCCACUACAA | 1525 |
| AM16963-SS | (NH2-C6)s(invAb)sccuggucaUfgGfAfguauuacgas(invAb) | 862 | CCUGGUCAUGGAGUAUUACGAT | 1583 |
| | (C6-SS-C6)dT | | | |
| AM17081-SS | (NH2-C6)cscscuagAfAfCfugucuucgsasa(invAb) | 863 | CCCUAGAACUGUCUUCGAAT | 1584 |
| | (C6-SS-C6)dT | | | |
| AM17650-SS | (NH2-C6)s(invAb)sccucgguaUfuUfaUfugucuguas(invAb) | 864 | CCUCGGUAUUUAUUGUCUGUAT | 1577 |
| | (C6-SS-C6)dT | | | |
| AM17651-SS | (NH2-C6)s(invAb)sccucgguaUfuUfAfuugucuguas(invAb) | 865 | CCUCGGUAUUUAUUGUCUGUAT | 1577 |
| | (C6-SS-C6)dT | | | |
| AM18528-SS | (NH2-C6)s(invAb)suuccucggUfaUfUfuauugucuas(invAb) | 866 | UUCCUCGGUAUUUAUUGUCUA | 1543 |
| AM19347-SS | (NH2-C6)s(invAb)sccuggucaUfgGfAfguauuacgas(invAb) | 867 | CCUGGUCAUGGAGUAUUACGA | 1579 |
| AM19421-SS | (NH2-C6)s(invAb)suuccucggUfaUfUfuauugucuaAlk | 868 | UUCCUCGGUAUUUAUUGUCUA | 1585 |
| AM19422-SS | (NH2-C6)s(invAb)suuccucggUfaUfUfuauugucusaAlk | 869 | UUCCUCGGUAUUUAUUGUCUA | 1585 |
| AM19573-SS | (NH2-C6)s(invAb)suuccucggUfaUfUfuauugucuas(invAb)s | 870 | UUCCUCGGUAUUUAUUGUCUAT | 1539 |
| | (C6-SS-C6)dT | | | |
| AM20339-SS | NH2-C6s(invAb)sccuggucaUfgGfAfguauuacga(invAb) | 871 | CCUGGUCAUGGAGUAUUACGAT | 1583 |
| | (C6-SS-C6)dT | | | |
| AM20340-SS | NH2-C6s(invAb)sccugggucaUfgGfAfguauuacgas(invAb) | 872 | CCUGGGUCAUGGAGUAUUACGAT | 1586 |
| | (C6-SS-C6)dT | | | |
| AM20342-SS | NH2-C6s(invAb)scuggucaUfgGfAfguauuacgas(invAb) | 874 | CUGGUCAUGGAGUAUUACGAT | 1587 |
| | (C6-SS-C6)dT | | | |

(A^{2N}) represents a 2-aminoadenine nucleotide; I represents an inosine (hypoxanthine) nucleotide

As shown in Table 4.1, above, the example DMPK RNAi agent sense strand nucleotide sequences are shown to further include, in some embodiments, reactive linking groups at both the 5′ terminal end and the 3′ terminal end of the sense strand. For example, certain of the DMPK RNAi agent sense strand sequences shown in Table 4.1 above have an (NH2-C6) linking group at the 5′ end of the nucleotide sequence. Similarly, certain of the DMPK RNAi agent nucleotide sequences shown in Table 4.1 above have a (C6-SS-C6) linking group near the 3′ end of the nucleotide sequence. Such reactive linking groups are positioned to facilitate the linking of targeting ligands, targeting groups, and/or PK/PD modulators to the DMPK RNAi agents disclosed herein. Linking or conjugation reactions are well known in the art and provide for formation of covalent linkages between two molecules or reactants. Suitable conjugation reactions for use in the scope of the inventions herein include, but are not limited to, amide coupling reaction, Michael addition reaction, hydrazone formation reaction, and click chemistry cycloaddition reaction.

In some embodiments, targeting ligands can be synthesized as a tetrafluorophenyl (TFP) ester, which react with an amino group (e.g., NH2-C6) to attach the targeting ligand to the DMPK RNAi agents disclosed herein. In some embodiments, targeting ligands are synthesized as azides, which can be conjugated to a propargyl or DBCO group, for example, via click chemistry cycloaddition reaction.

Additionally, the nucleotide sequences shown in Table 4.1 were synthesized with a dT nucleotide at the 3′ terminal end of the sense strand, followed by (3′→5′) a linker (e.g., C6-SS-C6). A suitable and commercially available dT-loaded resin can be used to initiate the synthesis of the oligonucleotide strand. The (C6-SS-C6) linker can, in some embodiments, then be used facilitate the linkage to additional components, such as, for example, a PK/PD modulator or one or more targeting ligands. As described herein, the C6-SS-C6 is first reduced cleaving among other things the dT residue off the molecule, which can then facilitate the conjugation of the desired PK/PD modulator. Table 4.2 below shows the nucleotide sequences identified in Table 4.1, above, but without the inclusion of the 3′ terminal dT nucleotide, as these properly reflect the sequence of the DMPK RNAi agents disclosed herein when delivered in vivo.

Further, Table 4.3 below, shows the nucleotide sequences identified in Table 4.1, above, but without the terminal linking groups present (i.e., the nucleotide sequences with only capping groups).

TABLE 4.2
DMPK RNAi Agent Sense Strand Sequences Shown Without 3′ Terminal dT

| | | | Underlying Base Sequence | |
|---|---|---|---|---|
| | | Seq | (5′ → 3′) | Seq |
| Sense | | ID | (Shown as an Unmodified | ID |
| Strand ID | Modified Sense Strand (5′ → 3′) | No. | Nucleotide Sequence) | No. |
| AM12526-SS-NT | (NAG37)s(invAb)sggaugaaaCfAfGfcugaaguigas(invAb) | 876 | GGAUGAAACAGCUGAAGUIGA | 1511 |
| AM12528-SS-NT | (NAG37)s(invAb)succcugaaCfCfCfuagaacuguas(invAb) | 877 | UCCCUGAACCCUAGAACUGUA | 1512 |
| AM12530-SS-NT | (NAG37)s(invAb)sgaacccuaGfAfAfcugucuucias(invAb) | 878 | GAACCCUAGAACUGUCUUCIA | 1513 |
| AM12532-SS-NT | (NAG37)s(invAb)sgacccuagAfAfCfugucuuciaas(invAb) | 879 | GACCCUAGAACUGUCUUCIAA | 1514 |
| AM12534-SS-NT | (NAG37)s(invAb)sacccuagaAfCfUfgucuuciacus(invAb) | 880 | ACCCUAGAACUGUCUUCIACU | 1515 |
| AM12536-SS-NT | (NAG37)s(invAb)scccuagaaCfUfGfucuuciacuas(invAb) | 881 | CCCUAGAACUGUCUUCIACUA | 1516 |
| AM12538-SS-NT | (NAG37)s(invAb)sugcuuacuCfGfGfgaaauuugcus(invAb) | 882 | UGCUUACUCGGGAAAUUUGCU | 1517 |
| AM12540-SS-NT | (NAG37)s(invAb)scgggaaauUfUfGfcuuuuiccaas(invAb) | 883 | CGGGAAAUUUGCUUUUICCAA | 1518 |
| AM12542-SS-NT | (NAG37)s(invAb)saccccaauCfCfAfcguuuuggaus(invAb) | 884 | ACCCCAAUCCACGUUUUGGAU | 1519 |
| AM12544-SS-NT | (NAG37)s(invAb)scccaauccAfCfGfuuuugiaugas(invAb) | 885 | CCCAAUCCACGUUUUGIAUGA | 1520 |
| AM13805-SS-NT | (NAG37)s(invAb)sgagggacgUfGfUfugguiaaugas(invAb) | 886 | GAGGGACGUGUUGGUIAAUGA | 1521 |
| AM13807-SS-NT | (NAG37)s(invAb)sggaugagaAfCfUfaccuiuaccus(invAb) | 887 | GGAUGAGAACUACCUIUACCU | 1522 |
| AM13809-SS-NT | (NAG37)s(invAb)sccuggucaUfGfGfaguauuacgus(invAb) | 888 | CCUGGUCAUGGAGUAUUACGU | 1523 |
| AM13811-SS-NT | (NAG37)s(invAb)sggacaucaAfAfCfccgacaacaus(invAb) | 889 | GGACAUCAAACCCGACAACAU | 1524 |
| AM13814-SS-NT | (NAG37)s(invAb)suggcaagaUfCfGfuccacuacaas(invAb) | 890 | UGGCAAGAUCGUCCACUACAA | 1525 |
| AM13817-SS-NT | (NAG37)s(invAb)sccccuuuaCfAfCfcggauuucgas(invAb) | 891 | CCCCUUUACACCGGAUUUCGA | 1526 |
| AM13819-SS-NT | (NAG37)s(invAb)scgacacauGfCfAfacuuciacuus(invAb) | 892 | CGACACAUGCAACUUCIACUU | 1527 |
| AM13821-SS-NT | (NAG37)s(invAb)sggaacccuAfGfAfacugucuucas(invAb) | 893 | GGAACCCUAGAACUGUCUUCA | 1528 |
| AM13823-SS-NT | (NAG37)s(invAb)sggaacccuAfGfAfacuguuuucas(invAb) | 894 | GGAACCCUAGAACUGUUUUCA | 1529 |
| AM13824-SS-NT | (NAG37)s(invAb)scugcuuacUfCfGfggaaauuugas(invAb) | 895 | CUGCUUACUCGGGAAAUUUGA | 1530 |
| AM13826-SS-NT | (NAG37)s(invAb)sgcgguuugGfa_2NUfaUfuuauugaas(invAb) | 896 | GCGGUUUGG(A^{2N})UAUUUAUUGAA | 1531 |
| AM13852-SS-NT | (NAG37)s(invAb)sgcauuccuCfGfGfuauuuauugus(invAb) | 897 | GCAUUCCUCGGUAUUUAUUGU | 1532 |
| AM13854-SS-NT | (NAG37)s(invAb)sgcauuccuCfGfGfuauuua_2Nuugus(invAb) | 898 | GCAUUCCUCGGUAUUU(A^{2N})UUGU | 1533 |
| AM13855-SS-NT | (NAG37)s(invAb)sgacccucgCfGfAfauaaaagicas(invAb) | 899 | GACCCUCGCGAAUAAAAGICA | 1534 |
| AM14132-SS-NT | (NH2-C6)s(invAb)saggggcgaGfGfUfgucgugcuuas(invAb) | 900 | AGGGGCGAGGUGUCGUGCUUA | 1541 |
| | (C6-SS-C6) | | | |
| AM14134-SS-NT | (NH2-C6)s(invAb)sggcagacgCfCfCfuucuacgcgas(invAb) | 901 | GGCAGACGCCCUUCUACGCGA | 1588 |
| | (C6-SS-C6) | | | |
| AM14136-SS-NT | (NH2-C6)s(invAb)sggcagaccCfCfCfuucuacgcgas(invAb) | 902 | GGCAGACCCCCUUCUACGCGA | 1589 |
| | (C6-SS-C6) | | | |
| AM14138-SS-NT | (NH2-C6)s(invAb)sccccgacaUfUfCfcucgguauuas(invAb) | 903 | CCCCGACAUUCCUCGGUAUUA | 1542 |
| | (C6-SS-C6) | | | |
| AM14140-SS-NT | (NH2-C6)s(invAb)suuccucggUfAfUfuuauugucuas(invAb) | 904 | UUCCUCGGUAUUUAUUGUCUA | 1543 |
| | (C6-SS-C6) | | | |
| AM14142-SS-NT | (NAG37)s(invAb)sgcgaggUfAfGfcgguagugaas(invAb) | 905 | GCGAGGUAGCGGUAGUGAA | 1590 |
| AM14151-SS-NT | (NAG37)s(invAb)saggggcgaGfGfUfgucgugcuuas(invAb) | 906 | AGGGGCGAGGUGUCGUGCUUA | 1541 |
| AM14155-SS-NT | (NAG37)s(invAb)sccccgacaUfUfCfcucgguauuas(invAb) | 907 | CCCCGACAUUCCUCGGUAUUA | 1542 |
| AM14157-SS-NT | (NAG37)s(invAb)suuccucggUfAfUfuuauugucuas(invAb) | 908 | UUCCUCGGUAUUUAUUGUCUA | 1543 |
| AM14438-SS-NT | (NAG37)s(invAb)sacucgggaAfAfUfuugcuuuugas(invAb) | 909 | ACUCGGGAAAUUUGCUUUUGA | 1544 |
| AM14440-SS-NT | (NAG37)s(invAb)sggaaauuuGfCfUfuuugucaaaas(invAb) | 910 | GGAAAUUUGCUUUUGUCAAAA | 1545 |
| AM14442-SS-NT | (NAG37)s(invAb)sucggcgguUfUfGfgauauuua_2Nuus(invAb) | 911 | UCGGCGGUUUGGAUAUUU(A^{2N})UU | 1546 |
| AM14444-SS-NT | (NAG37)s(invAb)scggcgguuUfGfGfauauuua_2Nuuas(invAb) | 912 | CGGCGGUUUGGAUAUUU(A^{2N})UUA | 1547 |
| AM14446-SS-NT | (NAG37)s(invAb)saaccccaaUfCfCfacguuuuggas(invAb) | 913 | AACCCCAAUCCACGUUUUGGA | 1548 |
| AM14448-SS-NT | (NAG37)s(invAb)sgauccacgUfUfUfuggauicacus(invAb) | 914 | GAUCCACGUUUUGGAUICACU | 1549 |
| AM14450-SS-NT | (NAG37)s(invAb)sacguuuugGfAfUfgcacuiagaas(invAb) | 915 | ACGUUUUGGAUGCACUIAGAA | 1550 |
| AM14452-SS-NT | (NAG37)s(invAb)scguuuuggAfUfGfcacuiagacas(invAb) | 916 | CGUUUUGGAUGCACUIAGACA | 1551 |
| AM14454-SS-NT | (NAG37)s(invAb)sauuccucgGfUfAfuuuauugucus(invAb) | 917 | AUUCCUCGGUAUUUAUUGUCU | 1552 |
| AM14456-SS-NT | (NAG37)s(invAb)sccucgguaUfUfUfauuguuuguas(invAb) | 918 | CCUCGGUAUUUAUUGUUUGUA | 1553 |
| AM14547-SS-NT | (NH2-C6)s(invAb)sgcauuccuCfGfGfuauuuauugus(invAb) | 919 | GCAUUCCUCGGUAUUUAUUGU | 1532 |
| | (C6-SS-C6) | | | |
| AM14549-SS-NT | (NH2-C6)s(invAb)sccuggucaUfGfGfaguauuacgus(invAb) | 920 | CCUGGUCAUGGAGUAUUACGU | 1523 |
| | (C6-SS-C6) | | | |
| AM14616-SS-NT | (NH2-C6)s(invAb)sguccucggUfAfUfuuauugucuas(invAb) | 921 | GUCCUCGGUAUUUAUUGUCUA | 1591 |
| | (C6-SS-C6) | | | |
| AM14618-SS-NT | (NH2-C6)s(invAb)suuccucggUfaUfuUfauugucuas(invAb) | 922 | UUCCUCGGUAUUUAUUGUCUA | 1543 |
| | (C6-SS-C6) | | | |
| AM14619-SS-NT | (NAG37)s(invAb)sccuggucaUfgGfaGfuauuacgus(invAb) | 923 | CCUGGUCAUGGAGUAUUACGU | 1523 |
| AM14621-SS-NT | (NAG37)s(invAb)sccuggucaUfgGfaguauuacgus(invAb) | 924 | CCUGGUCAUGGAGUAUUACGU | 1523 |
| AM14668-SS-NT | (NH2-C6)s(invAb)sccccgacaUfUfCfcucgguauuas(invAb) | 925 | CCCCGACAUUCCUCGGUAUUA | 1542 |
| AM14669-SS-NT | (NH2-C6)s(invAb)suuccucggUfAfUfuuauugucuas(invAb) | 926 | UUCCUCGGUAUUUAUUGUCUA | 1543 |
| AM15036-SS-NT | (NAG37)s(invAb)suggcaagaUfcGfuccacuacaas(invAb) | 927 | UGGCAAGAUCGUCCACUACAA | 1525 |
| AM15040-SS-NT | (NAG37)s(invAb)suggcaaGfaUfcGfuccacuacaas(invAb) | 928 | UGGCAAGAUCGUCCACUACAA | 1525 |
| AM15276-SS-NT | (NH2-C6)s(invAb)sguccucggUfaUfuUfauugucuas(invAb) | 929 | GUCCUCGGUAUUUAUUGUCUA | 1591 |
| | (C6-SS-C6) | | | |
| AM15283-SS-NT | (NH2-C6)s(invAb)suuccucggUfaUfUfuauugucuas(invAb) | 930 | UUCCUCGGUAUUUAUUGUCUA | 1543 |
| | (C6-SS-C6) | | | |
| AM15476-SS-NT | (NH2-C6)s(invAb)sacaaguggGfAfCfaugcuiaagas(invAb) | 931 | ACAAGUGGGACAUGCUIAAGA | 1592 |
| | (C6-SS-C6) | | | |
| AM15478-SS-NT | (NH2-C6)s(invAb)scaagugggAfCfAfugcuiaagaas(invAb) | 932 | CAAGUGGGACAUGCUIAAGAA | 1593 |
| | (C6-SS-C6) | | | |
| AM15480-SS-NT | (NH2-C6)s(invAb)scuuccaggAfUfGfagaacuacuus(invAb) | 933 | CUUCCAGGAUGAGAACUACUU | 1594 |
| | (C6-SS-C6) | | | |
| AM15482-SS-NT | (NH2-C6)s(invAb)scuuccaggAfUfGfagaacuaucus(invAb) | 934 | CUUCCAGGAUGAGAACUAUCU | 1595 |
| | (C6-SS-C6) | | | |
| AM15483-SS-NT | (NH2-C6)s(invAb)succaggauGfAfGfaacuaccuius(invAb) | 935 | UCCAGGAUGAGAACUACCUIU | 1596 |
| | (C6-SS-C6) | | | |
| AM15485-SS-NT | (NH2-C6)s(invAb)succaggauGfAfGfaacuacuugus(invAb) | 936 | UCCAGGAUGAGAACUACUUGU | 1597 |
| | (C6-SS-C6) | | | |
| AM15486-SS-NT | (NH2-C6)s(invAb)saggaugagAfAfCfuaccuiuacas(invAb) | 937 | AGGAUGAGAACUACCUIUACA | 1598 |
| | (C6-SS-C6) | | | |
| AM15488-SS-NT | (NH2-C6)s(invAb)scuaccuguAfCfCfuggucauigas(invAb) | 938 | CUACCUGUACCUGGUCAUIGA | 1599 |
| | (C6-SS-C6) | | | |
| AM15490-SS-NT | (NH2-C6)s(invAb)scuaccuguAfCfCfugguuauggas(invAb) | 939 | CUACCUGUACCUGGUUAUGGA | 1600 |
| | (C6-SS-C6) | | | |
| AM15491-SS-NT | (NH2-C6)s(invAb)sacacugcuGfAfGfcaaguuuigas(invAb) | 940 | ACACUGCUGAGCAAGUUUIGA | 1601 |
| | (C6-SS-C6) | | | |
| AM15510-SS-NT | (NAG37)s(invAb)scuacaaggAfGfCfaccucuuucus(invAb) | 941 | CUACAAGGAGCACCUCUUUCU | 1567 |
| AM15512-SS-NT | (NAG37)s(invAb)scuacaaggAfGfCfaccuuucucus(invAb) | 942 | CUACAAGGAGCACCUUUCUCU | 1568 |
| AM15513-SS-NT | (NAG37)s(invAb)scggacacaUfCfCfcuucuucuuus(invAb) | 943 | CGGACACAUCCCUUCUUCUUU | 1569 |
| AM15515-SS-NT | (NAG37)s(invAb)sgacacaucCfCfUfucuucuuugas(invAb) | 944 | GACACAUCCCUUCUUCUUUGA | 1570 |
| AM15517-SS-NT | (NAG37)s(invAb)sccuuuacaCfCfGfgauuucgaaas(invAb) | 945 | CCUUUACACCGGAUUUCGAAA | 1571 |
| AM15519-SS-NT | (NAG37)s(invAb)suacaccggAfUfUfucgaagiugas(invAb) | 946 | UACACCGGAUUUCGAAGIUGA | 1572 |
| AM15521-SS-NT | (NAG37)s(invAb)scaccgacaCfAfUfgcaacuucias(invAb) | 947 | CACCGACACAUGCAACUUCIA | 1573 |
| AM15523-SS-NT | (NAG37)s(invAb)sccgacacaUfGfCfaacuuciacus(invAb) | 948 | CCGACACAUGCAACUUCIACU | 1574 |
| AM15525-SS-NT | (NAG37)s(invAb)sacaugcaaCfUfUfcgacuuiguas(invAb) | 949 | ACAUGCAACUUCGACUUIGUA | 1575 |
| AM15836-SS-NT | (NAG37)s(invAb)sccuggucaUfgGfAfguauuacgus(invAb) | 950 | CCUGGUCAUGGAGUAUUACGU | 1523 |
| AM16408-SS-NT | (NH2-C6)s(invAb)sccucgguaUfUfUfauuguuuguas(invAb) | 951 | CCUCGGUAUUUAUUGUUUGUA | 1552 |
| | (C6-SS-C6) | | | |
| AM16410-SS-NT | (NH2-C6)s(invAb)sccucgguaUfUfUfauugucuguas(invAb) | 952 | CCUCGGUAUUUAUUGUCUGUA | 1602 |
| | (C6-SS-C6) | | | |
| AM16411-SS-NT | (NH2-C6)s(invAb)sccucgguaUfUfUfauugucuiuas(invAb) | 953 | CCUCGGUAUUUAUUGUCUIUA | 1603 |
| | (C6-SS-C6) | | | |
| AM16539-SS-NT | (NAG37)s(invAb)sccuggucaUfgGfAfguauuacgas(invAb) | 954 | CCUGGUCAUGGAGUAUUACGA | 1579 |
| AM16573-SS-NT | (NAG37)cscscuagAfAfCfugucuucgsasa | 955 | CCCUAGAACUGUCUUCGAA | 1580 |
| AM16576-SS-NT | (NAG37)s(invAb)suuccucggUfaUfUfuauugucuas(invAb) | 956 | UUCCUCGGUAUUUAUUGUCUA | 1543 |
| AM16701-SS-NT | (NAG37)s(invAb)scaccgacaCfaUfgCfaacuucias(invAb) | 957 | CACCGACACAUGCAACUUCIA | 1573 |
| AM16802-SS-NT | (NH2-C6)s(invAb)sgacccuagAfAfCfugucuuciaas(invAb) | 958 | GACCCUAGAACUGUCUUCIAA | 1514 |
| | (C6-SS-C6) | | | |
| AM16804-SS-NT | (NH2-C6)s(invAb)sgaacccuaGfAfAfcugucuucias(invAb) | 1959 | GAACCCUAGAACUGUCUUCIA | 1513 |
| | (C6-SS-C6) | | | |
| AM16907-SS-NT | (NAG37)s(invAb)suggcaagaUfcGfUfccacuacaas(invAb) | 960 | UGGCAAGAUCGUCCACUACAA | 1525 |
| AM16908-SS-NT | (NAG37)s(invAb)suggcaagaUfcGfuCfcacuacaas(invAb) | 961 | UGGCAAGAUCGUCCACUACAA | 1525 |
| AM16963-SS-NT | (NH2-C6)s(invAb)sccuggucaUfgGfAfguauuacgas(invAb) | 962 | CCUGGUCAUGGAGUAUUACGA | 1579 |
| | (C6-SS-C6) | | | |
| AM17081-SS-NT | (NH2-C6)cscscuagAfAfCfugucuucgsasa(invAb) | 963 | CCCUAGAACUGUCUUCGAA | 1580 |
| | (C6-SS-C6) | | | |
| AM17650-SS-NT | (NH2-C6)s(invAb)sccucgguaUfuUfaUfugucuguas(invAb) | 964 | CCUCGGUAUUUAUUGUCUGUA | 1602 |
| | (C6-SS-C6) | | | |
| AM17651-SS-NT | (NH2-C6)s(invAb)sccucgguaUfuUfAfuugucuguas(invAb) | 965 | CCUCGGUAUUUAUUGUCUGUA | 1602 |
| | (C6-SS-C6) | | | |
| AM18528-SS-NT | (NH2-C6)s(invAb)suuccucggUfaUfUfuauugucuas(invAb) | 966 | UUCCUCGGUAUUUAUUGUCUA | 1543 |
| AM19347-SS-NT | (NH2-C6)s(invAb)sccuggucaUfgGfAfguauuacgas(invAb) | 967 | CCUGGUCAUGGAGUAUUACGA | 1579 |
| AM19421-SS-NT | (NH2-C6)s(invAb)suuccucggUfaUfUfuauugucuaAlk | 968 | UUCCUCGGUAUUUAUUGUCUA | 1585 |
| AM19422-SS-NT | (NH2-C6)s(invAb)suuccucggUfaUfUfuauugucusaAlk | 969 | UUCCUCGGUAUUUAUUGUCUA | 1585 |
| AM19573-SS-NT | (NH2- | 970 | UUCCUCGGUAUUUAUUGUCUA | 1543 |
| | C6)s(invAb)suuccucggUfaUfUfuauugucuas(invAb)s | | | |
| | (C6-SS-C6) | | | |
| AM20339-SS-NT | NH2-C6s(invAb)sccuggucaUfgGfAfguauuacga(invAb) | 971 | CCUGGUCAUGGAGUAUUACGA | 1579 |
| | (C6-SS-C6) | | | |
| AM20340-SS-NT | NH2-C6s(invAb)sccugggucaUfgGfAfguauuacgas(invAb) | 972 | CCUGGGUCAUGGAGUAUUACGA | 1604 |
| | (C6-SS-C6) | | | |
| AM20342-SS-NT | NH2-C6s(invAb)scuggucaUfgGfAfguauuacgas(invAb) | 974 | CUGGUCAUGGAGUAUUACGA | 1605 |
| | (C6-SS-C6) | | | |

(A^{2N}) represents a 2-aminoadenine nucleotide; I represents an inosine (hypoxanthine) nucleotide

TABLE 4.3
DMPK RNAi Agent Sense Strand Sequences Shown Without Terminal Linking Groups or Targeting Groups

| | | | Underlying Base Sequence | |
|---|---|---|---|---|
| | | Seq | (5′ → 3′) | Seq |
| | | ID | (Shown as an Unmodified | ID |
| Sense Strand ID | Modified Sense Strand (5′ → 3′) | No. | Nucleotide Sequence) | No. |
| AM12526-SS-NT-NL | (invAb)sggaugaaaCfAfGfcugaaguigas(invAb) | 976 | GGAUGAAACAGCUGAAGUIGA | 1511 |
| AM12528-SS-NT-NL | (invAb)succcugaaCfCfCfuagaacuguas(invAb) | 977 | UCCCUGAACCCUAGAACUGUA | 1512 |
| AM12530-SS-NT-NL | (invAb)sgaacccuaGfAfAfcugucuucias(invAb) | 978 | GAACCCUAGAACUGUCUUCIA | 1513 |
| AM12532-SS-NT-NL | (invAb)sgacccuagAfAfCfugucuuciaas(invAb) | 979 | GACCCUAGAACUGUCUUCIAA | 1514 |
| AM12534-SS-NT-NL | (invAb)sacccuagaAfCfUfgucuuciacus(invAb) | 980 | ACCCUAGAACUGUCUUCIACU | 1515 |
| AM12536-SS-NT-NL | (invAb)scccuagaaCfUfGfucuuciacuas(invAb) | 981 | CCCUAGAACUGUCUUCIACUA | 1516 |
| AM12538-SS-NT-NL | (invAb)sugcuuacuCfGfGfgaaauuugcus(invAb) | 982 | UGCUUACUCGGGAAAUUUGCU | 1517 |
| AM12540-SS-NT-NL | (invAb)scgggaaauUfUfGfcuuuuiccaas(invAb) | 983 | CGGGAAAUUUGCUUUUICCAA | 1518 |
| AM12542-SS-NT-NL | (invAb)saccccaauCfCfAfcguuuuggaus(invAb) | 984 | ACCCCAAUCCACGUUUUGGAU | 1519 |
| AM12544-SS-NT-NL | (invAb)scccaauccAfCfGfuuuugiaugas(invAb) | 985 | CCCAAUCCACGUUUUGIAUGA | 1520 |
| AM13805-SS-NT-NL | (invAb)sgagggacgUfGfUfugguiaaugas(invAb) | 986 | GAGGGACGUGUUGGUIAAUGA | 1521 |
| AM13807-SS-NT-NL | (invAb)sggaugagaAfCfUfaccuiuaccus(invAb) | 987 | GGAUGAGAACUACCUIUACCU | 1522 |
| AM13809-SS-NT-NL | (invAb)sccuggucaUfGfGfaguauuacgus(invAb) | 988 | CCUGGUCAUGGAGUAUUACGU | 1523 |
| AM13811-SS-NT-NL | (invAb)sggacaucaAfAfCfccgacaacaus(invAb) | 989 | GGACAUCAAACCCGACAACAU | 1524 |
| AM13814-SS-NT-NL | (invAb)suggcaagaUfCfGfuccacuacaas(invAb) | 990 | UGGCAAGAUCGUCCACUACAA | 1525 |
| AM13817-SS-NT-NL | (invAb)sccccuuuaCfAfCfcggauuucgas(invAb) | 991 | CCCCUUUACACCGGAUUUCGA | 1526 |
| AM13819-SS-NT-NL | (invAb)scgacacauGfCfAfacuuciacuus(invAb) | 992 | CGACACAUGCAACUUCIACUU | 1527 |
| AM13821-SS-NT-NL | (invAb)sggaacccuAfGfAfacugucuucas(invAb) | 993 | GGAACCCUAGAACUGUCUUCA | 1528 |
| AM13823-SS-NT-NL | (invAb)sggaacccuAfGfAfacuguuuucas(invAb) | 994 | GGAACCCUAGAACUGUUUUCA | 1529 |
| AM13824-SS-NT-NL | (invAb)scugcuuacUfCfGfggaaauuugas(invAb) | 995 | CUGCUUACUCGGGAAAUUUGA | 1530 |
| AM13826-SS-NT-NL | (invAb)sgcgguuugGfa_2NUfaUfuuauugaas(invAb) | 996 | GCGGUUUGG(A^{2N})UAUUUAUUGAA | 1531 |
| AM13852-SS-NT-NL | (invAb)sgcauuccuCfGfGfuauuuauugus(invAb) | 997 | GCAUUCCUCGGUAUUUAUUGU | 1532 |
| AM13854-SS-NT-NL | (invAb)sgcauuccuCfGfGfuauuua_2Nuugus(invAb) | 998 | GCAUUCCUCGGUAUUU(A^{2N})UUGU | 1533 |
| AM13855-SS-NT-NL | (invAb)sgacccucgCfGfAfauaaaagicas(invAb) | 999 | GACCCUCGCGAAUAAAAGICA | 1534 |
| AM14132-SS-NT-NL | (invAb)saggggcgaGfGfUfgucgugcuuas(invAb) | 1000 | AGGGGCGAGGUGUCGUGCUUA | 1541 |
| AM14134-SS-NT-NL | (invAb)sggcagacgCfCfCfuucuacgcgas(invAb) | 1001 | GGCAGACGCCCUUCUACGCGA | 1588 |
| AM14136-SS-NT-NL | (invAb)sggcagaccCfCfCfuucuacgcgas(invAb) | 1002 | GGCAGACCCCCUUCUACGCGA | 1589 |
| AM14138-SS-NT-NL | (invAb)sccccgacaUfUfCfcucgguauuas(invAb) | 1003 | CCCCGACAUUCCUCGGUAUUA | 1542 |
| AM14140-SS-NT-NL | (invAb)suuccucggUfAfUfuuauugucuas(invAb) | 1004 | UUCCUCGGUAUUUAUUGUCUA | 1543 |
| AM14142-SS-NT-NL | (invAb)sgcgaggUfAfGfcgguagugaas(invAb) | 1005 | GCGAGGUAGCGGUAGUGAA | 1590 |
| AM14151-SS-NT-NL | (invAb)saggggcgaGfGfUfgucgugcuuas(invAb) | 1006 | AGGGGCGAGGUGUCGUGCUUA | 1541 |
| AM14155-SS-NT-NL | (invAb)sccccgacaUfUfCfcucgguauuas(invAb) | 1007 | CCCCGACAUUCCUCGGUAUUA | 1542 |
| AM14157-SS-NT-NL | (invAb)suuccucggUfAfUfuuauugucuas(invAb) | 1008 | UUCCUCGGUAUUUAUUGUCUA | 1543 |
| AM14438-SS-NT-NL | (invAb)sacucgggaAfAfUfuugcuuuugas(invAb) | 1009 | ACUCGGGAAAUUUGCUUUUGA | 1544 |
| AM14440-SS-NT-NL | (invAb)sggaaauuuGfCfUfuuugucaaaas(invAb) | 1010 | GGAAAUUUGCUUUUGUCAAAA | 1545 |
| AM14442-SS-NT-NL | (invAb)sucggcgguUfUfGfgauauuua_2Nuus(invAb) | 1011 | UCGGCGGUUUGGAUAUUU(A^{2N})UU | 1546 |
| AM14444-SS-NT-NL | (invAb)scggcgguuUfGfGfauauuua_2Nuuas(invAb) | 1012 | CGGCGGUUUGGAUAUUU(A^{2N})UUA | 1547 |
| AM14446-SS-NT-NL | (invAb)saaccccaaUfCfCfacguuuuggas(invAb) | 1013 | AACCCCAAUCCACGUUUUGGA | 1548 |
| AM14448-SS-NT-NL | (invAb)sgauccacgUfUfUfuggauicacus(invAb) | 1014 | GAUCCACGUUUUGGAUICACU | 1549 |
| AM14450-SS-NT-NL | (invAb)sacguuuugGfAfUfgcacuiagaas(invAb) | 1015 | ACGUUUUGGAUGCACUIAGAA | 1550 |
| AM14452-SS-NT-NL | (invAb)scguuuuggAfUfGfcacuiagacas(invAb) | 1016 | CGUUUUGGAUGCACUIAGACA | 1551 |
| AM14454-SS-NT-NL | (invAb)sauuccucgGfUfAfuuuauugucus(invAb) | 1017 | AUUCCUCGGUAUUUAUUGUCU | 1552 |
| AM14456-SS-NT-NL | (invAb)sccucgguaUfUfUfauuguuuguas(invAb) | 1018 | CCUCGGUAUUUAUUGUUUGUA | 1553 |
| AM14547-SS-NT-NL | (invAb)sgcauuccuCfGfGfuauuuauugus(invAb) | 1019 | GCAUUCCUCGGUAUUUAUUGU | 1532 |
| AM14549-SS-NT-NL | (invAb)sccuggucaUfGfGfaguauuacgus(invAb) | 1020 | CCUGGUCAUGGAGUAUUACGU | 1523 |
| AM14616-SS-NT-NL | (invAb)sguccucggUfAfUfuuauugucuas(invAb) | 1021 | GUCCUCGGUAUUUAUUGUCUA | 1591 |
| AM14618-SS-NT-NL | (invAb)suuccucggUfaUfuUfauugucuas(invAb) | 1022 | UUCCUCGGUAUUUAUUGUCUA | 1543 |
| AM14619-SS-NT-NL | (invAb)sccuggucaUfgGfaGfuauuacgus(invAb) | 1023 | CCUGGUCAUGGAGUAUUACGU | 1523 |
| AM14621-SS-NT-NL | (invAb)sccuggucaUfgGfaguauuacgus(invAb) | 1024 | CCUGGUCAUGGAGUAUUACGU | 1523 |
| AM14668-SS-NT-NL | (invAb)sccccgacaUfUfCfcucgguauuas(invAb) | 1025 | CCCCGACAUUCCUCGGUAUUA | 1542 |
| AM14669-SS-NT-NL | (invAb)suuccucggUfAfUfuuauugucuas(invAb) | 1026 | UUCCUCGGUAUUUAUUGUCUA | 1543 |
| AM15036-SS-NT-NL | (invAb)suggcaagaUfcGfuccacuacaas(invAb) | 1027 | UGGCAAGAUCGUCCACUACAA | 1525 |
| AM15040-SS-NT-NL | (invAb)suggcaaGfaUfcGfuccacuacaas(invAb) | 1028 | UGGCAAGAUCGUCCACUACAA | 1525 |
| AM15276-SS-NT-NL | (invAb)sguccucggUfaUfuUfauugucuas(invAb) | 1029 | GUCCUCGGUAUUUAUUGUCUA | 1591 |
| AM15283-SS-NT-NL | (invAb)suuccucggUfaUfUfuauugucuas(invAb) | 1030 | UUCCUCGGUAUUUAUUGUCUA | 1543 |
| AM15476-SS-NT-NL | (invAb)sacaaguggGfAfCfaugcuiaagas(invAb) | 1031 | ACAAGUGGGACAUGCUIAAGA | 1592 |
| AM15478-SS-NT-NL | (invAb)scaagugggAfCfAfugcuiaagaas(invAb) | 1032 | CAAGUGGGACAUGCUIAAGAA | 1593 |
| AM15480-SS-NT-NL | (invAb)scuuccaggAfUfGfagaacuacuus(invAb) | 1033 | CUUCCAGGAUGAGAACUACUU | 1594 |
| AM15482-SS-NT-NL | (invAb)scuuccaggAfUfGfagaacuaucus(invAb) | 1034 | CUUCCAGGAUGAGAACUAUCU | 1595 |
| AM15483-SS-NT-NL | (invAb)succaggauGfAfGfaacuaccuius(invAb) | 1035 | UCCAGGAUGAGAACUACCUIU | 1596 |
| AM15485-SS-NT-NL | (invAb)succaggauGfAfGfaacuacuugus(invAb) | 1036 | UCCAGGAUGAGAACUACUUGU | 1597 |
| AM15486-SS-NT-NL | (invAb)saggaugagAfAfCfuaccuiuacas(invAb) | 1037 | AGGAUGAGAACUACCUIUACA | 1598 |
| AM15488-SS-NT-NL | (invAb)scuaccuguAfCfCfuggucauigas(invAb) | 1038 | CUACCUGUACCUGGUCAUIGA | 1599 |
| AM15490-SS-NT-NL | (invAb)scuaccuguAfCfCfugguuauggas(invAb) | 1039 | CUACCUGUACCUGGUUAUGGA | 1600 |
| AM15491-SS-NT-NL | (invAb)sacacugcuGfAfGfcaaguuuigas(invAb) | 1040 | ACACUGCUGAGCAAGUUUIGA | 1601 |
| AM15510-SS-NT-NL | (invAb)scuacaaggAfGfCfaccucuuucus(invAb) | 1041 | CUACAAGGAGCACCUCUUUCU | 1567 |
| AM15512-SS-NT-NL | (invAb)scuacaaggAfGfCfaccuuucucus(invAb) | 1042 | CUACAAGGAGCACCUUUCUCU | 1568 |
| AM15513-SS-NT-NL | (invAb)scggacacaUfCfCfcuucuucuuus(invAb) | 1043 | CGGACACAUCCCUUCUUCUUU | 1569 |
| AM15515-SS-NT-NL | (invAb)sgacacaucCfCfUfucuucuuugas(invAb) | 1044 | GACACAUCCCUUCUUCUUUGA | 1570 |
| AM15517-SS-NT-NL | (invAb)sccuuuacaCfCfGfgauuucgaaas(invAb) | 1045 | CCUUUACACCGGAUUUCGAAA | 1571 |
| AM15519-SS-NT-NL | (invAb)suacaccggAfUfUfucgaagiugas(invAb) | 1046 | UACACCGGAUUUCGAAGIUGA | 1572 |
| AM15521-SS-NT-NL | (invAb)scaccgacaCfAfUfgcaacuucias(invAb) | 1047 | CACCGACACAUGCAACUUCIA | 1573 |
| AM15523-SS-NT-NL | (invAb)sccgacacaUfGfCfaacuuciacus(invAb) | 1048 | CCGACACAUGCAACUUCIACU | 1574 |
| AM15525-SS-NT-NL | (invAb)sacaugcaaCfUfUfcgacuuiguas(invAb) | 1049 | ACAUGCAACUUCGACUUIGUA | 1575 |
| AM15836-SS-NT-NL | (invAb)sccuggucaUfgGfAfguauuacgus(invAb) | 1050 | CCUGGUCAUGGAGUAUUACGU | 1523 |
| AM16408-SS-NT-NL | (invAb)sccucgguaUfUfUfauuguuuguas(invAb) | 1051 | CCUCGGUAUUUAUUGUUUGUA | 1553 |
| AM16410-SS-NT-NL | (invAb)sccucgguaUfUfUfauugucuguas(invAb) | 1052 | CCUCGGUAUUUAUUGUCUGUA | 1602 |
| AM16411-SS-NT-NL | (invAb)sccucgguaUfUfUfauugucuiuas(invAb) | 1053 | CCUCGGUAUUUAUUGUCUIUA | 1603 |
| AM16539-SS-NT-NL | (invAb)sccuggucaUfgGfAfguauuacgas(invAb) | 1054 | CCUGGUCAUGGAGUAUUACGA | 1579 |
| AM16573-SS-NT-NL | cscscuagAfAfCfugucuucgsasa | 1055 | CCCUAGAACUGUCUUCGAA | 1580 |
| AM16576-SS-NT-NL | (invAb)suuccucggUfaUfUfuauugucuas(invAb) | 1056 | UUCCUCGGUAUUUAUUGUCUA | 1543 |
| AM16701-SS-NT-NL | (invAb)scaccgacaCfaUfgCfaacuucias(invAb) | 1057 | CACCGACACAUGCAACUUCIA | 1573 |
| AM16802-SS-NT-NL | (invAb)sgacccuagAfAfCfugucuuciaas(invAb) | 1058 | GACCCUAGAACUGUCUUCIAA | 1514 |
| AM16804-SS-NT-NL | (invAb)sgaacccuaGfAfAfcugucuucias(invAb) | 1059 | GAACCCUAGAACUGUCUUCIA | 1513 |
| AM16907-SS-NT-NL | (invAb)suggcaagaUfcGfUfccacuacaas(invAb) | 1060 | UGGCAAGAUCGUCCACUACAA | 1525 |
| AM16908-SS-NT-NL | (invAb)suggcaagaUfcGfuCfcacuacaas(invAb) | 1061 | UGGCAAGAUCGUCCACUACAA | 1525 |
| AM16963-SS-NT-NL | (invAb)sccuggucaUfgGfAfguauuacgas(invAb) | 1062 | CCUGGUCAUGGAGUAUUACGA | 1579 |
| AM17081-SS-NT-NL | cscscuagAfAfCfugucuucgsasa(invAb) | 1063 | CCCUAGAACUGUCUUCGAA | 1580 |
| AM17650-SS-NT-NL | (invAb)sccucgguaUfuUfaUfugucuguas(invAb) | 1064 | CCUCGGUAUUUAUUGUCUGUA | 1602 |
| AM17651-SS-NT-NL | (invAb)sccucgguaUfuUfAfuugucuguas(invAb) | 1065 | CCUCGGUAUUUAUUGUCUGUA | 1602 |
| AM18528-SS-NT-NL | (invAb)suuccucggUfaUfUfuauugucuas(invAb) | 1066 | UUCCUCGGUAUUUAUUGUCUA | 1543 |
| AM19347-SS-NT-NL | (invAb)sccuggucaUfgGfAfguauuacgas(invAb) | 1067 | CCUGGUCAUGGAGUAUUACGA | 1579 |
| AM19421-SS-NT-NL | (invAb)suuccucggUfaUfUfuauugucuaAlk | 1068 | UUCCUCGGUAUUUAUUGUCUA | 1585 |
| AM19422-SS-NT-NL | (invAb)suuccucggUfaUfUfuauugucusaAlk | 1069 | UUCCUCGGUAUUUAUUGUCUA | 1585 |
| AM19573-SS-NT-NL | (invAb)suuccucggUfaUfUfuauugucuas(invAb)s | 1070 | UUCCUCGGUAUUUAUUGUCUA | 1543 |
| AM20339-SS-NT-NL | (invAb)sccuggucaUfgGfAfguauuacga(invAb) | 1071 | CCUGGUCAUGGAGUAUUACGA | 1579 |
| AM20340-SS-NT-NL | (invAb)sccugggucaUfgGfAfguauuacgas(invAb) | 1072 | CCUGGGUCAUGGAGUAUUACGA | 1604 |
| AM20342-SS-NT-NL | (invAb)scuggucaUfgGfAfguauuacgas(invAb) | 1074 | CUGGUCAUGGAGUAUUACGA | 1605 |

(A^{2N}) represents a 2-aminoadenine nucleotide; I represents an inosine (hypoxanthine) nucleotide

TABLE 4.4
DMPK RNAi Agent Sense Strand Sequences Shown As Modified Nucleotide Sequence Only (Without
Terminal Linking Groups Or Inverted Abasic Capping Moieties)

| | | | Underlying Base Sequence | |
|---|---|---|---|---|
| | | Seq | (5′ → 3′) | Seq |
| | | ID | (Shown as an Unmodified | ID |
| Sense Strand ID | Modified Sense Strand (5′ → 3′) | No | Nucleotide Sequence) | No |
| AM12526-SS-NT-NLA | ggaugaaaCfAfGfcugaaguiga | 1076 | GGAUGAAACAGCUGAAGUIGA | 1511 |
| AM12528-SS-NT-NLA | ucccugaaCfCfCfuagaacugua | 1077 | UCCCUGAACCCUAGAACUGUA | 1512 |
| AM12530-SS-NT-NLA | gaacccuaGfAfAfcugucuucia | 1078 | GAACCCUAGAACUGUCUUCIA | 1513 |
| AM12532-SS-NT-NLA | gacccuagAfAfCfugucuuciaa | 1079 | GACCCUAGAACUGUCUUCIAA | 1514 |
| AM12534-SS-NT-NLA | acccuagaAfCfUfgucuuciacu | 1080 | ACCCUAGAACUGUCUUCIACU | 1515 |
| AM12536-SS-NT-NLA | cccuagaaCfUfGfucuuciacua | 1081 | CCCUAGAACUGUCUUCIACUA | 1516 |
| AM12538-SS-NT-NLA | ugcuuacuCfGfGfgaaauuugcu | 1082 | UGCUUACUCGGGAAAUUUGCU | 1517 |
| AM12540-SS-NT-NLA | cgggaaauUfUfGfcuuuuiccaa | 1083 | CGGGAAAUUUGCUUUUICCAA | 1518 |
| AM12542-SS-NT-NLA | accccaauCfCfAfcguuuuggau | 1084 | ACCCCAAUCCACGUUUUGGAU | 1519 |
| AM12544-SS-NT-NLA | cccaauccAfCfGfuuuugiauga | 1085 | CCCAAUCCACGUUUUGIAUGA | 1520 |
| AM13805-SS-NT-NLA | gagggacgUfGfUfugguiaauga | 1086 | GAGGGACGUGUUGGUIAAUGA | 1521 |
| AM13807-SS-NT-NLA | ggaugagaAfCfUfaccuiuaccu | 1087 | GGAUGAGAACUACCUIUACCU | 1522 |
| AM13809-SS-NT-NLA | ccuggucaUfGfGfaguauuacgu | 1088 | CCUGGUCAUGGAGUAUUACGU | 1523 |
| AM13811-SS-NT-NLA | ggacaucaAfAfCfccgacaacau | 1089 | GGACAUCAAACCCGACAACAU | 1524 |
| AM13814-SS-NT-NLA | uggcaagaUfCfGfuccacuacaa | 1090 | UGGCAAGAUCGUCCACUACAA | 1525 |
| AM13817-SS-NT-NLA | ccccuuuaCfAfCfcggauuucga | 1091 | CCCCUUUACACCGGAUUUCGA | 1526 |
| AM13819-SS-NT-NLA | cgacacauGfCfAfacuuciacuu | 1092 | CGACACAUGCAACUUCIACUU | 1527 |
| AM13821-SS-NT-NLA | ggaacccuAfGfAfacugucuuca | 1093 | GGAACCCUAGAACUGUCUUCA | 1528 |
| AM13823-SS-NT-NLA | ggaacccuAfGfAfacuguuuuca | 1094 | GGAACCCUAGAACUGUUUUCA | 1529 |
| AM13824-SS-NT-NLA | cugcuuacUfCfGfggaaauuuga | 1095 | CUGCUUACUCGGGAAAUUUGA | 1530 |
| AM13826-SS-NT-NLA | gcgguuugGfa_2NUfaUfuuauugaa | 1096 | GCGGUUUGG(A^{2N})UAUUUAUUGAA | 1531 |
| AM13852-SS-NT-NLA | gcauuccuCfGfGfuauuuauugu | 1097 | GCAUUCCUCGGUAUUUAUUGU | 1532 |
| AM13854-SS-NT-NLA | gcauuccuCfGfGfuauuua_2Nuugu | 1098 | GCAUUCCUCGGUAUUU(A^{2N})UUGU | 1533 |
| AM13855-SS-NT-NLA | gacccucgCfGfAfauaaaagica | 1099 | GACCCUCGCGAAUAAAAGICA | 1534 |
| AM14132-SS-NT-NLA | aggggcgaGfGfUfgucgugcuua | 1100 | AGGGGCGAGGUGUCGUGCUUA | 1541 |
| AM14134-SS-NT-NLA | ggcagacgCfCfCfuucuacgcga | 1101 | GGCAGACGCCCUUCUACGCGA | 1588 |
| AM14136-SS-NT-NLA | ggcagaccCfCfCfuucuacgcga | 1102 | GGCAGACCCCCUUCUACGCGA | 1589 |
| AM14138-SS-NT-NLA | ccccgacaUfUfCfcucgguauua | 1103 | CCCCGACAUUCCUCGGUAUUA | 1542 |
| AM14140-SS-NT-NLA | uuccucggUfAfUfuuauugucua | 1104 | UUCCUCGGUAUUUAUUGUCUA | 1543 |
| AM14142-SS-NT-NLA | gcgaggUfAfGfcgguagugaa | 1105 | GCGAGGUAGCGGUAGUGAA | 1590 |
| AM14151-SS-NT-NLA | aggggcgaGfGfUfgucgugcuua | 1106 | AGGGGCGAGGUGUCGUGCUUA | 1541 |
| AM14155-SS-NT-NLA | ccccgacaUfUfCfcucgguauua | 1107 | CCCCGACAUUCCUCGGUAUUA | 1542 |
| AM14157-SS-NT-NLA | uuccucggUfAfUfuuauugucua | 1108 | UUCCUCGGUAUUUAUUGUCUA | 1543 |
| AM14438-SS-NT-NLA | acucgggaAfAfUfuugcuuuuga | 1109 | ACUCGGGAAAUUUGCUUUUGA | 1544 |
| AM14440-SS-NT-NLA | ggaaauuuGfCfUfuuugucaaaa | 1110 | GGAAAUUUGCUUUUGUCAAAA | 1545 |
| AM14442-SS-NT-NLA | ucggcgguUfUfGfgauauuua_2Nuu | 1111 | UCGGCGGUUUGGAUAUUU(A^{2N})UU | 1546 |
| AM14444-SS-NT-NLA | cggcgguuUfGfGfauauuua_2Nuua | 1112 | CGGCGGUUUGGAUAUUU(A^{2N})UUA | 1547 |
| AM14446-SS-NT-NLA | aaccccaaUfCfCfacguuuugga | 1113 | AACCCCAAUCCACGUUUUGGA | 1548 |
| AM14448-SS-NT-NLA | gauccacgUfUfUfuggauicacu | 1114 | GAUCCACGUUUUGGAUICACU | 1549 |
| AM14450-SS-NT-NLA | acguuuugGfAfUfgcacuiagaa | 1115 | ACGUUUUGGAUGCACUIAGAA | 1550 |
| AM14452-SS-NT-NLA | cguuuuggAfUfGfcacuiagaca | 1116 | CGUUUUGGAUGCACUIAGACA | 1551 |
| AM14454-SS-NT-NLA | auuccucgGfUfAfuuuauugucu | 1117 | AUUCCUCGGUAUUUAUUGUCU | 1552 |
| AM14456-SS-NT-NLA | ccucgguaUfUfUfauuguuugua | 1118 | CCUCGGUAUUUAUUGUUUGUA | 1553 |
| AM14547-SS-NT-NLA | gcauuccuCfGfGfuauuuauugu | 1119 | GCAUUCCUCGGUAUUUAUUGU | 1532 |
| AM14549-SS-NT-NLA | ccuggucaUfGfGfaguauuacgu | 1120 | CCUGGUCAUGGAGUAUUACGU | 1523 |
| AM14616-SS-NT-NLA | guccucggUfAfUfuuauugucua | 1121 | GUCCUCGGUAUUUAUUGUCUA | 1591 |
| AM14618-SS-NT-NLA | uuccucggUfaUfuUfauugucua | 1122 | UUCCUCGGUAUUUAUUGUCUA | 1543 |
| AM14619-SS-NT-NLA | ccuggucaUfgGfaGfuauuacgu | 1123 | CCUGGUCAUGGAGUAUUACGU | 1523 |
| AM14621-SS-NT-NLA | ccuggucaUfgGfaguauuacgu | 1124 | CCUGGUCAUGGAGUAUUACGU | 1523 |
| AM14668-SS-NT-NLA | ccccgacaUfUfCfcucgguauua | 1125 | CCCCGACAUUCCUCGGUAUUA | 1542 |
| AM14669-SS-NT-NLA | uuccucggUfAfUfuuauugucua | 1126 | UUCCUCGGUAUUUAUUGUCUA | 1543 |
| AM15036-SS-NT-NLA | uggcaagaUfcGfuccacuacaa | 1127 | UGGCAAGAUCGUCCACUACAA | 1525 |
| AM15040-SS-NT-NLA | uggcaaGfaUfcGfuccacuacaa | 1128 | UGGCAAGAUCGUCCACUACAA | 1525 |
| AM15276-SS-NT-NLA | guccucggUfaUfuUfauugucua | 1129 | GUCCUCGGUAUUUAUUGUCUA | 1591 |
| AM15283-SS-NT-NLA | uuccucggUfaUfUfuauugucua | 1130 | UUCCUCGGUAUUUAUUGUCUA | 1543 |
| AM15476-SS-NT-NLA | acaaguggGfAfCfaugcuiaaga | 1131 | ACAAGUGGGACAUGCUIAAGA | 1592 |
| AM15478-SS-NT-NLA | caagugggAfCfAfugcuiaagaa | 1132 | CAAGUGGGACAUGCUIAAGAA | 1593 |
| AM15480-SS-NT-NLA | cuuccaggAfUfGfagaacuacuu | 1133 | CUUCCAGGAUGAGAACUACUU | 1594 |
| AM15482-SS-NT-NLA | cuuccaggAfUfGfagaacuaucu | 1134 | CUUCCAGGAUGAGAACUAUCU | 1595 |
| AM15483-SS-NT-NLA | uccaggauGfAfGfaacuaccuiu | 1135 | UCCAGGAUGAGAACUACCUIU | 1596 |
| AM15485-SS-NT-NLA | uccaggauGfAfGfaacuacuugu | 1136 | UCCAGGAUGAGAACUACUUGU | 1597 |
| AM15486-SS-NT-NLA | aggaugagAfAfCfuaccuiuaca | 1137 | AGGAUGAGAACUACCUIUACA | 1598 |
| AM15488-SS-NT-NLA | cuaccuguAfCfCfuggucauiga | 1138 | CUACCUGUACCUGGUCAUIGA | 1599 |
| AM15490-SS-NT-NLA | cuaccuguAfCfCfugguuaugga | 1139 | CUACCUGUACCUGGUUAUGGA | 1600 |
| AM15491-SS-NT-NLA | acacugcuGfAfGfcaaguuuiga | 1140 | ACACUGCUGAGCAAGUUUIGA | 1601 |
| AM15510-SS-NT-NLA | cuacaaggAfGfCfaccucuuucu | 1141 | CUACAAGGAGCACCUCUUUCU | 1567 |
| AM15512-SS-NT-NLA | cuacaaggAfGfCfaccuuucucu | 1142 | CUACAAGGAGCACCUUUCUCU | 1568 |
| AM15513-SS-NT-NLA | cggacacaUfCfCfcuucuucuuu | 1143 | CGGACACAUCCCUUCUUCUUU | 1569 |
| AM15515-SS-NT-NLA | gacacaucCfCfUfucuucuuuga | 1144 | GACACAUCCCUUCUUCUUUGA | 1570 |
| AM15517-SS-NT-NLA | ccuuuacaCfCfGfgauuucgaaa | 1145 | CCUUUACACCGGAUUUCGAAA | 1571 |
| AM15519-SS-NT-NLA | uacaccggAfUfUfucgaagiuga | 1146 | UACACCGGAUUUCGAAGIUGA | 1572 |
| AM15521-SS-NT-NLA | caccgacaCfAfUfgcaacuucia | 1147 | CACCGACACAUGCAACUUCIA | 1573 |
| AM15523-SS-NT-NLA | ccgacacaUfGfCfaacuuciacu | 1148 | CCGACACAUGCAACUUCIACU | 1574 |
| AM15525-SS-NT-NLA | acaugcaaCfUfUfcgacuuigua | 1149 | ACAUGCAACUUCGACUUIGUA | 1575 |
| AM15836-SS-NT-NLA | ccuggucaUfgGfAfguauuacgu | 1150 | CCUGGUCAUGGAGUAUUACGU | 1523 |
| AM16408-SS-NT-NLA | ccucgguaUfUfUfauuguuugua | 1151 | CCUCGGUAUUUAUUGUUUGUA | 1553 |
| AM16410-SS-NT-NLA | ccucgguaUfUfUfauugucugua | 1152 | CCUCGGUAUUUAUUGUCUGUA | 1602 |
| AM16411-SS-NT-NLA | ccucgguaUfUfUfauugucuiua | 1153 | CCUCGGUAUUUAUUGUCUIUA | 1603 |
| AM16539-SS-NT-NLA | ccuggucaUfgGfAfguauuacga | 1154 | CCUGGUCAUGGAGUAUUACGA | 1579 |
| AM16573-SS-NT-NLA | cscscuagAfAfCfugucuucgsasa | 1155 | CCCUAGAACUGUCUUCGAA | 1580 |
| AM16576-SS-NT-NLA | uuccucggUfaUfUfuauugucua | 1156 | UUCCUCGGUAUUUAUUGUCUA | 1543 |
| AM16701-SS-NT-NLA | caccgacaCfaUfgCfaacuucia | 1157 | CACCGACACAUGCAACUUCIA | 1573 |
| AM16802-SS-NT-NLA | gacccuagAfAfCfugucuuciaa | 1158 | GACCCUAGAACUGUCUUCIAA | 1514 |
| AM16804-SS-NT-NLA | gaacccuaGfAfAfcugucuucia | 1159 | GAACCCUAGAACUGUCUUCIA | 1513 |
| AM16907-SS-NT-NLA | uggcaagaUfcGfUfccacuacaa | 1160 | UGGCAAGAUCGUCCACUACAA | 1525 |
| AM16908-SS-NT-NLA | uggcaagaUfcGfuCfcacuacaa | 1161 | UGGCAAGAUCGUCCACUACAA | 1525 |
| AM16963-SS-NT-NLA | ccuggucaUfgGfAfguauuacga | 1162 | CCUGGUCAUGGAGUAUUACGA | 1579 |
| AM17081-SS-NT-NLA | cscscuagAfAfCfugucuucgsasa | 1163 | CCCUAGAACUGUCUUCGAA | 1580 |
| AM17650-SS-NT-NLA | ccucgguaUfuUfaUfugucugua | 1164 | CCUCGGUAUUUAUUGUCUGUA | 1602 |
| AM17651-SS-NT-NLA | ccucgguaUfuUfAfuugucugua | 1165 | CCUCGGUAUUUAUUGUCUGUA | 1602 |
| AM18528-SS-NT-NLA | uuccucggUfaUfUfuauugucua | 1166 | UUCCUCGGUAUUUAUUGUCUA | 1543 |
| AM19347-SS-NT-NLA | ccuggucaUfgGfAfguauuacga | 1167 | CCUGGUCAUGGAGUAUUACGA | 1579 |
| AM19421-SS-NT-NLA | uuccucggUfaUfUfuauugucuaAlk | 1168 | UUCCUCGGUAUUUAUUGUCUA | 1585 |
| AM19422-SS-NT-NLA | uuccucggUfaUfUfuauugucusaAlk | 1169 | UUCCUCGGUAUUUAUUGUCUA | 1585 |
| AM19573-SS-NT-NLA | uuccucggUfaUfUfuauugucuas | 1170 | UUCCUCGGUAUUUAUUGUCUA | 1543 |
| AM20339-SS-NT-NLA | ccuggucaUfgGfAfguauuacga | 1171 | CCUGGUCAUGGAGUAUUACGA | 1579 |
| AM20340-SS-NT-NLA | ccugggucaUfgGfAfguauuacga | 1172 | CCUGGGUCAUGGAGUAUUACGA | 1604 |
| AM20342-SS-NT-NLA | cuggucaUfgGfAfguauuacga | 1174 | CUGGUCAUGGAGUAUUACGA | 1605 |

(A^{2N}) represents a 2-aminoadenine nucleotide; I represents an inosine (hypoxanthine) nucleotide

As discussed herein, in some embodiments, one or more targeting ligands and/or PK/PD modulators are linked or conjugated to the RNAi agent. In some embodiments, a targeting ligand (or targeting group) and/or a PK/PD modulator is linked to the 5′ end of the sense strand, the 3′ end of the sense strand, and/or to one or more internal nucleotides. The synthesis of the sense strand and/or the antisense strand can be designed such that reactive groups are readily available to facilitate linkage to additional components, such as a targeting ligand or PK/PD modulator. The following Table 4.5 depicts the sense strand of the DMPK RNAi agents disclosed above in Table 4.1 after linking to one or more targeting ligands and/or PK/PD modulators (collectively, shown below, as Z). Pharmacological moieties are linked to the DMPK RNAi agents using reactions described in Example 1, below. Following conjugation to targeting ligands, the linking groups may have the structure (NH—C6), (NH—C6) s, or (C6-S), the structure of each of which is shown in Table 6.1, below.

TABLE 4.5
DMPK RNAi Agent Sense Strand Sequences Showing Targeting Ligand and/or
PK/PD modulator Positions (Z = pharmacological moiety (e.g., targeting ligand, targeting
group, and/or PK/PD modulator))

| | | Seq ID |
|---|---|---|
| Sense Strand ID | Modified Sense Strand (5′ → 3′) | No. |
| AM12526-SS-Z | Z-(invAb)sggaugaaaCfAfGfcugaaguigas(invAb)-Z | 1176 |
| AM12528-SS-Z | Z-(invAb)succcugaaCfCfCfuagaacuguas(invAb)-Z | 1177 |
| AM12530-SS-Z | Z-(invAb)sgaacccuaGfAfAfcugucuucias(invAb)-Z | 1178 |
| AM12532-SS-Z | Z-(invAb)sgacccuagAfAfCfugucuuciaas(invAb)-Z | 1179 |
| AM12534-SS-Z | Z-(invAb)sacccuagaAfCfUfgucuuciacus(invAb)-Z | 1180 |
| AM12536-SS-Z | Z-(invAb)scccuagaaCfUfGfucuuciacuas(invAb)-Z | 1181 |
| AM12538-SS-Z | Z-(invAb)sugcuuacuCfGfGfgaaauuugcus(invAb)-Z | 1182 |
| AM12540-SS-Z | Z-(invAb)scgggaaauUfUfGfcuuuuiccaas(invAb)-Z | 1183 |
| AM12542-SS-Z | Z-(invAb)saccccaauCfCfAfcguuuuggaus(invAb)-Z | 1184 |
| AM12544-SS-Z | Z-(invAb)scccaauccAfCfGfuuuugiaugas(invAb)-Z | 1185 |
| AM13805-SS-Z | Z-(invAb)sgagggacgUfGfUfugguiaaugas(invAb)-Z | 1186 |
| AM13807-SS-Z | Z-(invAb)sggaugagaAfCfUfaccuiuaccus(invAb)-Z | 1187 |
| AM13809-SS-Z | Z-(invAb)sccuggucaUfGfGfaguauuacgus(invAb)-Z | 1188 |
| AM13811-SS-Z | Z-(invAb)sggacaucaAfAfCfccgacaacaus(invAb)-Z | 1189 |
| AM13813-SS-Z | Z-(invAb)sggacaucaAfAfCfccgauaacaus(invAb)-Z | 1190 |
| AM13814-SS-Z | Z-(invAb)suggcaagaUfCfGfuccacuacaas(invAb)-Z | 1191 |
| AM13816-SS-Z | Z-(invAb)suggcaagaUfCfGfuccauuacaas(invAb)-Z | 1192 |
| AM13817-SS-Z | Z-(invAb)sccccuuuaCfAfCfcggauuucgas(invAb)-Z | 1193 |
| AM13819-SS-Z | Z-(invAb)scgacacauGfCfAfacuuciacuus(invAb)-Z | 1194 |
| AM13821-SS-Z | Z-(invAb)sggaacccuAfGfAfacugucuucas(invAb)-Z | 1195 |
| AM13823-SS-Z | Z-(invAb)sggaacccuAfGfAfacuguuuucas(invAb)-Z | 1196 |
| AM13824-SS-Z | Z-(invAb)scugcuuacUfCfGfggaaauuugas(invAb)-Z | 1197 |
| AM13826-SS-Z | Z-(invAb)sgcgguuugGfa_2NUfaUfuuauugaas(invAb)-Z | 1198 |
| AM13852-SS-Z | Z-(invAb)sgcauuccuCfGfGfuauuuauugus(invAb)-Z | 1199 |
| AM13854-SS-Z | Z-(invAb)sgcauuccuCfGfGfuauuua_2Nuugus(invAb)-Z | 1200 |
| AM13855-SS-Z | Z-(invAb)sgacccucgCfGfAfauaaaagicas(invAb)-Z | 1201 |
| AM14132-SS-Z | Z-(NH-C6)s(invAb)saggggcgaGfGfUfgucgugcuuas(invAb)(C6-S)-Z | 1202 |
| AM14134-SS-Z | Z-(NH-C6)s(invAb)sggcagacgCfCfCfuucuacgcgas(invAb)(C6-S)-Z | 1203 |
| AM14136-SS-Z | Z-(NH-C6)s(invAb)sggcagaccCfCfCfuucuacgcgas(invAb)(C6-S)-Z | 1204 |
| AM14138-SS-Z | Z-(NH-C6)s(invAb)sccccgacaUfUfCfcucgguauuas(invAb)(C6-S)-Z | 1205 |
| AM14140-SS-Z | Z-(NH-C6)s(invAb)suuccucggUfAfUfuuauugucuas(invAb)(C6-S)-Z | 1206 |
| AM14142-SS-Z | Z-(invAb)sgcgaggUfAfGfcgguagugaadTdTs(invAb)-Z | 1207 |
| AM14144-SS-Z | Z-(invAb)sgcacuuCfGfCfcuuccaggaudTdTs(invAb)-Z | 1208 |
| AM14147-SS-Z | Z-(invAb)scagcgaggUfAfGfcgguagugaas(invAb)-Z | 1209 |
| AM14149-SS-Z | Z-(invAb)scugcacuuCfGfCfcuuccaggaus(invAb)-Z | 1210 |
| AM14151-SS-Z | Z-(invAb)saggggcgaGfGfUfgucgugcuuas(invAb)-Z | 1211 |
| AM14153-SS-Z | Z-(invAb)sggcagacgCfCfCfuucuacgcgas(invAb)-Z | 1212 |
| AM14155-SS-Z | Z-(invAb)sccccgacaUfUfCfcucgguauuas(invAb)-Z | 1213 |
| AM14157-SS-Z | Z-(invAb)suuccucggUfAfUfuuauugucuas(invAb)-Z | 1214 |
| AM14438-SS-Z | Z-(invAb)sacucgggaAfAfUfuugcuuuugas(invAb)-Z | 1215 |
| AM14440-SS-Z | Z-(invAb)sggaaauuuGfCfUfuuugucaaaas(invAb)-Z | 1216 |
| AM14442-SS-Z | Z-(invAb)sucggcgguUfUfGfgauauuua_2Nuus(invAb)-Z | 1217 |
| AM14444-SS-Z | Z-(invAb)scggcgguuUfGfGfauauuua_2Nuuas(invAb)-Z | 1218 |
| AM14446-SS-Z | Z-(invAb)saaccccaaUfCfCfacguuuuggas(invAb)-Z | 1219 |
| AM14448-SS-Z | Z-(invAb)sgauccacgUfUfUfuggauicacus(invAb)-Z | 1220 |
| AM14450-SS-Z | Z-(invAb)sacguuuugGfAfUfgcacuiagaas(invAb)-Z | 1221 |
| AM14452-SS-Z | Z-(invAb)scguuuuggAfUfGfcacuiagacas(invAb)-Z | 1222 |
| AM14454-SS-Z | Z-(invAb)sauuccucgGfUfAfuuuauugucus(invAb)-Z | 1223 |
| AM14456-SS-Z | Z-(invAb)sccucgguaUfUfUfauuguuuguas(invAb)-Z | 1224 |
| AM14458-SS-Z | Z-(invAb)scucgguauUfUfAfuugucuiucas(invAb)-Z | 1225 |
| AM14460-SS-Z | Z-(invAb)sgauugucuGfUfCfcccaucuagas(invAb)-Z | 1226 |
| AM14547-SS-Z | Z-(NH-C6)s(invAb)sgcauuccuCfGfGfuauuuauugus(invAb)(C6-S)-Z | 1227 |
| AM14549-SS-Z | Z-(NH-C6)s(invAb)sccuggucaUfGfGfaguauuacgus(invAb)(C6-S)-Z | 1228 |
| AM14616-SS-Z | Z-(NH-C6)s(invAb)sguccucggUfAfUfuuauugucuas(invAb)(C6-S)-Z | 1229 |
| AM14618-SS-Z | Z-(NH-C6)s(invAb)suuccucggUfaUfuUfauugucuas(invAb)(C6-S)-Z | 1230 |
| AM14619-SS-Z | Z-(invAb)sccuggucaUfgGfaGfuauuacgus(invAb)-Z | 1231 |
| AM14621-SS-Z | Z-(invAb)sccuggucaUfgGfaguauuacgus(invAb)-Z | 1232 |
| AM14668-SS-Z | Z-(NH-C6)s(invAb)sccccgacaUfUfCfcucgguauuas(invAb)-Z | 1233 |
| AM14669-SS-Z | Z-(NH-C6)s(invAb)suuccucggUfAfUfuuauugucuas(invAb)-Z | 1234 |
| AM15036-SS-Z | Z-(invAb)suggcaagaUfcGfuccacuacaas(invAb)-Z | 1235 |
| AM15040-SS-Z | Z-(invAb)suggcaaGfaUfcGfuccacuacaas(invAb)-Z | 1236 |
| AM15276-SS-Z | Z-(NH-C6)s(invAb)sguccucggUfaUfuUfauugucuas(invAb)(C6-S)-Z | 1237 |
| AM15283-SS-Z | Z-(NH-C6)s(invAb)suuccucggUfaUfUfuauugucuas(invAb)(C6-S)-Z | 1238 |
| AM15476-SS-Z | Z-(NH-C6)s(invAb)sacaaguggGfAfCfaugcuiaagas(invAb)(C6-S)-Z | 1239 |
| AM15478-SS-Z | Z-(NH-C6)s(invAb)scaagugggAfCfAfugcuiaagaas(invAb)(C6-S)-Z | 1240 |
| AM15480-SS-Z | Z-(NH-C6)s(invAb)scuuccaggAfUfGfagaacuacuus(invAb)(C6-S)-Z | 1241 |
| AM15482-SS-Z | Z-(NH-C6)s(invAb)scuuccaggAfUfGfagaacuaucus(invAb)(C6-S)-Z | 1242 |
| AM15483-SS-Z | Z-(NH-C6)s(invAb)succaggauGfAfGfaacuaccuius(invAb)(C6-S)-Z | 1243 |
| AM15485-SS-Z | Z-(NH-C6)s(invAb)succaggauGfAfGfaacuacuugus(invAb)(C6-S)-Z | 1244 |
| AM15486-SS-Z | Z-(NH-C6)s(invAb)saggaugagAfAfCfuaccuiuacas(invAb)(C6-S)-Z | 1245 |
| AM15488-SS-Z | Z-(NH-C6)s(invAb)scuaccuguAfCfCfuggucauigas(invAb)(C6-S)-Z | 1246 |
| AM15490-SS-Z | Z-(NH-C6)s(invAb)scuaccuguAfCfCfugguuauggas(invAb)(C6-S)-Z | 1247 |
| AM15491-SS-Z | Z-(NH-C6)s(invAb)sacacugcuGfAfGfcaaguuuigas(invAb)(C6-S)-Z | 1248 |
| AM15493-SS-Z | Z-(invAb)sugccaugaAfGfAfucaugaacaas(invAb)-Z | 1249 |
| AM15495-SS-Z | Z-(invAb)sccaugaagAfUfCfaugaacaagus(invAb)-Z | 1250 |
| AM15497-SS-Z | Z-(invAb)scaugaagaUfCfAfugaacaaguas(invAb)-Z | 1251 |
| AM15499-SS-Z | Z-(invAb)sugcugacaCfUfGfcugaicaagus(invAb)-Z | 1252 |
| AM15501-SS-Z | Z-(invAb)sugacacugCfUfGfagcaaguuuas(invAb)-Z | 1253 |
| AM15503-SS-Z | Z-(invAb)sgcaucaaaCfCfCfgacaacaucas(invAb)-Z | 1254 |
| AM15505-SS-Z | Z-(invAb)sgcaaacccGfAfCfaacauccuias(invAb)-Z | 1255 |
| AM15507-SS-Z | Z-(invAb)sgcaaacccGfAfCfaacaucuugas(invAb)-Z | 1256 |
| AM15508-SS-Z | Z-(invAb)scaagaucgUfCfCfacuacaaigas(invAb)-Z | 1257 |
| AM15510-SS-Z | Z-(invAb)scuacaaggAfGfCfaccucuuucus(invAb)-Z | 1258 |
| AM15512-SS-Z | Z-(invAb)scuacaaggAfGfCfaccuuucucus(invAb)-Z | 1259 |
| AM15513-SS-Z | Z-(invAb)scggacacaUfCfCfcuucuucuuus(invAb)-Z | 1260 |
| AM15515-SS-Z | Z-(invAb)sgacacaucCfCfUfucuucuuugas(invAb)-Z | 1261 |
| AM15517-SS-Z | Z-(invAb)sccuuuacaCfCfGfgauuucgaaas(invAb)-Z | 1262 |
| AM15519-SS-Z | Z-(invAb)suacaccggAfUfUfucgaagiugas(invAb)-Z | 1263 |
| AM15521-SS-Z | Z-(invAb)scaccgacaCfAfUfgcaacuucias(invAb)-Z | 1264 |
| AM15523-SS-Z | Z-(invAb)sccgacacaUfGfCfaacuuciacus(invAb)-Z | 1265 |
| AM15525-SS-Z | Z-(invAb)sacaugcaaCfUfUfcgacuuiguas(invAb)-Z | 1266 |
| AM15836-SS-Z | Z-(invAb)sccuggucaUfgGfAfguauuacgus(invAb)-Z | 1267 |
| AM16315-SS-Z | Z-(invAb)scaccgacaCfAfUfgcaacuuugas(invAb)-Z | 1268 |
| AM16318-SS-Z | Z-(invAb)scaccgacaCfaUfgcaacuucias(invAb)-Z | 1269 |
| AM16322-SS-Z | Z-(invAb)scaccgaCfaCfaUfgcaacuucias(invAb)-Z | 1270 |
| AM16408-SS-Z | Z-(NH-C6)s(invAb)sccucgguaUfUfUfauuguuuguas(invAb)(C6-S)-Z | 1271 |
| AM16410-SS-Z | Z-(NH-C6)s(invAb)sccucgguaUfUfUfauugucuguas(invAb)(C6-S)-Z | 1272 |
| AM16411-SS-Z | Z-(NH-C6)s(invAb)sccucgguaUfUfUfauugucuiuas(invAb)(C6-S)-Z | 1273 |
| AM16539-SS-Z | Z-(invAb)sccuggucaUfgGfAfguauuacgas(invAb)-Z | 1274 |
| AM16573-SS-Z | Z-cscscuagAfAfCfugucuucgsasa-Z | 1275 |
| AM16576-SS-Z | Z-(invAb)suuccucggUfaUfUfuauugucuas(invAb)-Z | 1276 |
| AM16701-SS-Z | Z-(invAb)scaccgacaCfaUfgCfaacuucias(invAb)-Z | 1277 |
| AM16802-SS-Z | Z-(NH-C6)s(invAb)sgacccuagAfAfCfugucuuciaas(invAb)(C6-S)-Z | 1278 |
| AM16804-SS-Z | Z-(NH-C6)s(invAb)sgaacccuaGfAfAfcugucuucias(invAb)(C6-S)-Z | 1279 |
| AM16907-SS-Z | Z-(invAb)suggcaagaUfcGfUfccacuacaas(invAb)-Z | 1280 |
| AM16908-SS-Z | Z-(invAb)suggcaagaUfcGfuCfcacuacaas(invAb)-Z | 1281 |
| AM16963-SS-Z | Z-(NH-C6)s(invAb)sccuggucaUfgGfAfguauuacgas(invAb)(C6-S)-Z | 1282 |
| AM17081-SS-Z | Z-(NH-C6)cscscuagAfAfCfugucuucgsasa(invAb)(C6-S)-Z | 1283 |
| AM17650-SS-Z | Z-(NH-C6)s(invAb)sccucgguaUfuUfaUfugucuguas(invAb)(C6-S)-Z | 1284 |
| AM17651-SS-Z | Z-(NH-C6)s(invAb)sccucgguaUfuUfAfuugucuguas(invAb)(C6-S)-Z | 1285 |
| AM18528-SS-Z | Z-(NH-C6)s(invAb)suuccucggUfaUfUfuauugucuas(invAb)-Z | 1286 |
| AM19347-SS-Z | Z-(NH-C6)s(invAb)sccuggucaUfgGfAfguauuacgas(invAb)-Z | 1287 |
| AM19421-SS-Z | Z-(NH-C6)s(invAb)suuccucggUfaUfUfuauugucuaAlk-Z | 1288 |
| AM19422-SS-Z | Z-(NH-C6)s(invAb)suuccucggUfaUfUfuauugucusaAlk-Z | 1289 |
| AM19573-SS-Z | Z-(NH-C6)s(invAb)suuccucggUfaUfUfuauugucuas(invAb)s(C6-S)-Z | 1290 |

TABLE 4.6
dMPK RNAi Agent Sense Strand Sequences Showing Targeting Ligand linked at
the 5′ terminal end and PK/PD modulator linked at the 3′ terminal end of the sense strand.
(TL = targeting ligand; PK = PK/PD modulator))

| | | Seq ID |
|---|---|---|
| Sense Strand ID | Modified Sense Strand (5′ → 3′) | No. |
| AM12526-SS-TP | TL-(invAb)sggaugaaaCfAfGfcugaaguigas(invAb)-PK | 1291 |
| AM12528-SS-TP | TL-(invAb)succcugaaCfCfCfuagaacuguas(invAb)-PK | 1292 |
| AM12530-SS-TP | TL-(invAb)sgaacccuaGfAfAfcugucuucias(invAb)-PK | 1293 |
| AM12532-SS-TP | TL-(invAb)sgacccuagAfAfCfugucuuciaas(invAb)-PK | 1294 |
| AM12534-SS-TP | TL-(invAb)sacccuagaAfCfUfgucuuciacus(invAb)-PK | 1295 |
| AM12536-SS-TP | TL-(invAb)scccuagaaCfUfGfucuuciacuas(invAb)-PK | 1296 |
| AM12538-SS-TP | TL-(invAb)sugcuuacuCfGfGfgaaauuugcus(invAb)-PK | 1297 |
| AM12540-SS-TP | TL-(invAb)scgggaaauUfUfGfcuuuuiccaas(invAb)-PK | 1298 |
| AM12542-SS-TP | TL-(invAb)saccccaauCfCfAfcguuuuggaus(invAb)-PK | 1299 |
| AM12544-SS-TP | TL-(invAb)scccaauccAfCfGfuuuugiaugas(invAb)-PK | 1300 |
| AM13805-SS-TP | TL-(invAb)sgagggacgUfGfUfugguiaaugas(invAb)-PK | 1301 |
| AM13807-SS-TP | TL-(invAb)sggaugagaAfCfUfaccuiuaccus(invAb)-PK | 1302 |
| AM13809-SS-TP | TL-(invAb)sccuggucaUfGfGfaguauuacgus(invAb)-PK | 1303 |
| AM13811-SS-TP | TL-(invAb)sggacaucaAfAfCfccgacaacaus(invAb)-PK | 1304 |
| AM13813-SS-TP | TL-(invAb)sggacaucaAfAfCfccgauaacaus(invAb)-PK | 1305 |
| AM13814-SS-TP | TL-(invAb)suggcaagaUfCfGfuccacuacaas(invAb)-PK | 1306 |
| AM13816-SS-TP | TL-(invAb)suggcaagaUfCfGfuccauuacaas(invAb)-PK | 1307 |
| AM13817-SS-TP | TL-(invAb)sccccuuuaCfAfCfcggauuucgas(invAb)-PK | 1308 |
| AM13819-SS-TP | TL-(invAb)scgacacauGfCfAfacuuciacuus(invAb)-PK | 1309 |
| AM13821-SS-TP | TL-(invAb)sggaacccuAfGfAfacugucuucas(invAb)-PK | 1310 |
| AM13823-SS-TP | TL-(invAb)sggaacccuAfGfAfacuguuuucas(invAb)-PK | 1311 |
| AM13824-SS-TP | TL-(invAb)scugcuuacUfCfGfggaaauuugas(invAb)-PK | 1312 |
| AM13826-SS-TP | TL-(invAb)sgcgguuugGfa_2NUfaUfuuauugaas(invAb)-PK | 1313 |
| AM13852-SS-TP | TL-(invAb)sgcauuccuCfGfGfuauuuauugus(invAb)-PK | 1314 |
| AM13854-SS-TP | TL-(invAb)sgcauuccuCfGfGfuauuua_2Nuugus(invAb)-PK | 1315 |
| AM13855-SS-TP | TL-(invAb)sgacccucgCfGfAfauaaaagicas(invAb)-PK | 1316 |
| AM14132-SS-TP | TL-(NH-C6)s(invAb)saggggcgaGfGfUfgucgugcuuas(invAb)(C6-S)- | 1317 |
| | PK | |
| AM14134-SS-TP | TL-(NH-C6)s(invAb)sggcagacgCfCfCfuucuacgcgas(invAb)(C6-S)- | 1318 |
| | PK | |
| AM14136-SS-TP | TL-(NH-C6)s(invAb)sggcagaccCfCfCfuucuacgcgas(invAb)(C6-S)- | 1319 |
| | PK | |
| AM14138-SS-TP | TL-(NH-C6)s(invAb)sccccgacaUfUfCfcucgguauuas(invAb)(C6-S)- | 1320 |
| | PK | |
| AM14140-SS-TP | TL-(NH-C6)s(invAb)suuccucggUfAfUfuuauugucuas(invAb)(C6-S)- | 1321 |
| | PK | |
| AM14142-SS-TP | TL-(invAb)sgcgaggUfAfGfcgguagugaadTdTs(invAb)-PK | 1322 |
| AM14144-SS-TP | TL-(invAb)sgcacuuCfGfCfcuuccaggaudTdTs(invAb)-PK | 1323 |
| AM14147-SS-TP | TL-(invAb)scagcgaggUfAfGfcgguagugaas(invAb)-PK | 1324 |
| AM14149-SS-TP | TL-(invAb)scugcacuuCfGfCfcuuccaggaus(invAb)-PK | 1325 |
| AM14151-SS-TP | TL-(invAb)saggggcgaGfGfUfgucgugcuuas(invAb)-PK | 1326 |
| AM14153-SS-TP | TL-(invAb)sggcagacgCfCfCfuucuacgcgas(invAb)-PK | 1327 |
| AM14155-SS-TP | TL-(invAb)sccccgacaUfUfCfcucgguauuas(invAb)-PK | 1328 |
| AM14157-SS-TP | TL-(invAb)suuccucggUfAfUfuuauugucuas(invAb)-PK | 1329 |
| AM14438-SS-TP | TL-(invAb)sacucgggaAfAfUfuugcuuuugas(invAb)-PK | 1330 |
| AM14440-SS-TP | TL-(invAb)sggaaauuuGfCfUfuuugucaaaas(invAb)-PK | 1331 |
| AM14442-SS-TP | TL-(invAb)sucggcgguUfUfGfgauauuua_2Nuus(invAb)-PK | 1332 |
| AM14444-SS-TP | TL-(invAb)scggcgguuUfGfGfauauuua_2Nuuas(invAb)-PK | 1333 |
| AM14446-SS-TP | TL-(invAb)saaccccaaUfCfCfacguuuuggas(invAb)-PK | 1334 |
| AM14448-SS-TP | TL-(invAb)sgauccacgUfUfUfuggauicacus(invAb)-PK | 1335 |
| AM14450-SS-TP | TL-(invAb)sacguuuugGfAfUfgcacuiagaas(invAb)-PK | 1336 |
| AM14452-SS-TP | TL-(invAb)scguuuuggAfUfGfcacuiagacas(invAb)-PK | 1337 |
| AM14454-SS-TP | TL-(invAb)sauuccucgGfUfAfuuuauugucus(invAb)-PK | 1338 |
| AM14456-SS-TP | TL-(invAb)sccucgguaUfUfUfauuguuuguas(invAb)-PK | 1339 |
| AM14458-SS-TP | TL-(invAb)scucgguauUfUfAfuugucuiucas(invAb)-PK | 1340 |
| AM14460-SS-TP | TL-(invAb)sgauugucuGfUfCfcccaucuagas(invAb)-PK | 1341 |
| AM14547-SS-TP | TL-(NH-C6)s(invAb)sgcauuccuCfGfGfuauuuauugus(invAb)(C6-S)- | 1342 |
| | PK | |
| AM14549-SS-TP | TL-(NH-C6)s(invAb)sccuggucaUfGfGfaguauuacgus(invAb)(C6-S)- | 1343 |
| | PK | |
| AM14616-SS-TP | TL-(NH-C6)s(invAb)sguccucggUfAfUfuuauugucuas(invAb)(C6-S)- | 1344 |
| | PK | |
| AM14618-SS-TP | TL-(NH-C6)s(invAb)suuccucggUfaUfuUfauugucuas(invAb)(C6-S)- | 1345 |
| | PK | |
| AM14619-SS-TP | TL-(invAb)sccuggucaUfgGfaGfuauuacgus(invAb)-PK | 1346 |
| AM14621-SS-TP | TL-(invAb)sccuggucaUfgGfaguauuacgus(invAb)-PK | 1347 |
| AM14668-SS-TP | TL-(NH-C6)s(invAb)sccccgacaUfUfCfcucgguauuas(invAb)-PK | 1348 |
| AM14669-SS-TP | TL-(NH-C6)s(invAb)suuccucggUfAfUfuuauugucuas(invAb)-PK | 1349 |
| AM15036-SS-TP | TL-(invAb)suggcaagaUfcGfuccacuacaas(invAb)-PK | 1350 |
| AM15040-SS-TP | TL-(invAb)suggcaaGfaUfcGfuccacuacaas(invAb)-PK | 1351 |
| AM15276-SS-TP | TL-(NH-C6)s(invAb)sguccucggUfaUfuUfauugucuas(invAb)(C6-S)- | 1352 |
| | PK | |
| AM15283-SS-TP | TL-(NH-C6)s(invAb)suuccucggUfaUfUfuauugucuas(invAb)(C6-S)- | 1353 |
| | PK | |
| AM15476-SS-TP | TL-(NH-C6)s(invAb)sacaaguggGfAfCfaugcuiaagas(invAb)(C6-S)- | 1354 |
| | PK | |
| AM15478-SS-TP | TL-(NH-C6)s(invAb)scaagugggAfCfAfugcuiaagaas(invAb)(C6-S)- | 1355 |
| | PK | |
| AM15480-SS-TP | TL-(NH-C6)s(invAb)scuuccaggAfUfGfagaacuacuus(invAb)(C6-S)- | 1356 |
| | PK | |
| AM15482-SS-TP | TL-(NH-C6)s(invAb)scuuccaggAfUfGfagaacuaucus(invAb)(C6-S)- | 1357 |
| | PK | |
| AM15483-SS-TP | TL-(NH-C6)s(invAb)succaggauGfAfGfaacuaccuius(invAb)(C6-S)- | 1358 |
| | PK | |
| AM15485-SS-TP | TL-(NH-C6)s(invAb)succaggauGfAfGfaacuacuugus(invAb)(C6-S)- | 1359 |
| | PK | |
| AM15486-SS-TP | TL-(NH-C6)s(invAb)saggaugagAfAfCfuaccuiuacas(invAb)(C6-S)- | 1360 |
| | PK | |
| AM15488-SS-TP | TL-(NH-C6)s(invAb)scuaccuguAfCfCfuggucauigas(invAb)(C6-S)- | 1361 |
| | PK | |
| AM15490-SS-TP | TL-(NH-C6)s(invAb)scuaccuguAfCfCfugguuauggas(invAb)(C6-S)- | 1362 |
| | PK | |
| AM15491-SS-TP | TL-(NH-C6)s(invAb)sacacugcuGfAfGfcaaguuuigas(invAb)(C6-S)- | 1363 |
| | PK | |
| AM15493-SS-TP | TL-(invAb)sugccaugaAfGfAfucaugaacaas(invAb)-PK | 1364 |
| AM15495-SS-TP | TL-(invAb)sccaugaagAfUfCfaugaacaagus(invAb)-PK | 1365 |
| AM15497-SS-TP | TL-(invAb)scaugaagaUfCfAfugaacaaguas(invAb)-PK | 1366 |
| AM15499-SS-TP | TL-(invAb)sugcugacaCfUfGfcugaicaagus(invAb)-PK | 1367 |
| AM15501-SS-TP | TL-(invAb)sugacacugCfUfGfagcaaguuuas(invAb)-PK | 1368 |
| AM15503-SS-TP | TL-(invAb)sgcaucaaaCfCfCfgacaacaucas(invAb)-PK | 1369 |
| AM15505-SS-TP | TL-(invAb)sgcaaacccGfAfCfaacauccuias(invAb)-PK | 1370 |
| AM15507-SS-TP | TL-(invAb)sgcaaacccGfAfCfaacaucuugas(invAb)-PK | 1371 |
| AM15508-SS-TP | TL-(invAb)scaagaucgUfCfCfacuacaaigas(invAb)-PK | 1372 |
| AM15510-SS-TP | TL-(invAb)scuacaaggAfGfCfaccucuuucus(invAb)-PK | 1373 |
| AM15512-SS-TP | TL-(invAb)scuacaaggAfGfCfaccuuucucus(invAb)-PK | 1374 |
| AM15513-SS-TP | TL-(invAb)scggacacaUfCfCfcuucuucuuus(invAb)-PK | 1375 |
| AM15515-SS-TP | TL-(invAb)sgacacaucCfCfUfucuucuuugas(invAb)-PK | 1376 |
| AM15517-SS-TP | TL-(invAb)sccuuuacaCfCfGfgauuucgaaas(invAb)-PK | 1377 |
| AM15519-SS-TP | TL-(invAb)suacaccggAfUfUfucgaagiugas(invAb)-PK | 1378 |
| AM15521-SS-TP | TL-(invAb)scaccgacaCfAfUfgcaacuucias(invAb)-PK | 1379 |
| AM15523-SS-TP | TL-(invAb)sccgacacaUfGfCfaacuuciacus(invAb)-PK | 1380 |
| AM15525-SS-TP | TL-(invAb)sacaugcaaCfUfUfcgacuuiguas(invAb)-PK | 1381 |
| AM15836-SS-TP | TL-(invAb)sccuggucaUfgGfAfguauuacgus(invAb)-PK | 1382 |
| AM16315-SS-TP | TL-(invAb)scaccgacaCfAfUfgcaacuuugas(invAb)-PK | 1383 |
| AM16318-SS-TP | TL-(invAb)scaccgacaCfaUfgcaacuucias(invAb)-PK | 1384 |
| AM16322-SS-TP | TL-(invAb)scaccgaCfaCfaUfgcaacuucias(invAb)-PK | 1385 |
| AM16408-SS-TP | TL-(NH-C6)s(invAb)sccucgguaUfUfUfauuguuuguas(invAb)(C6-S)- | 1386 |
| | PK | |
| AM16410-SS-TP | TL-(NH-C6)s(invAb)sccucgguaUfUfUfauugucuguas(invAb)(C6-S)- | 1387 |
| | PK | |
| AM16411-SS-TP | TL-(NH-C6)s(invAb)sccucgguaUfUfUfauugucuiuas(invAb)(C6-S)- | 1388 |
| | PK | |
| AM16539-SS-TP | TL-(invAb)sccuggucaUfgGfAfguauuacgas(invAb)-PK | 1389 |
| AM16573-SS-TP | TL-cscscuagAfAfCfugucuucgsasa-PK | 1390 |
| AM16576-SS-TP | TL-(invAb)suuccucggUfaUfUfuauugucuas(invAb)-PK | 1391 |
| AM16701-SS-TP | TL-(invAb)scaccgacaCfaUfgCfaacuucias(invAb)-PK | 1392 |
| AM16802-SS-TP | TL-(NH-C6)s(invAb)sgacccuagAfAfCfugucuuciaas(invAb)(C6-S)- | 1393 |
| | PK | |
| AM16804-SS-TP | TL-(NH-C6)s(invAb)sgaacccuaGfAfAfcugucuucias(invAb)(C6-S)- | 1394 |
| | PK | |
| AM16907-SS-TP | TL-(invAb)suggcaagaUfcGfUfccacuacaas(invAb)-PK | 1395 |
| AM16908-SS-TP | TL-(invAb)suggcaagaUfcGfuCfcacuacaas(invAb)-PK | 1396 |
| AM16963-SS-TP | TL-(NH-C6)s(invAb)sccuggucaUfgGfAfguauuacgas(invAb)(C6-S)- | 1397 |
| | PK | |
| AM17081-SS-TP | TL-(NH-C6)cscscuagAfAfCfugucuucgsasa(invAb)(C6-S)-PK | 1398 |
| AM17650-SS-TP | TL-(NH-C6)s(invAb)sccucgguaUfuUfaUfugucuguas(invAb)(C6-S)- | 1399 |
| | PK | |
| AM17651-SS-TP | TL-(NH-C6)s(invAb)sccucgguaUfuUfAfuugucuguas(invAb)(C6-S)- | 1400 |
| | PK | |
| AM18528-SS-TP | TL-(NH-C6)s(invAb)suuccucggUfaUfUfuauugucuas(invAb)-PK | 1401 |
| AM19347-SS-TP | TL-(NH-C6)s(invAb)sccuggucaUfgGfAfguauuacgas(invAb)-PK | 1402 |
| AM19421-SS-TP | TL-(NH-C6)s(invAb)suuccucggUfaUfUfuauugucuaAlk-PK | 1403 |
| AM19422-SS-TP | TL-(NH-C6)s(invAb)suuccucggUfaUfUfuauugucusaAlk-PK | 1404 |
| AM19573-SS-TP | TL-(NH-C6)s(invAb)suuccucggUfaUfUfuauugucuas(invAb)s(C6-S)- | 1405 |
| | PK | |

The DMPK RNAi agents described herein are formed by annealing an antisense strand with a sense strand. A sense strand containing a sequence listed in Table 2 or Table 4.1, 4.2, 4.3, 4.4, 4.5, 4.6, 5.4, or 5.5 can be hybridized to any antisense strand containing a sequence listed in Table 2, Table 3, or Table 5.4, provided the two sequences have a region of at least 85% complementarity over a contiguous 16, 17, 18, 19, 20, or 21 nucleotide sequence.

In some embodiments, the antisense strand of a DMPK RNAi agent disclosed herein differs by 0, 1, 2, or 3 nucleotides from any of the antisense strand sequences in Table 3. In some embodiments, the sense strand of a DMPK RNAi agent disclosed herein differs by 0, 1, 2, or 3 nucleotides from any of the sense strand sequences in Table 4.1, 4.2, 4.3, 4.4, 4.5, 4.6, 5.4, or 5.5.

In some embodiments, a DMPK RNAi agent antisense strand comprises a nucleotide sequence of any of the sequences in Table 2 or Table 3. In some embodiments, a DMPK RNAi agent antisense strand comprises the sequence of nucleotides (from 5′ end→3′ end) 1-17, 2-17, 1-18, 2-18, 1-19, 2-19, 1-20, 2-20, 1-21, 2-21, 1-22, 2-22, 1-23, 2-23, 1-24, or 2-24 of any of the sequences in Table 2, Table 3, or Table 5.4. In certain embodiments, a DMPK RNAi agent antisense strand comprises or consists of a modified sequence of any one of the modified sequences in Table 3.

In some embodiments, a DMPK RNAi agent sense strand comprises the nucleotide sequence of any of the sequences in Table 2 or Table 4.1, 4.2, 4.3, 4.4, 4.5, 4.6, 5.4, or 5.5. In some embodiments, a DMPK RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, 4-21, 1-22, 2-22, 3-22, 4-22, 1-23, 2-23, 3-23, 4-23, 1-24, 2-24, 3-24, or 4-24 of any of the sequences in Table 2 or Table 4.1, 4.2, 4.3, 4.4, 4.5, 4.6, 5.4, or 5.5. In certain embodiments, a DMPK RNAi agent sense strand comprises or consists of a modified sequence of any one of the modified sequences in Table 4.1, 4.2, 4.3, 4.4, 4.5, 4.6, 5.4, or 5.5.

For the DMPK RNAi agents disclosed herein, the nucleotide at position 1 of the antisense strand (from 5′ end→3′ end) can be perfectly complementary to a DMPK gene, or can be non-complementary to a DMPK gene. In some embodiments, the nucleotide at position 1 of the antisense strand (from 5′ end→3′ end) is a U, A, or dT (or a modified version thereof). In some embodiments, the nucleotide at position 1 of the antisense strand (from 5′ end→3′ end) forms an A: U or U: A base pair with the sense strand.

In some embodiments, a DMPK RNAi agent antisense strand comprises the sequence of nucleotides (from 5′ end→3′ end) 2-18 or 2-19 of any of the antisense strand sequences in Table 2 or Table 3. In some embodiments, a DMPK RNAi sense strand comprises the sequence of nucleotides (from 5′ end→3′ end) 1-17 or 1-18 of any of the sense strand sequences in Table 2 or Table 4.1, 4.2, 4.3, 4.4, 4.5, 4.6, 5.4, or 5.5.

In some embodiments, a DMPK RNAi agent includes (i) an antisense strand comprising the sequence of nucleotides (from 5′ end→3′ end) 2-18 or 2-19 of any of the antisense strand sequences in Table 2 or Table 3, and (ii) a sense strand comprising the sequence of nucleotides (from 5′ end→3′ end) 1-17 or 1-18 of any of the sense strand sequences in Table 2 or Table 4.1, 4.2, 4.3, 4.4, 4.5, 4.6, or 5.4.

A sense strand containing a sequence listed in Table 2 or Table 4 can be hybridized to any antisense strand containing a sequence listed in Table 2 or Table 3, provided the two sequences have a region of at least 85% complementarity over a contiguous 16, 17, 18, 19, 20, or 21 nucleotide sequence. In some embodiments, the DMPK RNAi agent has a sense strand consisting of the modified sequence of any of the modified sequences in Table 4.1, 4.2, 4.3, 4.4, 4.5, 4.6, 5.4, or 5.5 and an antisense strand consisting of the modified sequence of any of the modified sequences in Table 3 or Table 5.4. Certain representative sequence pairings are exemplified by the Duplex ID Nos. shown in Table 5.1, 5.2, 5.3, 5.4, 5.6, and 5.7.

In some embodiments, a DMPK RNAi agent comprises, consists of, or consists essentially of a duplex represented by any one of the Duplex ID Nos. presented herein. In some embodiments, a DMPK RNAi agent comprises the sense strand and antisense strand nucleotide sequences of any of the duplexes represented by any of the Duplex ID Nos. presented herein. In some embodiments, a DMPK RNAi agent comprises the sense strand and antisense strand nucleotide sequences of any of the duplexes represented by any of the Duplex ID Nos. presented herein and a targeting ligand, targeting group, and/or linking group wherein the targeting ligand, targeting group, and/or linking group is covalently linked (i.e., conjugated) to the sense strand or the antisense strand. In some embodiments, a DMPK RNAi agent includes the sense strand and antisense strand modified nucleotide sequences of any of the Duplex ID Nos. presented herein. In some embodiments, a DMPK RNAi agent comprises the sense strand and antisense strand modified nucleotide sequences of any of the Duplex ID Nos. presented herein and a targeting ligand, targeting group, and/or linking group, wherein the targeting ligand, targeting group, and/or linking group is covalently linked to the sense strand or the antisense strand.

In some embodiments, a DMPK RNAi agent comprises an antisense strand and a sense strand having the nucleotide sequences of any of the antisense strand/sense strand duplexes of Table 2 or Table 5.1 (or Table 5.2, Table 5.3, Table 5.4, Table 5.6, or Table 5.7), and further comprises a targeting group. In some embodiments, a DMPK RNAi agent comprises an antisense strand and a sense strand having the nucleotide sequences of any of the antisense strand/sense strand duplexes of Table 5.1 (or Table 5.2, or 5.3, Table 5.4, Table 5.6, or Table 5.7), and further comprises an integrin receptor ligand targeting group.

In some embodiments, a DMPK RNAi agent comprises an antisense strand and a sense strand having the nucleotide sequences of any of the antisense strand/sense strand duplexes of Table 5.1, 5.2, 5.3, 5.4, 5.6, or 5.7, and comprises one or more linking groups selected from the group consisting of (NH2-C₆), (C6—NH2), (C6-SS-C6), or (6-SS-6), each as defined in Table 6.1.

In some embodiments, a DMPK RNAi agent comprises an antisense strand and a sense strand having the modified nucleotide sequence of any of the antisense strand and/or sense strand nucleotide sequences in Table 3 or Table 4.1, 4.2, 4.3, 4.4, 4.5, 4.6, 5.4, or 5.5.

In some embodiments, a DMPK RNAi agent comprises an antisense strand and a sense strand having a modified nucleotide sequence of any of the antisense strand and/or sense strand nucleotide sequences of any of the duplexes Table 5.1 (or Table 5.2, 5.3, 5.4, 5.6 or 5.7), and further comprises an integrin targeting group.

In some embodiments, a DMPK RNAi agent comprises, consists of, or consists essentially of any of the duplexes of Table 5.1 (or Table 5.2, 5.3, 5.4, 5.6, or 5.7).

TABLE 5.1
DMPK RNAi Agents Duplexes with Corresponding
Sense and Antisense Strand ID Numbers

| | Antisense | Sense |
|---|---|---|
| Duplex ID | Strand ID | Strand ID |
| AD08910 | AM12527-AS | AM12526-SS |
| AD08911 | AM12529-AS | AM12528-SS |
| AD08912 | AM12531-AS | AM12530-SS |
| AD08913 | AM12533-AS | AM12532-SS |
| AD08914 | AM12535-AS | AM12534-SS |
| AD08915 | AM12537-AS | AM12536-SS |
| AD08916 | AM12539-AS | AM12538-SS |
| AD08917 | AM12541-AS | AM12540-SS |
| AD08918 | AM12543-AS | AM12542-SS |
| AD08919 | AM12545-AS | AM12544-SS |
| AD09697 | AM13806-AS | AM13805-SS |
| AD09698 | AM13808-AS | AM13807-SS |
| AD09699 | AM13810-AS | AM13809-SS |
| AD09700 | AM13812-AS | AM13811-SS |
| AD09701 | AM13812-AS | AM13813-SS |
| AD09702 | AM13815-AS | AM13814-SS |
| AD09703 | AM13815-AS | AM13816-SS |
| AD09704 | AM13818-AS | AM13817-SS |
| AD09705 | AM13820-AS | AM13819-SS |
| AD09706 | AM13822-AS | AM13821-SS |
| AD09707 | AM13822-AS | AM13823-SS |
| AD09708 | AM13825-AS | AM13824-SS |
| AD09709 | AM13827-AS | AM13826-SS |
| AD09721 | AM13853-AS | AM13852-SS |
| AD09722 | AM13853-AS | AM13854-SS |
| AD09723 | AM13856-AS | AM13855-SS |
| AD09910 | AM14133-AS | AM14132-SS |
| AD09911 | AM14135-AS | AM14134-SS |
| AD09913 | AM14139-AS | AM14138-SS |
| AD09914 | AM14141-AS | AM14140-SS |
| AD09915 | AM14143-AS | AM14142-SS |
| AD09916 | AM14145-AS | AM14144-SS |
| AD09918 | AM14148-AS | AM14147-SS |
| AD09919 | AM14150-AS | AM14149-SS |
| AD09920 | AM14152-AS | AM14151-SS |
| AD09921 | AM14154-AS | AM14153-SS |
| AD09922 | AM14156-AS | AM14155-SS |
| AD09923 | AM14158-AS | AM14157-SS |
| AD10129 | AM14439-AS | AM14438-SS |
| AD10130 | AM14441-AS | AM14440-SS |
| AD10131 | AM14443-AS | AM14442-SS |
| AD10132 | AM14445-AS | AM14444-SS |
| AD10133 | AM14447-AS | AM14446-SS |
| AD10134 | AM14449-AS | AM14448-SS |
| AD10135 | AM14451-AS | AM14450-SS |
| AD10136 | AM14453-AS | AM14452-SS |
| AD10137 | AM14455-AS | AM14454-SS |
| AD10138 | AM14457-AS | AM14456-SS |
| AD10139 | AM14459-AS | AM14458-SS |
| AD10140 | AM14461-AS | AM14460-SS |
| AD10207 | AM14548-AS | AM14547-SS |
| AD10208 | AM14550-AS | AM14549-SS |
| AD10254 | AM14612-AS | AM14140-SS |
| AD10255 | AM14613-AS | AM14140-SS |
| AD10256 | AM14614-AS | AM14140-SS |
| AD10257 | AM14615-AS | AM14140-SS |
| AD10258 | AM14617-AS | AM14616-SS |
| AD10259 | AM14141-AS | AM14618-SS |
| AD10260 | AM14612-AS | AM14618-SS |
| AD10261 | AM14620-AS | AM14619-SS |
| AD10262 | AM14620-AS | AM14621-SS |
| AD10263 | AM14622-AS | AM14619-SS |
| AD10264 | AM14623-AS | AM14619-SS |
| AD10265 | AM14624-AS | AM14619-SS |
| AD10300 | AM14139-AS | AM14668-SS |
| AD10301 | AM14141-AS | AM14669-SS |
| AD10302 | AM14550-AS | AM13809-SS |
| AD10303 | AM14158-AS | AM14140-SS |
| AD10555 | AM15037-AS | AM15036-SS |
| AD10556 | AM15038-AS | AM15036-SS |
| AD10557 | AM15039-AS | AM15036-SS |
| AD10558 | AM15037-AS | AM15040-SS |
| AD10559 | AM15038-AS | AM15040-SS |
| AD10560 | AM15039-AS | AM15040-SS |
| AD10561 | AM15041-AS | AM13814-SS |
| AD10562 | AM15042-AS | AM13814-SS |
| AD10715 | AM15277-AS | AM15276-SS |
| AD10716 | AM15278-AS | AM14618-SS |
| AD10717 | AM15279-AS | AM14618-SS |
| AD10718 | AM15280-AS | AM14618-SS |
| AD10719 | AM14615-AS | AM14618-SS |
| AD10720 | AM15281-AS | AM14618-SS |
| AD10721 | AM15282-AS | AM14618-SS |
| AD10722 | AM14141-AS | AM15283-SS |
| AD10723 | AM15280-AS | AM15283-SS |
| AD10724 | AM14615-AS | AM15283-SS |
| AD10725 | AM15281-AS | AM15283-SS |
| AD10726 | AM15282-AS | AM15283-SS |
| AD10727 | AM14158-AS | AM14618-SS |
| AD10850 | AM15477-AS | AM15476-SS |
| AD10851 | AM15479-AS | AM15478-SS |
| AD10852 | AM15481-AS | AM15480-SS |
| AD10853 | AM15481-AS | AM15482-SS |
| AD10854 | AM15484-AS | AM15483-SS |
| AD10855 | AM15484-AS | AM15485-SS |
| AD10856 | AM15487-AS | AM15486-SS |
| AD10857 | AM15489-AS | AM15488-SS |
| AD10858 | AM15489-AS | AM15490-SS |
| AD10859 | AM15492-AS | AM15491-SS |
| AD10860 | AM15494-AS | AM15493-SS |
| AD10861 | AM15496-AS | AM15495-SS |
| AD10862 | AM15498-AS | AM15497-SS |
| AD10863 | AM15500-AS | AM15499-SS |
| AD10864 | AM15502-AS | AM15501-SS |
| AD10865 | AM15504-AS | AM15503-SS |
| AD10866 | AM15506-AS | AM15505-SS |
| AD10867 | AM15506-AS | AM15507-SS |
| AD10868 | AM15509-AS | AM15508-SS |
| AD10869 | AM15511-AS | AM15510-SS |
| AD10870 | AM15511-AS | AM15512-SS |
| AD10871 | AM15514-AS | AM15513-SS |
| AD10872 | AM15516-AS | AM15515-SS |
| AD10873 | AM15518-AS | AM15517-SS |
| AD10874 | AM15520-AS | AM15519-SS |
| AD10875 | AM15522-AS | AM15521-SS |
| AD10876 | AM15524-AS | AM15523-SS |
| AD10877 | AM15526-AS | AM15525-SS |
| AD11086 | AM15780-AS | AM15283-SS |
| AD11087 | AM15781-AS | AM15283-SS |
| AD11130 | AM15833-AS | AM14619-SS |
| AD11131 | AM15834-AS | AM14619-SS |
| AD11132 | AM15835-AS | AM14619-SS |
| AD11133 | AM15837-AS | AM15836-SS |
| AD11134 | AM15838-AS | AM15836-SS |
| AD11135 | AM15833-AS | AM15836-SS |
| AD11136 | AM15834-AS | AM15836-SS |
| AD11137 | AM15835-AS | AM15836-SS |
| AD11138 | AM15837-AS | AM14621-SS |
| AD11139 | AM15833-AS | AM14621-SS |
| AD11140 | AM15834-AS | AM14621-SS |
| AD11141 | AM15835-AS | AM14621-SS |
| AD11486 | AM15522-AS | AM16315-SS |
| AD11487 | AM16316-AS | AM15521-SS |
| AD11488 | AM16317-AS | AM15521-SS |
| AD11489 | AM16319-AS | AM16318-SS |
| AD11490 | AM16320-AS | AM16318-SS |
| AD11491 | AM16321-AS | AM16318-SS |
| AD11492 | AM16320-AS | AM16322-SS |
| AD11493 | AM16321-AS | AM16322-SS |
| AD11548 | AM16409-AS | AM16408-SS |
| AD11549 | AM16409-AS | AM16410-SS |
| AD11550 | AM16409-AS | AM16411-SS |
| AD11633 | AM16537-AS | AM15836-SS |
| AD11634 | AM16538-AS | AM15836-SS |
| AD11635 | AM16540-AS | AM16539-SS |
| AD11636 | AM16541-AS | AM14619-SS |
| AD11637 | AM16542-AS | AM14619-SS |
| AD11638 | AM16542-AS | AM15836-SS |
| AD11639 | AM16543-AS | AM14619-SS |
| AD11640 | AM16543-AS | AM15836-SS |
| AD11641 | AM16543-AS | AM14621-SS |
| AD11642 | AM16544-AS | AM15836-SS |
| AD11643 | AM16544-AS | AM14621-SS |
| AD11667 | AM16574-AS | AM16573-SS |
| AD11668 | AM16575-AS | AM16573-SS |
| AD11669 | AM16577-AS | AM16576-SS |
| AD11670 | AM15780-AS | AM16576-SS |
| AD11749 | AM16702-AS | AM16701-SS |
| AD11750 | AM16319-AS | AM16701-SS |
| AD11751 | AM16320-AS | AM16701-SS |
| AD11752 | AM16321-AS | AM16701-SS |
| AD11753 | AM16702-AS | AM16318-SS |
| AD11754 | AM16703-AS | AM16318-SS |
| AD11755 | AM16704-AS | AM16318-SS |
| AD11756 | AM16702-AS | AM16322-SS |
| AD11757 | AM16319-AS | AM16322-SS |
| AD11833 | AM16803-AS | AM16802-SS |
| AD11834 | AM16805-AS | AM16804-SS |
| AD11891 | AM16905-AS | AM15036-SS |
| AD11892 | AM16906-AS | AM15036-SS |
| AD11893 | AM15037-AS | AM16907-SS |
| AD11894 | AM15038-AS | AM16907-SS |
| AD11895 | AM15039-AS | AM16907-SS |
| AD11896 | AM15037-AS | AM16908-SS |
| AD11897 | AM15038-AS | AM16908-SS |
| AD11898 | AM15039-AS | AM16908-SS |
| AD11899 | AM16909-AS | AM15036-SS |
| AD11900 | AM16910-AS | AM15036-SS |
| AD11953 | AM16962-AS | AM16539-SS |
| AD11954 | AM16540-AS | AM16963-SS |
| AD11955 | AM16962-AS | AM16963-SS |
| AD12039 | AM16575-AS | AM17081-SS |
| AD12244 | AM17362-AS | AM17361-SS |
| AD12245 | AM17364-AS | AM17363-SS |
| AD12246 | AM17365-AS | AM16539-SS |
| AD12247 | AM17362-AS | AM16701-SS |
| AD12248 | AM17364-AS | AM15036-SS |
| AD12473 | AM17649-AS | AM16410-SS |
| AD12474 | AM17649-AS | AM17650-SS |
| AD12475 | AM17649-AS | AM17651-SS |
| AD12476 | AM17652-AS | AM17651-SS |
| AD12477 | AM17653-AS | AM17650-SS |
| AD12478 | AM17654-AS | AM17651-SS |
| AD12858 | AM18250-AS | AM15283-SS |
| AD12859 | AM18251-AS | AM15283-SS |
| AD13066 | AM15780-AS | AM18528-SS |
| AD13072 | AM15780-AS | AM18534-SS |
| AD13372 | AM16577-AS | AM19026-SS |
| AD13494 | AM19163-AS | AM15283-SS |
| AD13646 | AM16962-AS | AM19347-SS |
| AD13736 | AM15780-AS | AM19421-SS |
| AD13737 | AM15780-AS | AM19422-SS |
| AD13838 | AM15780-AS | AM19573-SS |

TABLE 5.2
DMPK RNAi Agent Duplexes with Corresponding Sense and Antisense
Strand ID Numbers and Sequence ID numbers for the modified
and unmodified nucleotide sequences.

| | | AS | | | SS | |
|---|---|---|---|---|---|---|
| | | Modified | AS | | Modified | SS |
| | | Seq ID | Unmodified | | Seq ID | Unmodified |
| Duplex | AS ID | No: | Seq ID No: | SS ID | No: | Seq ID No: |
| AD08910 | AM12527-AS | 633 | 1443 | AM12526-SS | 876 | 1511 |
| AD08911 | AM12529-AS | 634 | 1444 | AM12528-SS | 877 | 1512 |
| AD08912 | AM12531-AS | 635 | 1445 | AM12530-SS | 878 | 1513 |
| AD08913 | AM12533-AS | 636 | 1446 | AM12532-SS | 879 | 1514 |
| AD08914 | AM12535-AS | 637 | 1447 | AM12534-SS | 880 | 1515 |
| AD08915 | AM12537-AS | 638 | 1448 | AM12536-SS | 881 | 1516 |
| AD08916 | AM12539-AS | 639 | 1449 | AM12538-SS | 882 | 1517 |
| AD08917 | AM12541-AS | 640 | 1450 | AM12540-SS | 883 | 1518 |
| AD08918 | AM12543-AS | 641 | 1451 | AM12542-SS | 884 | 1519 |
| AD08919 | AM12545-AS | 642 | 1452 | AM12544-SS | 885 | 1520 |
| AD09697 | AM13806-AS | 643 | 1453 | AM13805-SS | 886 | 1521 |
| AD09698 | AM13808-AS | 644 | 1454 | AM13807-SS | 887 | 1522 |
| AD09699 | AM13810-AS | 645 | 1455 | AM13809-SS | 888 | 1523 |
| AD09700 | AM13812-AS | 646 | 1456 | AM13811-SS | 889 | 1524 |
| AD09702 | AM13815-AS | 647 | 1457 | AM13814-SS | 890 | 1525 |
| AD09704 | AM13818-AS | 648 | 1458 | AM13817-SS | 891 | 1526 |
| AD09705 | AM13820-AS | 649 | 1459 | AM13819-SS | 892 | 1527 |
| AD09706 | AM13822-AS | 650 | 1460 | AM13821-SS | 893 | 1528 |
| AD09707 | AM13822-AS | 650 | 1460 | AM13823-SS | 894 | 1529 |
| AD09708 | AM13825-AS | 651 | 1461 | AM13824-SS | 895 | 1530 |
| AD09709 | AM13827-AS | 652 | 1462 | AM13826-SS | 896 | 1531 |
| AD09721 | AM13853-AS | 653 | 1463 | AM13852-SS | 897 | 1532 |
| AD09722 | AM13853-AS | 653 | 1463 | AM13854-SS | 898 | 1533 |
| AD09723 | AM13856-AS | 654 | 1464 | AM13855-SS | 899 | 1534 |
| AD09910 | AM14133-AS | 655 | 1465 | AM14132-SS | 900 | 1541 |
| AD09911 | AM14135-AS | 656 | 1466 | AM14134-SS | 901 | 1588 |
| AD09913 | AM14139-AS | 657 | 1467 | AM14138-SS | 903 | 1542 |
| AD09914 | AM14141-AS | 658 | 1468 | AM14140-SS | 904 | 1543 |
| AD09915 | AM14143-AS | 659 | 1469 | AM14142-SS | 905 | 1590 |
| AD09920 | AM14152-AS | 663 | 1465 | AM14151-SS | 906 | 1541 |
| AD09922 | AM14156-AS | 665 | 1467 | AM14155-SS | 907 | 1542 |
| AD09923 | AM14158-AS | 666 | 1468 | AM14157-SS | 908 | 1543 |
| AD10129 | AM14439-AS | 667 | 1471 | AM14438-SS | 909 | 1544 |
| AD10130 | AM14441-AS | 668 | 1472 | AM14440-SS | 910 | 1545 |
| AD10131 | AM14443-AS | 669 | 1473 | AM14442-SS | 911 | 1546 |
| AD10132 | AM14445-AS | 670 | 1474 | AM14444-SS | 912 | 1547 |
| AD10133 | AM14447-AS | 671 | 1475 | AM14446-SS | 913 | 1548 |
| AD10134 | AM14449-AS | 672 | 1476 | AM14448-SS | 914 | 1549 |
| AD10135 | AM14451-AS | 673 | 1477 | AM14450-SS | 915 | 1550 |
| AD10136 | AM14453-AS | 674 | 1478 | AM14452-SS | 916 | 1551 |
| AD10137 | AM14455-AS | 675 | 1479 | AM14454-SS | 917 | 1552 |
| AD10138 | AM14457-AS | 676 | 1480 | AM14456-SS | 918 | 1553 |
| AD10207 | AM14548-AS | 679 | 1463 | AM14547-SS | 919 | 1532 |
| AD10208 | AM14550-AS | 680 | 1455 | AM14549-SS | 920 | 1523 |
| AD10254 | AM14612-AS | 681 | 1468 | AM14140-SS | 904 | 1543 |
| AD10255 | AM14613-AS | 682 | 1468 | AM14140-SS | 904 | 1543 |
| AD10256 | AM14614-AS | 683 | 1468 | AM14140-SS | 904 | 1543 |
| AD10257 | AM14615-AS | 684 | 1468 | AM14140-SS | 904 | 1543 |
| AD10258 | AM14617-AS | 685 | 1483 | AM14616-SS | 921 | 1591 |
| AD10259 | AM14141-AS | 658 | 1468 | AM14618-SS | 922 | 1543 |
| AD10260 | AM14612-AS | 681 | 1468 | AM14618-SS | 922 | 1543 |
| AD10261 | AM14620-AS | 686 | 1455 | AM14619-SS | 923 | 1523 |
| AD10262 | AM14620-AS | 686 | 1455 | AM14621-SS | 924 | 1523 |
| AD10263 | AM14622-AS | 687 | 1455 | AM14619-SS | 923 | 1523 |
| AD10264 | AM14623-AS | 688 | 1455 | AM14619-SS | 923 | 1523 |
| AD10265 | AM14624-AS | 689 | 1455 | AM14619-SS | 923 | 1523 |
| AD10300 | AM14139-AS | 657 | 1467 | AM14668-SS | 925 | 1542 |
| AD10301 | AM14141-AS | 658 | 1468 | AM14669-SS | 926 | 1543 |
| AD10302 | AM14550-AS | 680 | 1455 | AM13809-SS | 888 | 1523 |
| AD10303 | AM14158-AS | 666 | 1468 | AM14140-SS | 904 | 1543 |
| AD10555 | AM15037-AS | 690 | 1457 | AM15036-SS | 927 | 1525 |
| AD10556 | AM15038-AS | 691 | 1457 | AM15036-SS | 927 | 1525 |
| AD10557 | AM15039-AS | 692 | 1457 | AM15036-SS | 927 | 1525 |
| AD10558 | AM15037-AS | 690 | 1457 | AM15040-SS | 928 | 1525 |
| AD10559 | AM15038-AS | 691 | 1457 | AM15040-SS | 928 | 1525 |
| AD10560 | AM15039-AS | 692 | 1457 | AM15040-SS | 928 | 1525 |
| AD10561 | AM15041-AS | 693 | 1457 | AM13814-SS | 890 | 1525 |
| AD10562 | AM15042-AS | 694 | 1457 | AM13814-SS | 890 | 1525 |
| AD10715 | AM15277-AS | 695 | 1483 | AM15276-SS | 929 | 1591 |
| AD10716 | AM15278-AS | 696 | 1468 | AM14618-SS | 922 | 1543 |
| AD10717 | AM15279-AS | 697 | 1468 | AM14618-SS | 922 | 1543 |
| AD10718 | AM15280-AS | 698 | 1468 | AM14618-SS | 922 | 1543 |
| AD10719 | AM14615-AS | 684 | 1468 | AM14618-SS | 922 | 1543 |
| AD10720 | AM15281-AS | 699 | 1468 | AM14618-SS | 922 | 1543 |
| AD10721 | AM15282-AS | 700 | 1468 | AM14618-SS | 922 | 1543 |
| AD10722 | AM14141-AS | 658 | 1468 | AM15283-SS | 930 | 1543 |
| AD10723 | AM15280-AS | 698 | 1468 | AM15283-SS | 930 | 1543 |
| AD10724 | AM14615-AS | 684 | 1468 | AM15283-SS | 930 | 1543 |
| AD10725 | AM15281-AS | 699 | 1468 | AM15283-SS | 930 | 1543 |
| AD10726 | AM15282-AS | 700 | 1468 | AM15283-SS | 930 | 1543 |
| AD10727 | AM14158-AS | 666 | 1468 | AM14618-SS | 922 | 1543 |
| AD10850 | AM15477-AS | 701 | 1484 | AM15476-SS | 931 | 1592 |
| AD10851 | AM15479-AS | 702 | 1485 | AM15478-SS | 932 | 1593 |
| AD10852 | AM15481-AS | 703 | 1486 | AM15480-SS | 933 | 1594 |
| AD10853 | AM15481-AS | 703 | 1486 | AM15482-SS | 934 | 1595 |
| AD10854 | AM15484-AS | 704 | 1487 | AM15483-SS | 935 | 1596 |
| AD10855 | AM15484-AS | 704 | 1487 | AM15485-SS | 936 | 1597 |
| AD10856 | AM15487-AS | 705 | 1488 | AM15486-SS | 937 | 1598 |
| AD10857 | AM15489-AS | 706 | 1489 | AM15488-SS | 938 | 1599 |
| AD10858 | AM15489-AS | 706 | 1489 | AM15490-SS | 939 | 1600 |
| AD10859 | AM15492-AS | 707 | 1490 | AM15491-SS | 940 | 1601 |
| AD10869 | AM15511-AS | 716 | 1499 | AM15510-SS | 941 | 1567 |
| AD10870 | AM15511-AS | 716 | 1499 | AM15512-SS | 942 | 1568 |
| AD10871 | AM15514-AS | 717 | 1500 | AM15513-SS | 943 | 1569 |
| AD10872 | AM15516-AS | 718 | 1501 | AM15515-SS | 944 | 1570 |
| AD10873 | AM15518-AS | 719 | 1502 | AM15517-SS | 945 | 1571 |
| AD10874 | AM15520-AS | 720 | 1503 | AM15519-SS | 946 | 1572 |
| AD10875 | AM15522-AS | 721 | 1504 | AM15521-SS | 947 | 1573 |
| AD10876 | AM15524-AS | 722 | 1505 | AM15523-SS | 948 | 1574 |
| AD10877 | AM15526-AS | 723 | 1506 | AM15525-SS | 949 | 1575 |
| AD11086 | AM15780-AS | 724 | 1468 | AM15283-SS | 930 | 1543 |
| AD11087 | AM15781-AS | 725 | 1468 | AM15283-SS | 930 | 1543 |
| AD11130 | AM15833-AS | 726 | 1455 | AM14619-SS | 923 | 1523 |
| AD11131 | AM15834-AS | 727 | 1455 | AM14619-SS | 923 | 1523 |
| AD11132 | AM15835-AS | 728 | 1455 | AM14619-SS | 923 | 1523 |
| AD11133 | AM15837-AS | 729 | 1455 | AM15836-SS | 950 | 1523 |
| AD11134 | AM15838-AS | 730 | 1455 | AM15836-SS | 950 | 1523 |
| AD11135 | AM15833-AS | 726 | 1455 | AM15836-SS | 950 | 1523 |
| AD11136 | AM15834-AS | 727 | 1455 | AM15836-SS | 950 | 1523 |
| AD11137 | AM15835-AS | 728 | 1455 | AM15836-SS | 950 | 1523 |
| AD11138 | AM15837-AS | 729 | 1455 | AM14621-SS | 924 | 1523 |
| AD11139 | AM15833-AS | 726 | 1455 | AM14621-SS | 924 | 1523 |
| AD11140 | AM15834-AS | 727 | 1455 | AM14621-SS | 924 | 1523 |
| AD11141 | AM15835-AS | 728 | 1455 | AM14621-SS | 924 | 1523 |
| AD11487 | AM16316-AS | 731 | 1504 | AM15521-SS | 947 | 1573 |
| AD11488 | AM16317-AS | 732 | 1504 | AM15521-SS | 947 | 1573 |
| AD11548 | AM16409-AS | 736 | 1480 | AM16408-SS | 951 | 1552 |
| AD11549 | AM16409-AS | 736 | 1480 | AM16410-SS | 952 | 1602 |
| AD11550 | AM16409-AS | 736 | 1480 | AM16411-SS | 953 | 1603 |
| AD11633 | AM16537-AS | 738 | 1455 | AM15836-SS | 950 | 1523 |
| AD11634 | AM16538-AS | 739 | 1455 | AM15836-SS | 950 | 1523 |
| AD11635 | AM16540-AS | 740 | 1507 | AM16539-SS | 654 | 1579 |
| AD11636 | AM16541-AS | 741 | 1455 | AM14619-SS | 923 | 1523 |
| AD11637 | AM16542-AS | 742 | 1455 | AM14619-SS | 923 | 1523 |
| AD11638 | AM16542-AS | 742 | 1455 | AM15836-SS | 950 | 1523 |
| AD11639 | AM16543-AS | 743 | 1455 | AM14619-SS | 923 | 1523 |
| AD11640 | AM16543-AS | 743 | 1455 | AM15836-SS | 950 | 1523 |
| AD11641 | AM16543-AS | 743 | 1455 | AM14621-SS | 924 | 1523 |
| AD11642 | AM16544-AS | 744 | 1455 | AM15836-SS | 950 | 1523 |
| AD11643 | AM16544-AS | 744 | 1455 | AM14621-SS | 924 | 1523 |
| AD11667 | AM16574-AS | 745 | 1508 | AM16573-SS | 955 | 1580 |
| AD11668 | AM16575-AS | 746 | 1508 | AM16573-SS | 955 | 1580 |
| AD11669 | AM16577-AS | 747 | 1468 | AM16576-SS | 956 | 1543 |
| AD11670 | AM15780-AS | 724 | 1468 | AM16576-SS | 956 | 1543 |
| AD11749 | AM16702-AS | 748 | 1504 | AM16701-SS | 957 | 1573 |
| AD11750 | AM16319-AS | 733 | 1504 | AM16701-SS | 957 | 1573 |
| AD11751 | AM16320-AS | 734 | 1504 | AM16701-SS | 957 | 1573 |
| AD11752 | AM16321-AS | 735 | 1504 | AM16701-SS | 957 | 1573 |
| AD11833 | AM16803-AS | 751 | 1446 | AM16802-SS | 958 | 1514 |
| AD11834 | AM16805-AS | 752 | 1445 | AM16804-SS | 959 | 1513 |
| AD11891 | AM16905-AS | 753 | 1457 | AM15036-SS | 927 | 1525 |
| AD11892 | AM16906-AS | 754 | 1457 | AM15036-SS | 927 | 1525 |
| AD11893 | AM15037-AS | 690 | 1457 | AM16907-SS | 960 | 1525 |
| AD11894 | AM15038-AS | 691 | 1457 | AM16907-SS | 960 | 1525 |
| AD11895 | AM15039-AS | 692 | 1457 | AM16907-SS | 960 | 1525 |
| AD11896 | AM15037-AS | 690 | 1457 | AM16908-SS | 961 | 1525 |
| AD11897 | AM15038-AS | 691 | 1457 | AM16908-SS | 961 | 1525 |
| AD11898 | AM15039-AS | 692 | 1457 | AM16908-SS | 961 | 1525 |
| AD11899 | AM16909-AS | 755 | 1457 | AM15036-SS | 927 | 1525 |
| AD11900 | AM16910-AS | 756 | 1457 | AM15036-SS | 927 | 1525 |
| AD11953 | AM16962-AS | 757 | 1507 | AM16539-SS | 954 | 1579 |
| AD11954 | AM16540-AS | 740 | 1507 | AM16963-SS | 962 | 1579 |
| AD11955 | AM16962-AS | 757 | 1507 | AM16963-SS | 962 | 1579 |
| AD12039 | AM16575-AS | 746 | 1508 | AM17081-SS | 963 | 1580 |
| AD12246 | AM17365-AS | 760 | 1507 | AM16539-SS | 954 | 1579 |
| AD12247 | AM17362-AS | 758 | 1504 | AM16701-SS | 957 | 1573 |
| AD12248 | AM17364-AS | 759 | 1457 | AM15036-SS | 927 | 1525 |
| AD12473 | AM17649-AS | 761 | 1480 | AM16410-SS | 952 | 1602 |
| AD12474 | AM17649-AS | 761 | 1480 | AM17650-SS | 964 | 1602 |
| AD12475 | AM17649-AS | 761 | 1480 | AM17651-SS | 965 | 1602 |
| AD12476 | AM17652-AS | 762 | 1480 | AM17651-SS | 965 | 1602 |
| AD12477 | AM17653-AS | 763 | 1480 | AM17650-SS | 964 | 1602 |
| AD12478 | AM17654-AS | 764 | 1480 | AM17651-SS | 965 | 1602 |
| AD12858 | AM18250-AS | 765 | 1623 | AM15283-SS | 930 | 1543 |
| AD12859 | AM18251-AS | 766 | 1468 | AM15283-SS | 930 | 1543 |
| AD13066 | AM15780-AS | 724 | 1468 | AM18528-SS | 966 | 1543 |
| AD13494 | AM19163-AS | 767 | 1623 | AM15283-SS | 930 | 1543 |
| AD13646 | AM16962-AS | 757 | 1507 | AM19347-SS | 967 | 1579 |
| AD13736 | AM15780-AS | 724 | 1468 | AM19421-SS | 968 | 1585 |
| AD13737 | AM15780-AS | 724 | 1468 | AM19422-SS | 969 | 1585 |
| AD13838 | AM15780-AS | 724 | 1468 | AM19573-SS | 970 | 1543 |

* Modified SS sequence is taken from Table 4.2 (shown without terminal dT added for manufacturability).

TABLE 5.3
DMPK RNAi Agents Duplexes with Corresponding
Sense and Antisense Strand ID Numbers Referencing
Position Targeted on DMPK Gene

| | | | Targeted DMPK |
|---|---|---|---|
| | Antisense | Sense | Gene Position |
| Duplex ID | Strand ID | Strand ID | (Of SEQ ID NO: 1) |
| AD08910 | AM12527-AS | AM12526-SS | 1909 |
| AD08911 | AM12529-AS | AM12528-SS | 2458 |
| AD08912 | AM12531-AS | AM12530-SS | 2463 |
| AD08913 | AM12533-AS | AM12532-SS | 2464 |
| AD08914 | AM12535-AS | AM12534-SS | 2465 |
| AD08915 | AM12537-AS | AM12536-SS | 2466 |
| AD08916 | AM12539-AS | AM12538-SS | 2948 |
| AD08917 | AM12541-AS | AM12540-SS | 2956 |
| AD08918 | AM12543-AS | AM12542-SS | 3117 |
| AD08919 | AM12545-AS | AM12544-SS | 3119 |
| AD09697 | AM13806-AS | AM13805-SS | 931 |
| AD09698 | AM13808-AS | AM13807-SS | 991 |
| AD09699 | AM13810-AS | AM13809-SS | 1009 |
| AD09700 | AM13812-AS | AM13811-SS | 1156 |
| AD09701 | AM13812-AS | AM13813-SS | 1156 |
| AD09702 | AM13815-AS | AM13814-SS | 1435 |
| AD09703 | AM13815-AS | AM13816-SS | 1435 |
| AD09704 | AM13818-AS | AM13817-SS | 1630 |
| AD09705 | AM13820-AS | AM13819-SS | 1660 |
| AD09706 | AM13822-AS | AM13821-SS | 2462 |
| AD09707 | AM13822-AS | AM13823-SS | 2462 |
| AD09708 | AM13825-AS | AM13824-SS | 2947 |
| AD09709 | AM13827-AS | AM13826-SS | 3055 |
| AD09721 | AM13853-AS | AM13852-SS | 3152 |
| AD09722 | AM13853-AS | AM13854-SS | 3152 |
| AD09723 | AM13856-AS | AM13855-SS | 3200 |
| AD09910 | AM14133-AS | AM14132-SS | 902 |
| AD09911 | AM14135-AS | AM14134-SS | 1395 |
| AD09913 | AM14139-AS | AM14138-SS | 3147 |
| AD09914 | AM14141-AS | AM14140-SS | 3155 |
| AD09915 | AM14143-AS | AM14142-SS | 820 |
| AD09916 | AM14145-AS | AM14144-SS | 974 |
| AD09918 | AM14148-AS | AM14147-SS | 820 |
| AD09919 | AM14150-AS | AM14149-SS | 974 |
| AD09920 | AM14152-AS | AM14151-SS | 902 |
| AD09921 | AM14154-AS | AM14153-SS | 1395 |
| AD09922 | AM14156-AS | AM14155-SS | 3147 |
| AD09923 | AM14158-AS | AM14157-SS | 3155 |
| AD10129 | AM14439-AS | AM14438-SS | 2953 |
| AD10130 | AM14441-AS | AM14440-SS | 2958 |
| AD10131 | AM14443-AS | AM14442-SS | 3052 |
| AD10132 | AM14445-AS | AM14444-SS | 3053 |
| AD10133 | AM14447-AS | AM14446-SS | 3116 |
| AD10134 | AM14449-AS | AM14448-SS | 3122 |
| AD10135 | AM14451-AS | AM14450-SS | 3127 |
| AD10136 | AM14453-AS | AM14452-SS | 3128 |
| AD10137 | AM14455-AS | AM14454-SS | 3154 |
| AD10138 | AM14457-AS | AM14456-SS | 3157 |
| AD10139 | AM14459-AS | AM14458-SS | 3158 |
| AD10140 | AM14461-AS | AM14460-SS | 3167 |
| AD10207 | AM14548-AS | AM14547-SS | 3152 |
| AD10208 | AM14550-AS | AM14549-SS | 1009 |
| AD10254 | AM14612-AS | AM14140-SS | 3155 |
| AD10255 | AM14613-AS | AM14140-SS | 3155 |
| AD10256 | AM14614-AS | AM14140-SS | 3155 |
| AD10257 | AM14615-AS | AM14140-SS | 3155 |
| AD10258 | AM14617-AS | AM14616-SS | 3155 |
| AD10259 | AM14141-AS | AM14618-SS | 3155 |
| AD10260 | AM14612-AS | AM14618-SS | 3155 |
| AD10261 | AM14620-AS | AM14619-SS | 1009 |
| AD10262 | AM14620-AS | AM14621-SS | 1009 |
| AD10263 | AM14622-AS | AM14619-SS | 1009 |
| AD10264 | AM14623-AS | AM14619-SS | 1009 |
| AD10265 | AM14624-AS | AM14619-SS | 1009 |
| AD10300 | AM14139-AS | AM14668-SS | 3147 |
| AD10301 | AM14141-AS | AM14669-SS | 3155 |
| AD10302 | AM14550-AS | AM13809-SS | 1009 |
| AD10303 | AM14158-AS | AM14140-SS | 3155 |
| AD10555 | AM15037-AS | AM15036-SS | 1435 |
| AD10556 | AM15038-AS | AM15036-SS | 1435 |
| AD10557 | AM15039-AS | AM15036-SS | 1435 |
| AD10558 | AM15037-AS | AM15040-SS | 1435 |
| AD10559 | AM15038-AS | AM15040-SS | 1435 |
| AD10560 | AM15039-AS | AM15040-SS | 1435 |
| AD10561 | AM15041-AS | AM13814-SS | 1435 |
| AD10562 | AM15042-AS | AM13814-SS | 1435 |
| AD10715 | AM15277-AS | AM15276-SS | 3155 |
| AD10716 | AM15278-AS | AM14618-SS | 3155 |
| AD10717 | AM15279-AS | AM14618-SS | 3155 |
| AD10718 | AM15280-AS | AM14618-SS | 3155 |
| AD10719 | AM14615-AS | AM14618-SS | 3155 |
| AD10720 | AM15281-AS | AM14618-SS | 3155 |
| AD10721 | AM15282-AS | AM14618-SS | 3155 |
| AD10722 | AM14141-AS | AM15283-SS | 3155 |
| AD10723 | AM15280-AS | AM15283-SS | 3155 |
| AD10724 | AM14615-AS | AM15283-SS | 3155 |
| AD10725 | AM15281-AS | AM15283-SS | 3155 |
| AD10726 | AM15282-AS | AM15283-SS | 3155 |
| AD10727 | AM14158-AS | AM14618-SS | 3155 |
| AD10850 | AM15477-AS | AM15476-SS | 882 |
| AD10851 | AM15479-AS | AM15478-SS | 883 |
| AD10852 | AM15481-AS | AM15480-SS | 985 |
| AD10853 | AM15481-AS | AM15482-SS | 985 |
| AD10854 | AM15484-AS | AM15483-SS | 987 |
| AD10855 | AM15484-AS | AM15485-SS | 987 |
| AD10856 | AM15487-AS | AM15486-SS | 990 |
| AD10857 | AM15489-AS | AM15488-SS | 1000 |
| AD10858 | AM15489-AS | AM15490-SS | 1000 |
| AD10859 | AM15492-AS | AM15491-SS | 1046 |
| AD10860 | AM15494-AS | AM15493-SS | 865 |
| AD10861 | AM15496-AS | AM15495-SS | 867 |
| AD10862 | AM15498-AS | AM15497-SS | 868 |
| AD10863 | AM15500-AS | AM15499-SS | 1041 |
| AD10864 | AM15502-AS | AM15501-SS | 1044 |
| AD10865 | AM15504-AS | AM15503-SS | 1158 |
| AD10866 | AM15506-AS | AM15505-SS | 1161 |
| AD10867 | AM15506-AS | AM15507-SS | 1161 |
| AD10868 | AM15509-AS | AM15508-SS | 1438 |
| AD10869 | AM15511-AS | AM15510-SS | 1450 |
| AD10870 | AM15511-AS | AM15512-SS | 1450 |
| AD10871 | AM15514-AS | AM15513-SS | 1574 |
| AD10872 | AM15516-AS | AM15515-SS | 1576 |
| AD10873 | AM15518-AS | AM15517-SS | 1632 |
| AD10874 | AM15520-AS | AM15519-SS | 1636 |
| AD10875 | AM15522-AS | AM15521-SS | 1657 |
| AD10876 | AM15524-AS | AM15523-SS | 1659 |
| AD10877 | AM15526-AS | AM15525-SS | 1664 |
| AD11086 | AM15780-AS | AM15283-SS | 3155 |
| AD11087 | AM15781-AS | AM15283-SS | 3155 |
| AD11130 | AM15833-AS | AM14619-SS | 1009 |
| AD11131 | AM15834-AS | AM14619-SS | 1009 |
| AD11132 | AM15835-AS | AM14619-SS | 1009 |
| AD11133 | AM15837-AS | AM15836-SS | 1009 |
| AD11134 | AM15838-AS | AM15836-SS | 1009 |
| AD11135 | AM15833-AS | AM15836-SS | 1009 |
| AD11136 | AM15834-AS | AM15836-SS | 1009 |
| AD11137 | AM15835-AS | AM15836-SS | 1009 |
| AD11138 | AM15837-AS | AM14621-SS | 1009 |
| AD11139 | AM15833-AS | AM14621-SS | 1009 |
| AD11140 | AM15834-AS | AM14621-SS | 1009 |
| AD11141 | AM15835-AS | AM14621-SS | 1009 |
| AD11486 | AM15522-AS | AM16315-SS | 1657 |
| AD11487 | AM16316-AS | AM15521-SS | 1657 |
| AD11488 | AM16317-AS | AM15521-SS | 1657 |
| AD11489 | AM16319-AS | AM16318-SS | 1657 |
| AD11490 | AM16320-AS | AM16318-SS | 1657 |
| AD11491 | AM16321-AS | AM16318-SS | 1657 |
| AD11492 | AM16320-AS | AM16322-SS | 1657 |
| AD11493 | AM16321-AS | AM16322-SS | 1657 |
| AD11548 | AM16409-AS | AM16408-SS | 3157 |
| AD11549 | AM16409-AS | AM16410-SS | 3157 |
| AD11550 | AM16409-AS | AM16411-SS | 3157 |
| AD11633 | AM16537-AS | AM15836-SS | 1009 |
| AD11634 | AM16538-AS | AM15836-SS | 1009 |
| AD11635 | AM16540-AS | AM16539-SS | 1009 |
| AD11636 | AM16541-AS | AM14619-SS | 1009 |
| AD11637 | AM16542-AS | AM14619-SS | 1009 |
| AD11638 | AM16542-AS | AM15836-SS | 1009 |
| AD11639 | AM16543-AS | AM14619-SS | 1009 |
| AD11640 | AM16543-AS | AM15836-SS | 1009 |
| AD11641 | AM16543-AS | AM14621-SS | 1009 |
| AD11642 | AM16544-AS | AM15836-SS | 1009 |
| AD11643 | AM16544-AS | AM14621-SS | 1009 |
| AD11667 | AM16574-AS | AM16573-SS | 2464 |
| AD11668 | AM16575-AS | AM16573-SS | 2464 |
| AD11669 | AM16577-AS | AM16576-SS | 3155 |
| AD11670 | AM15780-AS | AM16576-SS | 3155 |
| AD11749 | AM16702-AS | AM16701-SS | 1657 |
| AD11750 | AM16319-AS | AM16701-SS | 1657 |
| AD11751 | AM16320-AS | AM16701-SS | 1657 |
| AD11752 | AM16321-AS | AM16701-SS | 1657 |
| AD11753 | AM16702-AS | AM16318-SS | 1657 |
| AD11754 | AM16703-AS | AM16318-SS | 1657 |
| AD11755 | AM16704-AS | AM16318-SS | 1657 |
| AD11756 | AM16702-AS | AM16322-SS | 1657 |
| AD11757 | AM16319-AS | AM16322-SS | 1657 |
| AD11833 | AM16803-AS | AM16802-SS | 2464 |
| AD11834 | AM16805-AS | AM16804-SS | 2463 |
| AD11891 | AM16905-AS | AM15036-SS | 1435 |
| AD11892 | AM16906-AS | AM15036-SS | 1435 |
| AD11893 | AM15037-AS | AM16907-SS | 1435 |
| AD11894 | AM15038-AS | AM16907-SS | 1435 |
| AD11895 | AM15039-AS | AM16907-SS | 1435 |
| AD11896 | AM15037-AS | AM16908-SS | 1435 |
| AD11897 | AM15038-AS | AM16908-SS | 1435 |
| AD11898 | AM15039-AS | AM16908-SS | 1435 |
| AD11899 | AM16909-AS | AM15036-SS | 1435 |
| AD11900 | AM16910-AS | AM15036-SS | 1435 |
| AD11953 | AM16962-AS | AM16539-SS | 1009 |
| AD11954 | AM16540-AS | AM16963-SS | 1009 |
| AD11955 | AM16962-AS | AM16963-SS | 1009 |
| AD12039 | AM16575-AS | AM17081-SS | 2464 |
| AD12244 | AM17362-AS | AM17361-SS | 1657 |
| AD12245 | AM17364-AS | AM17363-SS | 1435 |
| AD12246 | AM17365-AS | AM16539-SS | 1009 |
| AD12247 | AM17362-AS | AM16701-SS | 1657 |
| AD12248 | AM17364-AS | AM15036-SS | 1435 |
| AD12473 | AM17649-AS | AM16410-SS | 3157 |
| AD12474 | AM17649-AS | AM17650-SS | 3157 |
| AD12475 | AM17649-AS | AM17651-SS | 3157 |
| AD12476 | AM17652-AS | AM17651-SS | 3157 |
| AD12477 | AM17653-AS | AM17650-SS | 3157 |
| AD12478 | AM17654-AS | AM17651-SS | 3157 |
| AD12858 | AM18250-AS | AM15283-SS | 3155 |
| AD12859 | AM18251-AS | AM15283-SS | 3155 |
| AD13066 | AM15780-AS | AM18528-SS | 3155 |
| AD13072 | AM15780-AS | AM18534-SS | 3155 |
| AD13372 | AM16577-AS | AM19026-SS | 3155 |
| AD13494 | AM19163-AS | AM15283-SS | 3155 |
| AD13646 | AM16962-AS | AM19347-SS | 1009 |
| AD13736 | AM15780-AS | AM19421-SS | 3155 |
| AD13737 | AM15780-AS | AM19422-SS | 3155 |
| AD13838 | AM15780-AS | AM19573-SS | 3155 |

As described herein, in some embodiments, the duplexed sense strand nucleotide sequence and antisense strand nucleotide sequence can be linked to certain targeting ligands and/or PK/PD modulators. Certain exemplary targeting ligands and/or PK/PD modulators we linked as shown in the following Table 5.4, which shows fully conjugated duplexes and have an “AC” identification prefix.

TABLE 5.4
Conjugate ID Numbers With Chemically Modified Antisense and Sense Strands (including Linkers and Conjugates)

| | | SEQ | | |
|---|---|---|---|---|
| AC ID | Sense Strand (Fully Modified with Conjugated Targeting | ID | | SEQ ID |
| Number | Ligand)(5′ → 3′) | NO: | Antisense Strand (5′ → 3′) | NO: |
| AC001727 | avb6-pep 1-(NH- | 1406 | cPrpusAfsasGfcAfcGfaCfaCfcUfcGfcCfcCfsu | 655 |
| | C6)s(invAb)saggggcgaGfGfUfgucgugcuuas(invAb)(C6-S)-LP238b | | | |
| AC001728 | avb6-pep1-(NH- | 1407 | cPrpusCfsgsCfgUfaGfaAfgGfgCfgUfcUfgCfsc | 656 |
| | C6)s(invAb)sggcagacgCfCfCfuucuacgcgas(invAb)(C6-S)-LP238b | | | |
| AC001730 | avb6-pep 1-(NH- | 1408 | cPrpusAfsasUfaCfcGfaGfgAfaUfgUfcGfgGfsg | 657 |
| | C6)s(invAb)sccccgacaUfUfCfcucgguauuas(invAb)(C6-S)-LP238b | | | |
| AC001731 | avb6-pep 1-(NH- | 1409 | cPrpusAfsgsAfcAfaUfaAfaUfaCfcGfaGfgAfsa | 658 |
| | C6)s(invAb)suuccucggUfAfUfuuauugucuas(invAb)(C6-S)-LP238b | | | |
| AC001889 | avb6-pep 1-(NH- | 1410 | cPrpasCfsasAfuAfaAfuAfcCfgAfgGfaAfuGfsc | 679 |
| | C6)s(invAb)sgcauuccuCfGfGfuauuuauugus(invAb)(C6-S)-LP238b | | | |
| AC001890 | avb6-pep 1-(NH- | 1411 | cPrpasCfsgsUfaAfuAfcUfcCfaUfgAfcCfaGfsg | 680 |
| | C6)s(invAb)sccuggucaUfGfGfaguauuacgus(invAb)(C6-S)-LP238b | | | |
| AC001934 | avb6-pep1-(NH- | 1409 | cPrpusAfsgsaCfaauaaaUfaCfcGfaggasa | 681 |
| | C6)s(invAb)suuccucggUfAfUfuuauugucuas(invAb)(C6-S)-LP238b | | | |
| AC001935 | avb6-pep 1-(NH- | 1409 | cPrpusAfsgsacAfauaaaUfaCfcGfaggasa | 682 |
| | C6)s(invAb)suuccucggUfAfUfuuauugucuas(invAb)(C6-S)-LP238b | | | |
| AC001936 | avb6-pep 1-(NH- | 1409 | cPrpusAfsgsacaaUfaaaUfaCfcGfaggasa | 683 |
| | C6)s(invAb)suuccucggUfAfUfuuauugucuas(invAb)(C6-S)-LP238b | | | |
| AC001937 | avb6-pep1-(NH- | 1409 | cPrpusAfsgsacaauAfaaUfaCfcGfaggasa | 684 |
| | C6)s(invAb)suuccucggUfAfUfuuauugucuas(invAb)(C6-S)-LP238b | | | |
| AC001938 | avb6-pep 1-(NH- | 1412 | cPrpusAfsgsaCfaauaaaUfaCfcGfaggasc | 685 |
| | C6)s(invAb)sguccucggUfAfUfuuauugucuas(invAb)(C6-S)-LP238b | | | |
| AC001939 | avb6-pep 1-(NH- | 1413 | cPrpusAfsgsAfcAfaUfaAfaUfaCfcGfaGfgAfsa | 658 |
| | C6)s(invAb)suuccucggUfaUfuUfauugucuas(invAb)(C6-S)-LP238b | | | |
| AC001940 | avb6-pep 1-(NH- | 1413 | cPrpusAfsgsaCfaauaaaUfaCfcGfaggasa | 681 |
| | C6)s(invAb)suuccucggUfaUfuUfauugucuas(invAb)(C6-S)-LP238b | | | |
| AC002180 | avb6-pep 1-(NH- | 1414 | cPrpusAfsgsAfcAfaUfaAfaUfaCfcGfaGfgAfsc | 695 |
| | C6)s(invAb)sguccucggUfaUfuUfauugucuas(invAb)(C6-S)-LP238b | | | |
| AC002181 | avb6-pep 1-(NH- | 1413 | cPrpusAfsgAfcAfaUfaAfaUfaCfcGfaGfgsAfsa | 696 |
| | C6)s(invAb)suuccucggUfaUfuUfauugucuas(invAb)(C6-S)-LP238b | | | |
| AC002182 | avb6-pep 1-(NH- | 1413 | cPrpusAfsgAfcAfaUfaAfaUfaCfcGfaGfgAfsa | 697 |
| | C6)s(invAb)suuccucggUfaUfuUfauugucuas(invAb)(C6-S)-LP238b | | | |
| AC002183 | avb6-pep 1-(NH- | 1413 | cPrpusAfsgsacaAfuaaaUfaCfcGfaggasa | 698 |
| | C6)s(invAb)suuccucggUfaUfuUfauugucuas(invAb)(C6-S)-LP238b | | | |
| AC002184 | avb6-pep 1-(NH- | 1413 | cPrpusAfsgsacaauAfaaUfaCfcGfaggasa | 684 |
| | C6)s(invAb)suuccucggUfaUfuUfauugucuas(invAb)(C6-S)-LP238b | | | |
| AC002185 | avb6-pep1-(NH- | 1413 | cPrpusAfsgsacaauaAfaUfaCfcGfaggasa | 699 |
| | C6)s(invAb)suuccucggUfaUfuUfauugucuas(invAb)(C6-S)-LP238b | | | |
| AC002186 | avb6-pep 1-(NH- | 1413 | cPrpusAfsgsacaAfuAfAfaUfaCfcGfaggasa | 700 |
| | C6)s(invAb)suuccucggUfaUfuUfauugucuas(invAb)(C6-S)-LP238b | | | |
| AC002187 | avb6-pep 1-(NH- | 1415 | cPrpusAfsgsAfcAfaUfaAfaUfaCfcGfaGfgAfsa | 658 |
| | C6)s(invAb)suuccucggUfaUfUfuauugucuas(invAb)(C6-S)-LP238b | | | |
| AC002188 | avb6-pep 1-(NH- | 1415 | cPrpusAfsgsacaAfuaaaUfaCfcGfaggasa | 698 |
| | C6)s(invAb)suuccucggUfaUfUfuauugucuas(invAb)(C6-S)-LP238b | | | |
| AC002189 | avb6-pep 1-(NH- | 1415 | cPrpusAfsgsacaauAfaaUfaCfcGfaggasa | 684 |
| | C6)s(invAb)suuccucggUfaUfUfuauugucuas(invAb)(C6-S)-LP238b | | | |
| AC002190 | avb6-pep 1-(NH- | 1415 | cPrpusAfsgsacaauaAfaUfaCfcGfaggasa | 699 |
| | C6)s(invAb)suuccucggUfaUfUfuauugucuas(invAb)(C6-S)-LP238b | | | |
| AC002191 | avb6-pep 1-(NH- | 1415 | cPrpusAfsgsacaAfuAfAfaUfaCfcGfaggasa | 700 |
| | C6)s(invAb)suuccucggUfaUfUfuauugucuas(invAb)(C6-S)-LP238b | | | |
| AC002192 | avb6-pep 1-(NH- | 1413 | usAfsgsAfcAfaUfaAfaUfaCfcGfaGfgAfsa | 666 |
| | C6)s(invAb)suuccucggUfaUfuUfauugucuas(invAb)(C6-S)-LP238b | | | |
| AC002250 | avb6-pep 1-(NH- | 1416 | usCfsusUfcAfgCfaUfgUfcCfcAfcUfuGfsu | 701 |
| | C6)s(invAb)sacaaguggGfAfCfaugcuiaagas(invAb)(C6-S)-LP238b | | | |
| AC002251 | avb6-pep 1-(NH- | 1417 | usUfscsUfuCfaGfcAfuGfuCfcCfaCfuUfsg | 702 |
| | C6)s(invAb)scaagugggAfCfAfugcuiaagaas(invAb)(C6-S)-LP238b | | | |
| AC002252 | avb6-pep 1-(NH- | 1418 | asGfsgsUfaGfuUfcUfcAfuCfcUfgGfaAfsg | 703 |
| | C6)s(invAb)scuuccaggAfUfGfagaacuacuus(invAb)(C6-S)-LP238b | | | |
| AC002253 | avb6-pep 1-(NH- | 1419 | asGfsgsUfaGfuUfcUfcAfuCfcUfgGfaAfsg | 703 |
| | C6)s(invAb)scuuccaggAfUfGfagaacuaucus(invAb)(C6-S)-LP238b | | | |
| AC002254 | avb6-pep 1-(NH- | 1420 | asCfsasGfgUfaGfuUfcUfcAfuCfcUfgGfsa | 704 |
| | C6)s(invAb)succaggauGfAfGfaacuaccuius(invAb)(C6-S)-LP238b | | | |
| AC002255 | avb6-pep1-(NH- | 1421 | asCfsasGfgUfaGfuUfcUfcAfuCfcUfgGfsa | 704 |
| | C6)s(invAb)succaggauGfAfGfaacuacuugus(invAb)(C6-S)-LP238b | | | |
| AC002256 | avb6-pep 1-(NH- | 1422 | usGfsusAfcAfgGfuAfgUfuCfuCfaUfcCfsu | 705 |
| | C6)s(invAb)saggaugagAfAfCfuaccuiuacas(invAb)(C6-S)-LP238b | | | |
| AC002257 | avb6-pep 1-(NH- | 1423 | usCfscsAfuGfaCfcAfgGfuAfcAfgGfuAfsg | 706 |
| | C6)s(invAb)scuaccuguAfCfCfuggucauigas(invAb)(C6-S)-LP238b | | | |
| AC002258 | avb6-pep 1-(NH- | 1424 | usCfscsAfuGfaCfcAfgGfuAfcAfgGfuAfsg | 706 |
| | C6)s(invAb)scuaccuguAfCfCfugguuauggas(invAb)(C6-S)-LP238b | | | |
| AC002259 | avb6-pep1-(NH- | 1425 | usCfscsAfaAfcUfuGfcUfcAfgCfaGfuGfsu | 707 |
| | C6)s(invAb)sacacugcuGfAfGfcaaguuuigas(invAb)(C6-S)-LP238b | | | |
| AC002260 | avb6-pep 1-(NH- | 1426 | usAfsgsAfcAfaUfaAfaUfaCfcGfaGfgAfsa | 666 |
| | C6)s(invAb)suuccucggUfaUfuUfauugucuas(invAb)(C6-S)- | | | |
| | LP238bdT | | | |
| AC002324 | avb6-pep 1-(NH- | 1415 | cPrpusAfsgacaauaAfaUfaCfcGfaggasa | 724 |
| | C6)s(invAb)suuccucggUfaUfUfuauugucuas(invAb)(C6-S)-LP238b | | | |
| AC002325 | avb6-pep 1-(NH- | 1415 | usAfsgsacaauaAfaUfaCfcGfaggasa | 725 |
| | C6)s(invAb)suuccucggUfaUfUfuauugucuas(invAb)(C6-S)-LP238b | | | |
| AC002593 | avb6-pep1-(NH- | 1427 | cPrpusAfscsAfgAfcAfaUfaAfaUfaCfcGfaGfsg | 736 |
| | C6)s(invAb)sccucgguaUfUfUfauuguuuguas(invAb)(C6-S)-LP238b | | | |
| AC002594 | avb6-pep 1-(NH- | 1428 | cPrpusAfscsAfgAfcAfaUfaAfaUfaCfcGfaGfsg | 736 |
| | C6)s(invAb)sccucgguaUfUfUfauugucuguas(invAb)(C6-S)-LP238b | | | |
| AC002595 | avb6-pep 1-(NH- | 1429 | cPrpusAfscsAfgAfcAfaUfaAfaUfaCfcGfaGfsg | 736 |
| | C6)s(invAb)sccucgguaUfUfUfauugucuiuas(invAb)(C6-S)-LP238b | | | |
| AC002602 | avb6-pep 1-(NH- | 1430 | cPrpusUfscsGfaAfgAfcAfgUfuCfuAfgGfgUfs | 751 |
| | C6)s(invAb)sgacccuagAfAfCfugucuuciaas(invAb)(C6-S)-LP238b | | C | |
| AC002603 | avb6-pep1-(NH- | 1431 | cPrpusCfsgsAfaGfaCfaGfuUfcUfaGfgGfuUfsc | 752 |
| | C6)s(invAb)sgaacccuaGfAfAfcugucuucias(invAb)(C6-S)-LP238b | | | |
| AC002637 | avb6-pep1-(NH-C6)sC6cscscuagAfAfCfugucuucgsasa(invAb)(C6- | 1432 | cPrpusUfscgaAfgacaguuCfuAfgggsusu | 746 |
| | S)-LP238b | | | |
| AC002689 | avb6-pep 1-(NH- | 1433 | cPrpusCfsgsaagUfugcaUfgUfgUfcggusg | 758 |
| | C6)s(invAb)scaccgacaCfaUfgCfaacuucias(invAb)(C6-S)-LP238b | | | |
| AC002690 | avb6-pep 1-(NH- | 1434 | cPrpusUfsgsuAfguggacGfaUfcUfugccsa | 759 |
| | C6)s(invAb)suggcaagaUfcGfuccacuacaas(invAb)(C6-S)-LP238b | | | |
| AC002691 | avb6-pep 1-(NH- | 1435 | cPrpusCfsGfuAfauacUfcCfaUfgAfccagsg | 757 |
| | C6)s(invAb)sccuggucaUfgGfAfguauuacgas(invAb)(C6-S)-LP238b | | | |
| AC002830 | avb6-pep 1-(NH- | 1428 | cPrpusAfscsagaCfaauaAfaUfaCfcgagsg | 761 |
| | C6)s(invAb)sccucgguaUfUfUfauugucuguas(invAb)(C6-S)-LP238b | | | |
| AC002831 | avb6-pep1-(NH- | 1436 | cPrpusAfscsagaCfaauaAfaUfaCfcgagsg | 761 |
| | C6)s(invAb)sccucgguaUfuUfaUfugucuguas(invAb)(C6-S)-LP238b | | | |
| AC002832 | avb6-pep1-(NH- | 1437 | cPrpusAfscsagaCfaauaAfaUfaCfcgagsg | 761 |
| | C6)s(invAb)sccucgguaUfuUfAfuugucuguas(invAb)(C6-S)-LP238b | | | |
| AC002833 | avb6-pep 1-(NH- | 1437 | cPrpusAfscagaCfaauaAfaUfaCfcgagsg | 762 |
| | C6)s(invAb)sccucgguaUfuUfAfuugucuguas(invAb)(C6-S)-LP238b | | | |
| AC002834 | avb6-pep 1-(NH- | 1436 | cPrpusAfscsaGfacaauaAfaUfaCfcgagsg | 763 |
| | C6)s(invAb)sccucgguaUfuUfaUfugucuguas(invAb)(C6-S)-LP238b | | | |
| AC002835 | avb6-pep 1-(NH- | 1437 | cPrpusAfscagacaaUfaAfaUfaCfcgagsg | 764 |
| | C6)s(invAb)sccucgguaUfuUfAfuugucuguas(invAb)(C6-S)-LP238b | | | |
| AC002921 | avb6-pep 1-(NH- | 1415 | dTssAfsgacaauaAfaUfaCfcGfaggasa | 765 |
| | C6)s(invAb)suuccucggUfaUfUfuauugucuas(invAb)(C6-S)-LP238b | | | |
| AC002922 | avb6-pep 1-(NH- | 1415 | UssAfsgacaauaAfaUfaCfcGfaggasa | 766 |
| | C6)s(invAb)suuccucggUfaUfUfuauugucuas(invAb)(C6-S)-LP238b | | | |
| AC003000 | avb6-pep 1-(NH- | 1415 | usAfsgacaauaAfaUfaCfcGfaggasa | 747 |
| | C6)s(invAb)suuccucggUfaUfUfuauugucuas(invAb)(C6-S)-LP238b | | | |
| AC003068 | avb6-pep 1-(NH- | 1415 | dTssAfgacaauaAfaUfaCfcGfaggasa | 767 |
| | C6)s(invAb)suuccucggUfaUfUfuauugucuas(invAb)(C6-S)-LP238b | | | |
| AC003224 | avb6-pep1-(NH- | 1438 | cPrpusAfsgacaauaAfaUfaCfcGfaggasa | 724 |
| | C6)s(invAb)suuccucggUfaUfUfuauugucuas(invAb)s(C6-S)-LP238b | | | |

TABLE 5.5
DMPK Agent Sense Strand Sequences (Shown with Targeting Ligand Conjugate.)

| Strand ID | Modified Sense Strand (5′ → 3′) | SEQ ID NO. |
|---|---|---|
| CS002215 | avb6-pep1-(NH-C6)s(invAb)saggggcgaGfGfUfgucgugcuuas(invAb)(C6-S)-LP238b | 1406 |
| CS002217 | avb6-pep1-(NH-C6)s(invAb)sggcagacgCfCfCfuucuacgcgas(invAb)(C6-S)-LP238b | 1407 |
| CS002221 | avb6-pep1-(NH-C6s)(invAb)sccccgacaUfUfCfcucgguauuas(invAb)(C6-S)-LP238b | 1408 |
| CS002223 | avb6-pep1-(NH-C6)s(invAb)suuccucggUfAfUfuuauugucuas(invAb)(C6-S)-LP238b | 1409 |
| CS002411 | avb6-pep1-(NH-C6)s(invAb)sgcauuccuCfGfGfuauuuauugus(invAb)(C6-S)-LP238b | 1410 |
| CS002413 | avb6-pep1-(NH-C6)s(invAb)sccuggucaUfGfGfaguauuacgus(invAb)(C6-S)-LP238b | 1411 |
| CS002472 | avb6-pep1-(NH-C6)s(invAb)sguccucggUfAfUfuuauugucuas(invAb)(C6-S)-LP238b | 1412 |
| CS002474 | avb6-pep1-(NH-C6)s(invAb)suuccucggUfaUfuUfauugucuas(invAb)(C6-S)-LP238b | 1413 |
| CS002769 | avb6-pep1-(NH-C6)s(invAb)sguccucggUfaUfuUfauugucuas(invAb)(C6-S)-LP238b | 1414 |
| CS002776 | avb6-pep1-(NH-C6)s(invAb)suuccucggUfaUfUfuauugucuas(invAb)(C6-S)-LP238b | 1415 |
| CS002852 | avb6-pep1-(NH-C6)s(invAb)sacaaguggGfAfCfaugcuiaagas(invAb)(C6-S)-LP238b | 1416 |
| CS002854 | avb6-pep1-(NH-C6)s(invAb)scaagugggAfCfAfugcuiaagaas(invAb)(C6-S)-LP238b | 1417 |
| CS002856 | avb6-pep1-(NH-C6)s(invAb)scuuccaggAfUfGfagaacuacuus(invAb)(C6-S)-LP238b | 1418 |
| CS002858 | avb6-pep1-(NH-C6)s(invAb)scuuccaggAfUfGfagaacuaucus(invAb)(C6-S)-LP238b | 1419 |
| CS002859 | avb6-pep1-(NH-C6)s(invAb)succaggauGfAfGfaacuaccuius(invAb)(C6-S)-LP238b | 1420 |
| CS002861 | avb6-pep1-(NH-C6)s(invAb)succaggauGfAfGfaacuacuugus(invAb)(C6-S)-LP238b | 1421 |
| CS002862 | avb6-pep1-(NH-C6)s(invAb)saggaugagAfAfCfuaccuiuacas(invAb)(C6-S)-LP238b | 1422 |
| CS002864 | avb6-pep1-(NH-C6)s(invAb)scuaccuguAfCfCfuggucauigas(invAb)(C6-S)-LP238b | 1423 |
| CS002866 | avb6-pep1-(NH-C6)s(invAb)scuaccuguAfCfCfugguuauggas(invAb)(C6-S)-LP238b | 1424 |
| CS002867 | avb6-pep1-(NH-C6)s(invAb)sacacugcuGfAfGfcaaguuuigas(invAb)(C6-S)-LP238b | 1425 |
| CS002869 | avb6-pep1-(NH-C6)s(invAb)suuccucggUfaUfuUfauugucuas(invAb)(C6-S)-LP238bdT | 1426 |
| CS003303 | avb6-pep1-(NH-C6)s(invAb)sccucgguaUfUfUfauuguuuguas(invAb)(C6-S)-LP238b | 1427 |
| CS003305 | avb6-pep1-(NH-C6)s(invAb)sccucgguaUfUfUfauugucuguas(invAb)(C6-S)-LP238b | 1428 |
| CS003306 | avb6-pep1-(NH-C6)s(invAb)sccucgguaUfUfUfauugucuiuas(invAb)(C6-S)-LP238b | 1429 |
| CS003317 | avb6-pep1-(NH-C6)s(invAb)sgacccuagAfAfCfugucuuciaas(invAb)(C6-S)-LP238b | 1430 |
| CS003319 | avb6-pep1-(NH-C6)s(invAb)sgaacccuaGfAfAfcugucuucias(invAb)(C6-S)-LP238b | 1431 |
| CS003361 | avb6-pep1-(NH-C6)cscscuagAfAfCfugucuucgsasa(invAb)(C6-S)-LP238b | 1432 |
| CS003421 | avb6-pep1-(NH-C6)s(invAb)scaccgacaCfaUfgCfaacuucias(invAb)(C6-S)-LP238b | 1433 |
| CS003423 | avb6-pep1-(NH-C6)s(invAb)suggcaagaUfcGfuccacuacaas(invAb)(C6-S)-LP238b | 1434 |
| CS003425 | avb6-pep1-(NH-C6)s(invAb)sccuggucaUfgGfAfguauuacgas(invAb)(C6-S)-LP238b | 1435 |
| CS003602 | avb6-pep1-(NH-C6)s(invAb)sccucgguaUfuUfaUfugucuguas(invAb)(C6-S)-LP238b | 1436 |
| CS003603 | avb6-pep1-(NH-C6)s(invAb)sccucgguaUfuUfAfuugucuguas(invAb)(C6-S)-LP238b | 1437 |
| CS004056 | avb6-pep1-(NH-C6)s(invAb)suuccucggUfaUfUfuauugucuas(invAb)s(C6-S)-LP238b | 1438 |
| CS004328 | avb6-pep1-(NH-C6)s(invAb)sccuggucaUfgGfAfguauuacga(invAb)(C6-S)-LP238b | 1439 |
| CS004329 | avb6-pep1-(NH-C6)s(invAb)sccugggucaUfgGfAfguauuacgas(invAb)(C6-S)-LP238b | 1440 |
| CS004331 | avb6-pep1-(NH-C6)s(invAb)scuggucaUfgGfAfguauuacgas(invAb)(C6-S)-LP238b | 1441 |
| CS006574 | avb6-pep1-(NH-C6)s(invAb)sccuggucaUfgGfAfguauuacgas(invAb) | 1442 |

TABLE 5.6
DMPK RNAi Agent Duplexes with Corresponding Sense and Antisense
Strand ID Numbers and Sequence ID numbers for the modified and unmodified
nucleotide sequences. (Shown with Targeting Ligand Conjugates.)

| | | AS | | | SS | |
|---|---|---|---|---|---|---|
| | | Modified | AS | | Modified | SS |
| | | Seq ID | Unmodified | | Seq ID | Unmodified |
| Duplex | AS ID | No: | Seq ID No: | SS ID | No: | Seq ID No: |
| AC001727 | AM14133-AS | 655 | 1465 | CS002215 | 1406 | 1541 |
| AC001728 | AM14135-AS | 656 | 1466 | CS002217 | 1407 | 1588 |
| AC001730 | AM14139-AS | 657 | 1467 | CS002221 | 1408 | 1542 |
| AC001731 | AM14141-AS | 658 | 1468 | CS002223 | 1409 | 1543 |
| AC001889 | AM14548-AS | 679 | 1463 | CS002411 | 1410 | 1532 |
| AC001890 | AM14550-AS | 680 | 1455 | CS002413 | 1411 | 1523 |
| AC001934 | AM14612-AS | 681 | 1468 | CS002223 | 1409 | 1543 |
| AC001935 | AM14613-AS | 682 | 1468 | CS002223 | 1409 | 1543 |
| AC001936 | AM14614-AS | 683 | 1468 | CS002223 | 1409 | 1543 |
| AC001937 | AM14615-AS | 684 | 1468 | CS002223 | 1409 | 1543 |
| AC001938 | AM14617-AS | 685 | 1483 | CS002472 | 1412 | 1591 |
| AC001939 | AM14141-AS | 658 | 1468 | CS002474 | 1413 | 1543 |
| AC001940 | AM14612-AS | 681 | 1468 | CS002474 | 1413 | 1543 |
| AC002180 | AM15277-AS | 695 | 1483 | CS002769 | 1414 | 1591 |
| AC002181 | AM15278-AS | 696 | 1468 | CS002474 | 1413 | 1543 |
| AC002182 | AM15279-AS | 697 | 1468 | CS002474 | 1413 | 1543 |
| AC002183 | AM15280-AS | 698 | 1468 | CS002474 | 1413 | 1543 |
| AC002184 | AM14615-AS | 684 | 1468 | CS002474 | 1413 | 1543 |
| AC002185 | AM15281-AS | 699 | 1468 | CS002474 | 1413 | 1543 |
| AC002186 | AM15282-AS | 700 | 1468 | CS002474 | 1413 | 1543 |
| AC002187 | AM14141-AS | 658 | 1468 | CS002776 | 1415 | 1543 |
| AC002188 | AM15280-AS | 698 | 1468 | CS002776 | 1415 | 1543 |
| AC002189 | AM14615-AS | 684 | 1468 | CS002776 | 1415 | 1543 |
| AC002190 | AM15281-AS | 699 | 1468 | CS002776 | 1415 | 1543 |
| AC002191 | AM15282-AS | 700 | 1468 | CS002776 | 1415 | 1543 |
| AC002192 | AM14158-AS | 666 | 1468 | CS002474 | 1413 | 1543 |
| AC002250 | AM15477-AS | 701 | 1484 | CS002852 | 1416 | 1592 |
| AC002251 | AM15479-AS | 702 | 1485 | CS002854 | 1417 | 1593 |
| AC002252 | AM15481-AS | 703 | 1486 | CS002856 | 1418 | 1594 |
| AC002253 | AM15481-AS | 703 | 1486 | CS002858 | 1419 | 1595 |
| AC002254 | AM15484-AS | 704 | 1487 | CS002859 | 1420 | 1596 |
| AC002255 | AM15484-AS | 704 | 1487 | CS002861 | 1421 | 1597 |
| AC002256 | AM15487-AS | 705 | 1488 | CS002862 | 1422 | 1598 |
| AC002257 | AM15489-AS | 706 | 1489 | CS002864 | 1423 | 1599 |
| AC002258 | AM15489-AS | 706 | 1489 | CS002866 | 1424 | 1600 |
| AC002259 | AM15492-AS | 707 | 1490 | CS002867 | 1425 | 1601 |
| AC002260 | AM14158-AS | 666 | 1468 | CS002869 | 1426 | 1539 |
| AC002324 | AM15780-AS | 724 | 1468 | CS002776 | 1415 | 1543 |
| AC002325 | AM15781-AS | 725 | 1468 | CS002776 | 1415 | 1543 |
| AC002593 | AM16409-AS | 736 | 1480 | CS003303 | 1427 | 1553 |
| AC002594 | AM16409-AS | 736 | 1480 | CS003305 | 1428 | 1602 |
| AC002595 | AM16409-AS | 736 | 1480 | CS003306 | 1429 | 1603 |
| AC002602 | AM16803-AS | 751 | 1446 | CS003317 | 1430 | 1514 |
| AC002603 | AM16805-AS | 752 | 1445 | CS003319 | 1431 | 1513 |
| AC002637 | AM16575-AS | 746 | 1508 | CS003361 | 1432 | 1580 |
| AC002689 | AM17362-AS | 758 | 1504 | CS003421 | 1433 | 1573 |
| AC002690 | AM17364-AS | 759 | 1457 | CS003423 | 1434 | 1525 |
| AC002691 | AM16962-AS | 757 | 1507 | CS003425 | 1435 | 1579 |
| AC002830 | AM17649-AS | 761 | 1480 | CS003305 | 1428 | 1602 |
| AC002831 | AM17649-AS | 761 | 1480 | CS003602 | 1436 | 1602 |
| AC002832 | AM17649-AS | 761 | 1480 | CS003603 | 1437 | 1602 |
| AC002833 | AM17652-AS | 762 | 1480 | CS003603 | 1437 | 1602 |
| AC002834 | AM17653-AS | 763 | 1480 | CS003602 | 1436 | 1602 |
| AC002835 | AM17654-AS | 764 | 1480 | CS003603 | 1437 | 1602 |
| AC002921 | AM18250-AS | 765 | 1623 | CS002776 | 1415 | 1543 |
| AC002922 | AM18251-AS | 766 | 1468 | CS002776 | 1415 | 1543 |
| AC003000 | AM16577-AS | 747 | 1468 | CS002776 | 1415 | 1543 |
| AC003068 | AM19163-AS | 767 | 1623 | CS002776 | 1415 | 1543 |
| AC003224 | AM15780-AS | 724 | 1468 | CS004056 | 1438 | 1543 |

TABLE 5.7
DMPK RNAi Agent Conjugate Duplex ID Numbers
Referencing Position Targeted on DMPK Gene

| | | | | Targeted DMPK |
|---|---|---|---|---|
| | | | Corresponding | Gene Position |
| Duplex | AS ID | SS ID | AD Duplex | (Of SEQ ID NO: 1) |
| AC001727 | AM14133-AS | CS002215 | AD09910 | 902 |
| AC001728 | AM14135-AS | CS002217 | AD09911 | 1395 |
| AC001730 | AM14139-AS | CS002221 | AD09913 | 3147 |
| AC001731 | AM14141-AS | CS002223 | AD09914 | 3155 |
| AC001889 | AM14548-AS | CS002411 | AD10207 | 3152 |
| AC001890 | AM14550-AS | CS002413 | AD10208 | 1009 |
| AC001934 | AM14612-AS | CS002223 | AD10254 | 3155 |
| AC001935 | AM14613-AS | CS002223 | AD10255 | 3155 |
| AC001936 | AM14614-AS | CS002223 | AD10256 | 3155 |
| AC001937 | AM14615-AS | CS002223 | AD10257 | 3155 |
| AC001938 | AM14617-AS | CS002472 | AD10258 | 3155 |
| AC001939 | AM14141-AS | CS002474 | AD10259 | 3155 |
| AC001940 | AM14612-AS | CS002474 | AD10260 | 3155 |
| AC002180 | AM15277-AS | CS002769 | AD10715 | 3155 |
| AC002181 | AM15278-AS | CS002474 | AD10716 | 3155 |
| AC002182 | AM15279-AS | CS002474 | AD10717 | 3155 |
| AC002183 | AM15280-AS | CS002474 | AD10718 | 3155 |
| AC002184 | AM14615-AS | CS002474 | AD10719 | 3155 |
| AC002185 | AM15281-AS | CS002474 | AD10720 | 3155 |
| AC002186 | AM15282-AS | CS002474 | AD10721 | 3155 |
| AC002187 | AM14141-AS | CS002776 | AD10722 | 3155 |
| AC002188 | AM15280-AS | CS002776 | AD10723 | 3155 |
| AC002189 | AM14615-AS | CS002776 | AD10724 | 3155 |
| AC002190 | AM15281-AS | CS002776 | AD10725 | 3155 |
| AC002191 | AM15282-AS | CS002776 | AD10726 | 3155 |
| AC002192 | AM14158-AS | CS002474 | AD10727 | 3155 |
| AC002250 | AM15477-AS | CS002852 | AD10850 | 882 |
| AC002251 | AM15479-AS | CS002854 | AD10851 | 883 |
| AC002252 | AM15481-AS | CS002856 | AD10852 | 985 |
| AC002253 | AM15481-AS | CS002858 | AD10853 | 985 |
| AC002254 | AM15484-AS | CS002859 | AD10854 | 987 |
| AC002255 | AM15484-AS | CS002861 | AD10855 | 987 |
| AC002256 | AM15487-AS | CS002862 | AD10856 | 990 |
| AC002257 | AM15489-AS | CS002864 | AD10857 | 1000 |
| AC002258 | AM15489-AS | CS002866 | AD10858 | 1000 |
| AC002259 | AM15492-AS | CS002867 | AD10859 | 1046 |
| AC002260 | AM14158-AS | CS002869 | AD10727 | 3155 |
| AC002324 | AM15780-AS | CS002776 | AD11086 | 3155 |
| AC002325 | AM15781-AS | CS002776 | AD11087 | 3155 |
| AC002593 | AM16409-AS | CS003303 | AD11548 | 3157 |
| AC002594 | AM16409-AS | CS003305 | AD11549 | 3157 |
| AC002595 | AM16409-AS | CS003306 | AD11550 | 3157 |
| AC002602 | AM16803-AS | CS003317 | AD11833 | 2464 |
| AC002603 | AM16805-AS | CS003319 | AD11834 | 2463 |
| AC002637 | AM16575-AS | CS003361 | AD12039 | 2464 |
| AC002689 | AM17362-AS | CS003421 | AD12244 | 1657 |
| AC002690 | AM17364-AS | CS003423 | AD12245 | 1435 |
| AC002691 | AM16962-AS | CS003425 | AD11955 | 1009 |
| AC002830 | AM17649-AS | CS003305 | AD12473 | 3157 |
| AC002831 | AM17649-AS | CS003602 | AD12474 | 3157 |
| AC002832 | AM17649-AS | CS003603 | AD12475 | 3157 |
| AC002833 | AM17652-AS | CS003603 | AD12476 | 3157 |
| AC002834 | AM17653-AS | CS003602 | AD12477 | 3157 |
| AC002835 | AM17654-AS | CS003603 | AD12478 | 3157 |
| AC002921 | AM18250-AS | CS002776 | AD12858 | 3155 |
| AC002922 | AM18251-AS | CS002776 | AD12859 | 3155 |
| AC003000 | AM16577-AS | CS002776 | AD13372 | 3155 |
| AC003068 | AM19163-AS | CS002776 | AD13494 | 3155 |
| AC003224 | AM15780-AS | CS004056 | AD13838 | 3155 |

In some embodiments, a DMPK RNAi agent is prepared or provided as a salt, mixed salt, a free-acid, or a free base. In some embodiments, a DMPK RNAi agent is prepared as a pharmaceutically acceptable salt. In some embodiments, a DMPK RNAi agent is prepared as a pharmaceutically acceptable sodium salt. Such forms that are well known in the art are within the scope of the inventions disclosed herein. The RNAi agents described herein, upon delivery to a cell expressing a DMPK gene, inhibit or knockdown expression of one or more DMPK genes in vivo and/or in vitro.

In some embodiments, described herein are compositions that include a combination or cocktail of at least two DMPK RNAi agents having different sequences. In some embodiments, the two or more DMPK RNAi agents are each separately and independently linked to targeting groups. In some embodiments, the two or more DMPK RNAi agents are each linked to targeting groups that include or consist of targeting ligands. In some embodiments, the two or more DMPK RNAi agents are each linked to targeting groups.

### Targeting Groups, Linking Groups, and Delivery Vehicles

In some embodiments, a DMPK RNAi agent contains or is conjugated to one or more non-nucleotide groups including, but not limited to, a targeting group, a linking group, a pharmacokinetic/pharmacodynamic (PK/PD) modulator, a delivery polymer, or a delivery vehicle. The non-nucleotide group can enhance targeting, delivery, or attachment of the RNAi agent. Examples of linking groups are provided in Table 6.1, and examples of targeting groups or targeting ligands are provided in Tables 6.2 and 6.3. The non-nucleotide group can be covalently linked to the 3′ and/or 5′ end of either the sense strand and/or the antisense strand. In some embodiments, a DMPK RNAi agent contains a non-nucleotide group linked to the 3′ and/or 5′ end of the sense strand. In some embodiments, a non-nucleotide group is linked to the 5′ end of a DMPK RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting ligands enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers.

The DMPK RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the DMPK RNAi agents disclosed herein are synthesized having an NH2-C₆ group (represented as (NH2-C₆) in the modified sequences herein) at the 5′-terminus of the sense strand of the RNAi agent. The terminal amino group subsequently can be reacted to form a conjugate with, for example, a group that includes a targeting ligand. In some embodiments, the DMPK RNAi agents disclosed herein are synthesized having one or more alkyne groups at the 5′-terminus of the sense strand of the RNAi agent. The terminal alkyne group(s) can subsequently be reacted to form a conjugate with, for example, a group that includes a targeting ligand.

In some embodiments, RNAi agents comprise a targeting group, which includes 2 or more targeting ligands. In some embodiments, a targeting group may be conjugated at the 5′ or 3′ end of the sense strand of an RNAi agent. In some embodiments, a targeting group may be conjugated to an internal nucleotide on an RNAi agent. In some embodiments, a targeting group may consist of two targeting ligands linked together, referred to as a “bidentate” targeting group. In some embodiments, a targeting group may consist of three targeting ligands linked together, referred to as a “tridentate” targeting group. In some embodiments, a targeting group may consist of four targeting ligands linked together, referred to as a “tetradentate” targeting group.

In some embodiments, the use of a targeting ligand facilitates cell-specific targeting to cells having desired receptors on its respective surface, and binding of the targeting ligand can facilitate entry of the therapeutic agent, such as an RNAi agent, to which it is linked, into cells such as skeletal muscle cells. Targeting ligands can be monomeric or monovalent (e.g., having a single targeting moiety) or multimeric or multivalent (e.g., having multiple targeting moieties). The targeting group can be attached to the 3′ and/or 5′ end of the RNAi oligonucleotide using methods known in the art.

Embodiments of the present disclosure include pharmaceutical compositions for delivering a DMPK RNAi agent to a skeletal muscle cell in vivo. Such pharmaceutical compositions can include, for example, a DMPK RNAi agent conjugated to a targeting group that comprises a targeting ligand.

In some embodiments, the DMPK RNAi agents disclosed herein can reduce DMPK gene expression in one or more of the following tissues: paraspinal, facial, torso, abdominal, and limb muscle tissues, including for example, in the triceps, biceps, quadriceps, pectoralis, gastrocnemius, soleus, masseter, EDL (extensor digitorum longus), TA (Tibialis anterior), trapezius, and/or diaphragm.

In some embodiments, a linking group is conjugated to the RNAi agent. The linking group facilitates covalent linkage of the agent to a targeting group, pharmacokinetic modulator, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include, but are not limited to: C6-SS-C6, 6-SS-6, reactive groups such as primary amines (e.g., NH2-C6) and alkynes, alkyl groups, abasic residues/nucleotides, amino acids, tri-alkyne functionalized groups, ribitol, and/or PEG groups.

A linker or linking group is a connection between two atoms that links one chemical group (such as an RNAi agent) or segment of interest to another chemical group (such as a targeting group, pharmacokinetic modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not be limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description.

In some embodiments, targeting groups are linked to the DMPK RNAi agents without the use of an additional linker. In some embodiments, the targeting group is designed having a linker readily present to facilitate the linkage to a DMPK RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

Any of the DMPK RNAi agent nucleotide sequences listed in Tables 2, 3, and 4.1, 4.2, 4.3, 4.4, 4.5, 4.6, 5.4, and 5.5, whether modified or unmodified, can contain 3′ and/or 5′ targeting group(s), linking group(s), and/or pharmacokinetic modulator(s). Any of the DMPK RNAi agent sequences listed in Tables 3 and 4.1, 4.2, 4.3, 4.4, 4.5, 4.6, 5.4, and 5.5, or are otherwise described herein, which contain a 3′ or 5′ targeting group, linking group, or pharmacokinetic modulator can alternatively contain no 3′ or 5′ targeting group, linking group, or PK/PD modulator, or can contain a different 3′ or 5′ targeting group, linking group, or PK/PD modulator including, but not limited to, those depicted in Tables 6.1, 6.2, 6.3, 6.4, 6.5, 6.6 or 6.7. Any of the DMPK RNAi agent duplexes listed in Table 5.1 (or Table 5.2, 5.3, 5.4, 5.6, or 5.7), whether modified or unmodified, can further comprise a targeting group, linking group, or PK/PD modulator, including, but not limited to, those depicted in Tables 6.1, 6.2, 6.3, 6.4, 6.5, 6.6, or 6.7, and in some embodiments the targeting group, linking group and/or PK/PD modulator can be attached to the 3′ or 5′ terminus of either the sense strand or the antisense strand of the DMPK RNAi agent duplex.

Examples of certain modified nucleotides and linking groups are provided in Table 6.1.

TABLE 6.1
Structures Representing Various Modified Nucleotides and Linkage Groups

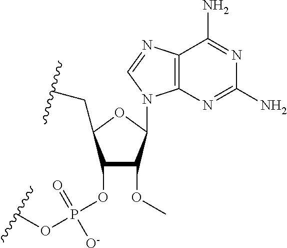

a_2N

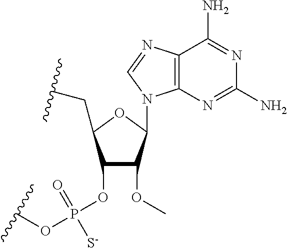

a_2Ns

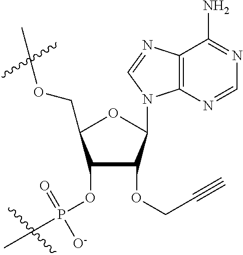

aAlk

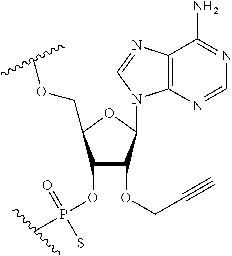

aAlks

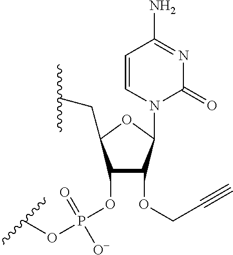

cAlk

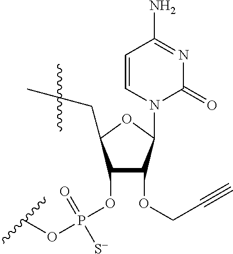

cAlks

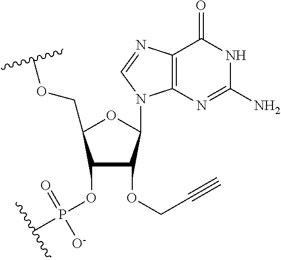

gAlk

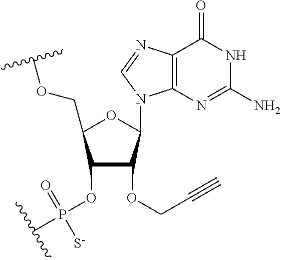

gAlks

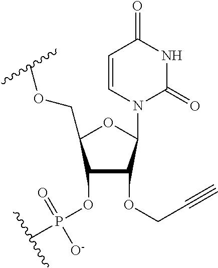

uAlk

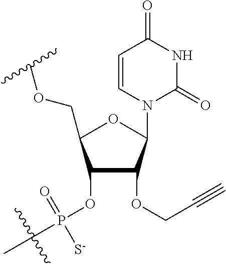

uAlks

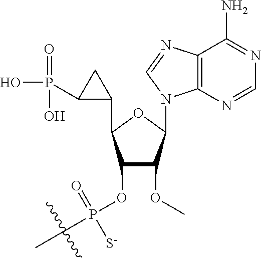

cPrpas

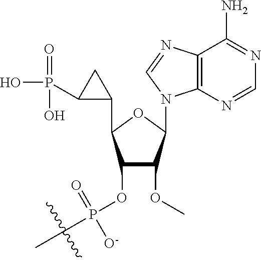

cPrpa

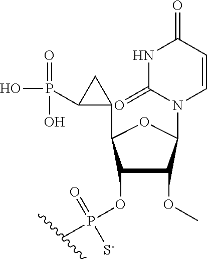

cPrpus

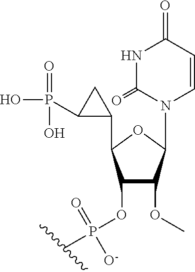

cPrpu
When positioned internally:

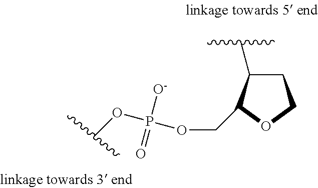

(invAb)
When positioned internally:

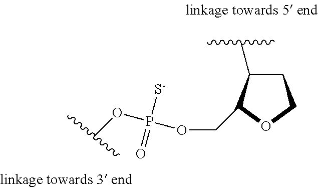

(invAb)s
When positioned at the 3′ terminal end:
linkage towards 5′ end

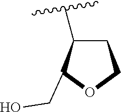

(invAb)
When positioned at the 3′ terminal end of oligonucleotide:

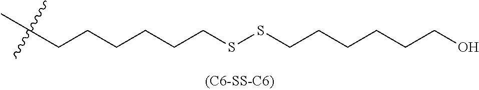 [SMILES: *CCCCCCSSCCCCCCO]

When positioned internally:

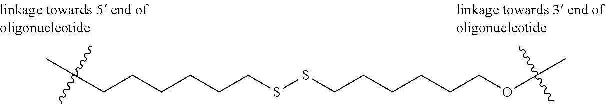 [SMILES: *CCCCCCSSCCCCCCO*]

(C6-SS-C6)

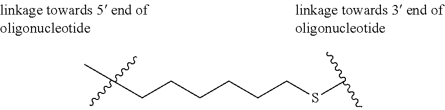 [SMILES: *CCCCCCS*]

(C6-S)
When positioned at the 3′ terminal end:

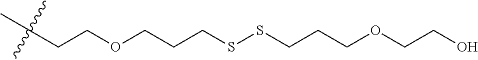 [SMILES: *CCOCCCSSCCCOCCO]

(6-SS-6)
When positioned internally in oligonucleotide:

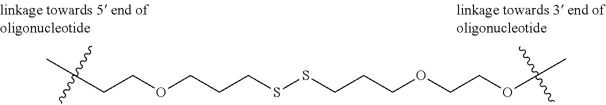 [SMILES: *CCOCCCSSCCCOCCO*]

(6-SS-6)

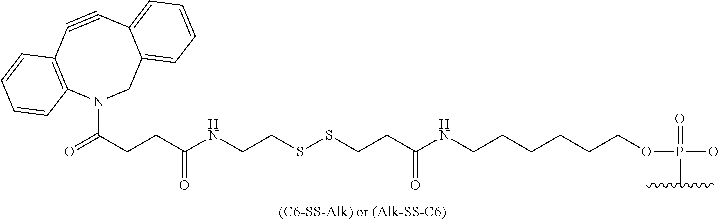 [SMILES: *P(=O)([O-])OCCCCCCNC(=O)CCSSCCNC(=O)CCC(=O)N1Cc2ccccc2C#Cc2ccccc21]

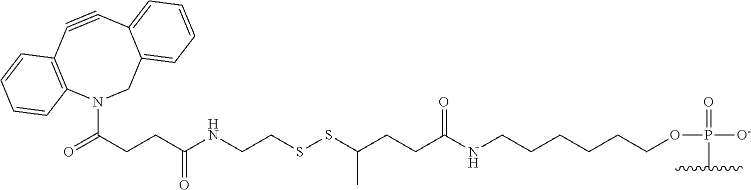 [SMILES: *P(=O)([O-])OCCCCCCNC(=O)CCC(C)SSCCNC(=O)CCC(=O)N1Cc2ccccc2C#Cc2ccccc21]

(C6-SS-Alk-Me)

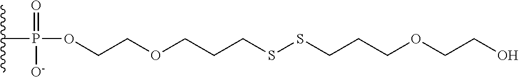 [SMILES: *P(=O)([O-])OCCOCCCSSCCCOCCO]

(PEG-C3-SS)

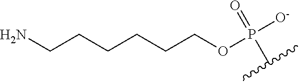 [SMILES: *P(=O)([O-])OCCCCCCN]

(NH2-C6)

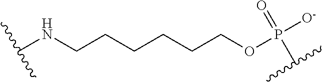 [SMILES: *NCCCCCCOP(*)(=O)[O-]]

(NH-C6)

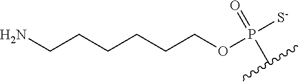 [SMILES: *P(=O)([S-])OCCCCCCN]

(NH2-C6)s

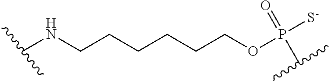 [SMILES: *NCCCCCCOP(*)(=O)[S-]]

(NH-C6)s

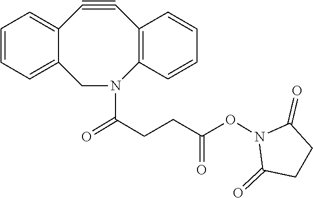 [SMILES: O=C(CCC(=O)N1Cc2ccccc2C#Cc2ccccc21)ON1C(=O)CCC1=O]

DBCO-NHS (BroadPharm ® BP-22231)

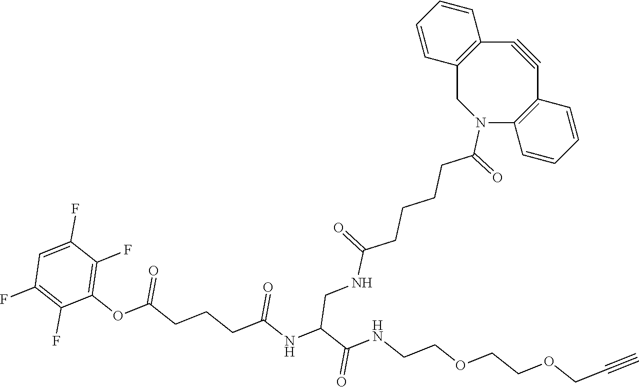 [SMILES: C#CCOCCOCCNC(=O)C(CNC(=O)CCCCC(=O)N1Cc2ccccc2C#Cc2ccccc21)NC(=O)CCCC(=O)Oc1c(F)c(F)cc(F)c1F]

Linker-1

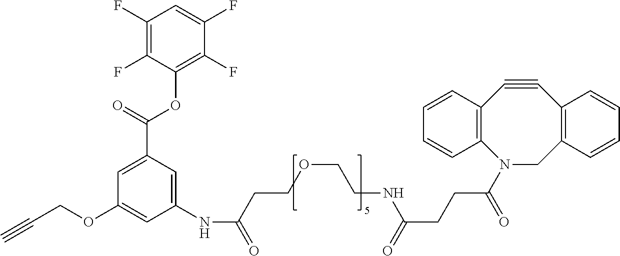

Linker-2

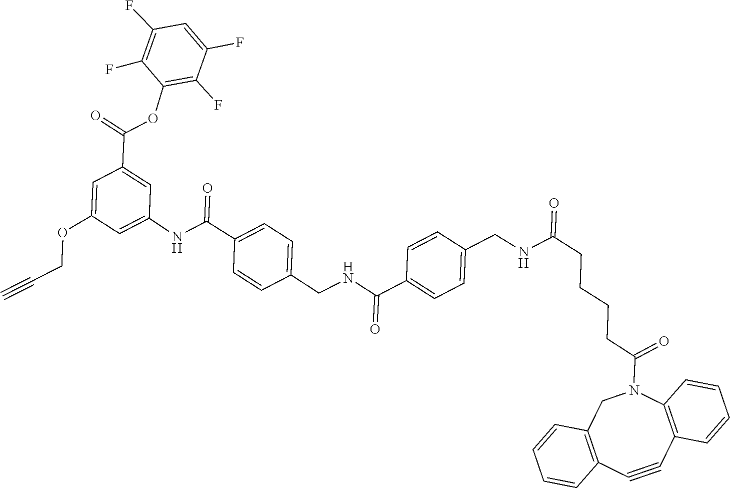 [SMILES: C#CCOc1cc(NC(=O)c2ccc(CNC(=O)c3ccc(CNC(=O)CCCCC(=O)N4Cc5ccccc5C#Cc5ccccc54)cc3)cc2)cc(C(=O)Oc2c(F)c(F)cc(F)c2F)c1]

Linker-3

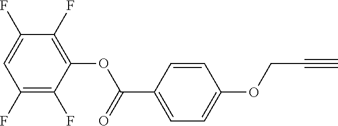 [SMILES: C#CCOc1ccc(C(=O)Oc2c(F)c(F)cc(F)c2F)cc1]

Linker-4

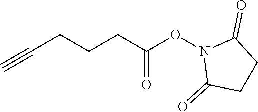

Linker-5 (Activate Scientific ® AS28942)

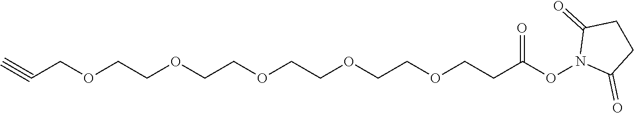 [SMILES: C#CCOCCOCCOCCOCCOCCC(=O)ON1C(=O)CCC1=O]

Linker-6 (BroadPharm ® BP-20907)

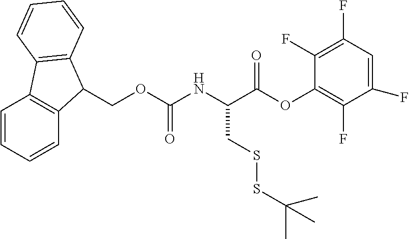 [SMILES: CC(C)(C)SSC[C@H](NC(=O)OCC1c2ccccc2-c2ccccc21)C(=O)Oc1c(F)c(F)cc(F)c1F]

Linker-7

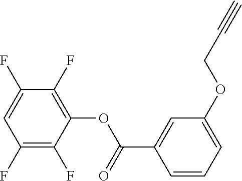 [SMILES: C#CCOc1cccc(C(=O)Oc2c(F)c(F)cc(F)c2F)c1]

Linker-8

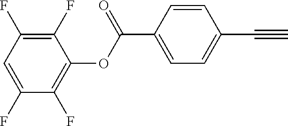 [SMILES: C#Cc1ccc(C(=O)Oc2c(F)c(F)cc(F)c2F)cc1]

Linker-9

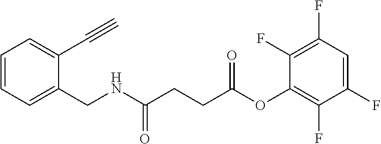 [SMILES: C#Cc1ccccc1CNC(=O)CCC(=O)Oc1c(F)c(F)cc(F)c1F]

Linker-10

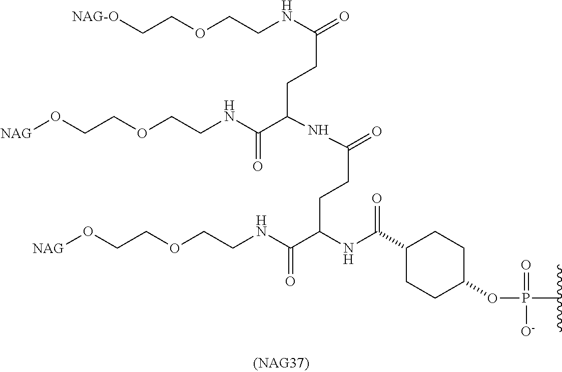

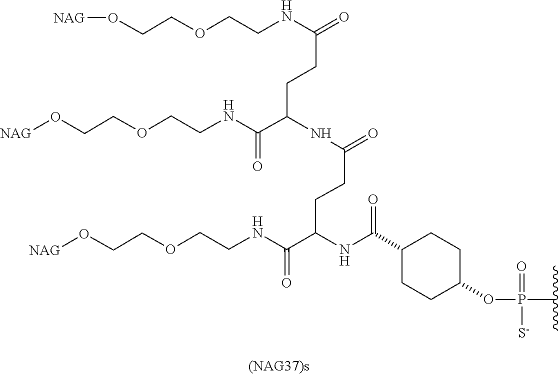

Alternatively, other linking groups known in the art may be used. In many instances, linking groups can be commercially acquired or alternatively, are incorporated into commercially available nucleotide phosphoramidites.

In some embodiments, a targeting ligand is linked to the DMPK RNAi agents disclosed herein. Examples of certain targeting ligands are provided in Table 6.2:

TABLE 6.2
Structures Representing Targeting Ligands

| Com- | |
|---|---|
| pound | |
| Num- | |
| ber | Formula |
| SM40a | 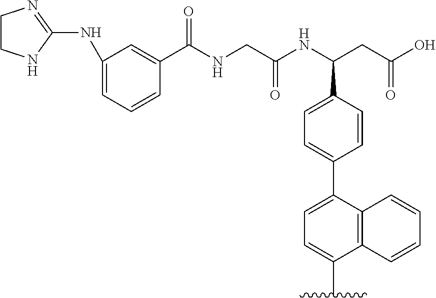 [SMILES: *c1ccc(-c2ccc([C@H](CC(=O)O)NC(=O)CNC(=O)c3cccc(NC4=NCCN4)c3)cc2)c2ccccc12] |
| SM41a | 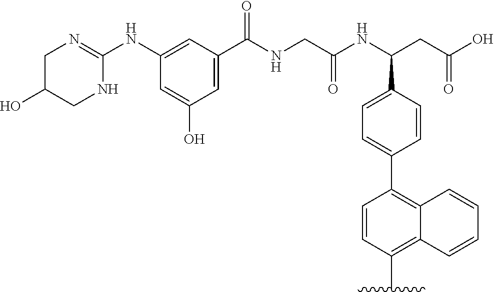 [SMILES: *c1ccc(-c2ccc([C@H](CC(=O)O)NC(=O)CNC(=O)c3cc(O)cc(NC4=NCC(O)CN4)c3)cc2)c2ccccc12] |
| SM42a | 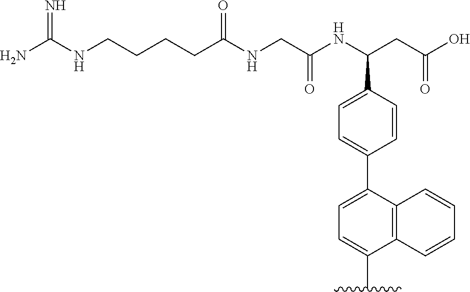 [SMILES: *c1ccc(-c2ccc([C@H](CC(=O)O)NC(=O)CNC(=O)CCCCNC(=N)N)cc2)c2ccccc12] |
| SM43a | 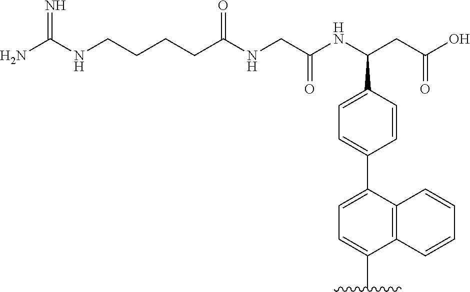 [SMILES: *c1ccc(-c2ccc([C@H](CC(=O)O)NC(=O)CNC(=O)CCCCNC(=N)N)cc2)c2ccccc12] |
| SM44a | 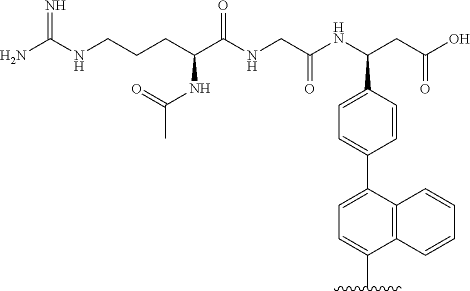 [SMILES: *c1ccc(-c2ccc([C@H](CC(=O)O)NC(=O)CNC(=O)[C@H](CCCNC(=N)N)NC(C)=O)cc2)c2ccccc12] |
| SM45a | 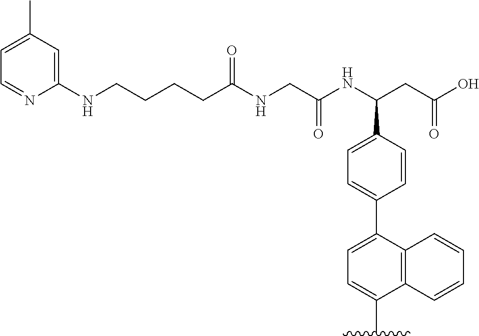 [SMILES: *c1ccc(-c2ccc([C@H](CC(=O)O)NC(=O)CNC(=O)CCCCNc3cc(C)ccn3)cc2)c2ccccc12] |
| SM46a | 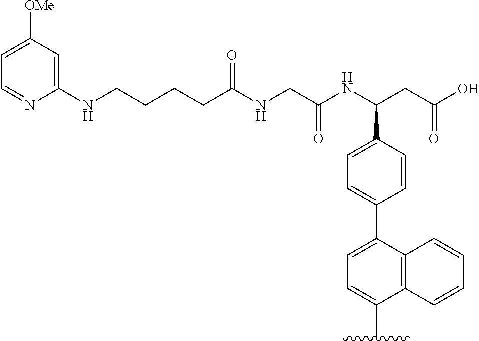 [SMILES: *c1ccc(-c2ccc([C@H](CC(=O)O)NC(=O)CNC(=O)CCCCNc3cc(OC)ccn3)cc2)c2ccccc12] |
| SM47a | 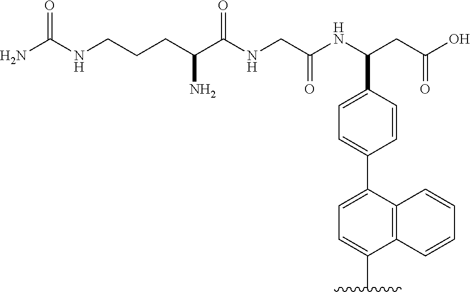 |
| SM48a | 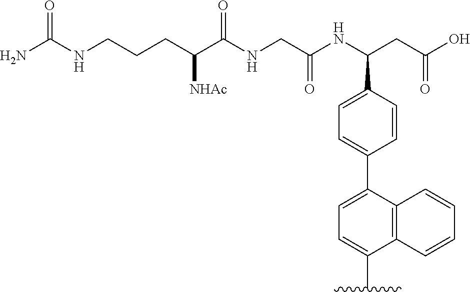 [SMILES: *c1ccc(-c2ccc([C@H](CC(=O)O)NC(=O)CNC(=O)[C@H](CCCNC(N)=O)NC(C)=O)cc2)c2ccccc12] |
| SM49a | 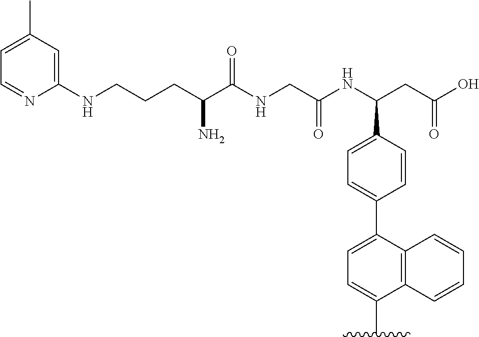 [SMILES: *c1ccc(-c2ccc([C@H](CC(=O)O)NC(=O)CNC(=O)[C@@H](N)CCCNc3cc(C)ccn3)cc2)c2ccccc12] |
| SM50a | 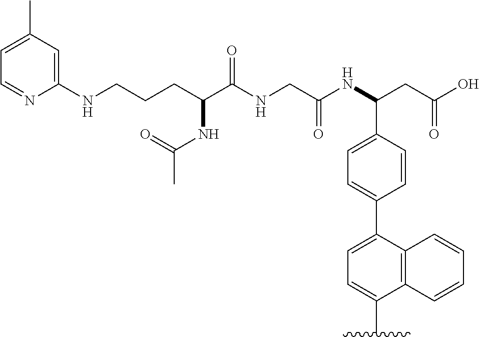 [SMILES: *c1ccc(-c2ccc([C@H](CC(=O)O)NC(=O)CNC(=O)[C@H](CCCNc3cc(C)ccn3)NC(C)=O)cc2)c2ccccc12] |
| SM51a | 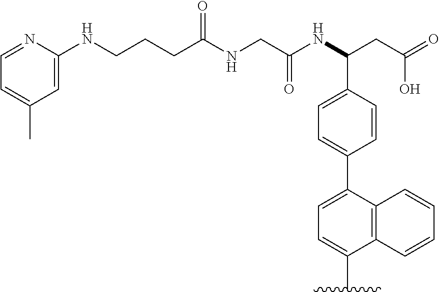 |
| SM52a | 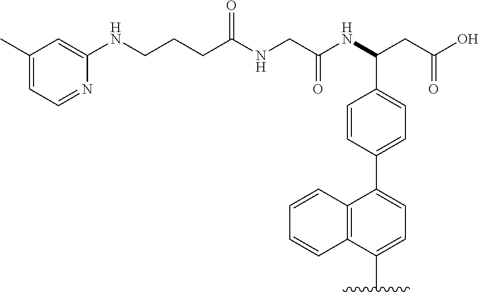 |
| SM53a | 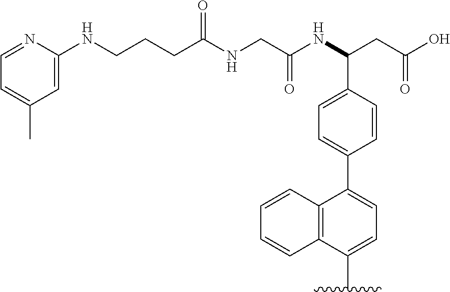 [SMILES: *c1ccc(-c2ccc([C@H](CC(=O)O)NC(=O)CNC(=O)CCCNc3cc(C)ccn3)cc2)c2ccccc12] |
| SM54a | 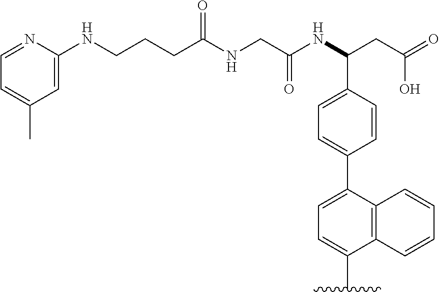 [SMILES: *c1ccc(-c2ccc([C@H](CC(=O)O)NC(=O)CNC(=O)CCCNc3cc(C)ccn3)cc2)c2ccccc12] |
| SM55a | 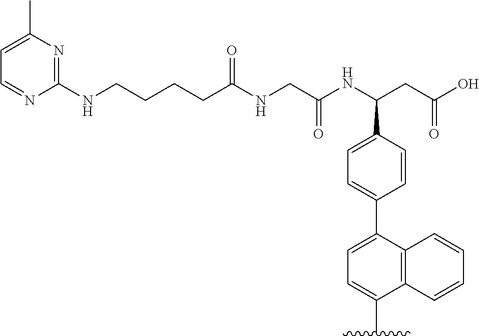 [SMILES: *c1ccc(-c2ccc([C@H](CC(=O)O)NC(=O)CNC(=O)CCCCNc3nccc(C)n3)cc2)c2ccccc12] |
| SM56a | 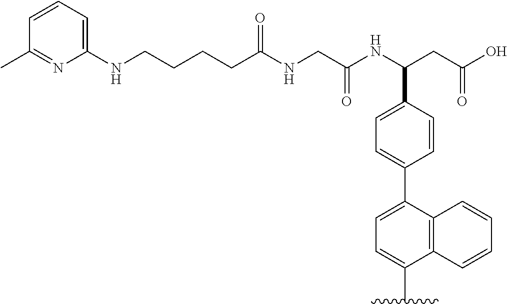 [SMILES: *c1ccc(-c2ccc([C@H](CC(=O)O)NC(=O)CNC(=O)CCCCNc3cccc(C)n3)cc2)c2ccccc12] |
| SM57a | 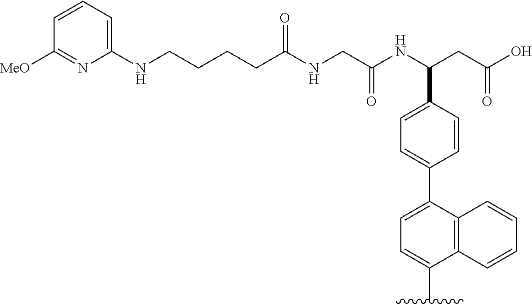 [SMILES: *c1ccc(-c2ccc([C@H](CC(=O)O)NC(=O)CNC(=O)CCCCNc3cccc(OC)n3)cc2)c2ccccc12] |
| SM58a | 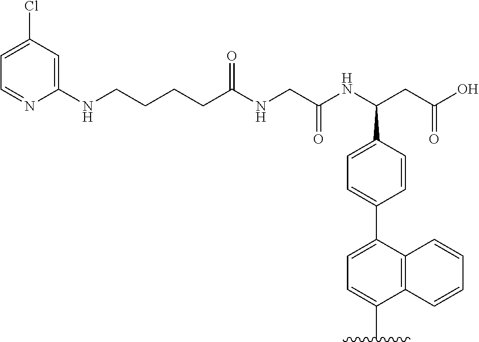 [SMILES: *c1ccc(-c2ccc([C@H](CC(=O)O)NC(=O)CNC(=O)CCCCNc3cc(Cl)ccn3)cc2)c2ccccc12] |
| SM59a | 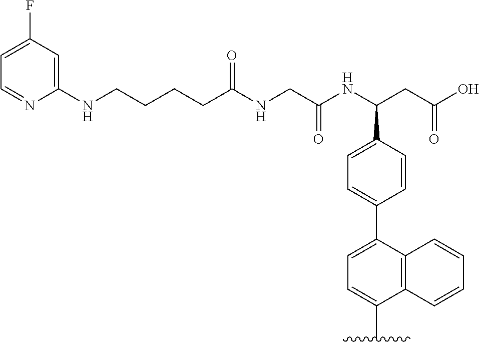 [SMILES: *c1ccc(-c2ccc([C@H](CC(=O)O)NC(=O)CNC(=O)CCCCNc3cc(F)ccn3)cc2)c2ccccc12] |
| SM60a | 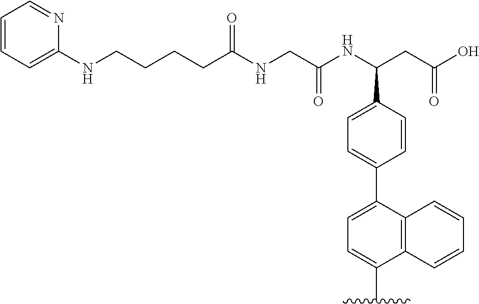 [SMILES: *c1ccc(-c2ccc([C@H](CC(=O)O)NC(=O)CNC(=O)CCCCNc3ccccn3)cc2)c2ccccc12] |
| Peptide 1a | 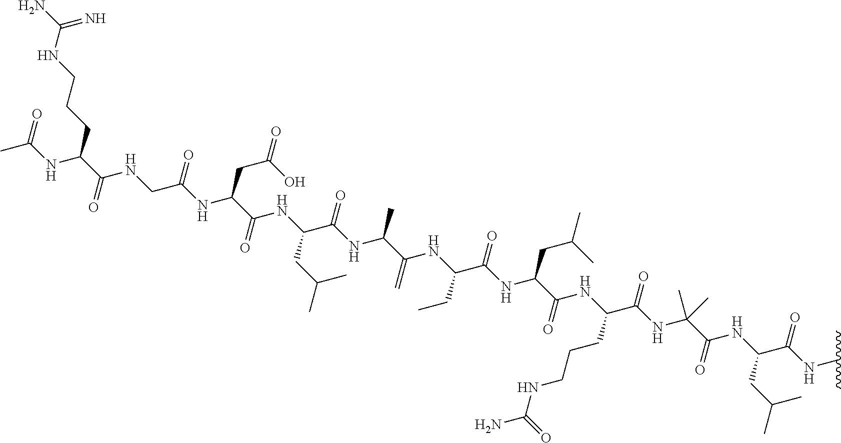 [SMILES: *NC(=O)[C@H](CC(C)C)NC(=O)C(C)(C)NC(=O)[C@H](CCCNC(N)=O)NC(=O)[C@H](CC(C)C)NC(=O)[C@H](CC)NC(=C)[C@H](C)NC(=O)[C@H](CC(C)C)NC(=O)[C@H](CC(=O)O)NC(=O)CNC(=O)[C@H](CCCNC(=N)N)NC(C)=O] |

or a pharmaceutically acceptable salt thereof, wherein [symbol] indicates the point of connection to the DMPK RNAi agents. In some embodiments, a PEG or other linking group is incorporated between the RNAi agent and the targeting ligand.

In some embodiments, the targeting groups in Table 6.2 are synthesized with reactive groups allowing for efficient coupling of a targeting ligand that includes one or more targeting groups to the RNAi agents disclosed herein. In some embodiments, the targeting groups identified in Table 6.2 are synthesized as azides to facilitate linkage to the RNAi agent.

In some embodiments, the DMPK RNAi agents are linked to a targeting ligand having a structure disclosed in Table 6.3:

TABLE 6.3
Example targeting ligands for combination with DMPK RNAi agents.

| Compound | |
|---|---|
| Number | Formula |
| 40b | 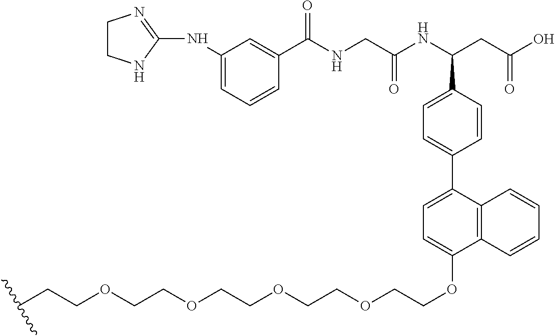 [SMILES: *CCOCCOCCOCCOCCOc1ccc(-c2ccc([C@H](CC(=O)O)NC(=O)CNC(=O)c3cccc(NC4=NCCN4)c3)cc2)c2ccccc12] |
| 41b | 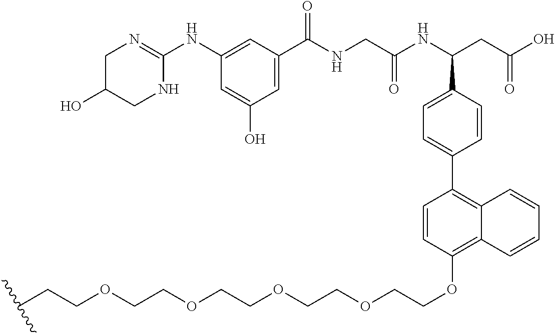 [SMILES: *CCOCCOCCOCCOCCOc1ccc(-c2ccc([C@H](CC(=O)O)NC(=O)CNC(=O)c3cc(O)cc(NC4=NCC(O)CN4)c3)cc2)c2ccccc12] |
| 42b | 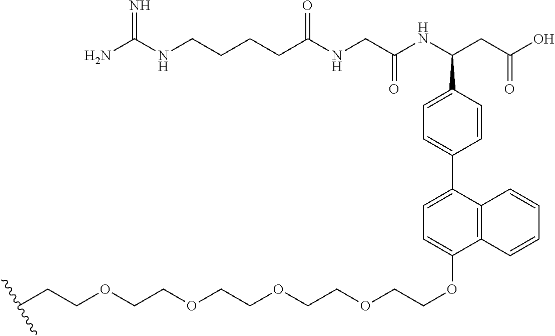 [SMILES: *CCOCCOCCOCCOCCOc1ccc(-c2ccc([C@H](CC(=O)O)NC(=O)CNC(=O)CCCCNC(=N)N)cc2)c2ccccc12] |
| 43b | 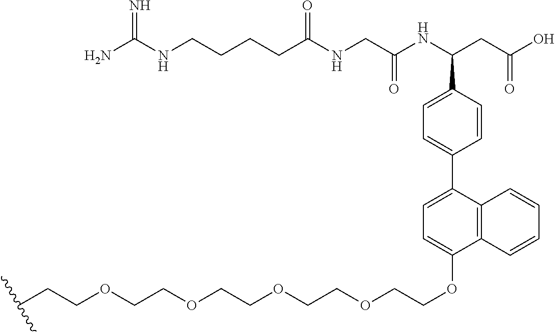 [SMILES: *CCOCCOCCOCCOCCOc1ccc(-c2ccc([C@H](CC(=O)O)NC(=O)CNC(=O)CCCCNC(=N)N)cc2)c2ccccc12] |
| 44b | 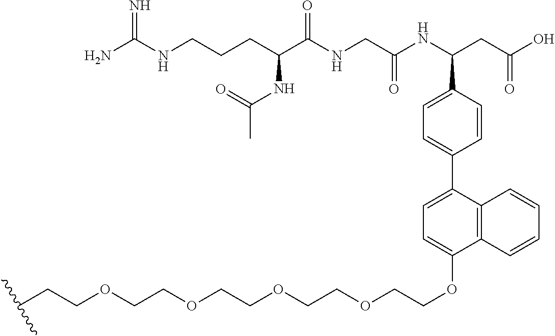 [SMILES: *CCOCCOCCOCCOCCOc1ccc(-c2ccc([C@H](CC(=O)O)NC(=O)CNC(=O)[C@H](CCCNC(=N)N)NC(C)=O)cc2)c2ccccc12] |
| 45b | 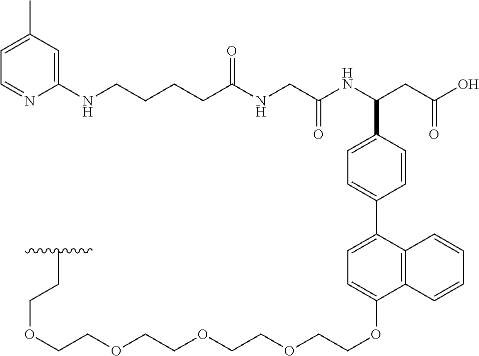 [SMILES: *CCOCCOCCOCCOCCOc1ccc(-c2ccc([C@H](CC(=O)O)NC(=O)CNC(=O)CCCCNc3cc(C)ccn3)cc2)c2ccccc12] |
| 46b | 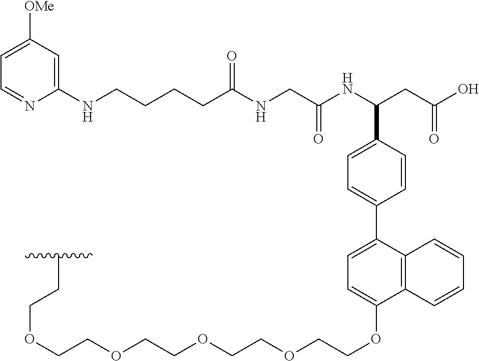 [SMILES: *CCOCCOCCOCCOCCOc1ccc(-c2ccc([C@H](CC(=O)O)NC(=O)CNC(=O)CCCCNc3cc(OC)ccn3)cc2)c2ccccc12] |
| 47b | 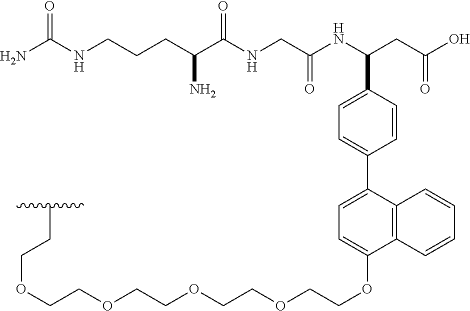 [SMILES: *CCOCCOCCOCCOCCOc1ccc(-c2ccc([C@H](CC(=O)O)NC(=O)CNC(=O)[C@@H](N)CCCNC(N)=O)cc2)c2ccccc12] |
| 48b | 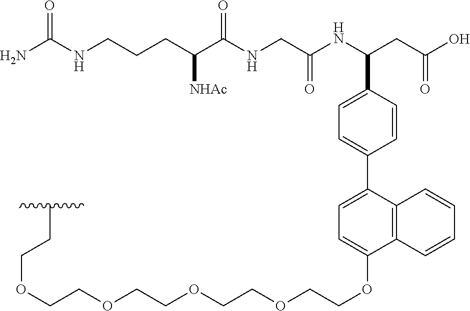 |
| 49b | 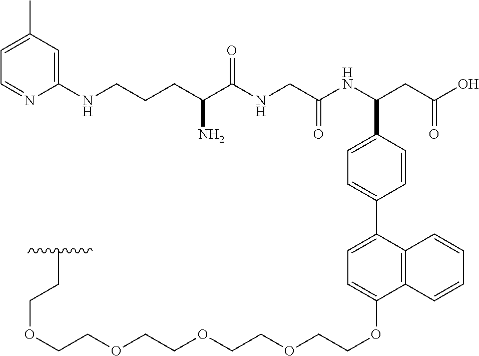 [SMILES: *CCOCCOCCOCCOCCOc1ccc(-c2ccc([C@H](CC(=O)O)NC(=O)CNC(=O)[C@@H](N)CCCNc3cc(C)ccn3)cc2)c2ccccc12] |
| 50b | 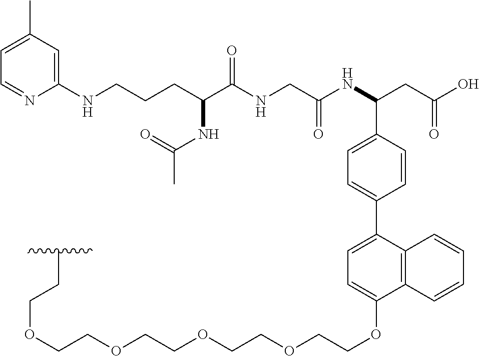 [SMILES: *CCOCCOCCOCCOCCOc1ccc(-c2ccc([C@H](CC(=O)O)NC(=O)CNC(=O)[C@H](CCCNc3cc(C)ccn3)NC(C)=O)cc2)c2ccccc12] |
| 51b | 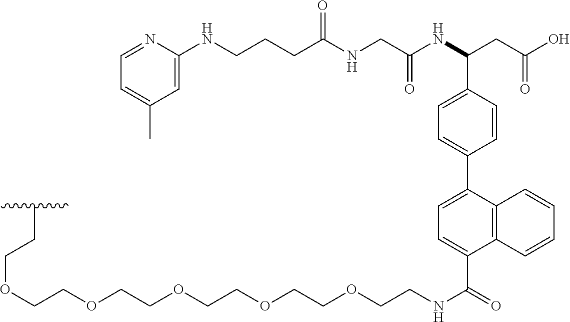 [SMILES: *CCOCCOCCOCCOCCOCCNC(=O)c1ccc(-c2ccc([C@H](CC(=O)O)NC(=O)CNC(=O)CCCNc3cc(C)ccn3)cc2)c2ccccc12] |
| 52b | 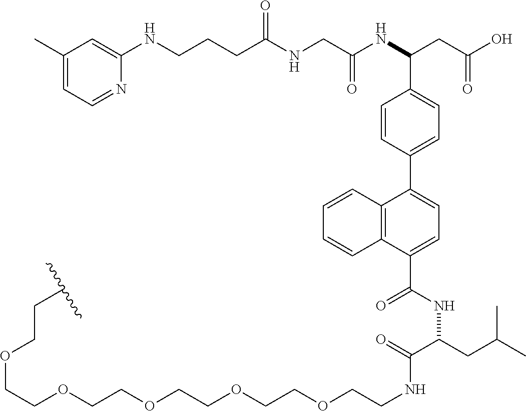 [SMILES: *CCOCCOCCOCCOCCOCCNC(=O)[C@@H](CC(C)C)NC(=O)c1ccc(-c2ccc([C@H](CC(=O)O)NC(=O)CNC(=O)CCCNc3cc(C)ccn3)cc2)c2ccccc12] |
| 53b | 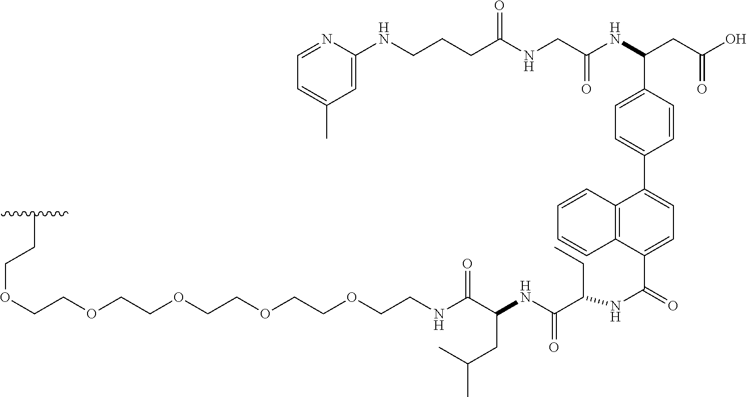 [SMILES: *CCOCCOCCOCCOCCOCCNC(=O)[C@H](CC(C)C)NC(=O)[C@H](CC)NC(=O)c1ccc(-c2ccc([C@H](CC(=O)O)NC(=O)CNC(=O)CCCNc3cc(C)ccn3)cc2)c2ccccc12] |
| 54b | 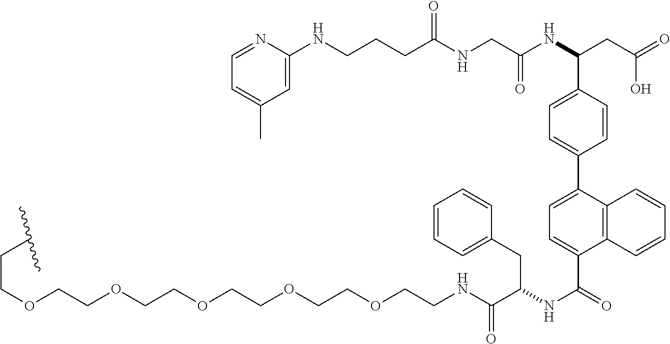 [SMILES: *CCOCCOCCOCCOCCOCCNC(=O)[C@H](Cc1ccccc1)NC(=O)c1ccc(-c2ccc([C@H](CC(=O)O)NC(=O)CNC(=O)CCCNc3cc(C)ccn3)cc2)c2ccccc12] |
| 55b | 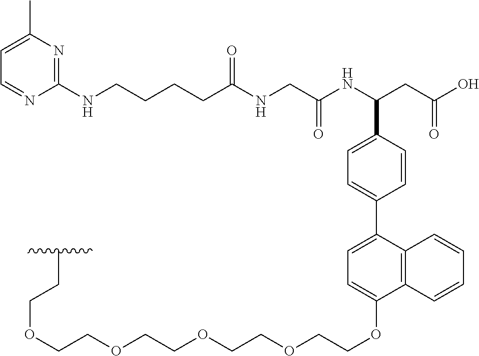 |
| 56b | 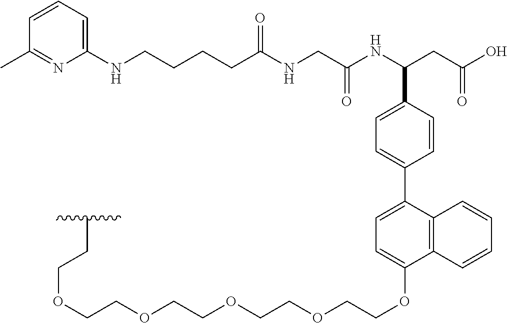 |
| 57b | 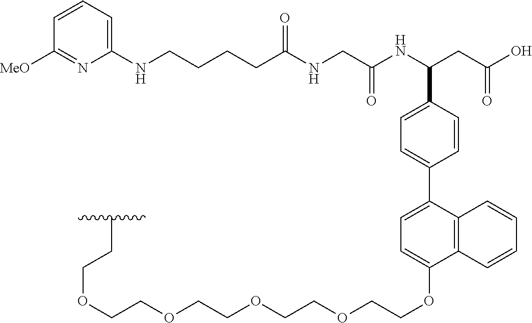 [SMILES: *CCOCCOCCOCCOCCOc1ccc(-c2ccc([C@H](CC(=O)O)NC(=O)CNC(=O)CCCCNc3cccc(OC)n3)cc2)c2ccccc12] |
| 58b | 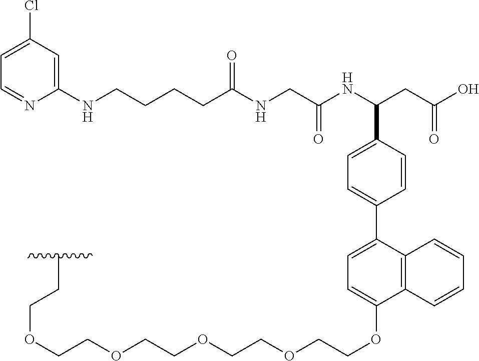 |
| 59b | 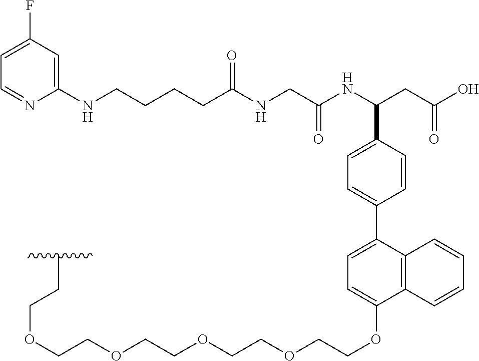 |
| 60b | 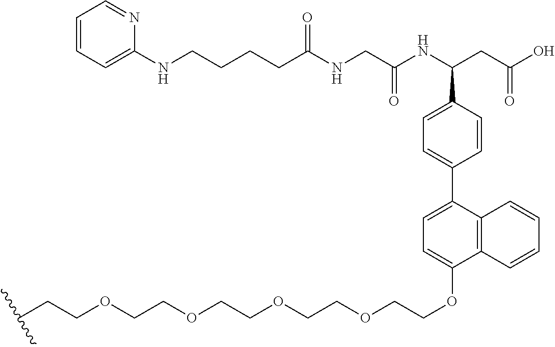 [SMILES: *CCOCCOCCOCCOCCOc1ccc(-c2ccc([C@H](CC(=O)O)NC(=O)CNC(=O)CCCCNc3ccccn3)cc2)c2ccccc12] |
| αvβ6 Peptide 1 | 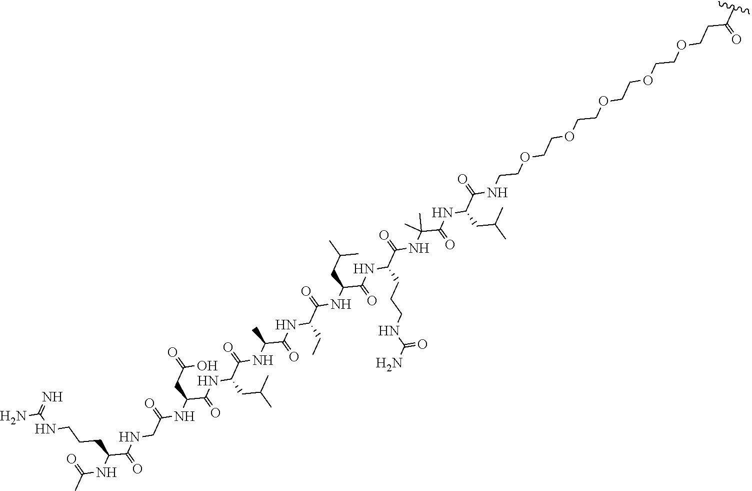 [SMILES: *C(=O)CCOCCOCCOCCOCCOCCNC(=O)[C@H](CC(C)C)NC(=O)C(C)(C)NC(=O)[C@H](CCCNC(N)=O)NC(=O)[C@H](CC(C)C)NC(=O)[C@H](CC)NC(=O)[C@H](C)NC(=O)[C@H](CC(C)C)NC(=O)[C@H](CC(=O)O)NC(=O)CNC(=O)[C@H](CCCNC(=N)N)NC(C)=O] |
| (NAG37) | 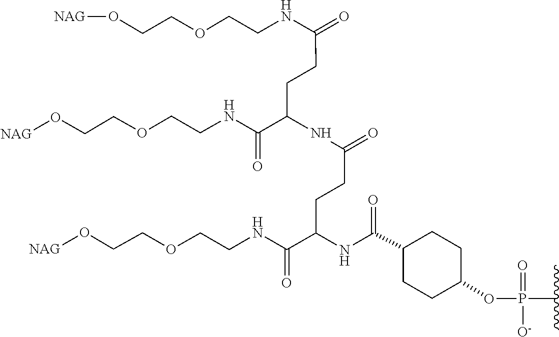 |
| (NAG37)s | 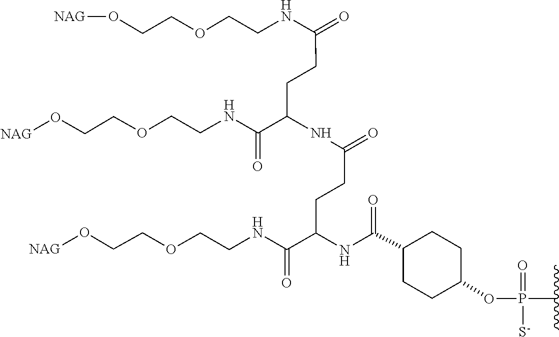 |

or a pharmaceutically acceptable salt thereof, wherein [symbol] indicates the point of connection to the DMPK RNAi agents.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art for nucleic acid delivery. The RNAi agents can also be chemically conjugated to targeting groups, lipids (including, but not limited to cholesteryl and cholesteryl derivatives), encapsulating in nanoparticles, liposomes, micelles, conjugating to polymers or DPCs (see, for example WO 2000/053722, WO 2008/022309, WO 2011/104169, and WO 2012/083185, WO 2013/032829, WO 2013/158141, each of which is incorporated herein by reference), by iontophoresis, or by incorporation into other delivery vehicles or systems available in the art such as hydrogels, cyclodextrins, biodegradable nanocapsules, bioadhesive microspheres, or proteinaceous vectors. In some embodiments the RNAi agents can be conjugated to antibodies having affinity for skeletal muscle cells. In some embodiments, the RNAi agents can be linked to targeting ligands that have affinity for skeletal muscle cells or receptors present on skeletal muscle cells.

### Pharmacokinetic/Pharmacodynamic (PK/PD) Modulators

In some embodiments, the DMPK RNAi agents disclosed herein are further or alternatively linked to one or more PK/PD modulators. Examples of certain pharmacodynamic/pharmacokinetic (PK/PD) modulators suitable for use with the RNAi agents disclosed herein are provided in Table 6.4. In Table 6.4, PK/PD modulators were acquired from commercial suppliers where indicated and were otherwise synthesized using commercially available materials:

TABLE 6.4
Exemplary PK/PD Modulator Compounds.

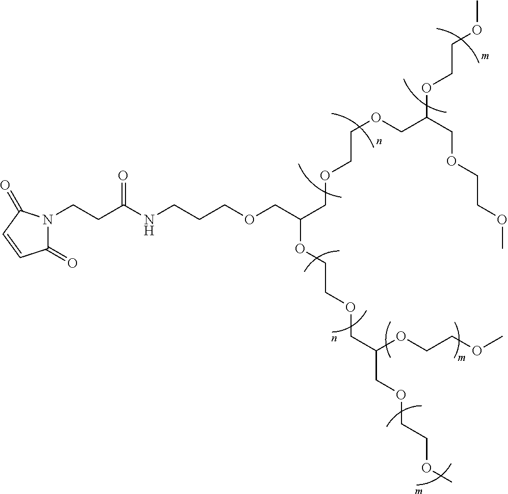

PEG40K (2 × 2-arm),
wherein n and m are integers, and the molecular weight of the PEG groups is about 40
kilodaltons
NOF, Sunbright ® GL4-400MA

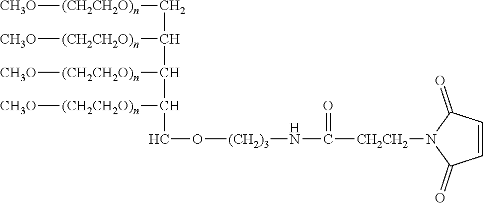

PEG40K (4-arm),
wherein n is an integer, and the molecular weight of the PEG groups is about 40 kilodaltons
NOF, Sunbright ® XY4-400MA

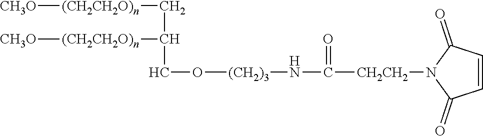

PEG40K (2-arm),
wherein n is an integer, and the molecular weight of the PEG groups is about 40 kilodaltons
NOF, Sunbright ® GL2-400MA

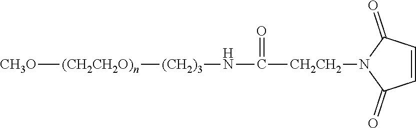

PEG40K,
wherein n is an integer, and the molecular weight of the PEG groups is about 40 kilodaltons
NOF, Sunbright ® ME-400MA

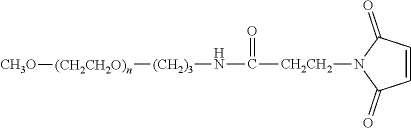

PEG10K,
wherein n is an integer, and the molecular weight of the PEG groups is about 10 kilodaltons
NOF, Sunbright ® ME-100MA

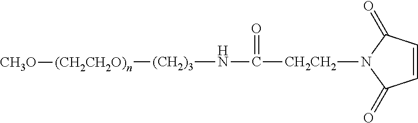

PEG5K,
wherein n is an integer, and the molecular weight of the PEG groups is about 5 kilodaltons
NOF, Sunbright ® ME-050MA

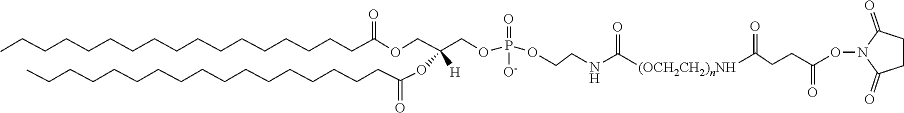

DSPE-PEG5K-NHS (Naonsoft Polymers ™ #SKU 1544)
(1,2-distearoyl-sn-glycero-3-phosphoethanolamine-N-[succinimidyl(polyethylene glycol)]),
wherein n is an integer and the molecular weight of the PEG groups is about 5 kilodaltons

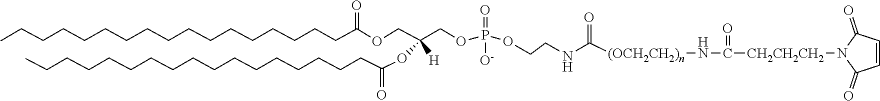

DSPE-PEG5K-MAL (Naonsoft Polymers ™ SKU #2049)
1,2-distearoyl-sn-glycero-3-phosphoethanolamine-N-[maleimide(polyethylene glycol)],
Wherein n is an integer and the molecular weight of the PEG groups is about 5 kilodaltons

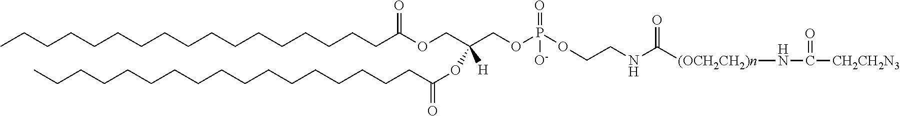

DSPE-PEG5K-N3 (Naonsoft Polymers ™ SKU #2274)
1,2-distearoyl-sn-glycero-3-phosphoethanolamine-N-[azido(polyethylene glycol)], wherein
n is an integer and the molecular weight of the PEG groups is about 5 kilodaltons

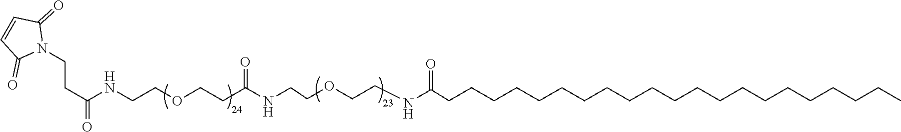

PEG47 + C22

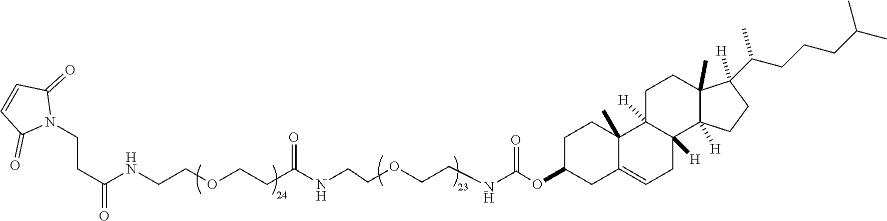

PEG47 + CLS (cholesterol)

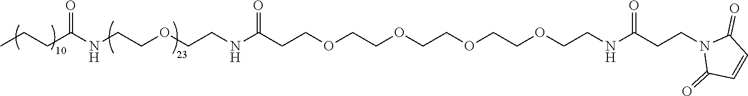

PEG23 + C22

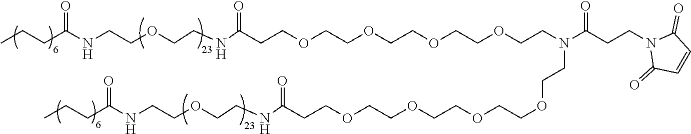

Bis(PEG23 + C14)

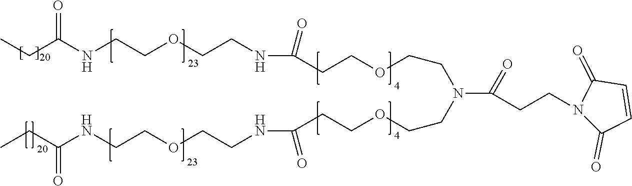

Bis(PEG23 + C22)

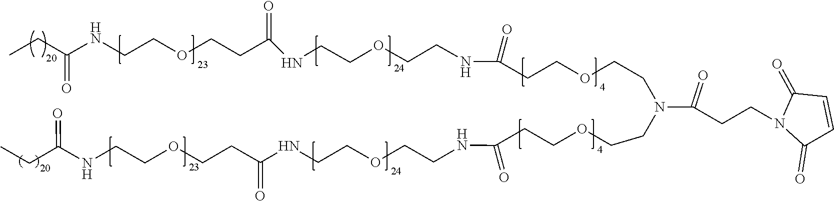

Bis(PEG47 + C22)

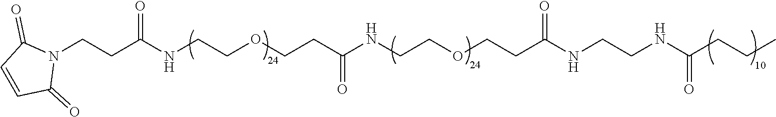

PEG48 + C22

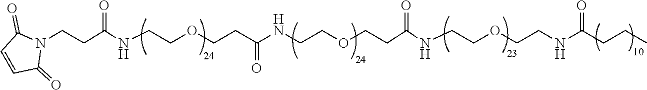

PEG71 + C22

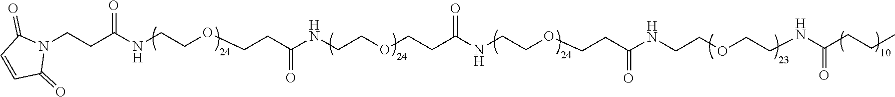

PEG95 + C22

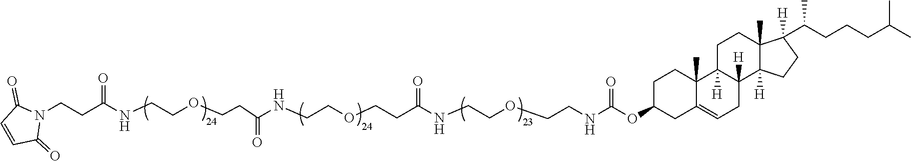

PEG71 + CLS

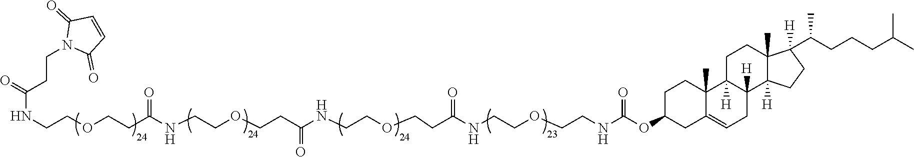

PEG95 + CLS

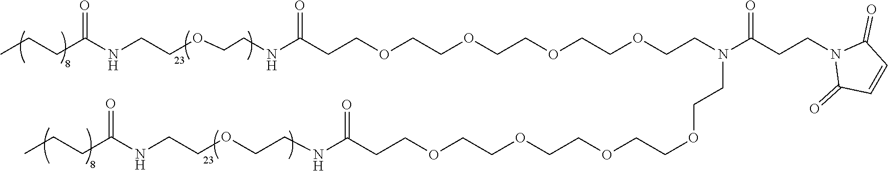

Bis(PEG23 + C18)

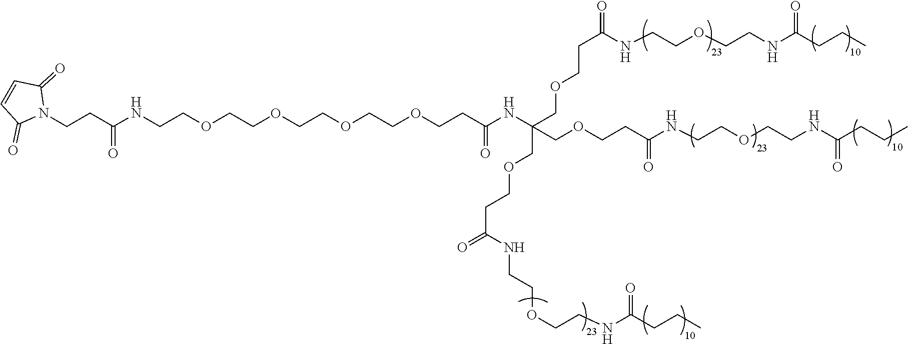

Tris(PEG23 + C22)

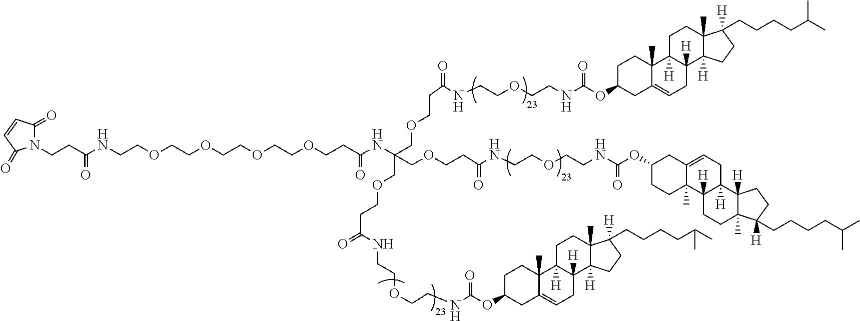

Tris(PEG23 + CLS)

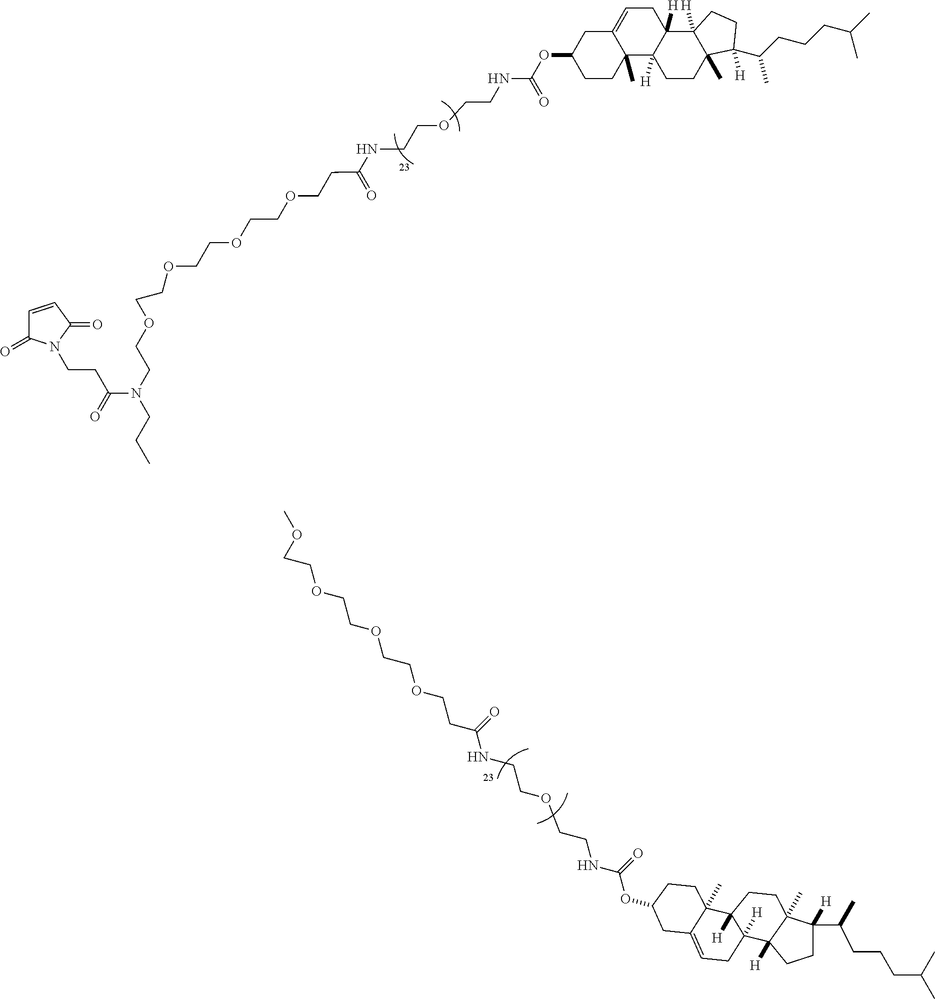

Bis(PEG23 + CLS)

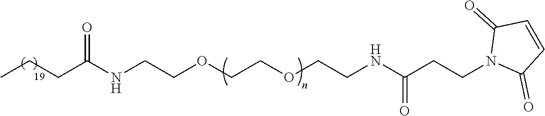

PEG5K + C22
wherein n is an integer and the molecular weight of the PEG units is about 5 kilodaltons

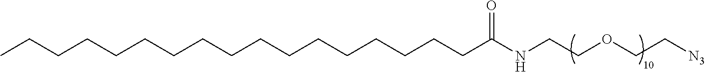

C18

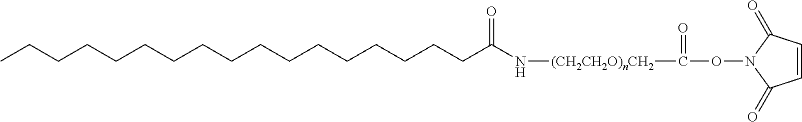

(NHS)-PEG1K + C18 (Nanosoft Polymers ™ SKU #10668-1000)
wherein n is an integer and the molecular weight of the PEG units is about 1 kilodalton

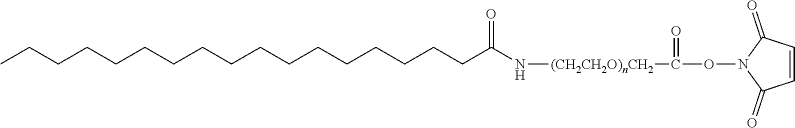

(NHS)-PEG2K + C18 (Nanosoft Polymers ™ SKU #10668-2000)
wherein n is an integer and the molecular weight of the PEG units is about 2 kilodaltons

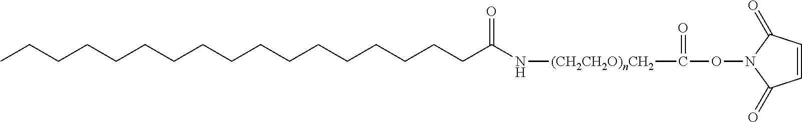

(NHS)-PEG5K + C18 (Nanosoft Polymers ™ SKU #10668-5000)
wherein n is an integer and the molecular weight of the PEG units is about 5 kilodaltons

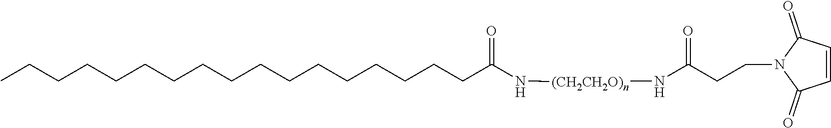

(MAL)-PEG5K + C18 (Nanosoft Polymers ™ SKU #10647)
wherein n is an integer and the molecular weight of the PEG units is about 5 kilodaltons

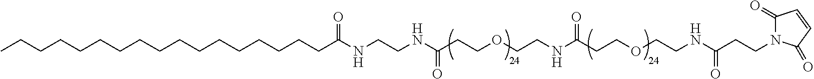

PEG48 + C18

In some embodiments, the PK/PD modulators of Table 6.4 have the following structures following conjugation to the DMPK RNAi agents as shown in Table 6.5:

TABLE 6.5
Example PK/PD modulators conjugated to DMPK RNAi agents.

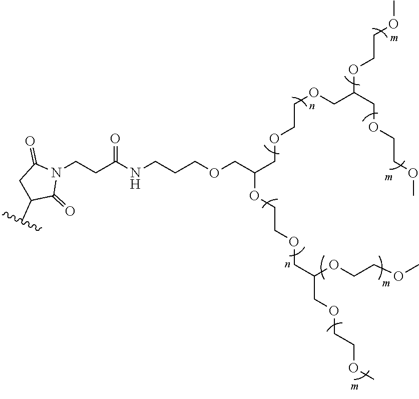

PEG40K (2 × 2-arm),
wherein n and m are integers, and the molecular weight of the PEG groups is about 40
kilodaltons

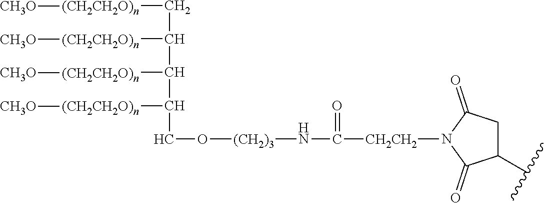

PEG40K (4-arm),
wherein n is an integer, and the molecular weight of the PEG groups is about 40 kilodaltons

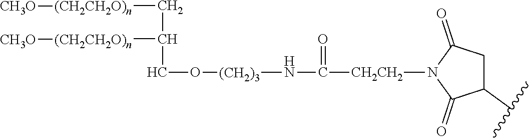

PEG40K (2-arm),
wherein n is an integer, and the molecular weight of the PEG groups is about 40 kilodaltons

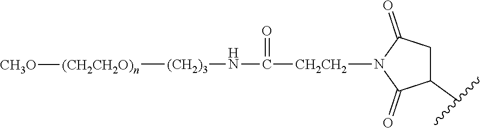

PEG40K,
wherein n is an integer, and the molecular weight of the PEG groups is about 40 kilodaltons

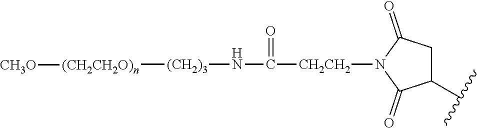

PEG10K,
wherein n is an integer, and the molecular weight of the PEG groups is about 10 kilodaltons

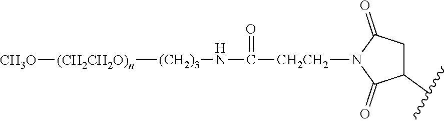

PEG5K,
wherein n is an integer, and the molecular weight of the PEG groups is about 5 kilodaltons

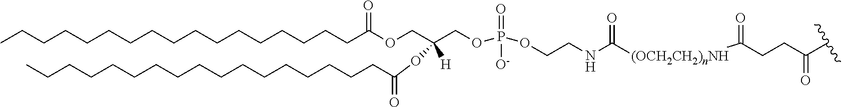

DSPE-PEG5K-NHS
wherein n is an integer and the molecular weight of the PEG groups is about 5 kilodaltons

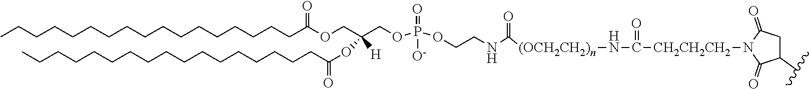

DSPE-PEG5K-MAL
Wherein n is an integer and the molecular weight of the PEG groups is about 5 kilodaltons

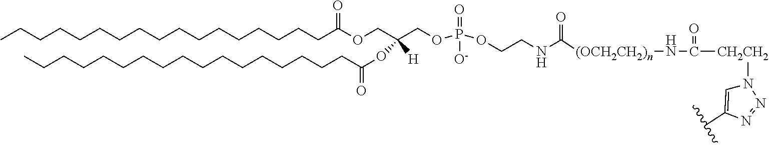

DSPE-PEG5K-N3
wherein n is an integer and the molecular weight of the PEG groups is about 5 kilodaltons

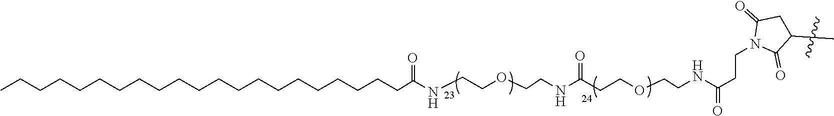

PEG47 + C22

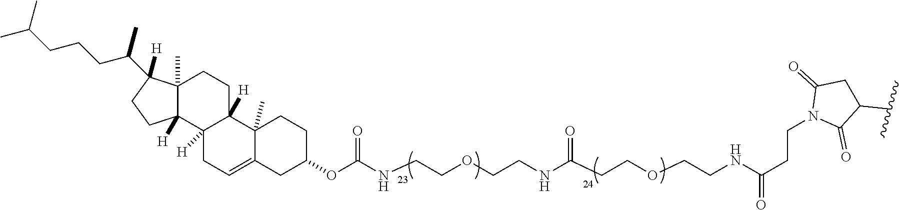

PEG47 + CLS (cholesterol)

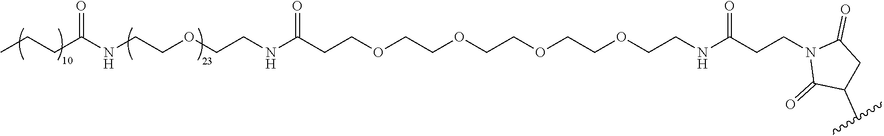

PEG23 + C22

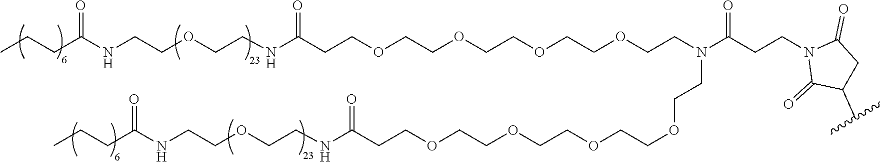

Bis(PEG23 + C14)

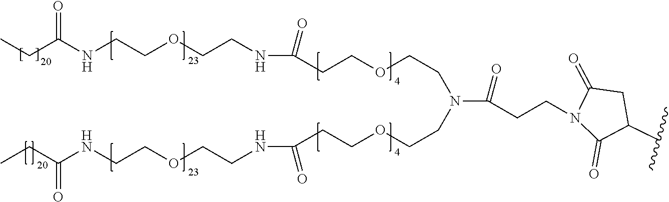

Bis(PEG23 + C22)

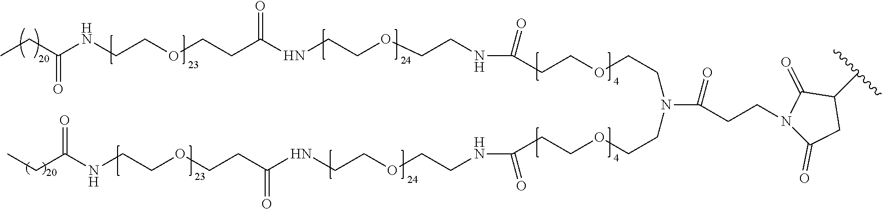

Bis(PEG47 + C22)

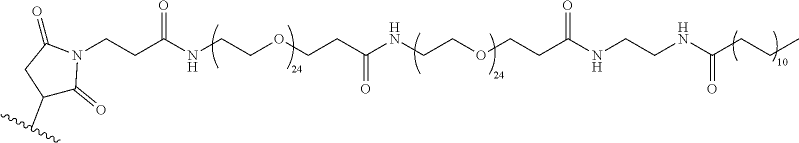

PEG48 + C22

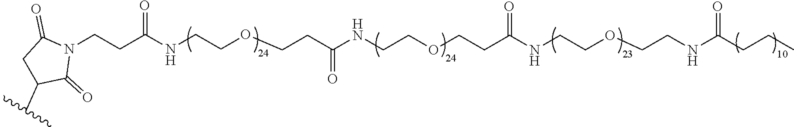

PEG71 + C22

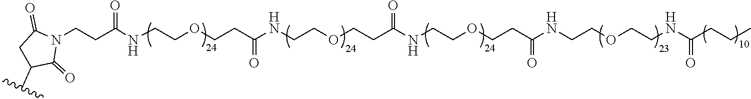

PEG95 + C22

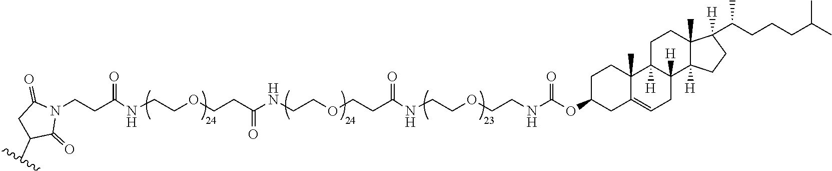

PEG71 + CLS

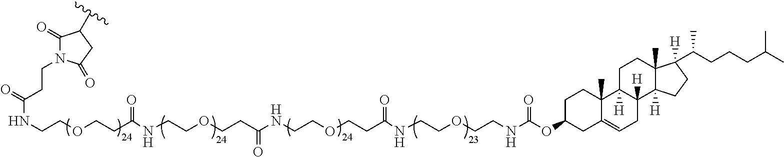

PEG95 + CLS

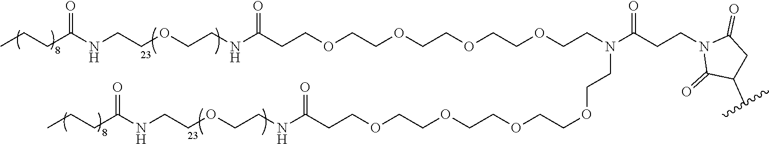

Bis(PEG23 + C18)

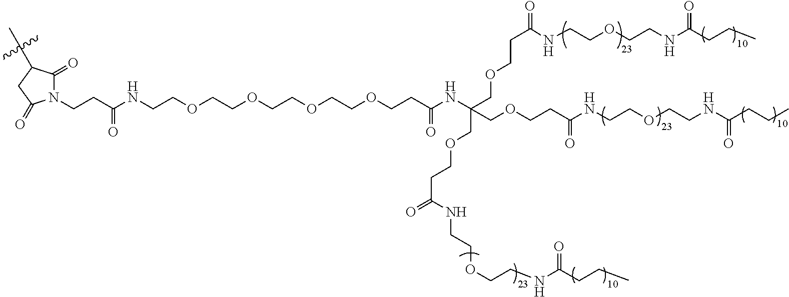

Tris(PEG23 + C22)

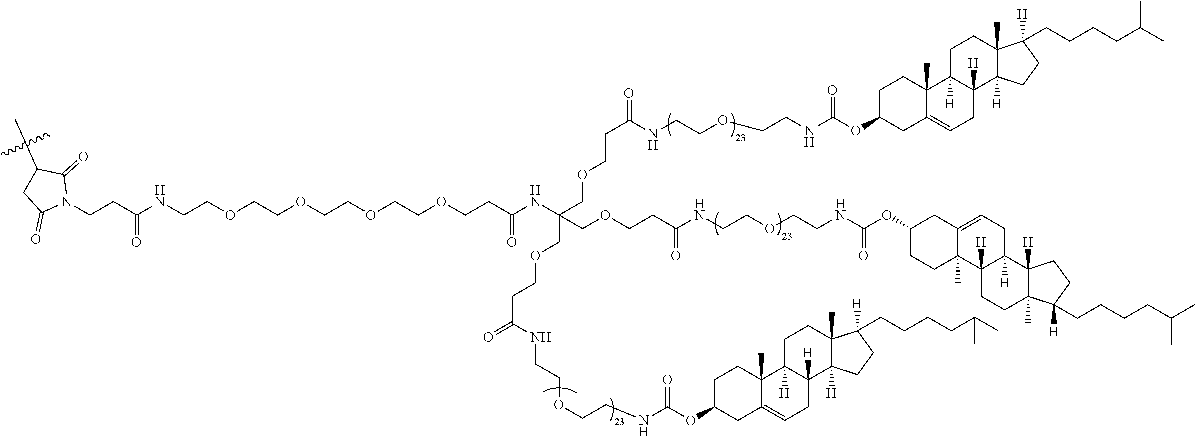

Tris(PEG23 + CLS)

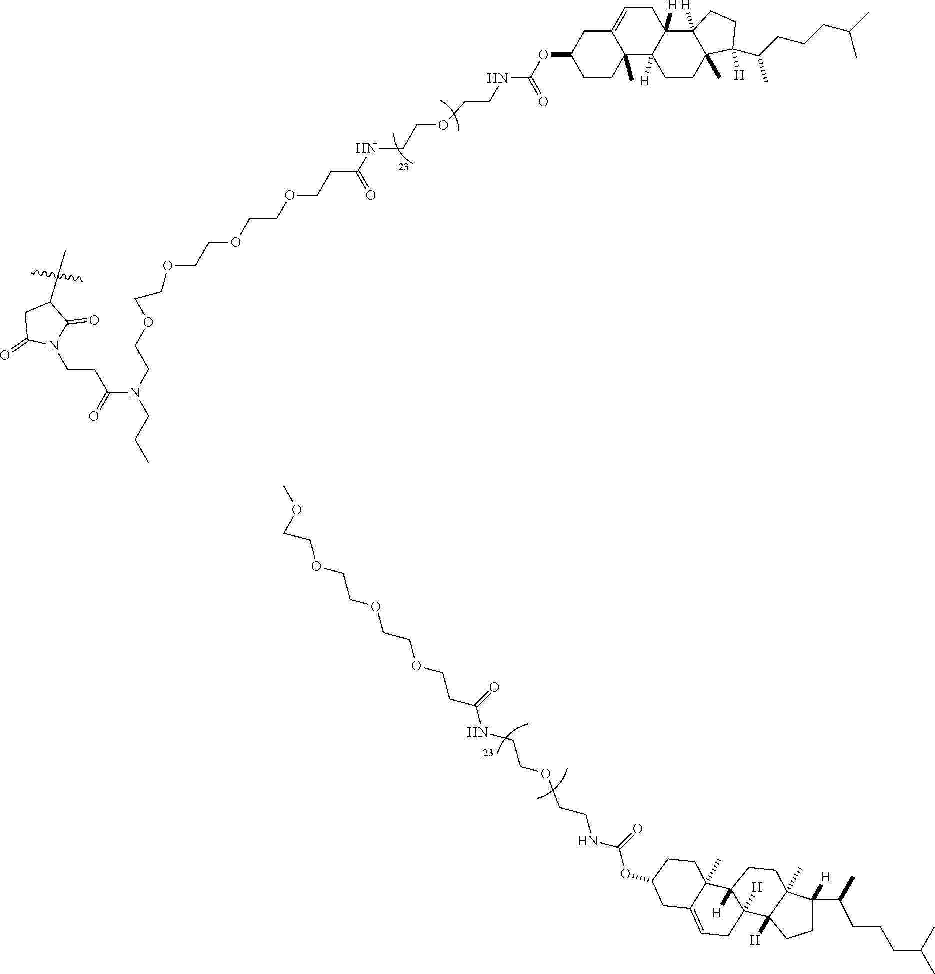

Bis(PEG23 + CLS)

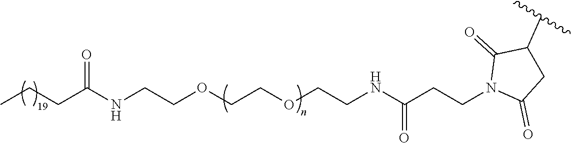

PEG5K + C22
wherein n is an integer and the molecular weight of the PEG units is about 5 kilodaltons

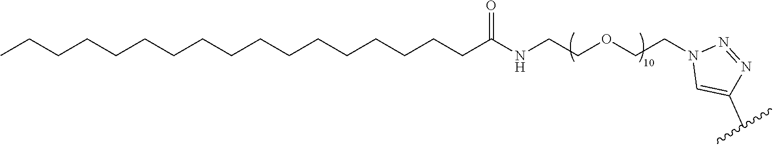

C18

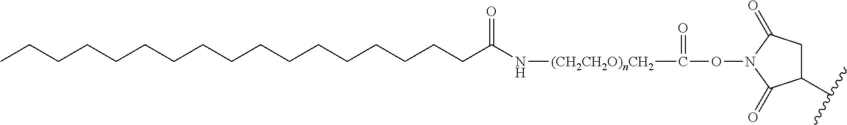

(NHS)-PEG1K + C18
wherein n is an integer and the molecular weight of the PEG units is about 1 kilodalton

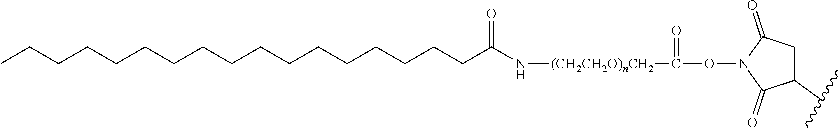

(NHS)-PEG2K + C18
wherein n is an integer and the molecular weight of the PEG units is about 2 kilodaltons

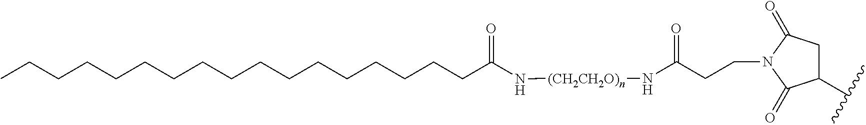

(NHS)-PEG5K + C18
wherein n is an integer and the molecular weight of the PEG units is about 5 kilodaltons

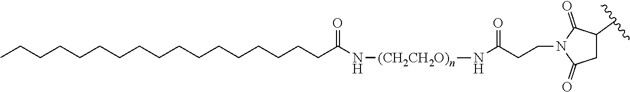

(MAL)-PEG5K + C18
wherein n is an integer and the molecular weight of the PEG units is about 5 kilodaltons

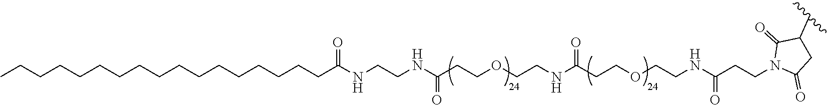

PEG48 + C18

or a pharmaceutically acceptable salt thereof, wherein [symbol] indicates the point of connection to the DMPK RNAi agents.

In other embodiments, the PK/PD modulator that may be conjugated to the DMPK RNAi agents described herein may be selected from the group consisting of the PK/PD modulators in Table 6.6:

TABLE 6.6
Example PK/PD modulators for conjugating to DMPK RNAi agents (Compound
number appears before structure.)
LP1-p

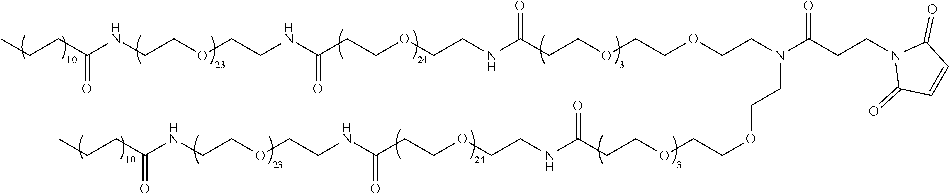

LP28-p

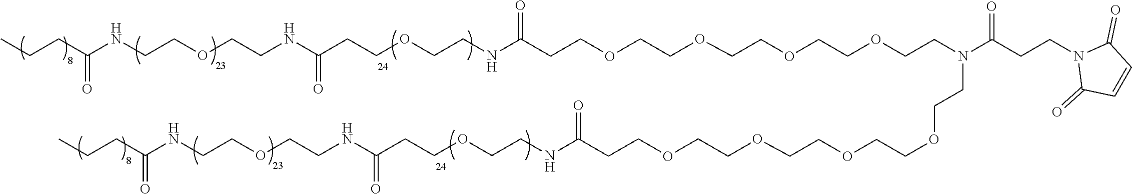

LP29-p

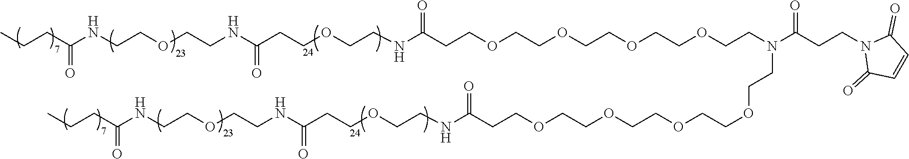

LP38-p

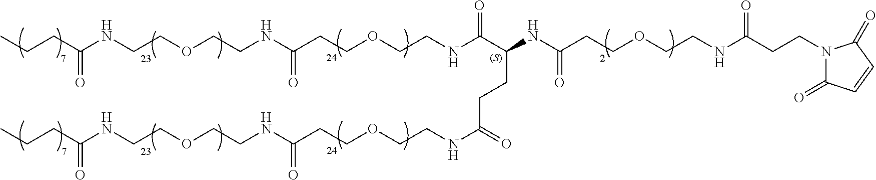

LP39-p

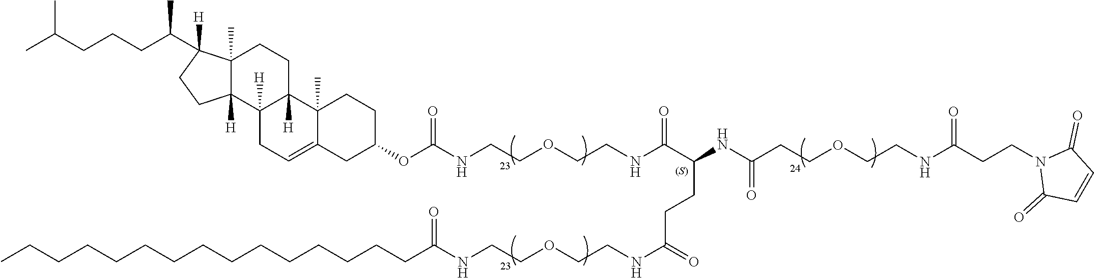

LP41-p

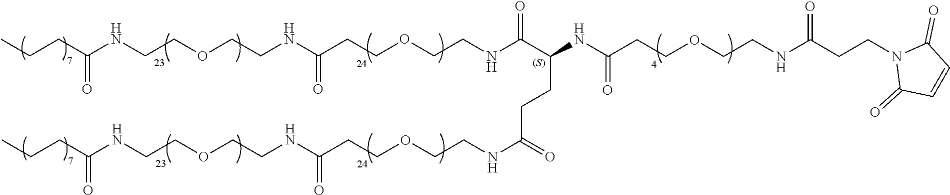

LP42-p

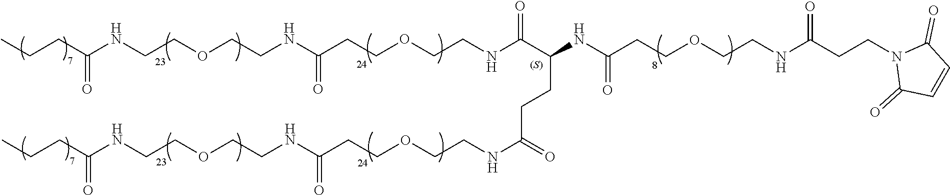

LP43-p

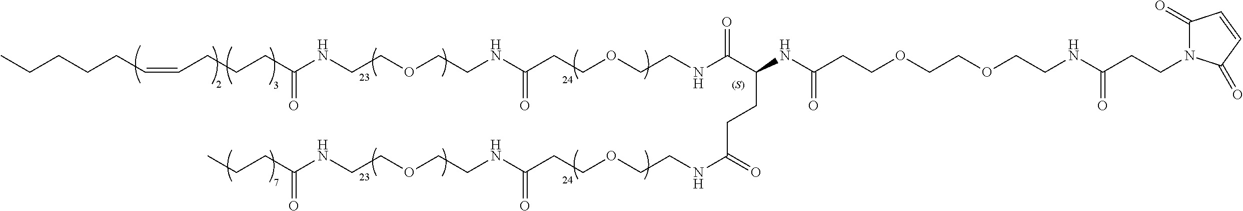

LP44-p

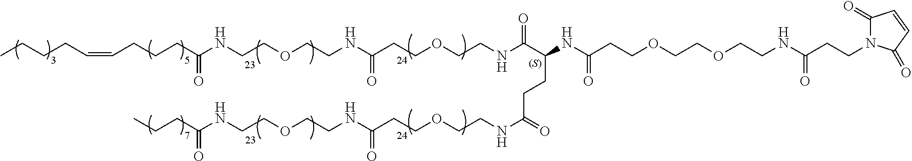

LP45-p

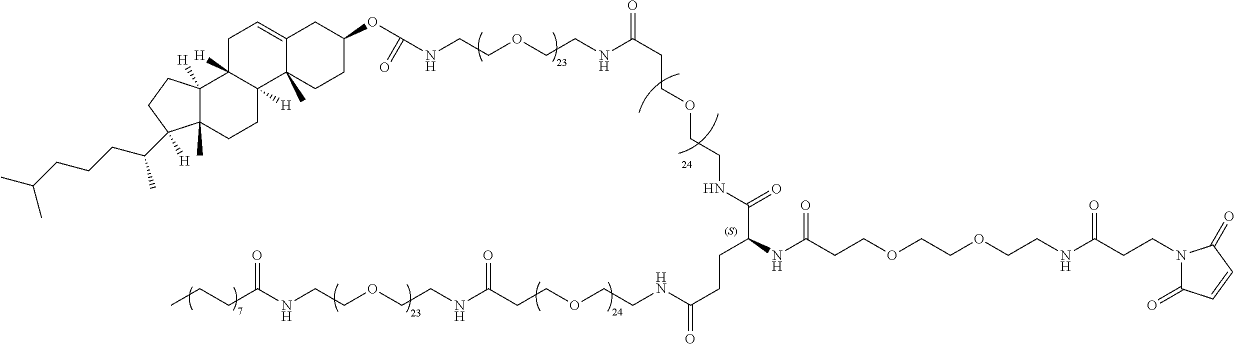

LP47-p

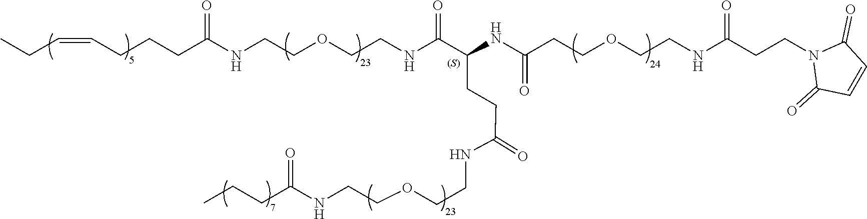

LP48-p

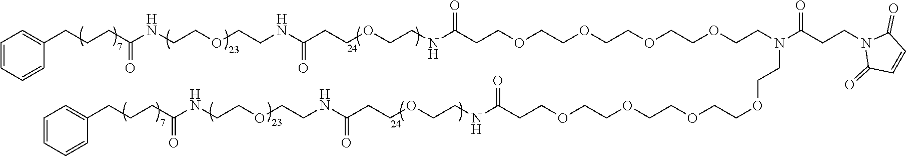

LP49-p

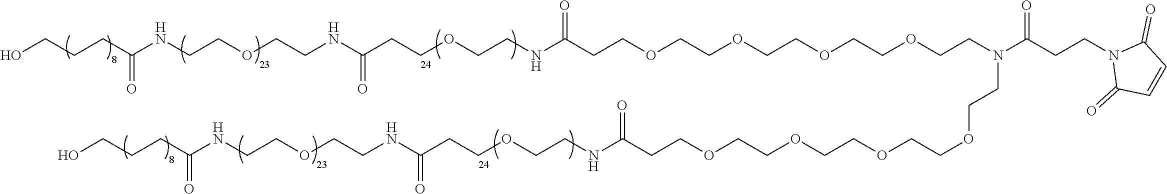

LP53-p

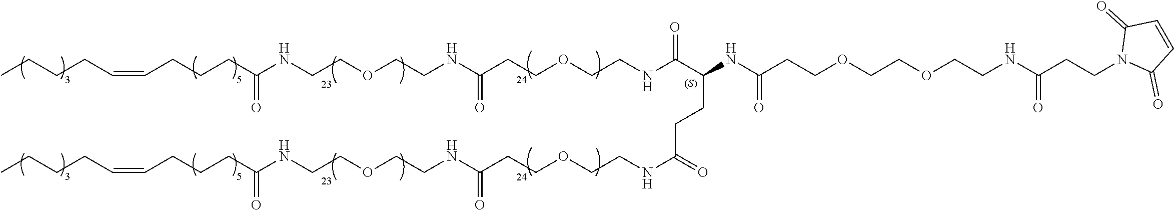

LP54-p

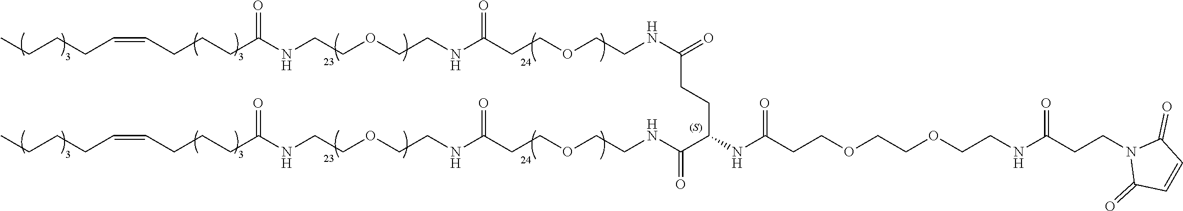

LP55-p

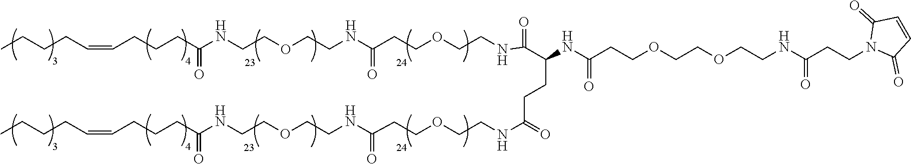

LP56-p

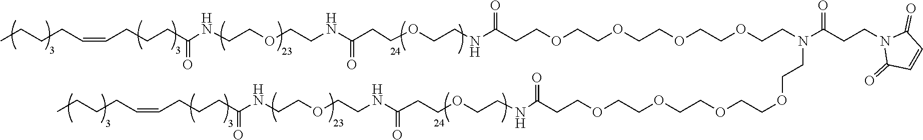

LP57-p

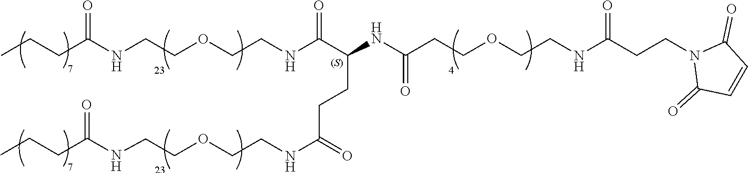

LP58-p

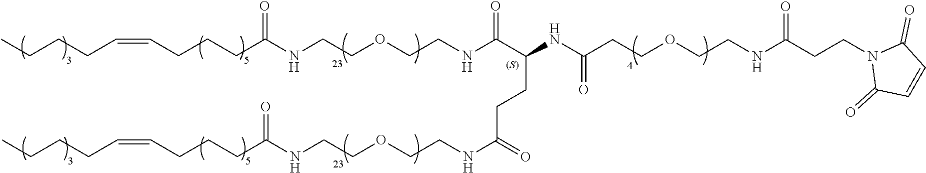

LP59-p

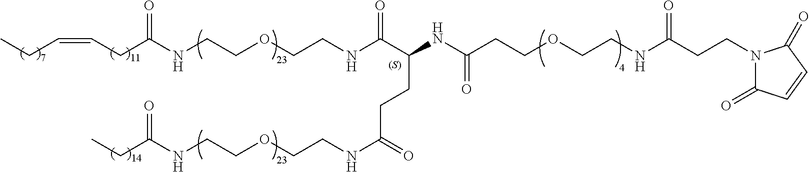

LP60-p

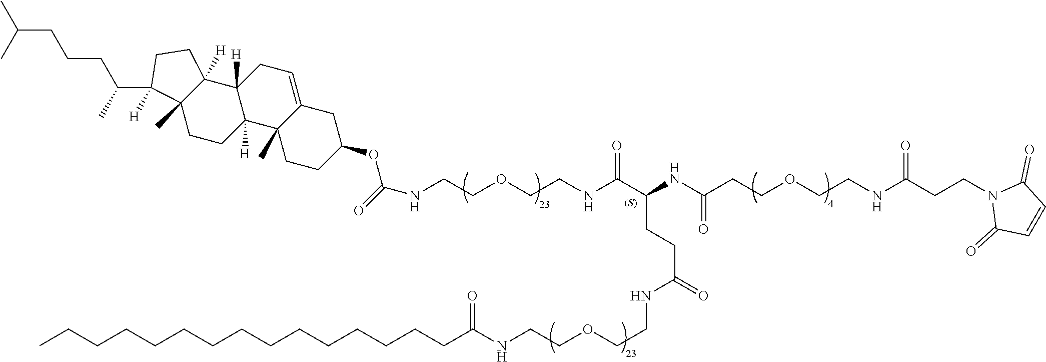

LP61-p

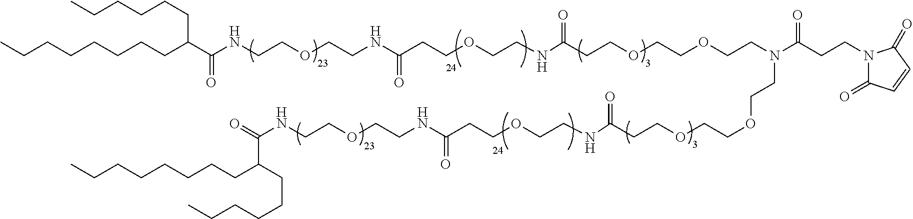

LP62-p

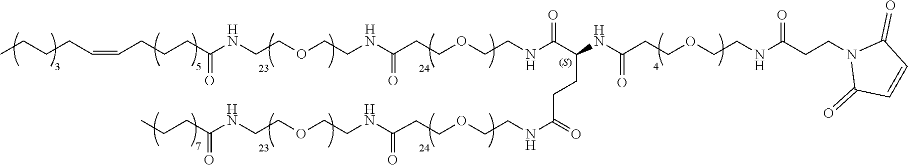

LP87-p

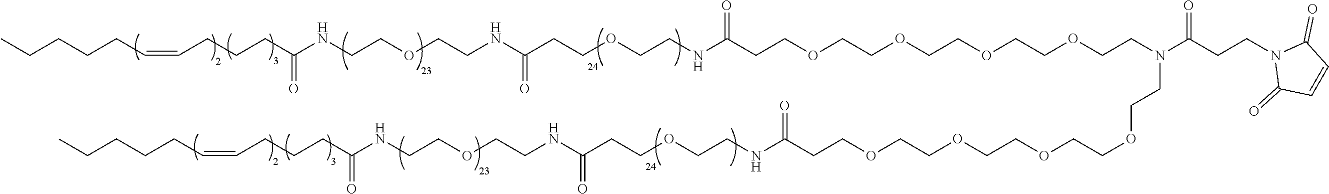

LP89-p

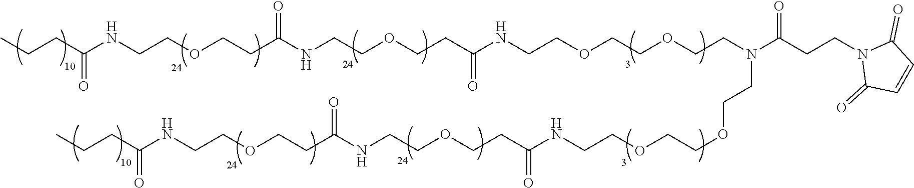

LP90-p

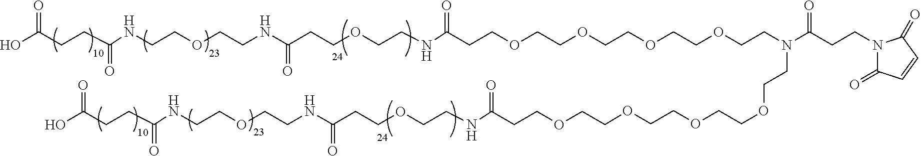

LP92-p

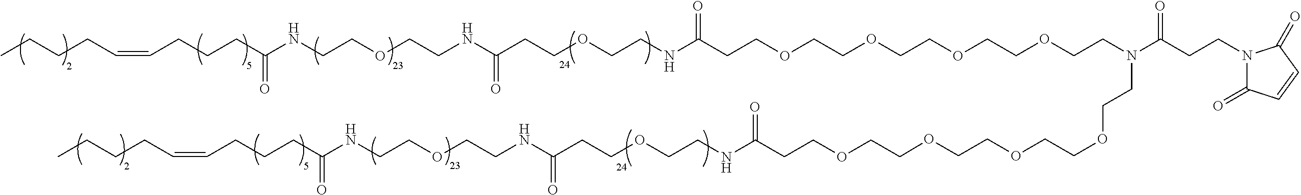

LP93-p

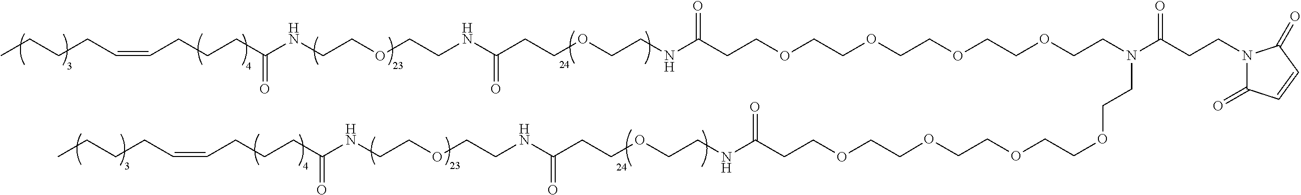

LP94-p

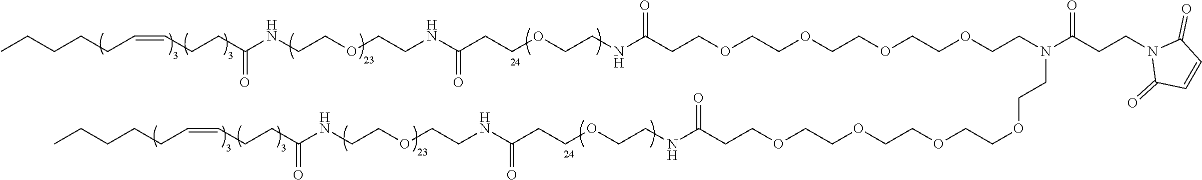

LP95-p

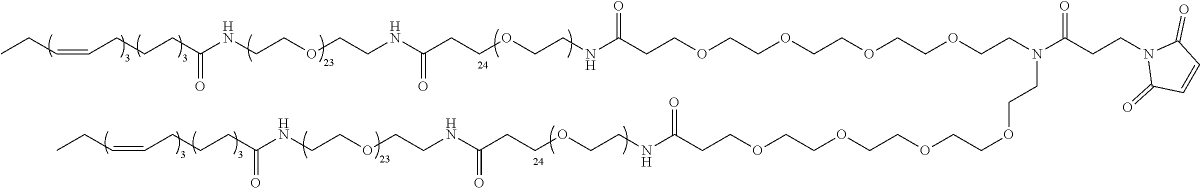

LP101-p

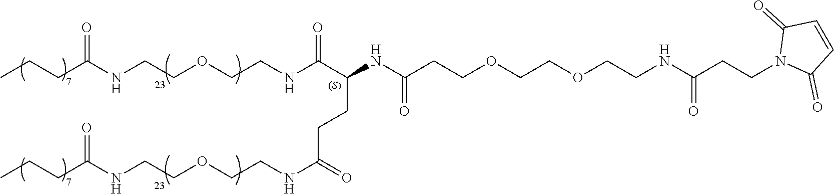

LP102-p

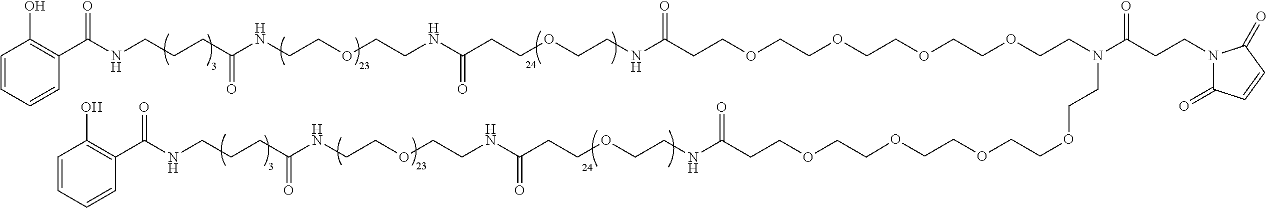

LP103-p

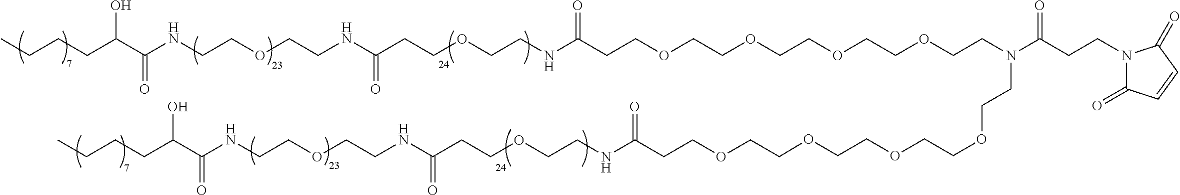

LP104-p

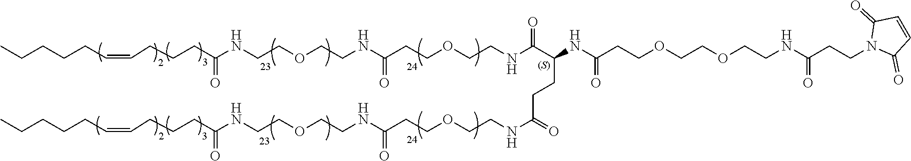

LP106-p

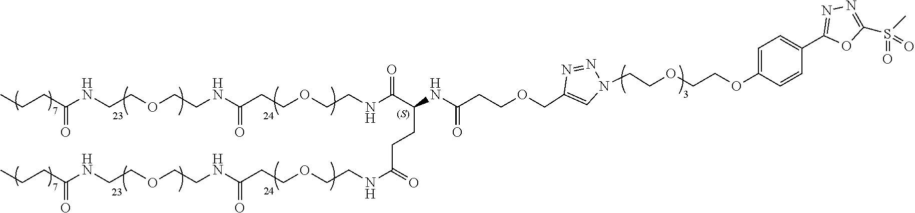

LP107-p

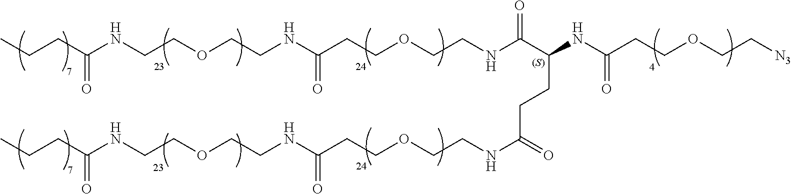

LP108-p

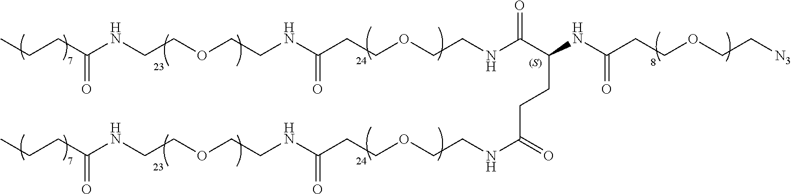

LP109-p

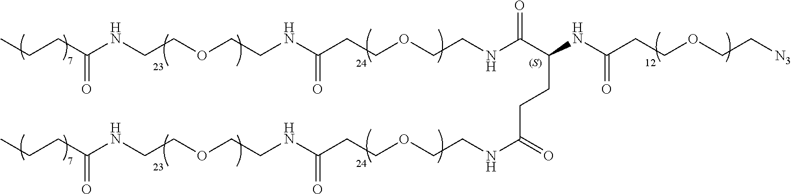

LP110-p

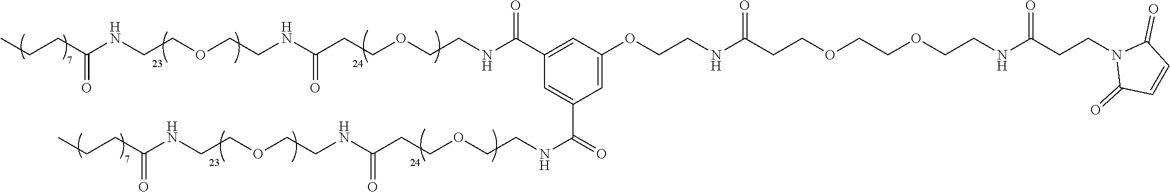

LP111-p

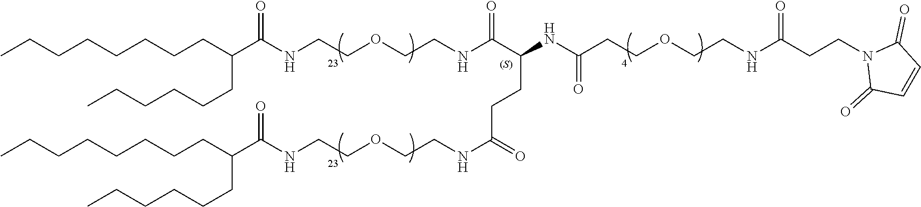

LP124-p

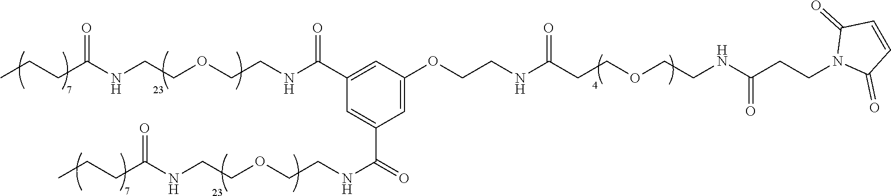

LP130-p

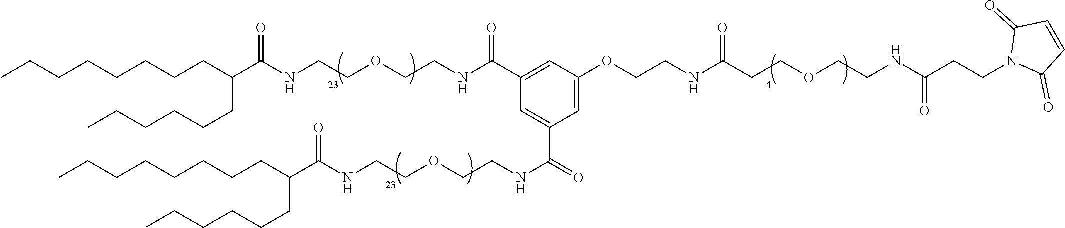

LP143-p

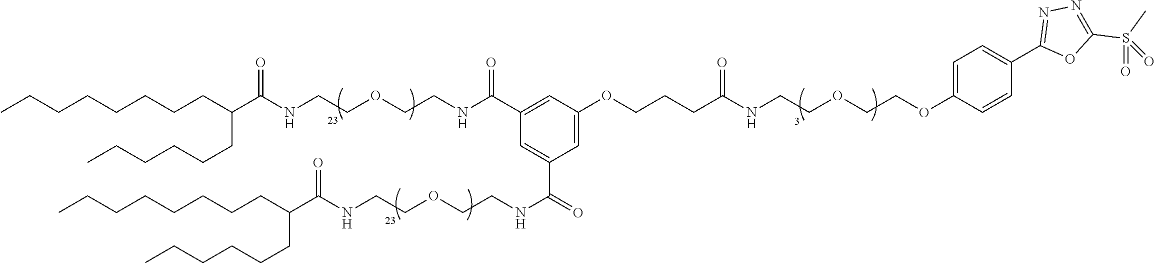

LP210-p

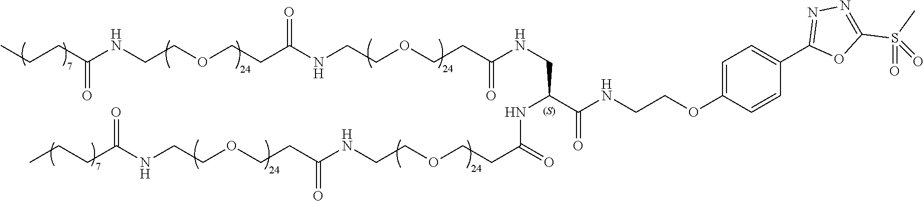

LP217-p

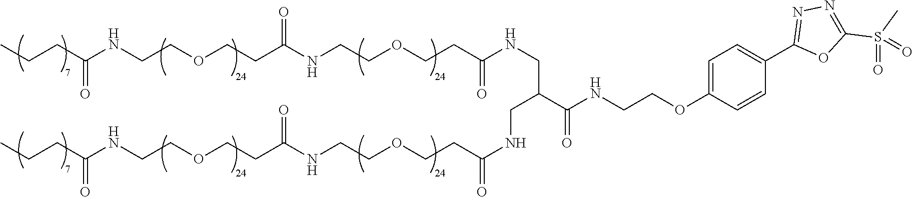

LP220-p

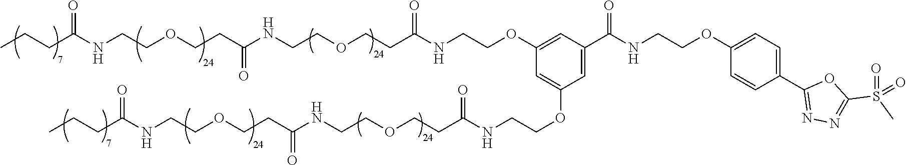

LP221-p

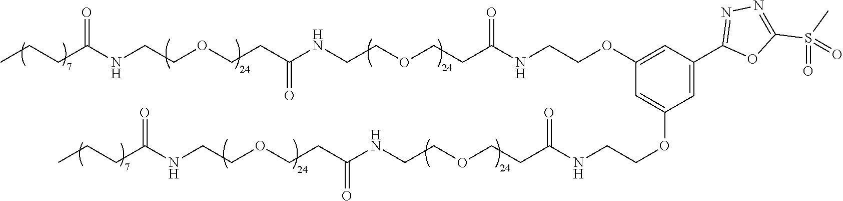

LP223-p

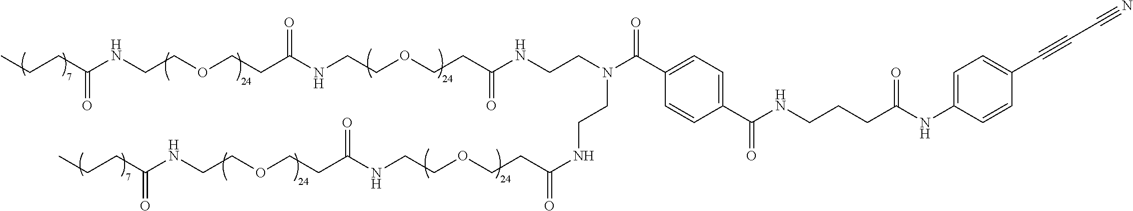

LP224-p

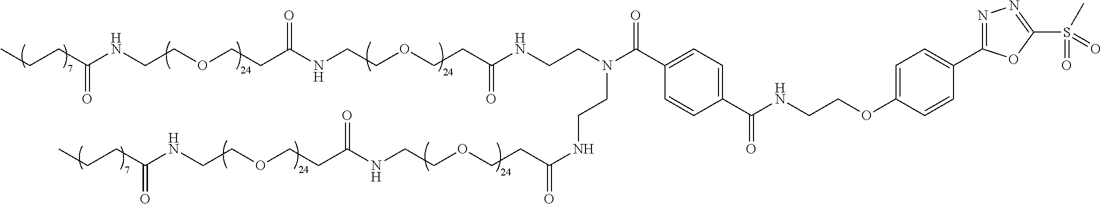

LP225-p

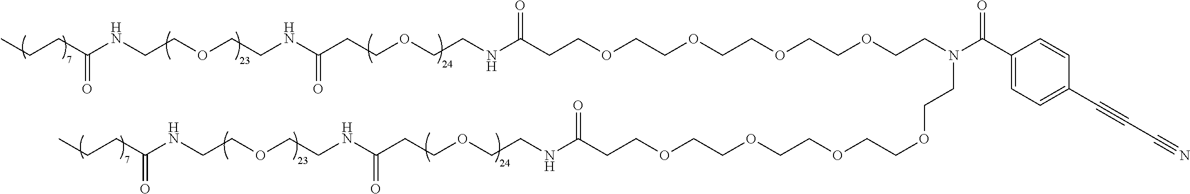

LP226-p

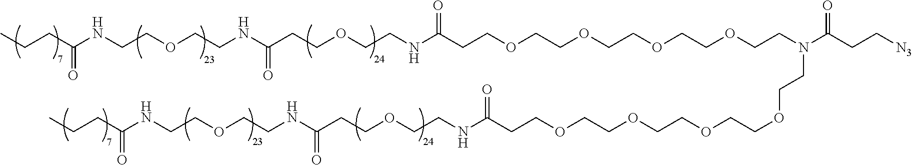

LP238-p

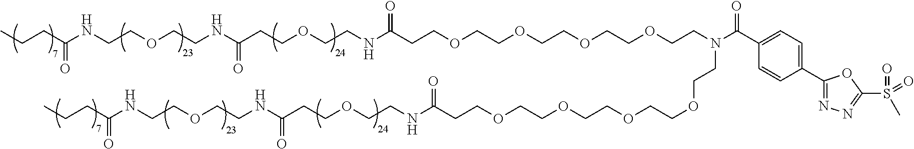

LP240-p

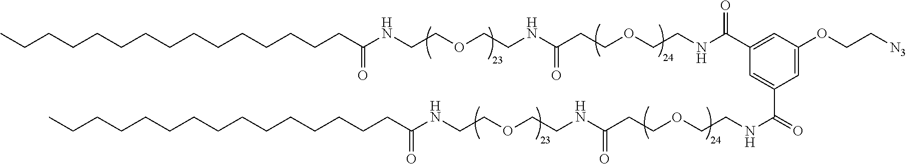

LP246-p

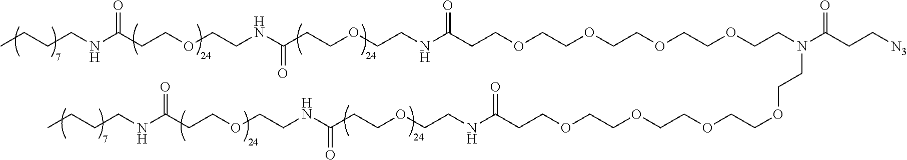

LP247-p

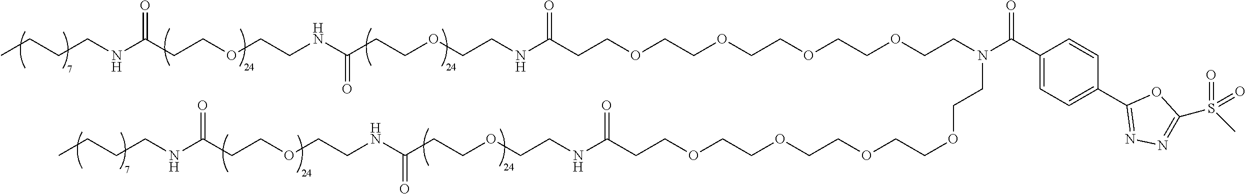

LP339-p

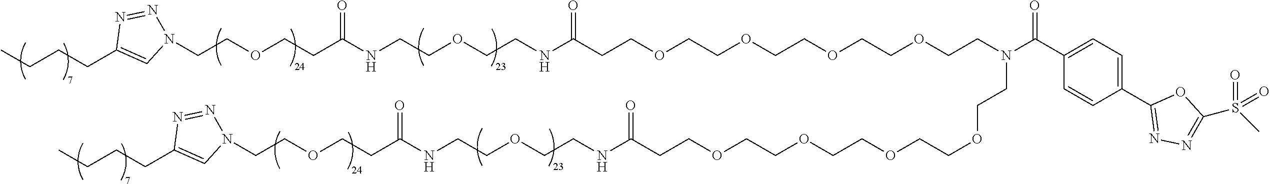

LP340-p

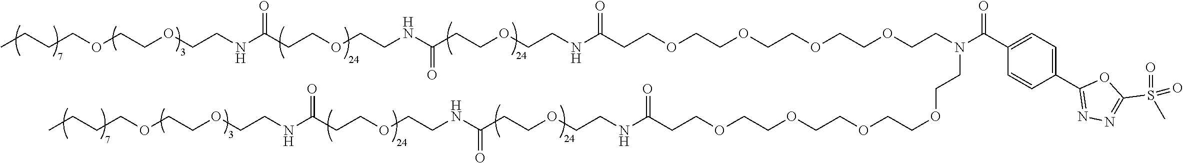

LP357-p

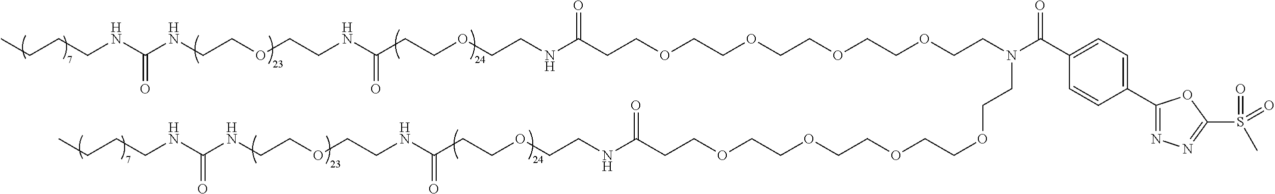

LP358-p

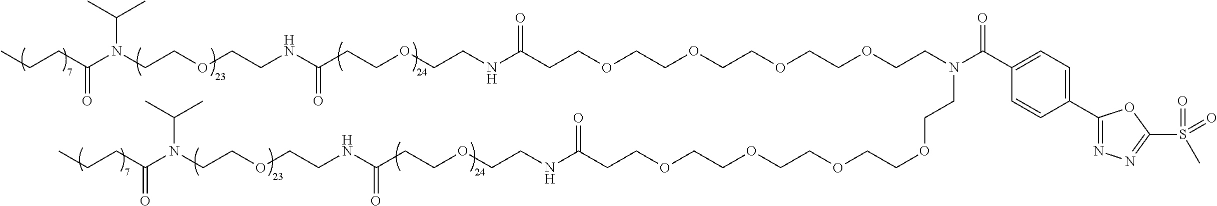

In some embodiments, the PK/PD modulators of Table 6.6 have the following structures following conjugation to the DMPK RNAi agents as shown in Table 6.7:

TABLE 6.7
Example PK/PD modulators for use with DMPK RNAi agents

| | |
|---|---|
| 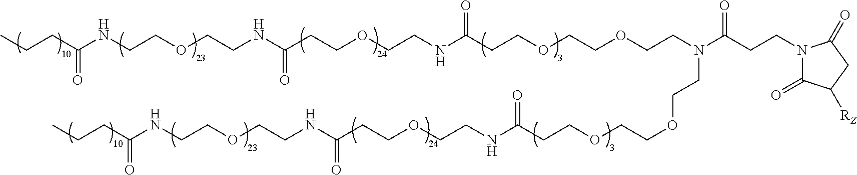 | LP 1b |
| 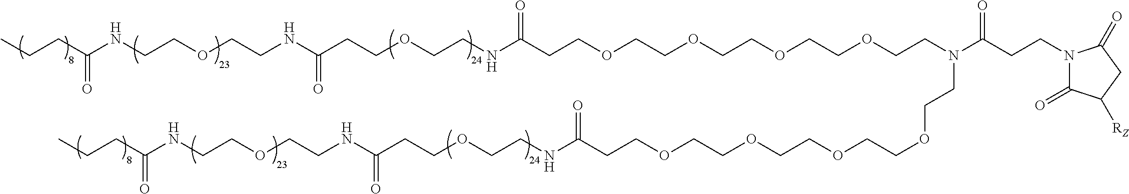 | LP 28b |
| 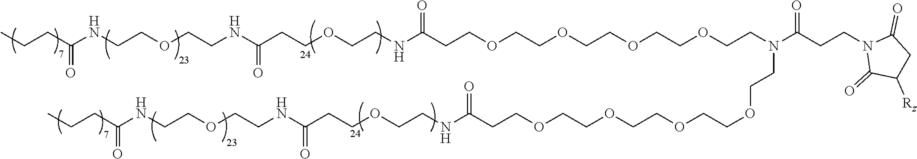 | LP 29b |
| 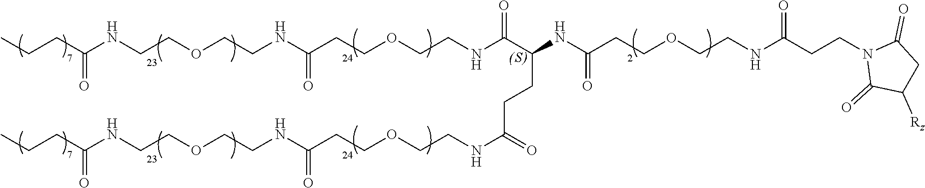 | LP 38b |
| 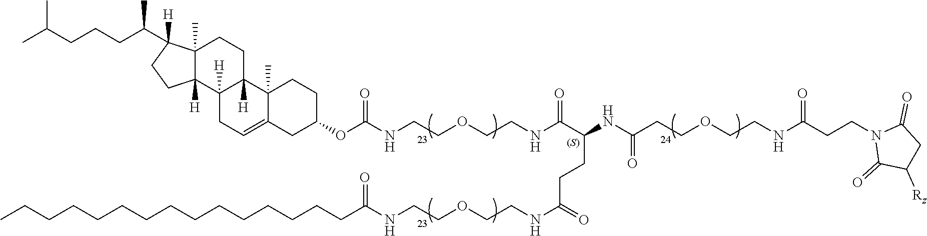 | LP 39b |
| 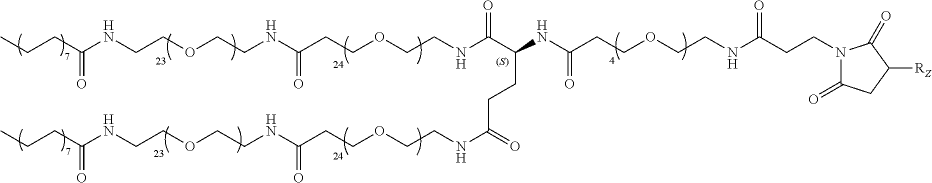 | LP 41b |
| 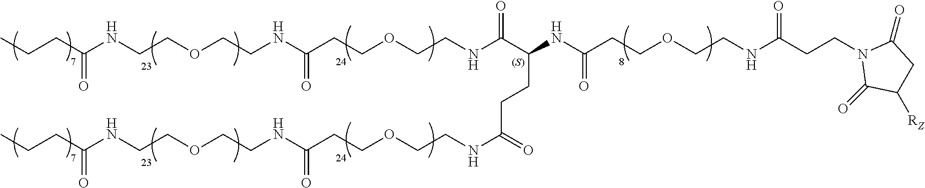 | LP 42b |
| 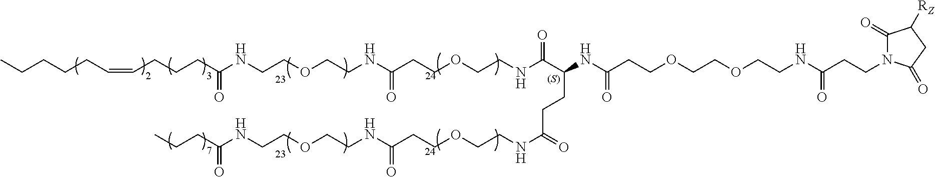 | LP 43b |
| 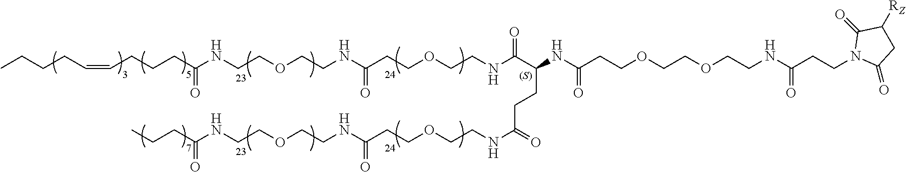 | LP 44b |
| 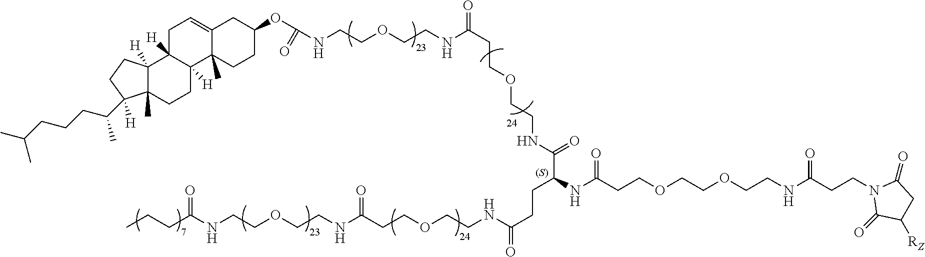 | LP 45b |
| 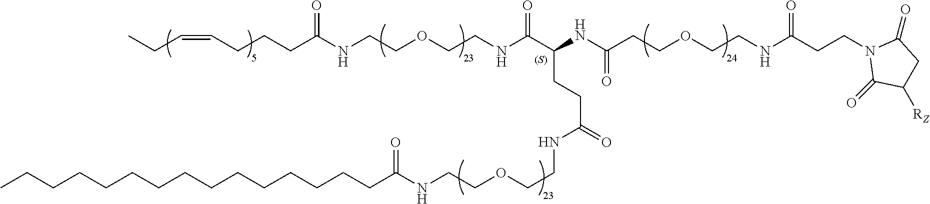 | LP 47b |
| 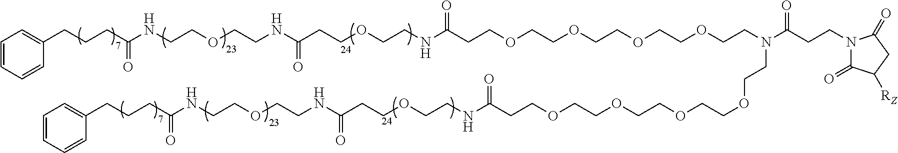 | LP 48b |
| 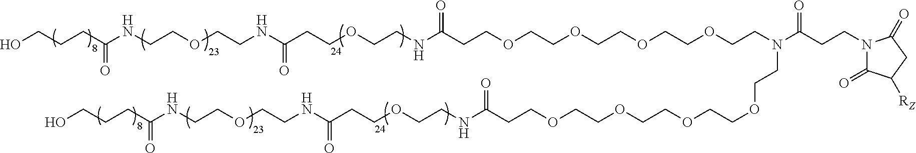 | LP 49b |
| 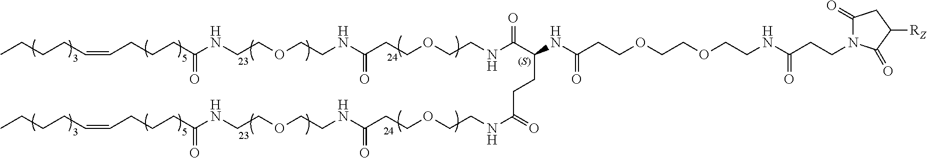 | LP 53b |
| 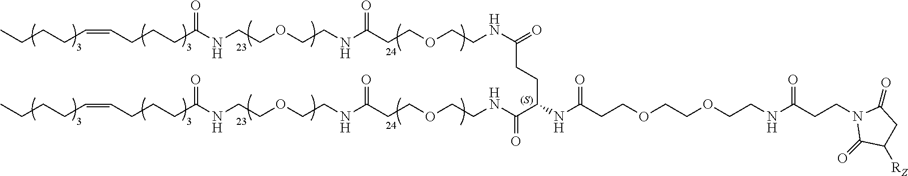 | LP 54b |
| 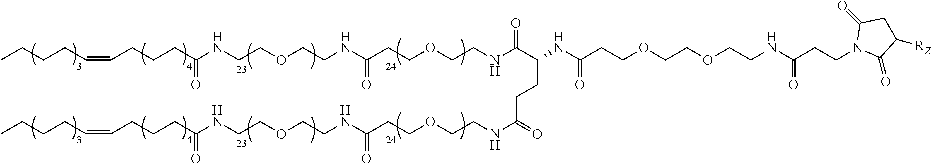 | LP 55b |
| 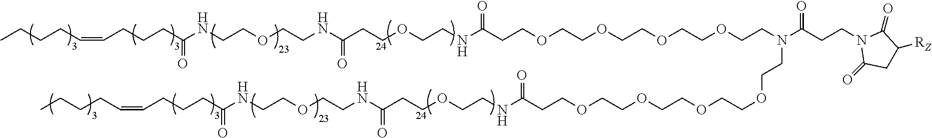 | LP 56b |
| 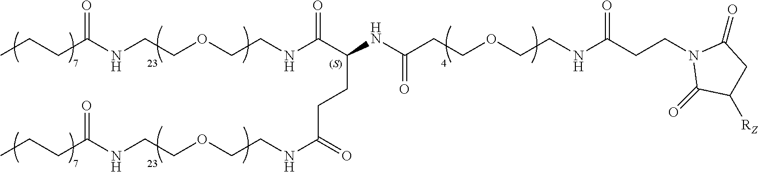 | LP 57b |
| 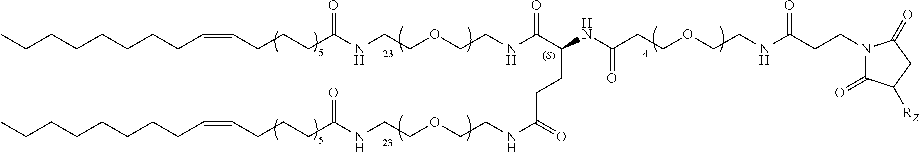 | LP 58b |
| 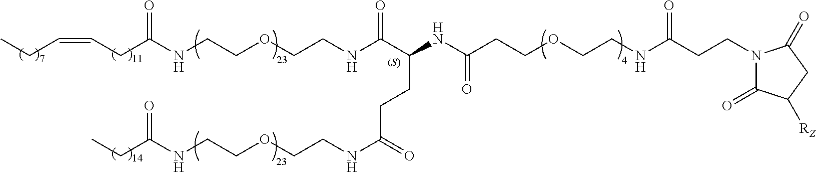 | LP 59b |
| 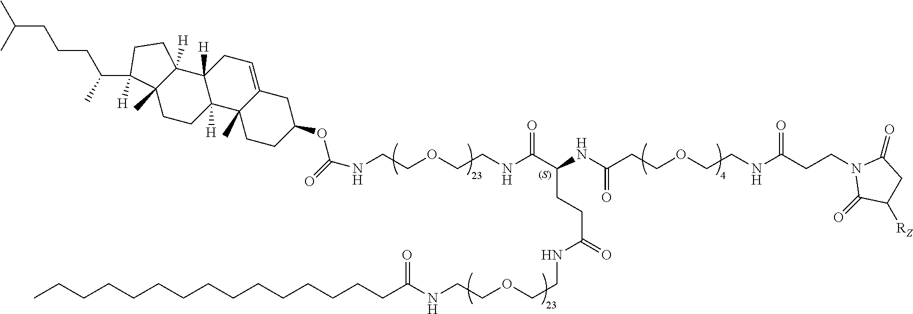 | LP 60b |
| 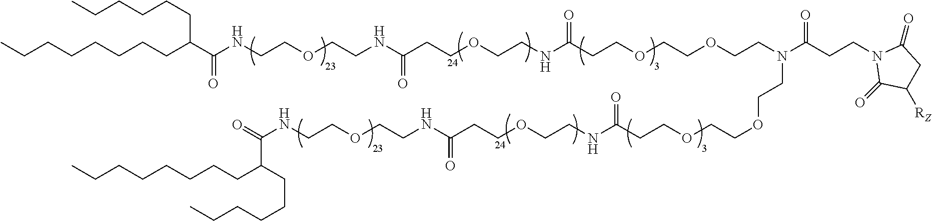 | LP 61b |
| 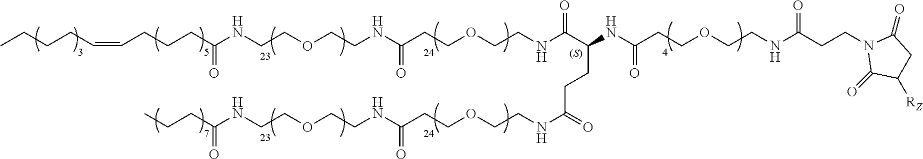 | LP 62b |
| 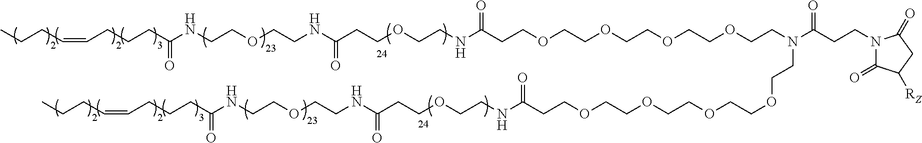 | LP 87b |
| 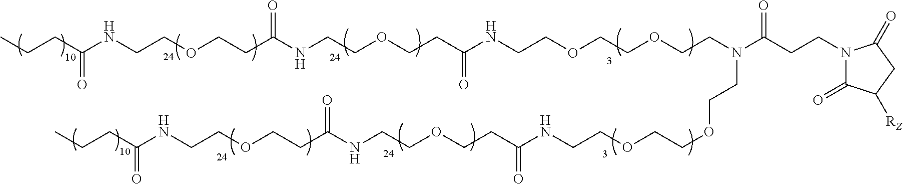 | LP 89b |
| 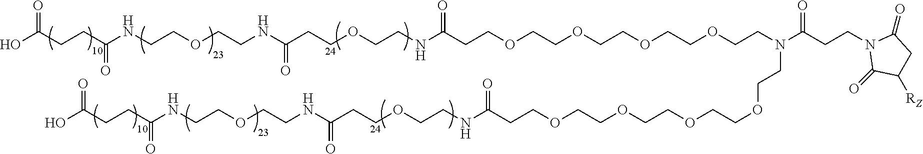 | LP 90b |
| 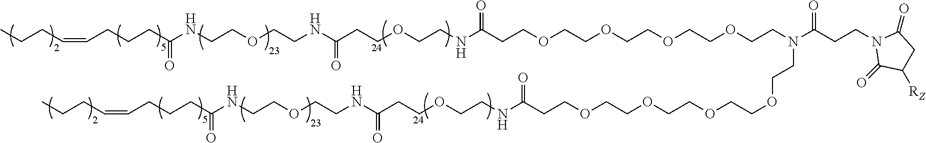 | LP 92b |
| 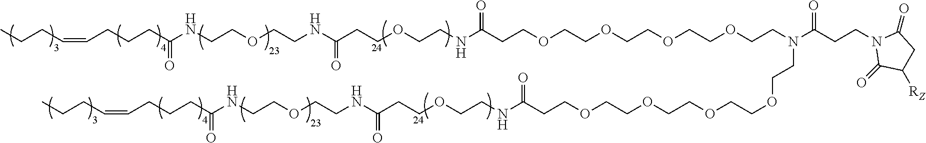 | LP 93b |
| 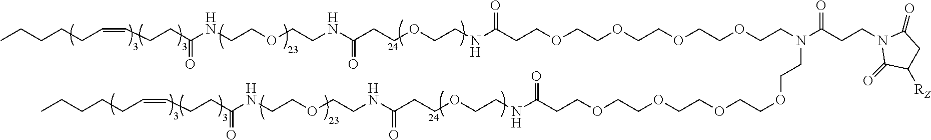 | LP 94b |
| 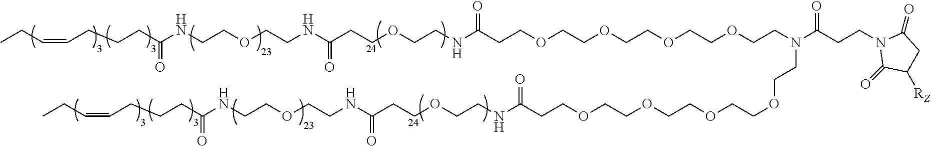 | LP 95b |
| 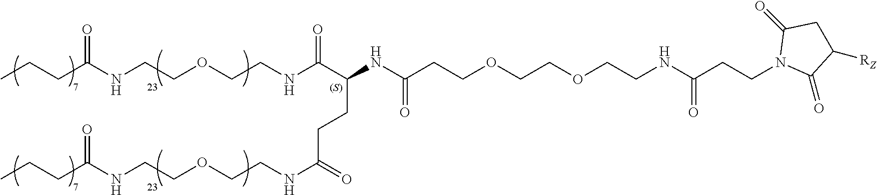 | LP 101b |
| 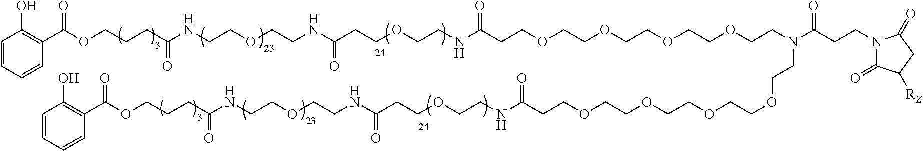 | LP 102b |
| 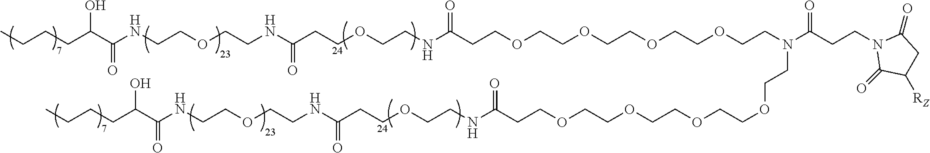 | LP 103b |
| 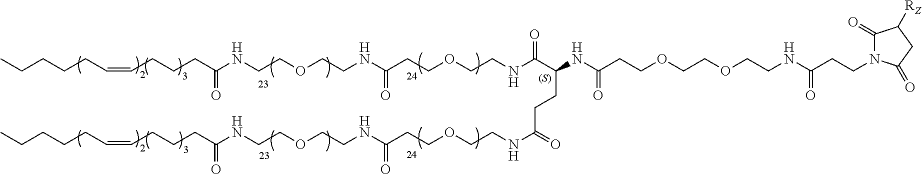 | LP 104b |
| 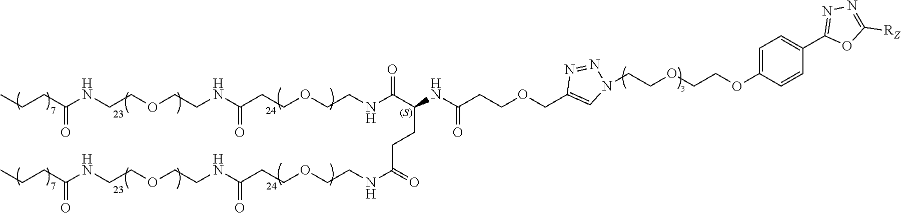 | LP 106b |
| 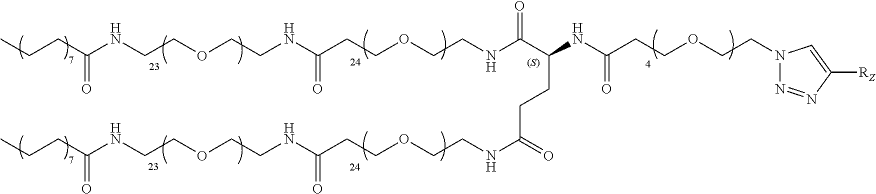 | LP 107b |
| 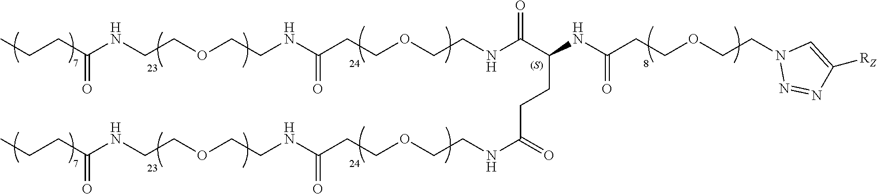 | LP 108b |
| 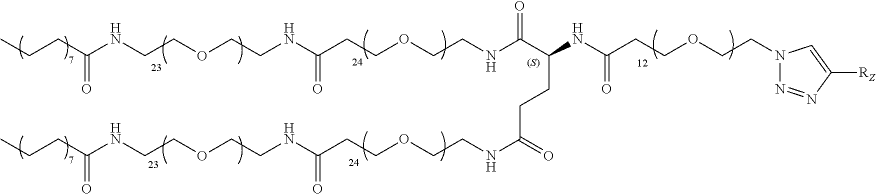 | LP 109b |
| 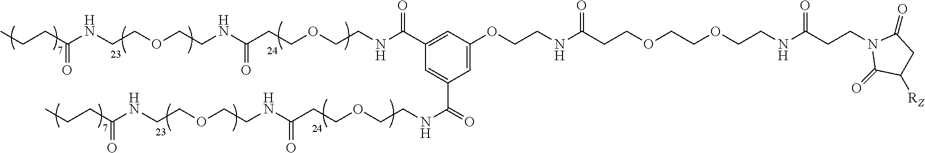 | LP 110b |
| 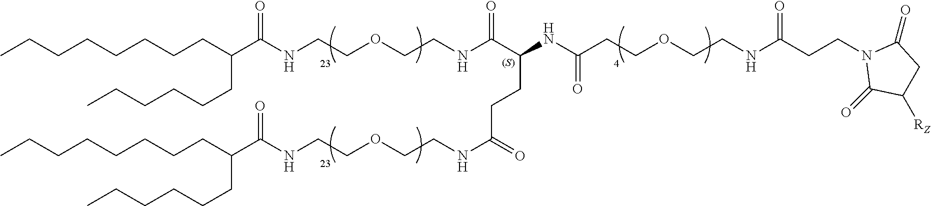 | LP 111b |
| 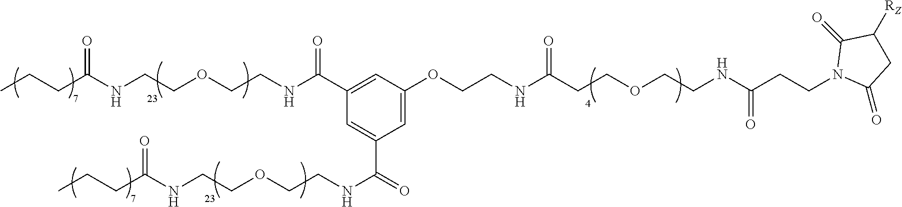 | LP 124b |
| 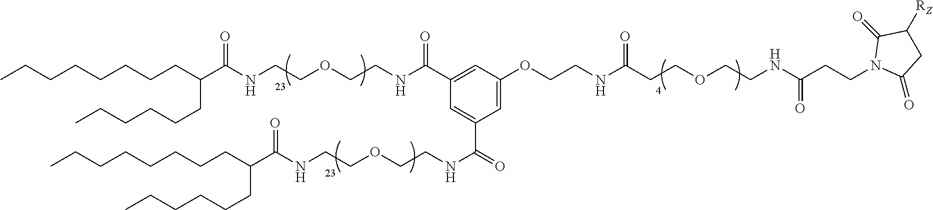 | LP 130b |
| 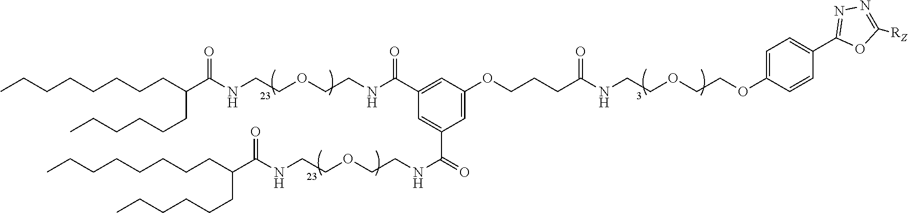 | LP 143b |
| 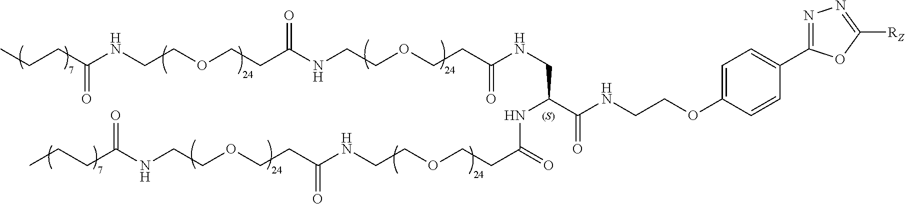 | LP 210b |
| 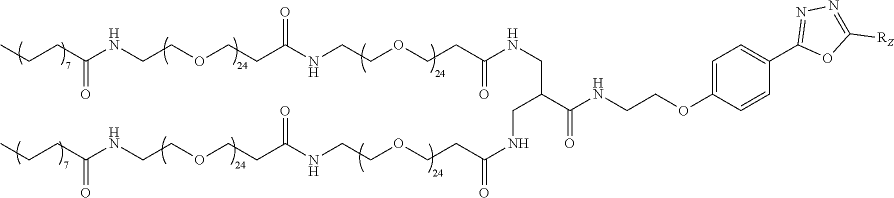 | LP 217b |
| 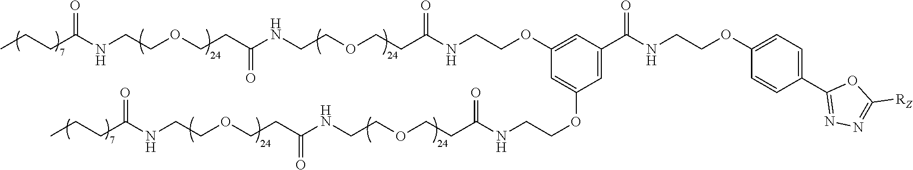 | LP 220b |
| 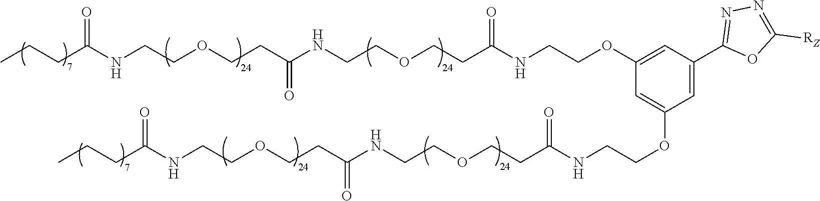 | LP 221b |
| 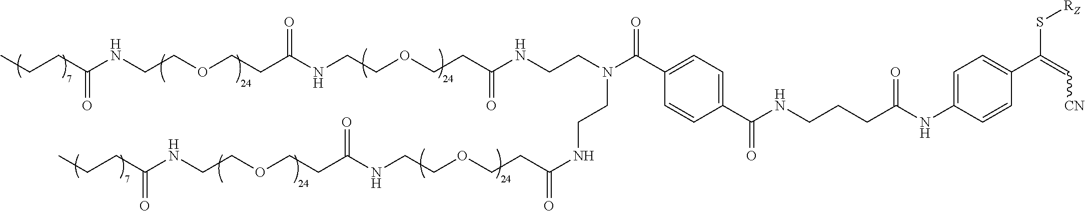 | LP 223b |
| 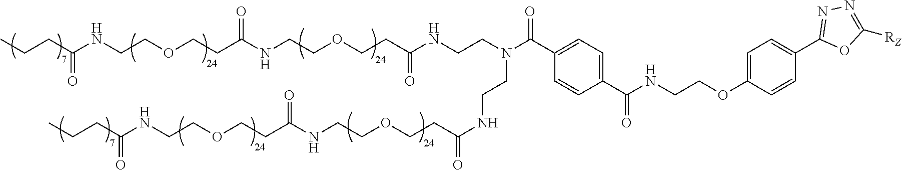 | LP 224b |
| 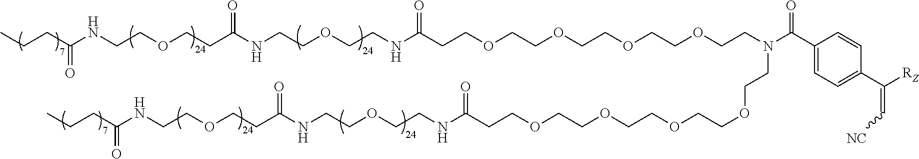 | LP 225b |
| 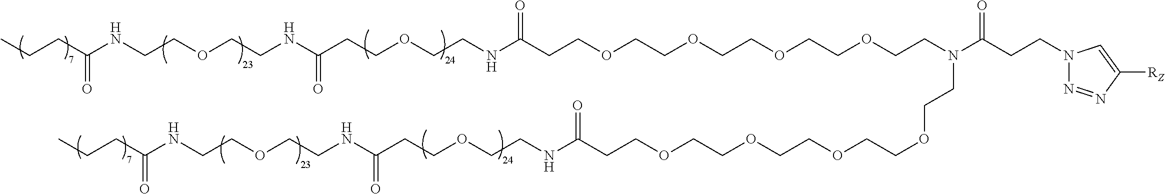 | LP 226b |
| 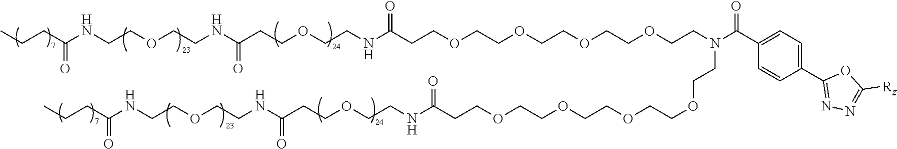 | LP 238b |
| 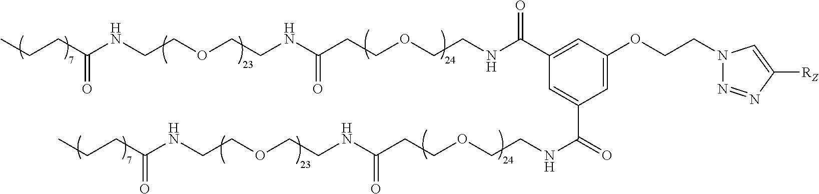 | LP 240b |
| 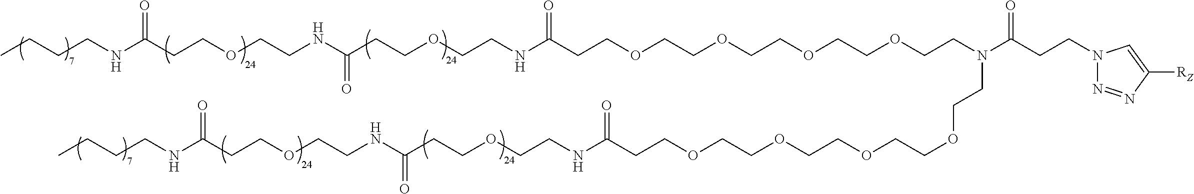 | LP 246b |
| 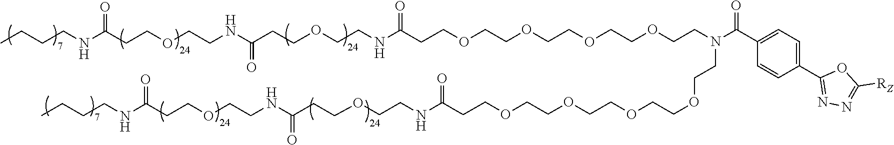 | LP 247b |
| 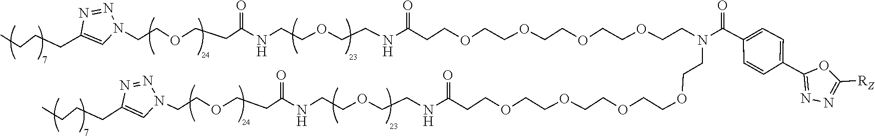 | LP 339b |
| 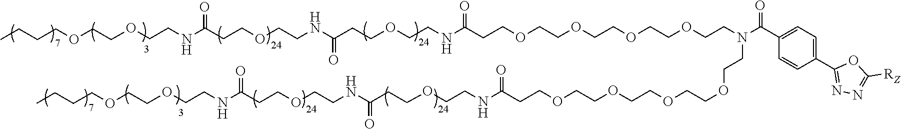 | LP 340b |
| 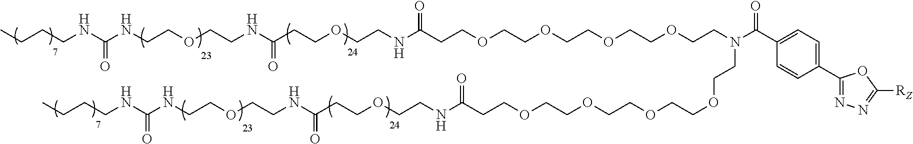 | LP 357b |
| 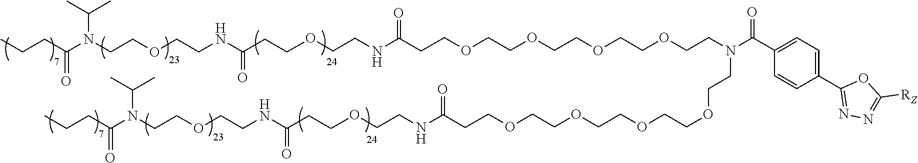 | LP 358b |

wherein R_{Z} represents the remainder of the DMPK RNAi agent.

In some embodiments, DMPK RNAi agents may comprise one or more PK/PD modulators. In some embodiments, the DMPK RNAi agents disclosed herein comprise one, two, three, four, five, six, seven or more PK/PD modulators.

PK/PD modulators may be conjugated to a DMPK RNAi agent using any known method in the art. Many PK/PD modulators, including several of those above, are commercially available. In some embodiments, such as several of the compounds shown in Table 6.4, PK/PD modulators can include a maleimide moiety and be reacted with an RNAi agent comprising a disulfide linkage to form an RNAi agent comprising a PK/PD modulator. The disulfide may be reduced, and added to a maleimide by way of a Michael-Addition reaction. An example reaction scheme is shown below:

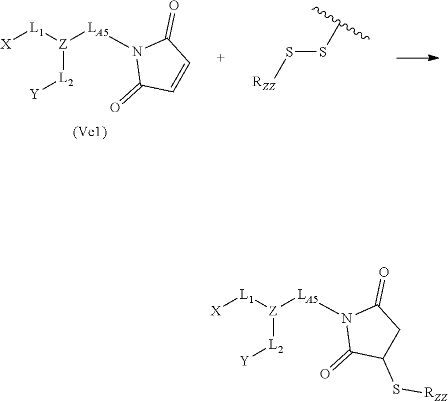

wherein R_{ZZ} comprises an RNAi agent, and [symbol] indicates a point of connection to any suitable group known in the art. In some instances of the reaction scheme above,

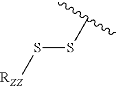

is attached to an alkyl group such as hexyl (C₆H₁₃).

In some embodiments, PK/PD modulator precursors may comprise a sulfone moiety and may react with a disulfide. An example reaction scheme is shown below:

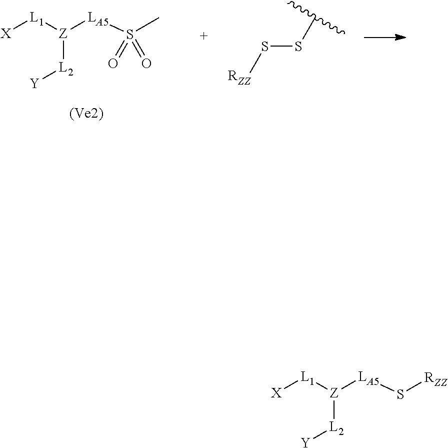

wherein R_{ZZ} comprises an RNAi agent, and [symbol] indicates a point of connection to any suitable group known in the art. In some instances of the reaction scheme above,

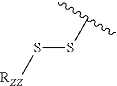

is attached to an alkyl group such as hexyl (C₆H₁₃).

In some embodiments, PK/PD modulator precursors may comprise an azide moiety and be reacted with an RNAi agent comprising an alkyne to form a compound comprising a PK/PD modulator conjugated to an RNAi agent according to the general reaction scheme below:

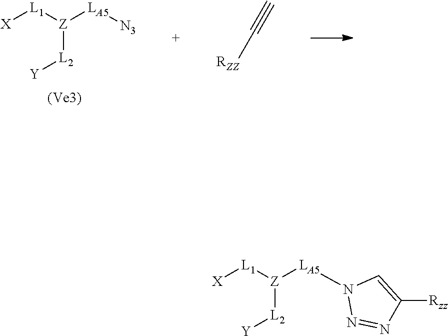

wherein R_{ZZ} comprises an RNAi agent.

In some embodiments, PK/PD modulator precursors may comprise an alkyne moiety and be reacted with an RNAi agent comprising a disulfide to form a compound comprising a PK/PD modulator conjugated to an RNAi agent according to the general reaction scheme below:

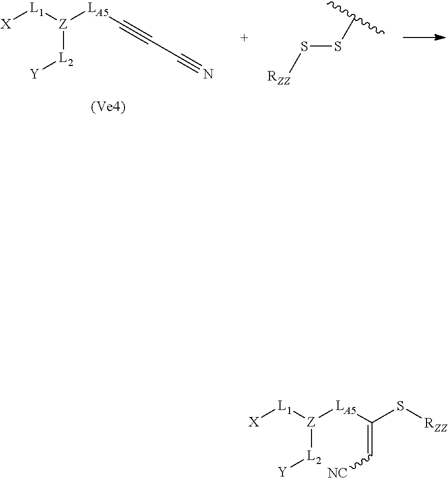

wherein R_{ZZ} comprises an RNAi agent, and [symbol] indicates a point of connection to any suitable group known in the art. In some instances of the reaction scheme above,

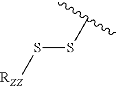

is attached to an alkyl group such as hexyl (C₆H₁₃).

In some embodiments, PK/PD modulators may be conjugated to the 5′ end of the sense or antisense strand, the 3′ end of the sense or antisense strand, or to an internal nucleotide of a DMPK RNAi agent. In some embodiments, a DMPK RNAi agent is synthesized with a disulfide-containing moiety at the 3′ end of the sense strand, and a PK/PD modulator may be conjugated to the 3′ end of the sense strand using the general synthetic scheme shown above.

### Pharmaceutical Compositions and Formulations

The DMPK RNAi agents disclosed herein can be prepared as pharmaceutical compositions or formulations (also referred to herein as “medicaments”). In some embodiments, pharmaceutical compositions include at least one DMPK RNAi agent. These pharmaceutical compositions are particularly useful in the inhibition of the expression of DMPK mRNA in a target cell, a group of cells, a tissue, or an organism. The pharmaceutical compositions can be used to treat a subject having a disease, disorder, or condition that would benefit from reduction in the level of the target mRNA, or inhibition in expression of the target gene. In some embodiments, the disease to be treated is myotonic dystrophy type 1. The pharmaceutical compositions can be used to treat a subject at risk of developing a disease or disorder that would benefit from reduction of the level of the target mRNA or an inhibition in expression the target gene. In one embodiment, the method includes administering a DMPK RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions that include a DMPK RNAi agent, thereby forming a pharmaceutical formulation or medicament suitable for in vivo delivery to a subject, including a human.

In some embodiments, one or more of the described DMPK RNAi agents are administered to a mammal in a pharmaceutically acceptable carrier or diluent. In some embodiments, the mammal is a human. The pharmaceutical compositions including one or more DMPK RNAi agents can be administered in a number of ways depending upon whether local or systemic treatment is desired. Administration can be, but is not limited to, for example, intravenous, intraarterial, subcutaneous (SQ), intraperitoneal, subdermal (e.g., via an implanted device), and intraparenchymal administration.

The pharmaceutical compositions that include a DMPK RNAi agent and methods disclosed herein decrease the level of the target mRNA in a cell, group of cells, group of cells, tissue, organ, or subject, including by administering to the subject a therapeutically effective amount of a herein described DMPK RNAi agent, thereby inhibiting the expression of DMPK mRNA in the subject. In some embodiments, the subject has been previously identified or diagnosed as having a disease or disorder that is mediated at least in part by DMPK expression. In some embodiments, the subject has been previously identified or diagnosed as having a condition, disease, or disorder that would benefit from a reduction of DMPK protein levels in one or more cells or tissues, and more specifically a reduction in mutant DMPK-CUG protein levels. In some embodiments, the subject has been previously diagnosed with having one or more skeletal muscular diseases such as myotonic dystrophy type 1. In some embodiments, the subject has been suffering from symptoms associated with one or more skeletal muscle diseases.

In some embodiments, the described pharmaceutical compositions that include a DMPK RNAi agent are used for treating or managing clinical presentations in a subject that would benefit from the inhibition of expression of DMPK. In some embodiments, a therapeutically or prophylactically effective amount of one or more of pharmaceutical compositions is administered to a subject in need of such treatment. In some embodiments, administration of any of the disclosed DMPK RNAi agents can be used to decrease the number, severity, and/or frequency of symptoms of a disease in a subject.

The described pharmaceutical compositions that include a DMPK RNAi agent can be used to treat at least one symptom in a subject having a disease or disorder that would benefit from reduction or inhibition in expression of DMPK mRNA. In some embodiments, the subject is administered a therapeutically effective amount of one or more pharmaceutical compositions that include a DMPK RNAi agent thereby treating the symptom.

The route of administration is the path by which a DMPK RNAi agent is brought into contact with the body. In general, methods of administering drugs, oligonucleotides, and nucleic acids, for treatment of a mammal are well known in the art and can be applied to administration of the compositions described herein. The DMPK RNAi agents disclosed herein can be administered via any suitable route in a preparation appropriately tailored to the particular route. In some embodiments, the pharmaceutical compositions can be administered by injection, for example, intravenously, intramuscularly, intracutaneously, subcutaneously, intraarticularly, or intraperitoneally, or topically.

The pharmaceutical compositions including a DMPK RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In some embodiments, the compositions are administered via subcutaneous injection, intramuscular injection, or intravenous administration.

In some embodiments, the pharmaceutical compositions described herein comprise one or more pharmaceutically acceptable excipients. The pharmaceutical compositions described herein are formulated for administration to a subject.

In some embodiments, pharmaceutical formulations that include the DMPK RNAi agents disclosed herein suitable for SQ or IV administration can be prepared in an aqueous sodium phosphate buffer (e.g., the DMPK RNAi agent formulated in 0.5 mM sodium phosphate monobasic, 0.5 mM sodium phosphate dibasic, in water). In some embodiments, pharmaceutical formulations that include the DMPK RNAi agents disclosed herein suitable for SQ or IV administration can be prepared in water for injection.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., DMPK RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor® EL™ (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The DMPK RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, analgesics, antihistamines, or anti-inflammatory agents (e.g., acetaminophen, NSAIDs, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic, or preventive result.

In some embodiments, the methods disclosed herein further comprise the step of administering a second therapeutic or treatment in addition to administering an RNAi agent disclosed herein. In some embodiments, the second therapeutic is another DMPK RNAi agent (e.g., a DMPK RNAi agent that targets a different sequence within the DMPK target). In other embodiments, the second therapeutic can be a small molecule drug, an antibody, an antibody fragment, and/or an aptamer.

Generally, an effective amount of a DMPK RNAi agent disclosed herein will be in the range of from about 0.0001 to about 20 mg/kg of body weight/dose, e.g., from about 0.5 to about 10 mg/kg of body weight/dose. The amount administered and dosing frequency (e.g., daily, bi-weekly, weekly, monthly, quarterly, or semi-annually) will likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level to rapidly achieve the desired blood-level or tissue level, or the initial dosage can be smaller than the optimum.

In some embodiments, an effective amount of a DMPK RNAi agent disclosed herein can be administered as a fixed dose from about 0.0001 mg to about 2,500 mg of DMPK RNAi agent. In some embodiments, an effective amount of a DMPK RNAi agent disclosed herein can be administered as a fixed dose from about 100 mg, about 150 mg, about 200 mg, about 250 mg, about 300 mg, about 350 mg, about 400 mg, about 450 mg, about 500 mg, about 600 mg, about 700 mg, about 750 mg, about 800 mg, about 900 mg, or about 1,000 mg.

For treatment of disease or for formation of a medicament or composition for treatment of a disease, the pharmaceutical compositions described herein including a DMPK RNAi agent can be combined with an excipient or with a second therapeutic agent or treatment including, but not limited to: a second or other RNAi agent, a small molecule drug, an antibody, an antibody fragment, peptide, and/or an aptamer.

The described DMPK RNAi agents, when added to pharmaceutically acceptable excipients or adjuvants, can be packaged into kits, containers, packs, or dispensers. The pharmaceutical compositions described herein can be packaged, for example, in pre-filled syringes or vials.

### Methods of Treatment and Inhibition of Expression

The DMPK RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from a reduction and/or inhibition in expression of DMPK mRNA.

In some embodiments, the RNAi agents disclosed herein can be used to treat a subject (e.g., a human) having a disease or disorder for which the subject would benefit from reduction in DMPK protein levels, more specifically a reduction in mutant DMPK-CUG protein levels, including but not limited to, for example, myotonic dystrophy type 1. Treatment of a subject can include therapeutic and/or prophylactic treatment. The subject is administered a therapeutically effective amount of any one or more DMPK RNAi agents described herein. The subject can be a human, patient, or human patient. The subject may be an adult, adolescent, child, or infant. Administration of a pharmaceutical composition described herein can be to a human being or animal.

In some embodiments, the described DMPK RNAi agents are used to treat at least one symptom mediated at least in part by DMPK protein levels, in a subject. The subject is administered a therapeutically effective amount of any one or more of the described DMPK RNAi agents. In some embodiments, the subject is administered a prophylactically effective amount of any one or more of the described RNAi agents, thereby treating the subject by preventing or inhibiting the at least one symptom.

In certain embodiments, the present disclosure provides methods for treatment of diseases, disorders, conditions, or pathological states mediated at least in part by DMPK gene expression, in a patient in need thereof, wherein the methods include administering to the patient any of the DMPK RNAi agents described herein.

In some embodiments, the DMPK RNAi agents are used to treat or manage a clinical presentation or pathological state in a subject, wherein the clinical presentation or pathological state is mediated at least in part by DMPK expression. The subject is administered a therapeutically effective amount of one or more of the DMPK RNAi agents or DMPK RNAi agent-containing compositions described herein. In some embodiments, the method comprises administering a composition comprising a DMPK RNAi agent described herein to a subject to be treated.

In some embodiments, the gene expression level or mRNA level of a DMPK gene in certain skeletal muscle cells of subject to whom a described DMPK RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the DMPK RNAi agent or to a subject not receiving the DMPK RNAi agent. In some embodiments, the DMPK protein levels (including mutant DMPK-CUG protein levels) of a subject to whom a described DMPK RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the DMPK RNAi agent or to a subject not receiving the DMPK RNAi agent. The gene expression level, protein level, and/or mRNA level in the subject may be reduced in a cell, group of cells, tissue, and/or other fluid of the subject. In some embodiments, the DMPK mRNA levels in certain skeletal muscle cells or skeletal muscle tissues in a subject to whom a described DMPK RNAi agent has been administered is reduced by at least about 30%, 35%, 40%, 45%, 50%, 55%, 60%, 65%, 70%, 75%, 80%, 85%, 90%, 95%, or 98% relative to the subject prior to being administered the DMPK RNAi agent or to a subject not receiving the DMPK RNAi agent. In some embodiments, the level of DMPK protein (including mutant DMPK-CUG protein levels) in the skeletal muscle cells and/or skeletal muscle tissue of a subject to whom a described DMPK RNAi agent has been administered is reduced by at least about 30%, 35%, 40%, 45%, 50%, 55%, 60%, 65%, 70%, 75%, 80%, 85%, 90%, 95%, or 98% relative to the subject prior to being administered the DMPK RNAi agent or to a subject not receiving the DMPK RNAi agent.

As noted herein the DMPK protein level (including mutant DMPK-CUG protein levels) and/or DMPK mRNA level in the subject may be reduced in a cell, group of cells, tissue, blood, and/or other fluid (e.g., serum) of the subject, as would be understood by the person of ordinary skill in the art. For example, in some embodiments, the level of DMPK mRNA of a subject to whom a described DMPK RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the DMPK RNAi agent or to a subject not receiving the DMPK RNAi agent in one or more skeletal muscle cells or skeletal muscle tissues. In some embodiments, the level of DMPK mRNA and/or DMPK protein in a subset of skeletal muscle cells, of a subject to whom a described DMPK RNAi agent has been administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the DMPK RNAi agent or to a subject not receiving the DMPK RNAi agent.

In some embodiments, the DMPK RNAi agents can reduce DMPK gene expression in one or more of the following muscle tissues: triceps, biceps, quadriceps, gastrocnemius, soleus, masseter EDL (extensor digitorum longus), TA (Tibialis anterior), trapezius, and/or diaphragm.

A reduction in gene expression, mRNA, and protein levels can be assessed by any methods known in the art. For example, the Examples set forth herein provide appropriate ways for measuring DMPK protein levels (including mutant DMPK-CUG protein levels) and DMPK mRNA levels in a subject. Reduction or decrease in DMPK mRNA levels and/or DMPK protein levels (including mutant DMPK-CUG protein levels), are collectively referred to herein as a reduction or decrease in DMPK or inhibiting or reducing the expression of a DMPK gene. The Examples set forth herein illustrate known methods for assessing inhibition of DMPK gene expression.

### Cells, Tissues, Organs, and Non-Human Organisms

Cells, tissues, organs, and non-human organisms that include at least one of the DMPK RNAi agents described herein are contemplated. The cell, tissue, organ, or non-human organism is made by delivering the RNAi agent to the cell, tissue, organ, or non-human organism.

The above provided embodiments and items are now illustrated with the following, non-limiting examples.

### EXAMPLES

### Example 1. Synthesis of DMPK RNAi Agents

The DMPK RNAi agents disclosed herein were synthesized in accordance with the following:
A. Synthesis. The sense and antisense strands of the DMPK RNAi agents were synthesized according to phosphoramidite technology on solid phase used in oligonucleotide synthesis. Depending on the scale, a MerMade96ER (Bioautomation), a MerMade12® (Bioautomation), or an OP Pilot 100 (GE Healthcare) was used. Syntheses were performed on a solid support made of controlled pore glass (CPG, 500 Å or 600 Å, obtained from Prime Synthesis, Aston, PA, USA). All RNA and 2′-modified RNA phosphoramidites were purchased from Thermo Fisher Scientific (Milwaukee, WI, USA). Specifically, the 2′-O-methyl phosphoramidites that were used included the following: (5′-O-dimethoxytrityl-N6-(benzoyl)-2′-O-methyl-adenosine-3′-O-(2-cyanoethyl-N,N-diisopropylamino)phosphoramidite, 5′-O-dimethoxy-trityl-N4-(acetyl)-2′-O-methyl-cytidine-3′-O-(2-cyanoethyl-N,N-diisopropyl-amino)phosphoramidite, (5′-O-dimethoxytrityl-N2-(isobutyryl)-2′-O-methyl-guanosine-3′-O-(2-cyanoethyl-N,N-diisopropylamino)phosphoramidite, and 5′-O-dimethoxytrityl-2′-O-methyl-uridine-3′-O-(2-cyanoethyl-N,N-diisopropylamino)phosphoramidite. The 2′-deoxy-2′-fluoro-phosphoramidites carried the same protecting groups as the 2′-O-methyl RNA amidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino)phosphoramidites were purchased from ChemGenes (Wilmington, MA, USA). UNA phosphoramidites include 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite. The cyclopropyl phosphonate phosphoramidites were synthesized in accordance with International Patent Application Publication No. WO 2017/214112 and Erich F. Altenhofer et al., Synthesis of a novel cyclopropyl phosphonate nucleotide as a phosphate mimic, Chemical Communications (June 2021) (DOI: 10.1039/d1cc02328d). TFA aminolink phosphoramidites were also commercially purchased (ThermoFisher).
B. Cleavage and deprotection of support bound oligomer. After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).
C. Purification. Crude oligomers were purified by anionic exchange HPLC using a TSKgel SuperQ-5 PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 16/40 column packed with Sephadex G-25 fine with a running buffer of 100 mM ammonium bicarbonate, pH 6.7 and 20% Acetonitrile or filtered water. Alternatively, pooled fractions were desalted and exchanged into an appropriate buffer or solvent system via tangential flow filtration.
D. Annealing. Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor and the dilution factor to determine the duplex concentration. The conversion factor used was either 0.050 mg/(mL·cm) or experimentally determined.
E. Synthesis of SM45-p for conjugation to RNAi agents; (S)-3-(4-(4-((14-azido-3,6,9,12-tetraoxatetradecyl)oxy) naphthalen-1-yl)phenyl)-3-(2-(5-((4-methylpyridin-2-yl)amino) pentanamido) acetamido) propanoic acid

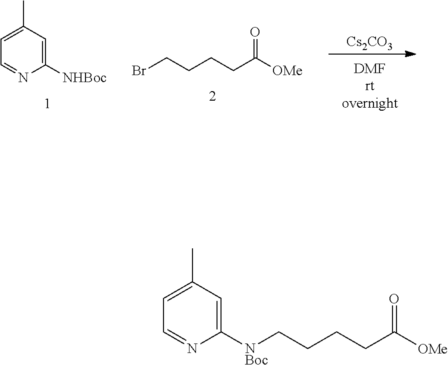

To a solution of compound 1 (0.50 g) in DMF under N₂ (g) at rt was added Cs₂CO₃ (0.94 g). Compound 2 (0.49 g) was then added slowly dropwise. The reaction was stirred overnight. Approx. 50% conversion to desired product by LC-MS was then confirmed. The reaction mixture was quenched with NaHCO₃ (10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash using silica gel as the stationary phase with a gradient of hex to EtOAc (0-70%), in which product eluted at 16% B. The product was concentrated under vacuum to provide a clear oil (0.35 g, 45.0% yield). LC-MS: calculated [M+H]+ 323.19 m/z, observed 328.38 m/z.

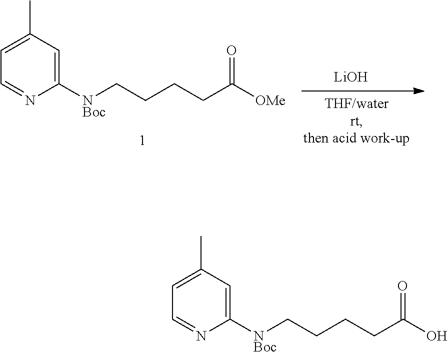 [SMILES: COC(=O)CCCCN(C(=O)OC(C)(C)C)c1cc(C)ccn1 ; "LiOH" ; Cc1ccnc(N(CCCCC(=O)O)C(=O)OC(C)(C)C)c1]

To a solution of compound 1 (0.35 g) in 1:1 THF/water was added LiOH (0.078 g) at rt under normal atmosphere. The reaction was stirred at rt until full conversion was observed by LC-MS. After 1 h, the reaction mixture was acidified with 6 N HCl to a pH of ~3. The product was extracted with EtOAc (3×15 mL). The combined organic phase was dried over Na₂SO₄, filtered, and concentrated, providing a clear, colorless oil (0.32 g, 94.9% yield). No isolation was necessary. LC-MS: calculated [M+H]+ 309.17 m/z, observed 309.24 m/z.

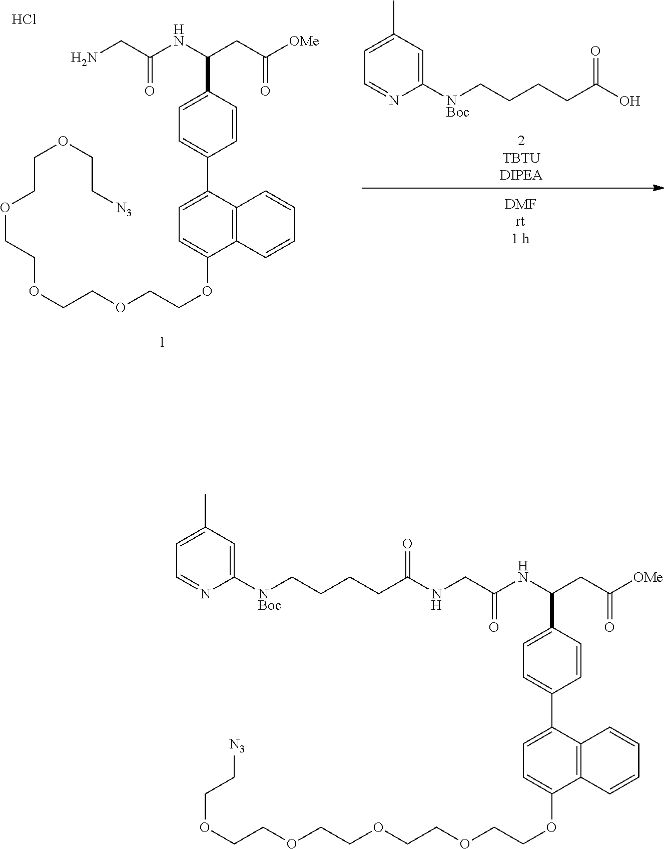 [SMILES: COC(=O)C[C@H](NC(=O)CN)c1ccc(-c2ccc(OCCOCCOCCOCCOCCN=[N+]=[N-])c3ccccc23)cc1 ; Cl ; Cc1ccnc(N(CCCCC(=O)O)C(=O)OC(C)(C)C)c1 ; "DMFrt1 h" ; "2TBTUDIPEA" ; COC(=O)C[C@H](NC(=O)CNC(=O)CCCCN(C(=O)OC(C)(C)C)c1cc(C)ccn1)c1ccc(-c2ccc(OCCOCCOCCOCCOCCN=[N+]=[N-])c3ccccc23)cc1]

To a solution of compounds 1 (0.10 g) and 2 (0.049 g) in DMF was added TBTU (0.058 g) and then DIPEA (0.079 mL) under ambient conditions. Reaction was stirred for 1 h until full conversion was observed by LC-MS. The reaction mixture was then quenched with NaHCO₃ (10 mL). The product was extracted with EtOAc (3×15 mL) and then washed with water (3×10 mL) and brine (10 mL). The combined organic phase was dried over Na₂SO₄, filtered, and concentrated. The residue was purified by CombiFlash® using silica gel as the stationary phase with a gradient of DCM to 20% MeOH in DCM (0-70%), in which product eluted at 23% B. The product was concentrated under vacuum to provide a clear colorless oil (0.088 g, yield 63.6%.)

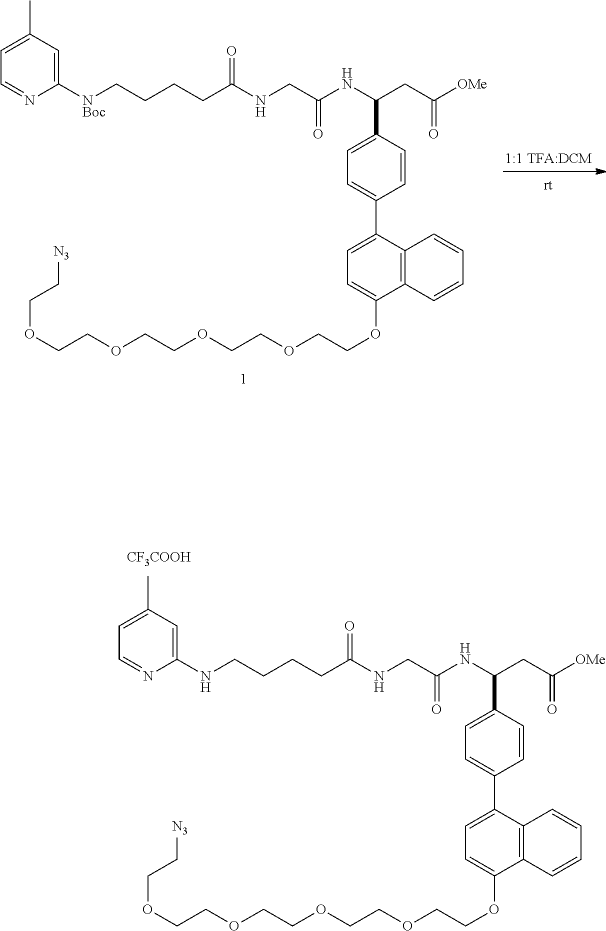 [SMILES: COC(=O)C[C@H](NC(=O)CNC(=O)CCCCN(C(=O)OC(C)(C)C)c1cc(C)ccn1)c1ccc(-c2ccc(OCCOCCOCCOCCOCCN=[N+]=[N-])c3ccccc23)cc1 ; "CF3COOH" ; COC(=O)C[C@H](NC(=O)CNC(=O)CCCCNc1cc(C)ccn1)c1ccc(-c2ccc(OCCOCCOCCOCCOCCN=[N+]=[N-])c3ccccc23)cc1]

To a solution of compound 1 (0.088 g) in DCM was added TFA (0.22 mL) at rt. The reaction was stirred under ambient conditions. Reaction was stirred for 5 h until full conversion was confirmed via LC-MS. The reaction mixture was azeotroped with PhMe and concentrated under vacuum. No isolation was necessary. Concentration provided a clear colorless oil (0.10 g, yield 113%.) LC-MS: calculated [M+H]+ 814.41 m/z, observed 814.63 m/z.

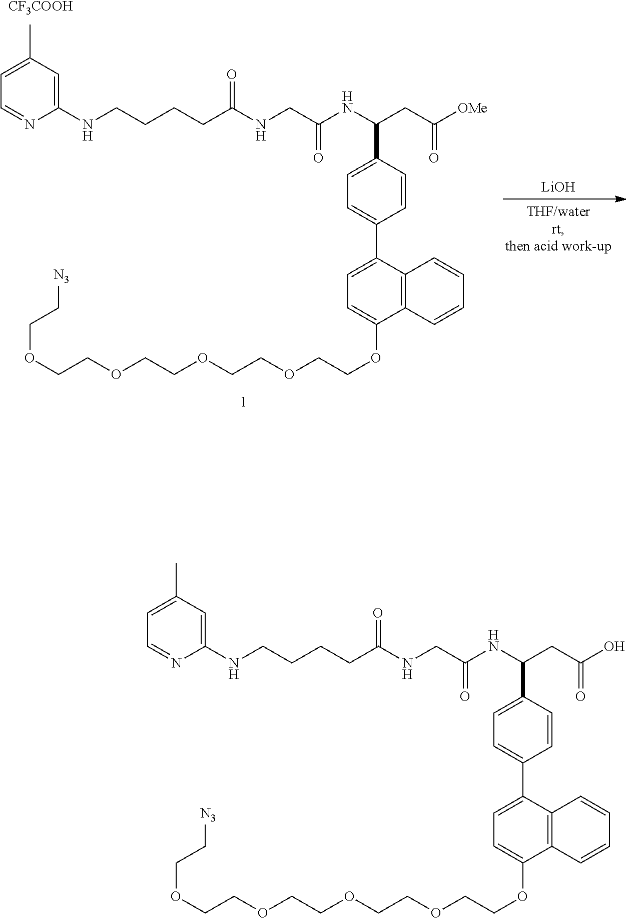 [SMILES: "CF3COOH" ; COC(=O)C[C@H](NC(=O)CNC(=O)CCCCNc1cc(C)ccn1)c1ccc(-c2ccc(OCCOCCOCCOCCOCCN=[N+]=[N-])c3ccccc23)cc1 ; "LiOH" ; Cc1ccnc(NCCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 1 (0.10 g) in 1:1 THF/water was added LiOH (0.0078 g) at rt under normal atmosphere. The reaction was stirred at rt until full conversion was observed by LC-MS. After 4 h, the reaction mixture was acidifed with 6 N HCl to a pH of ~3. The product was extracted with 20% CF₃CH₂OH/DCM (3×15 mL). The combined organic phase was dried over Na₂SO₄, filtered, and concentrated, providing a light yellow solid (0.104 g, yield 119%.) LC-MS: calculated [M+H]+800.39 m/z, observed 800.76 m/z.
F. Synthesis of Activated-Ester Skeletal Muscle Cell Receptor Peptide (αvβ6 Peptide 1/avb6-pep1) for conjugation to RNAi agents

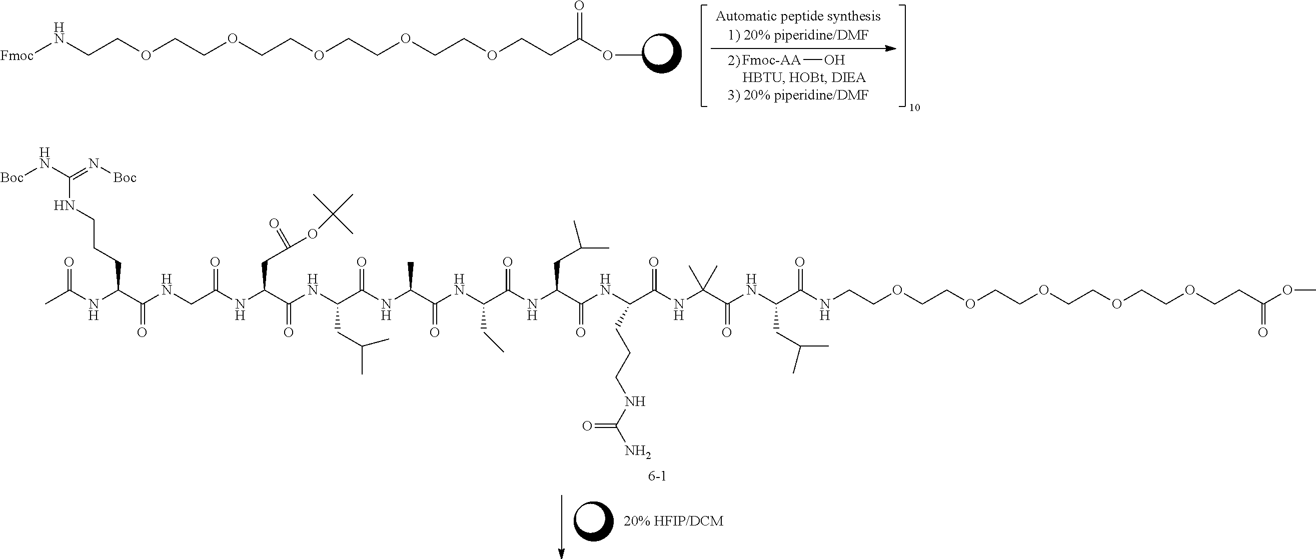

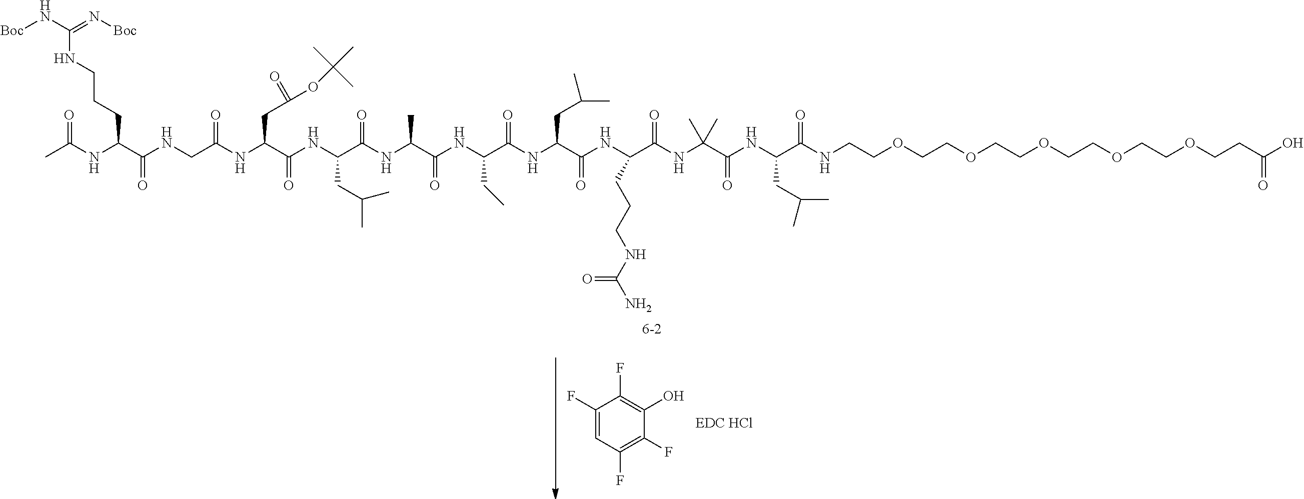 [SMILES: CC[C@H](NC(=O)[C@H](C)NC(=O)[C@H](CC(C)C)NC(=O)[C@H](CC(=O)OC(C)(C)C)NC(=O)CNC(=O)[C@H](CCCN/C(=N\C(=O)OC(C)(C)C)NC(=O)OC(C)(C)C)NC(C)=O)C(=O)N[C@@H](CC(C)C)C(=O)N[C@@H](CCCNC(N)=O)C(=O)NC(C)(C)C(=O)N[C@@H](CC(C)C)C(=O)NCCOCCOCCOCCOCCOCCC(=O)O ; Oc1c(F)c(F)cc(F)c1F]

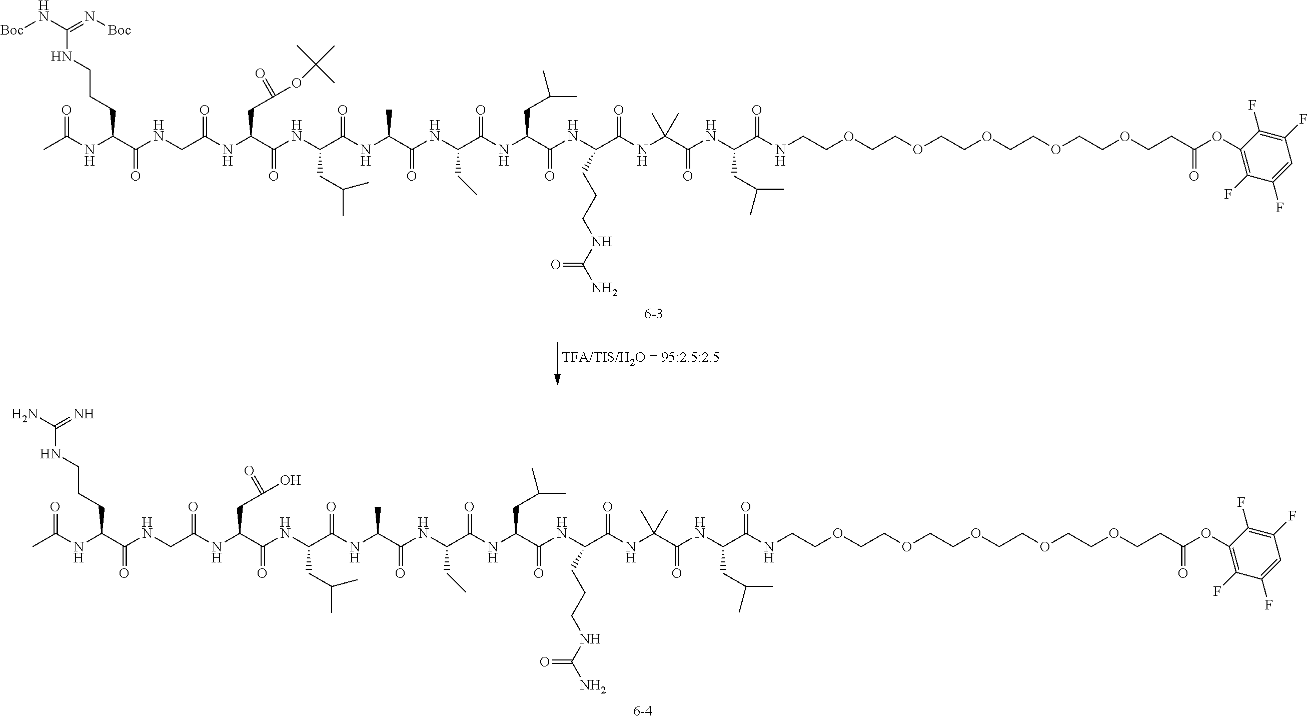 [SMILES: CC[C@H](NC(=O)[C@H](C)NC(=O)[C@H](CC(C)C)NC(=O)[C@H](CC(=O)OC(C)(C)C)NC(=O)CNC(=O)[C@H](CCCN/C(=N\C(=O)OC(C)(C)C)NC(=O)OC(C)(C)C)NC(C)=O)C(=O)N[C@@H](CC(C)C)C(=O)N[C@@H](CCCNC(N)=O)C(=O)NC(C)(C)C(=O)N[C@@H](CC(C)C)C(=O)NCCOCCOCCOCCOCCOCCC(=O)Oc1c(F)c(F)cc(F)c1F ; CC[C@H](NC(=O)[C@H](C)NC(=O)[C@H](CC(C)C)NC(=O)[C@H](CC(=O)O)NC(=O)CNC(=O)[C@H](CCCNC(=N)N)NC(C)=O)C(=O)N[C@@H](CC(C)C)C(=O)N[C@@H](CCCNC(N)=O)C(=O)NC(C)(C)C(=O)N[C@@H](CC(C)C)C(=O)NCCOCCOCCOCCOCCOCCC(=O)Oc1c(F)c(F)cc(F)c1F]

Peptide 1 was prepared by modification of Arg-Gly-Asp(tBu)-Leu-Ala-Abu-Leu-Cit-Aib-Leu-Peg₅-CO₂-2-Cl-Trt resin 1 that was obtained using general Fmoc peptide chemistry on CS Bio peptide synthesizer utilizing Fmoc-Peg₅-CO₂H preloaded 2-Cl-Trt resin on (0.79 mmol/g) at 4.1 mmol scale as described above. Following cleavage from resin the peptide 6-2 was converted into tetrafluorophenyl ester 6-3, and the crude product was used in the next step without purification.

Final deprotection was done by treatment of crude peptide 6-3 with deprotection cocktail TFA/TIS/H₂O=90:5:5 (80 mL) for 1.5 h. The reaction mixture was added dropwise to methyl tert-butyl ether (700 mL), and the resulting precipitate was collected by centrifugation. The pellets were washed with additional methyl tert-butyl ether (500 mL). The residue was purified by RP-HPLC (Phenomenex Gemini C18 250×50 mm, 10 micron, 60 mL/min, 30-45% ACN gradient in water containing 0.1% TFA, approx. 1 gram of crude per run), affording 4.25 g of pure peptide 6-4.
G. Conjugation of Targeting Ligands. Either prior to or after annealing, the 5′ or 3′ amine functionalized sense strand is conjugated to a targeting ligand, either directly or via the use of a linker such as an alkyne functionalized linker (for example, DBCO or Linkers 1-10 as shown in Table 6.1), which can then be used to facilitate the conjugation to the targeting ligand(s).

The following generally describes the conjugation of activated ester functionalized linkers, including DBCO and Linkers 1-10, to the single strand or annealed duplex: Amine-functionalized duplex was dissolved in 90% DMSO/10% H₂O, at ~50-70 mg/mL. 40 equivalents triethylamine was added, followed by 3 equivalents (L4). The reaction was monitored by RP-HPLC. Once complete, the conjugate was precipitated twice in a solvent system of 1× phosphate buffered saline/acetonitrile (1:14 ratio), and dried.

i. Conjugation of Targeting Ligands to Propargyl Linkers

Either prior to or after annealing, the 5′ or 3′ tridentate alkyne functionalized sense strand is conjugated to the αvβ6 Integrin Ligands. The following example describes the conjugation of αvβ6 integrin ligands to the annealed duplex: Stock solutions of 0.5M Tris(3-hydroxypropyltriazolylmethyl)amine (THPTA), 0.5M of Cu(II) sulfate pentahydrate (Cu(II)SO₄·5 H₂O) and 2M solution of sodium ascorbate were prepared in deionized water. A 75 mg/mL solution in DMSO of αvβ6 integrin ligand was made. In a 1.5 mL centrifuge tube containing tri-alkyne functionalized duplex (3 mg, 75 μL, 40 mg/mL in deionized water, ~15,000 g/mol), 25 μL of 1M Hepes pH 8.5 buffer is added. After vortexing, 35 μL of DMSO was added and the solution is vortexed. αvβ6 integrin ligand was added to the reaction (6 eq/duplex, 2 eq/alkyne, ~15 μL) and the solution is vortexed. Using pH paper, pH was checked and confirmed to be PH~8. In a separate 1.5 mL centrifuge tube, 50 μL of 0.5M THPTA was mixed with 10 μL of 0.5M Cu(II)SO₄·5 H₂O, vortexed, and incubated at room temp for 5 min. After 5 min, THPTA/Cu solution (7.2 μL, 6 eq 5:1 THPTA: Cu) was added to the reaction vial, and vortexed. Immediately afterwards, 2M ascorbate (5 μL, 50 eq per duplex, 16.7 per alkyne) was added to the reaction vial and vortexed. Once the reaction was complete (typically complete in 0.5-1 h), the reaction was immediately purified by non-denaturing anion exchange chromatography.

ii. Conjugation of Targeting Ligands to Amine-Functionalized Sense Strand

The following procedure may be used to conjugate an activated ester-functionalized targeting ligand such as αvβ6 peptide 1 to an amine functionalized RNAi agent comprising an amine, such as C6-NH2, NH2-C6, or (NH2-C6)s, as shown in Table 6.1, above.

An annealed, lyophilized RNAi agent was dissolved in DMSO and 10% water (v/v %) at 25 mg/mL. Then 50-100 equivalents TEA and three equivalents of activated ester targeting ligand were added to the mixture. The reaction was allowed to stir for 1-2 hours while monitored by RP-HPLC-MS (mobile phase A: 100 mM HFIP, 14 mM TEA; mobile phase B: Acetonitrile; column: XBridge C18). After the reaction was complete, 12 mL of acetonitrile was added followed by 0.4 mL of PBS and then the mixture was centrifuged. The solid pellet was collected and dissolved in 0.4 mL of 1×PBS and then 12 mL of acetonitrile was added. The resulting pellet was collected and dried on high vacuum for 1 hour.

### H. Synthesis of PK/PD Modulators

### PEG48+C22

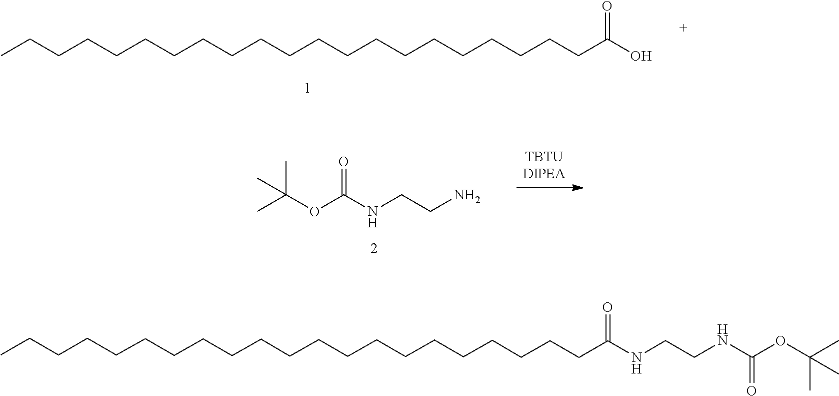 [SMILES: CCCCCCCCCCCCCCCCCCCCCC(=O)O ; CC(C)(C)OC(=O)NCCN ; CCCCCCCCCCCCCCCCCCCCCC(=O)NCCNC(=O)OC(C)(C)C]

To a solution of compound 1 (350 mg, 1.027 mmol, 1.0 equiv.), compound 2 (181 mg, 1.130 mmol, 1.1 equiv.) and diisopropylethylamine (0.537 mL, 3.082 mmol, 3.0 equiv.) in anhydrous DMF (3 mL) was added TBTU (396 mg, 1.233 mmol, 1.2 equiv.) at room temperature. The reaction was kept at room temperature for 2 hrs. The reaction was quenched with saturated NaHCO₃ aqueous solution (20 mL) and the aqueous was extracted with dicholoromethane (3×10 mL). The organic phase was combined, dried over anhydrous Na₂SO₄, and concentrated. The product was purified by CombiFlash® and was eluted with 4-5% methanol in dichloromethane. LC-MS: calculated [M+H]+ 483.44, found 483.67.

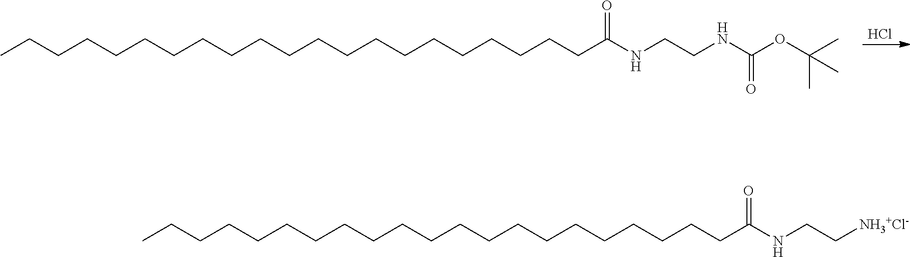

To a solution of compound 1 (290 mg, 0.600 mmol, 1.0 equiv.) in anhydrous 1,4-dioxane (1 mL) was added HCl solution in dioxane (0.751 mL, 3.003 mmol, 5.0 equiv.) at room temperature. The reaction was kept at room temperature for 3 hrs and the solvent was concentrated. The product was used directly without further purification. LC-MS: calculated [M+H]+ 383.39, found 383.57.

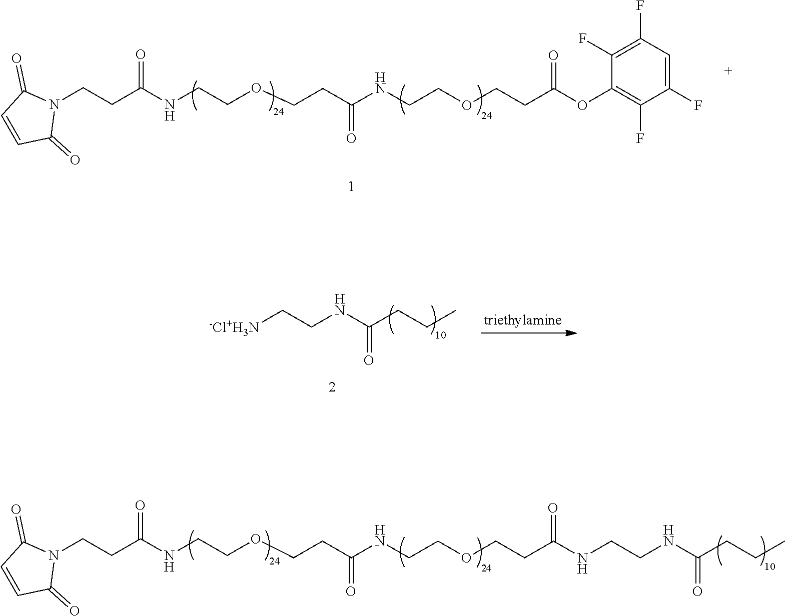

To a solution of compound 1 (83 mg, 0.0322 mmol, 1.0 equiv.) and compound 2 (13.5 mg, 0.322 mmol, 1.0 equiv.) in anhydrous DMF (2 mL) was added triethylamine (0.014 mL, 0.0967 mmol, 3.0 equiv.) at room temperature. The reaction was kept at room temperature for 3 hrs and the solvent was concentrated. The product was separated by CombiFlash and was eluted with 10-15% methanol in dicholoromethane. LC-MS: calculated [M+4H]+/4 698.18, found 698.49, calculated [M+3H]+/3 930.58, found 930.61.

### Synthesis of LP29-p

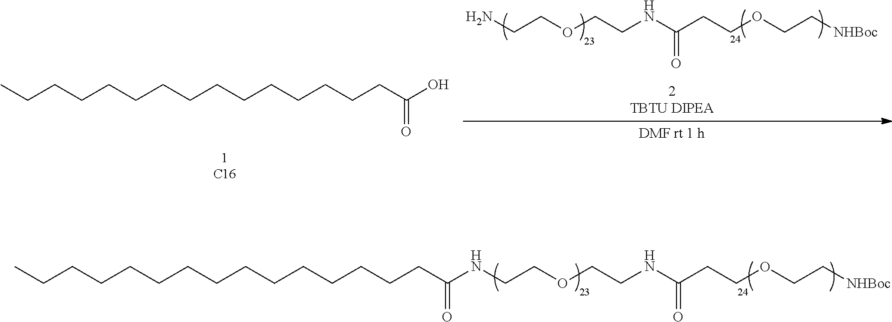

To a solution of compounds 1 (40 mg) and 2 (334 mg) in DMF was added TBTU (50.1 mg) and then DIPEA (0.082 mL) under ambient conditions. The reaction was stirred until full conversion was observed by LC-MS. The reaction mixture was then directly concentrated for isolation. The residue was purified by CombiFlash using silica gel as the stationary phase with a gradient of DCM to 20% MeOH in DCM (0-80%) over 20-30 min., in which product eluted at 71% B. The product was concentrated under vacuum to provide a white oily residue. LC-MS: calculated [M+H]+ 2539.62 m/z, observed 1288.21 (+2/2, +H₂O) m/z.

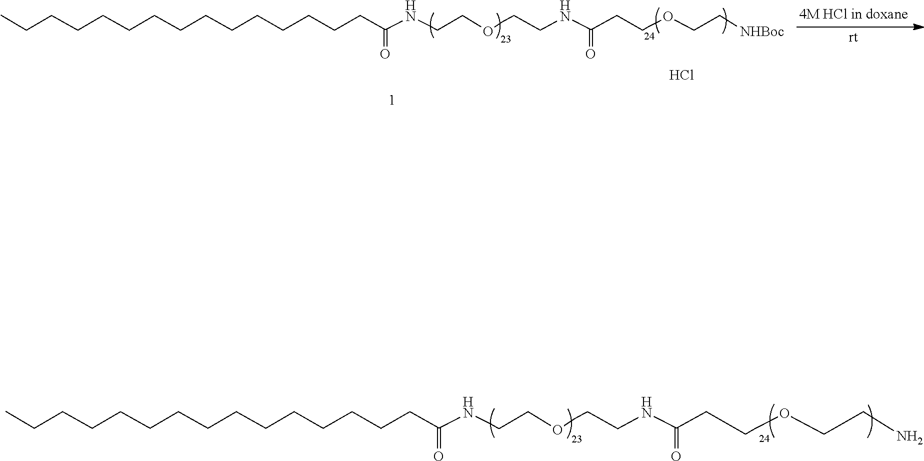

To compound 1 (147 mg) was added 4 M HCl/dioxane (21.2 mg) at room temperature. The reaction was stirred under ambient conditions. The reaction was stirred overnight until full conversion was confirmed via LC-MS. The reaction mixture was azeotroped with PhMe and concentrated under vacuum overnight to provide an oil. LC-MS: calculated [M+H]+ 2439.57 m/z, observed 611.16 (+4/4) m/z.

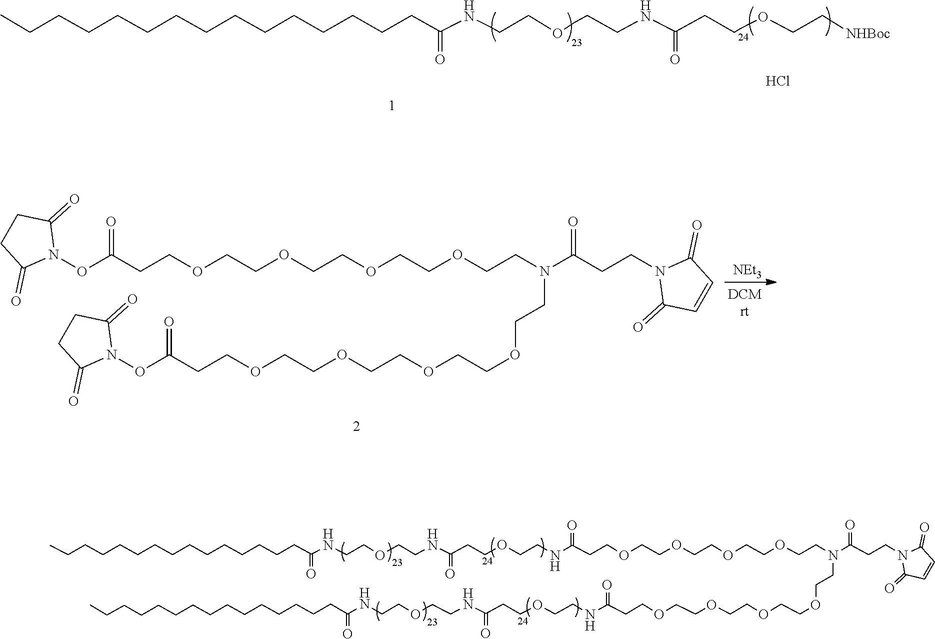

A solution of compound 1 (143 mg) and NEt₃ (0.024 mL) in anh. DCM was prepared and stirred under sparging nitrogen atmosphere. Compound 2 (23.4 mg) was then added to the reaction mixture. The reaction mixture was stirred at room temperature until full conversion was observed by LC-MS.

The reaction mixture was directly concentrated for isolation. The residue was purified by CombiFlash using silica gel as the stationary phase and was eluted with a gradient of DCM to 20% MeOH in DCM (0-100% B). Product eluted at 54% B. LC-MS: calculated [M+H]+ 5506.42 m/z, observed 1854.41 (+3/3, +H₂O) m/z.

### Synthesis of LP38-p

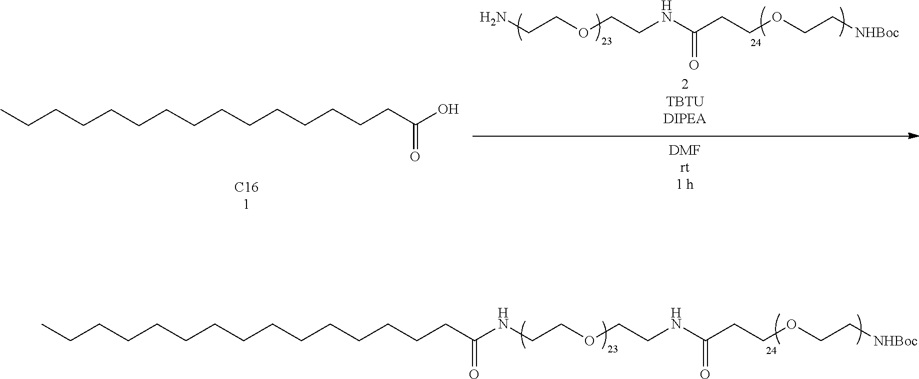

To a solution of compounds 1 (35 mg) and 2 (299 mg) in DMF was added TBTU (43.8 mg) and then DIPEA (0.071 mL) under ambient conditions. Reaction was stirred until full conversion was observed by LC-MS. The reaction mixture was then directly concentrated for isolation. The residue was purified by CombiFlash using silica gel as the stationary phase with a gradient of DCM to 20% MeOH in DCM (0-100%) over 20-30 min., in which product eluted at 56% B. The product was concentrated under vacuum to provide a white oily residue. LC-MS: calculated [M+H]+ 2539.62 m/z, observed 1288.07 (+2/2, +H2O) m/z.

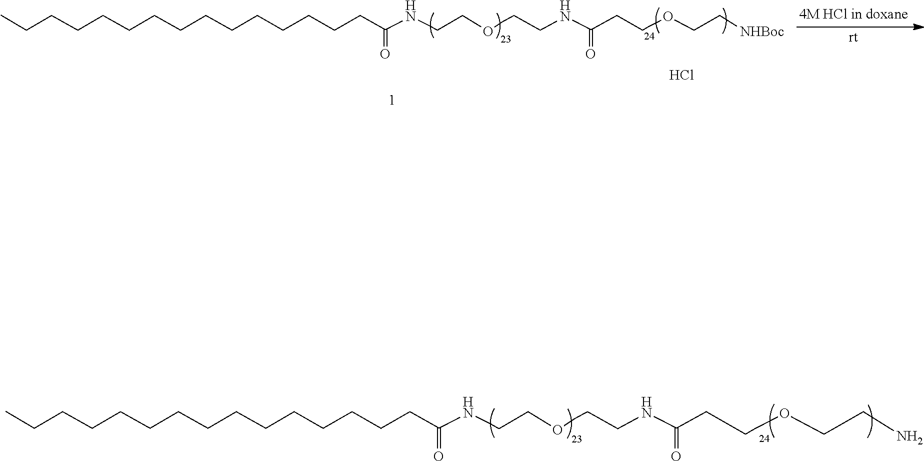

To compound 1 (186 mg) was added 4 M HCl/dioxane (26.7 mg) at room temperature. The reaction was stirred under ambient conditions. The reaction was stirred overnight until full conversion was confirmed via LC-MS. The reaction mixture was azeotroped with PhMe and concentrated under vacuum overnight to provide an oil. LC-MS: calculated [M+H]+ 2439.57 m/z, observed 1220.97 (+2/2) m/z.

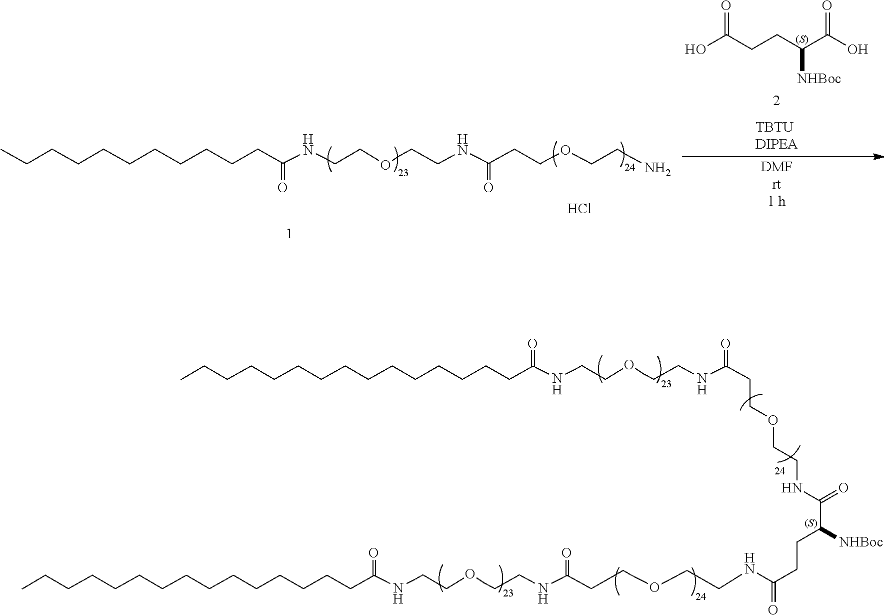

To a solution of compound 1 (181 mg), TBTU (24 mg), and DIEA (0.033 mL) in DMF was added 2 (8.7 mg) under ambient conditions. Reaction was stirred until full conversion was observed by LC-MS. The reaction mixture was then directly concentrated for isolation. The residue was purified by CombiFlash using silica gel as the stationary phase with a gradient of DCM to 20% MeOH in DCM (0-100%) over 20-30 min., in which product eluted at 65% B. The product was concentrated under vacuum to provide a white oily residue. LC-MS: calculated [M+H]+ 5089.22 m/z, observed 1036.24 (+5/5, +H₂O) m/z.

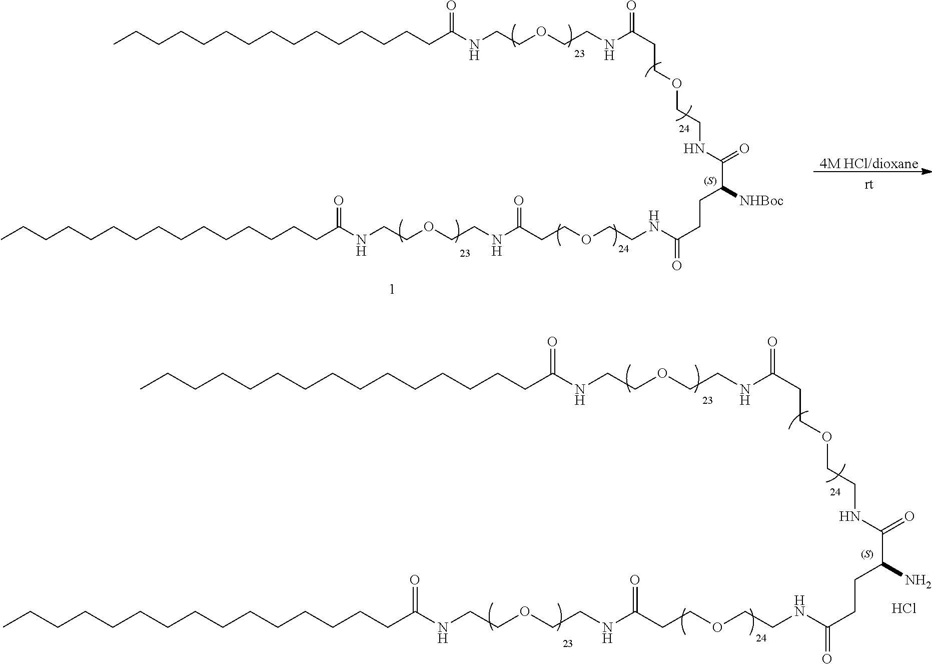

To compound 1 (130 mg) was added 4 M HCl/dioxane (9.3 mg) at rt. The reaction was stirred under ambient conditions. Reaction was stirred overnight until full conversion was confirmed via LC-MS. The reaction mixture was azeotroped with PhMe and concentrated under vacuum overnight to provide an oil. LC-MS: calculated [M+H]+ 4989.17 m/z, observed 1248.58 (+4/4) m/z.

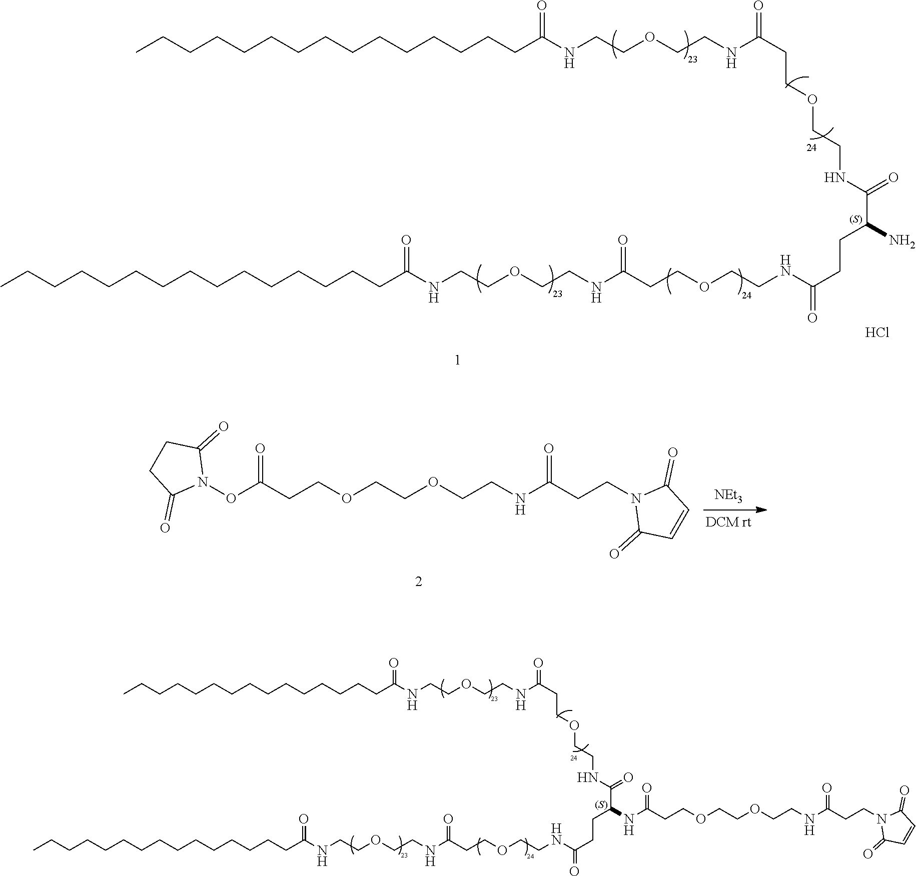

A solution of compound 1 (128 mg) and NEt₃ (0.018 mL) in anhydrous DCM under sparging N2 (g) was prepared at room temperature. Compound 2 (10.3 mg) was then added slowly. The reaction mixture was allowed to stir until full conversion was observed by LC-MS. The reaction mixture was allowed to stir until full conversion was observed by LC-MS. The reaction mixture was then directly concentrated. The residue was purified by CombiFlash using silica gel as the stationary phase with a gradient of DCM to 20% MeOH/DCM (0-100%) over 30 min., in which product eluted at 100% B. Product was concentrated to provide a white solid. LC-MS: calculated [M+H]+ 5299.28 m/z, observed 1786.62 (+3/3, +H₂O) m/z.

### Synthesis of LP238-p

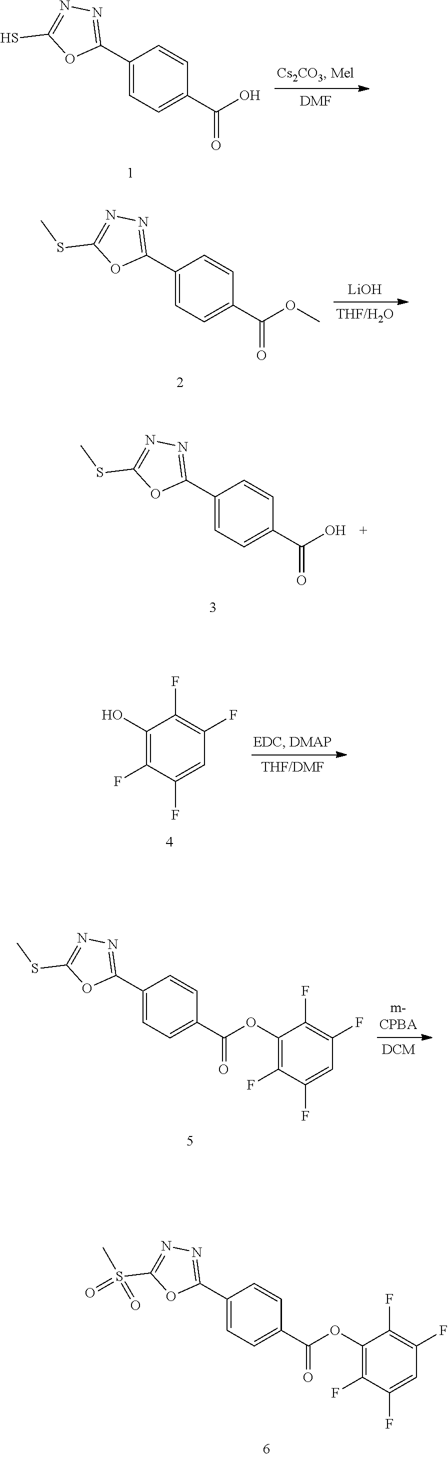

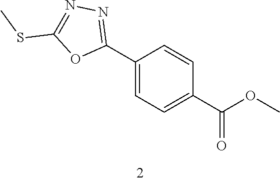

To a suspension of compound 1 (5.00 g, 22.50 mmol) and Cs₂CO₃ (25.66 g, 78.75 mmol) in anhydrous DMF (80 mL) was added methyl iodide (4.20 mL, 67.50 mmol) at room temperature. The reaction mixture was stirred at room temperature for 48 hours. The reaction was quenched with water (200 mL) and the mixture was extracted with EtOAc (3×100 mL). The organic phase was combined and washed with water and brine. The organic layer was dried over anhydrous Na₂SO₄ and concentrated. Compound 2 was obtained as a light yellow solid, 5.41 g, 96%. Compound 2 was used directly without further purification. LC-MS: [M+H] calculated 251.05, found 251.18.

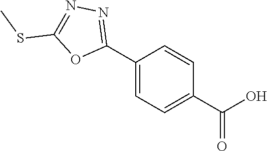 [SMILES: CSc1nnc(-c2ccc(C(=O)O)cc2)o1]

To a solution of compound 2 (5.41 g, 21.62 mmol) in THF/H₂O (50 mL/50 mL) was added LiOH (2.59 g, 108.08 mmol) at room temperature. The reaction mixture was stirred at room temperature for 1 hour. After removing THE under vacuum, the pH was adjusted to ~2 by [C] HCl. Then EtOAc (3×60 mL) was used to extract. The organic layers were combined, washed with brine, then dried over anhydrous Na₂SO₄, and concentrated. Compound 3 was obtained as an off-white solid, 5 g, 98%. Compound 3 was used directly without further purification. LC-MS: calculated [M+H] 237.03, found 237.26.

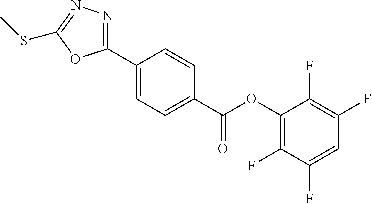 [SMILES: CSc1nnc(-c2ccc(C(=O)Oc3c(F)c(F)cc(F)c3F)cc2)o1]

To a solution of compound 3 (5.81 g, 24.60 mmol) in THF/DMF (80 mL/20 mL) was added EDC (7.07 g, 36.90 mmol), DMAP (0.30 g, 2.46 mmol) and compound 4 (6.13 g, 36.90 mmol) at room temperature. The reaction mixture was stirred at room temperature overnight. After removing solvent under vacuum, the residue was loaded on a 120 g column and compound 5 was eluted with 0-50% EtOAc in hexanes. Compound 5 was obtained as a white solid, 9.36 g, 99%. LC-MS: calculated [M+H] 385.03, found 385.46.

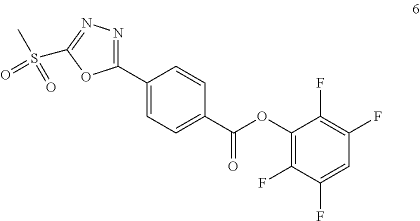 [SMILES: CS(=O)(=O)c1nnc(-c2ccc(C(=O)Oc3c(F)c(F)cc(F)c3F)cc2)o1]

To a solution of compound 5 (2.29 g, 5.96 mmol) in DCM (110 mL) was added 70% m-CPBA (5.14 g, 27.79 mmol) at 0° C. The reaction mixture was stirred at room temperature for 6 hours. Another 1.8 g m-CPBA was added at room temperature. The reaction mixture was stirred at room temperature overnight. After filtration, the solvent was removed under vacuum. The residue was recrystallized from DCM/EtOAc (50 mL/50 mL) twice. Compound 6 was obtained as white needle crystal, 1.93 g, 78%. LC-MS: calculated [M+H] 417, found 417.

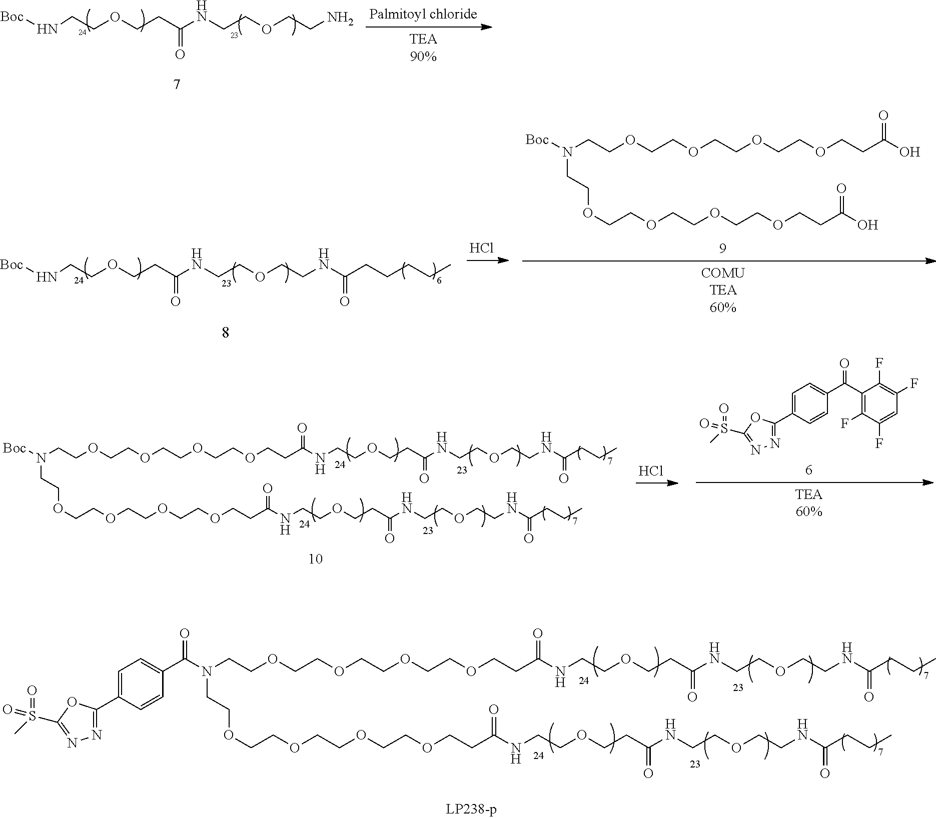

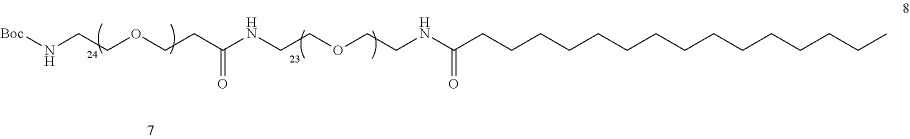

To a solution of compound 7 (10.00 g, 4.34 mmol) in DCM (100 mL) was added palmitoyl chloride (1.31 g, 4.78 mmol) and TEA at 0° C. The reaction mixture was stirred at room temperature overnight and then the solvent was removed under vacuum. The residue was purified by silica gel chromatography using 0-20% MeOH in DCM. Compound 8 was obtained as a white solid, 10.0 g, 90%.

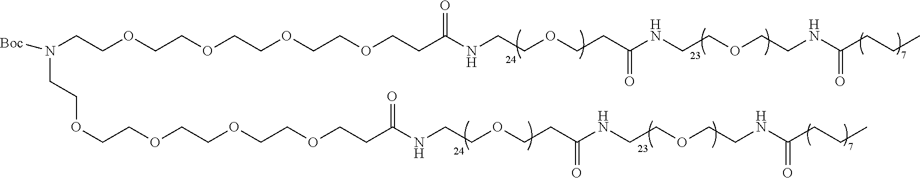

Compound 8 (9.56 g, 3.76 mmol) was dissolved in 25 mL 4N HCl/dioxane and stirred at room temperature for 1 hour. All solvent was removed and the residue was dried under vacuum for 2 hours. The residue was re-dissolved in 150 mL DCM and TEA was added, followed by compound 9 (1.10 g, 1.79 mmol), and COMU (1.69 g, 3.94 mmol). The reaction mixture was stirred at room temperature overnight. After a standard workup (1N HCl, Sat. bicarb, brine wash), DCM was removed. Compound 10 was purified by a 120 g column using 0-20% MeOH in DCM to obtain 5.90 g, 60%.

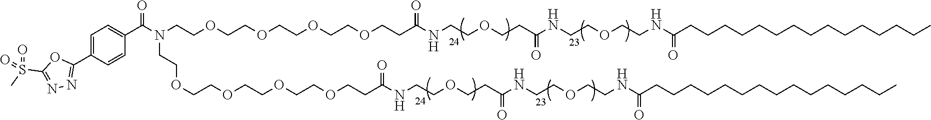

Compound 10 (4.50 g, 0.82 mmol) was dissolved in 20 mL 4N HCl/dioxane and stirred at room temperature for 1 hour. All solvent was removed and the residue was dried under vacuum for 2 hours. The residue was re-dissolved in 100 mL DCM and TEA was added, followed by compound 6 (0.69 g, 1.65 mmol). The reaction mixture was stirred at room temperature overnight. TEA was removed by a 1H HCl wash and the organic layer was concentrated. Crude LP238-p was purified by silica gel chromatography using 0-20% MeOH in DCM. 2.80 g (60%) of LP238-p was obtained as a light yellow solid.

### I. Conjugation of PK/PD Modulators to RNAi Agents

Either prior to or after annealing and prior to or after conjugation of one or more targeting ligands, one or more lipid PK/PD modulator precursors can be linked to the RNAi agents disclosed herein. The following describes the general conjugation process used to link lipid PK/PD modulator precursors to the constructs set forth in the Examples depicted herein.

### A. Conjugation of a Maleimide-Containing Lipid PK/PD Modulator Precursor

The following describes the general process used to link a maleimide-containing lipid PK/PD modulator precursor to the (C6-SS-C6) or (6-SS-6) functionalized sense strand of an RNAi agent by undertaking a dithiothreitol reduction of disulfide followed by a thiol-Michael Addition of the respective maleimide-containing lipid PK/PD modulator precursor: In a vial, functionalized sense strand was dissolved at 50 mg/mL in sterilized water. Then 20 equivalents of each of 0.1M Hepes pH 8.5 buffer and dithiothreitol were added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the maleimide-containing lipid PK/PD modulator precursor was added at 1.5 equivalents. The mixture was allowed to react for 30 minutes. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

### B. Conjugation of a Sulfone-Containing Lipid PK/PD Modulator Precursor

In a vial, functionalized sense strand was dissolved at 50 mg/mL in sterilized water. Then 20 equivalents of each of 0.1M Hepes pH 8.5 buffer and dithiothreitol are added. The mixture was allowed to react for one hour, then the conjugate was precipitated in acetonitrile and PBS, and the solids were centrifuged into a pellet.

The pellet was brought up in a 70/30 mixture of DMSO/water at a solids concentration of 30 mg/mL. Then, the sulfone-containing lipid PK/PD modulator precursor was added at 1.5 equivalents. The vial was purged with N₂, and heated to 40° C. while stirring. The mixture was allowed to react for one hour. The product was purified on an AEX-HPLC (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile; solid phase TSKgel-30; 1.5 cm×10 cm.) The solvent was removed by rotary evaporator, and desalted with a 3K spin column using 2×10 mL exchanges with sterilized water. The solid product was dried using lyophilization and stored for later use.

### C. Conjugation of an Azide-Containing Lipid PK/PD Modulator Precursor

One molar equivalent of TG-TBTA resin loaded with Cu(I) was weighed into a glass vial. The vial was purged with N₂ for 15 minutes. Then, functionalized sense strand was dissolved in a separate vial in sterilized water at a concentration of 100 mg/mL. Then two equivalents of the azide-containing lipid PK/PD modulator precursor (50 mg/mL in DMF) is added to the vial. Then TEA, DMF and water are added until the final reaction conditions are 33 mM TEA, 60% DMF, and 20 mg/mL of the conjugated product. The solution was then transferred to the vial with resin via a syringe. The N2 purge was removed and the vial was sealed and moved to a stir plate at 40° C. The mixture was allowed to react for 16 hours. The resin was filtered off using a 0.45 μm filter.

The product was purified using AEX purification (mobile phase A: 25 mM TRIS pH=7.2, 1 mM EDTA, 50% acetonitrile; mobile phase B: 25 mM TRIS pH=7.2, 1 mM EDTA, 500 mM NaBr, 50% acetonitrile solid phase TSKgel-30; 1.5 cm×10 cm.) The acetonitrile was removed using a rotary evaporator, and desalted with a 3K spin column using 2×10 mL exchanges with sterilized water. The solid product was dried using lyophilization and stored for later use.

### D. Conjugation of an Alkyne-Containing Lipid PK/PD Modulator Precursor

The following describes the general process used to link an activated alkyne-containing lipid PK/PD modulator precursor to the (C6-SS-C6) or (6-SS-6) functionalized sense strand of an RNAi agent by undertaking a dithiothreitol reduction of disulfide followed by addition to an alkyne-containing PK/PD modulator precursor: In a vial, 10 mg of siRNA comprising the (C6-SS-C6) or (6-SS-6) functionalized sense strand was dissolved at 50 mg/mL in sterilized water. Then 20 equivalents of each of 0.1M Hepes pH 8.5 buffer and dithiothreitol (1M in sterilized water) were added. The mixture was allowed to react for one hour, then purified on XBridge BEH C4 Column using a mobile phase A of 100 mM HFIP, 14 mM, and TEA, and a mobile phase B of Acetonitrile using the following formula, wherein % B indicates the amount of mobile phase B while the remainder is mobile phase A.

| Time | % B |
|---|---|
| 0 | 3 |
| 8 | 70 |
| 10 | 90 |
| 11 | 90 |
| 11.1 | 3 |
| 13 | 3 |

The product was precipitated once by adding 12 mL of acetonitrile and 0.4 mL 1×PBS, and the resulting solid was centrifuged into a pellet. The pellet was re-dissolved in 0.4 mL 1×PBS and 12 mL of acetonitrile. The pellet was dried on high vacuum for one hour.

The pellet was brought up in a vial a 70/30 mixture of DMSO/water at a solids concentration of 30 mg/mL. Then, the alkyne-containing lipid PK/PD modulator precursor was added at 2 equivalents relative to siRNA. Then 10 equivalents of TEA was added. The vial was purged using N2, and the reaction mixture was heated to 40° C. while stirring. The mixture was allowed to react for one hour. The product was purified using anion-exchange HPLC using a TSKgel-30 packed column, 1.5 cm×10 cm, using a mobile phase A of 25 mM TRIS pH-7.2, 1 mM EDTA, 50% Acetonitrile, and a mobile phase B of 25 mM TRIS pH=7.2, 1 mM EDTA, 500 mM NaBr, 50% Acetonitrile using the following formula, wherein % B indicates the amount of mobile phase B while the remainder is mobile phase A.

| Time | % B |
|---|---|
| 4 | 10 |
| 7 | 80 |
| 10.5 | 80 |
| 11 | 10 |
| 14 | 10 |

The fractions containing the product were collected, and acetonitrile was removed using a rotary evaporator. The product was desalted with a 3K spin column, using 2×10 mL exchanges with sterilized water. The product was then dried using lyophilization and stored for later use.

### J. Synthesis of Linker 4

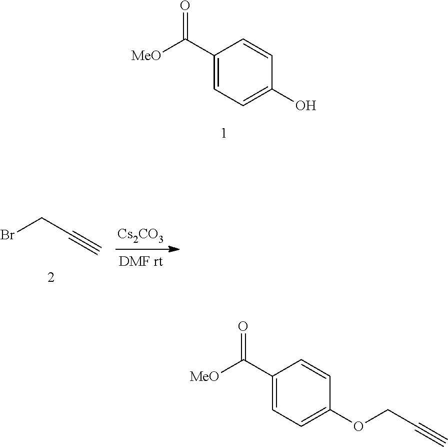

To a solution of compound 1 (3.00 g) in DMF was added Cs₂CO₃ (7.71 g) at rt. Compound 2 (1.85 mL) was then added slowly. Reaction was stirred overnight under N₂ (g). Approx. full conversion to desired product by LC-MS was then confirmed. The reaction mixture was quenched with NaHCO₃ (10 mL). The product was extracted with EtOAc (5×10 mL) and then washed with water (3×8 mL) and brine (8 mL). The combined organic phase was dried over Na₂SO₄, filtered, and concentrated. The residue was purified by CombiFlash using silica gel as the stationary phase with a gradient of hex to EtOAc (0-30%), in which product eluted at 14% B. The product was concentrated under vacuum to provide a white solid. LC-MS: calculated [M+H]+ 191.06 m/z, observed 191.23 m/z.

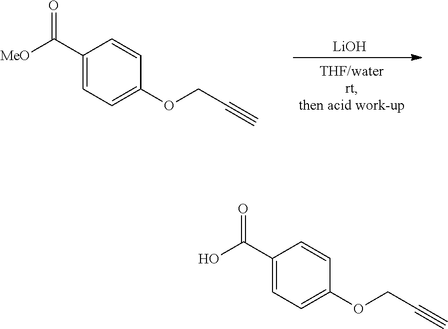

To a solution of compound 1 (2.87 g) in 1:1 THF/water was added LiOH (1.08 g) at rt under normal atmosphere. The reaction was stirred until full conversion was observed by LC-MS. Residual starting material was extracted via EtOAc, and then aqueous phase was acidifed with 6 N HCl to a pH of ~3. Product crashed out as white solid and was filtered over vacuum and washed with water. Due to its wet/sticky nature, solvent was required to transfer the solid to a round bottom flask; material was transferred via MeOH and DCM. Due to poor solvation in either and the combination, material was not able to be dried over Na₂SO₄ and was correspondingly merely concentrated under vacuum to provide a white, fluffy crystalline solid. No isolation was necessary. LC-MS: calculated [M+H]+ 177.05 m/z, observed 177.19 m/z.

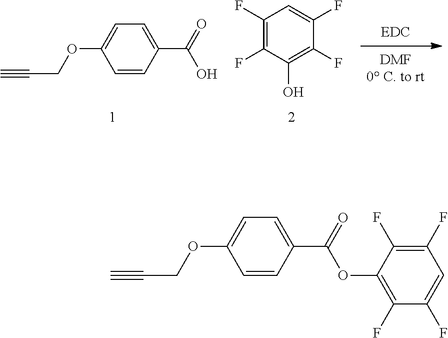

To a solution of compounds 1 (1.00 g) and 2 (1.04 g) in DMF (10.0 mL) under N₂ (g) was added EDC (1.20 g) at rt. The reaction mixture was allowed to stir until full conversion was observed by LC-MS. Due to an inability to successfully observe product after overnight stirring, reaction mixture was quenched with NaHCO₃, in which crash-out followed. Precipitate was confirmed to contain starting materials via LC-MS and was filtered over vacuum, attempted to be resuspended in MeOH/DCM, and then concentrated under vacuum. Mixture was then resolvated in DMF, dried over Na₂SO₄, and filtered over vacuum, rinsing with DMF. EDC was readded to filtrate (reaction mixture), and mixture was allowed to stir overnight at rt. The reaction mixture was directly concentrated and azeotroped with MeOH and PhMe for isolation. The residue was purified by CombiFlash® using silica gel as the stationary phase and was eluted with a gradient of DCM to 20% of MeOH/DCM (0-15% B). Product eluted at 0% B to provide a white solid. LC-MS: calculated [M+H]+ 325.04 m/z, observed 325.35 m/z.

### Example 2. hDMPK-GLuc AAV Mouse Model

To evaluate certain DMPK RNAi agents, a DMPK-GLuc (Gaussia Luciferase) AAV (Adeno-associated virus) mouse model was used. Six- to eight-week-old male C57BL/6 mice were transduced with DMPK-GLuc AAV serotype 8, administered at least 14 days prior to administration of a DMPK RNAi agent or control. Two types of DMPK-GLuc AAV were used. The genome of the first DMPK-GLuc AAV contains the 548-1918 region of the human DMPK cDNA sequence (GenBank NM_001081563.2) inserted into the 3′ UTR of the GLuc reporter gene sequence. The genome of the second DMPK-GLuc AAV contains the 1891-3243 region of the human DMPK cDNA sequence (GenBank NM_001081563.2) inserted into the 3′ UTR of the GLuc reporter gene sequence. 4.8E12 to 5.0E12 GC/kg of the respective virus in PBS in a total volume of 10 mL/kg animal's body weight was injected into mice via the tail vein to create hDMPK-GLuc AAV model mice. Inhibition of expression of DMPK by a DMPK RNAi agent results in concomitant inhibition of GLuc expression, which is measured. Prior to administration of a treatment (between day-7 and day 1 pre-dose), GLuc expression levels in serum were measured by the Pierce™ Gaussia Luciferase Glow Assay Kit (Thermo Fisher Scientific), and the mice were grouped according to average GLuc levels.

Mice were anesthetized with 2-3% isoflurane and blood samples were collected from the submandibular area into serum separation tubes (Sarstedt AG & Co., Nümbrecht, Germany). Blood was allowed to coagulate at ambient temperature for 20 min. The tubes were centrifuged at 8,000×g for 3 min to separate the serum and stored at 4° C. Serum was collected and measured by the Pierce™ Gaussia Luciferase Glow Assay Kit according to the manufacturer's instructions. Serum GLuc levels for each animal can be normalized to the control group of mice injected with vehicle control in order to account for the non-treatment related shift in DMPK expression with this model. To do so, first, the GLuc level for each animal at a time point was divided by the pre-treatment level of expression in that animal (Day 1) in order to determine the ratio of expression “normalized to pre-treatment”. Expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

To evaluate the activity of DMPK RNAi agents in a DMPK-AAV model as described in the Examples below, certain DMPK RNAi agents were conjugated to an N-acetyl-galactosamine containing targeting ligand having the chemical structure referred to as NAG37 (see Table 6.3). NAG37 is known to have high affinity to bind to asialoglycoprotein receptors that are abundantly expressed on liver cells, including hepatocytes (see, e.g., International Patent Application Publication No. WO2018044350A1). The use of NAG37-conjugated DMPK RNAi agents was to evaluate the expression of AAV-DMPK in the liver.

### Example 3. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 2.0 mg/kg (mpk), 4.0 mg/kg (mpk) of a DMPK RNAi agent or saline without a DMPK RNAi agent to be used as a control, according to the following Table 7.

TABLE 7
DMPK RNAi Agent and Dosing for Example 2

| Group ID | Dosing Regimen |
|---|---|
| Group 1 Saline | Single SQ injection on day 1 |
| Group 2 2.0 mg/kg AD09699 | Single SQ injection on day 1 |
| Group 3 4.0 mg/kg AD09699 | Single SQ injection on day 1 |
| Group 4 2.0 mg/kg AD09915 | Single SQ injection on day 1 |
| Group 5 2.0 mg/kg AD09920 | Single SQ injection on day 1 |
| Group 6 2.0 mg/kg AD09697 | Single SQ injection on day 1 |
| Group 7 2.0 mg/kg AD09698 | Single SQ injection on day 1 |
| Group 8 2.0 mg/kg AD09700 | Single SQ injection on day 1 |
| Group 9 2.0 mg/kg AD09702 | Single SQ injection on day 1 |
| Group 10 2.0 mg/kg AD09704 | Single SQ injection on day 1 |
| Group 11 2.0 mg/kg AD09705 | Single SQ injection on day 1 |
| Group 12 2.0 mg/kg AD08910 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 8.

TABLE 8
Average DMPK Normalized to Pre-Treatment and Control in
AAV-hDMPK Mice from Example 3

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.103 |
| Group 2 2.0 mg/kg AD09699 | 1.000 | 0.000 | 0.722 | 0.115 |
| Group 3 4.0 mg/kg AD09699 | 1.000 | 0.000 | 0.655 | 0.014 |
| Group 4 2.0 mg/kg AD09915 | 1.000 | 0.000 | 0.924 | 0.128 |
| Group 5 2.0 mg/kg AD09920 | 1.000 | 0.000 | 1.025 | 0.247 |
| Group 6 2.0 mg/kg AD09697 | 1.000 | 0.000 | 0.749 | 0.247 |
| Group 7 2.0 mg/kg AD09698 | 1.000 | 0.000 | 0.944 | 0.067 |
| Group 8 2.0 mg/kg AD09700 | 1.000 | 0.000 | 0.731 | 0.069 |
| Group 9 2.0 mg/kg AD09702 | 1.000 | 0.000 | 0.690 | 0.096 |
| Group 10 2.0 mg/kg AD09704 | 1.000 | 0.000 | 0.776 | 0.116 |
| Group 11 2.0 mg/kg AD09705 | 1.000 | 0.000 | 0.748 | 0.117 |
| Group 12 2.0 mg/kg AD08910 | 1.000 | 0.000 | 1.141 | 0.310 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.174 | 1.000 | 0.162 |
| Group 2 2.0 mg/kg AD09699 | 0.618 | 0.099 | 0.743 | 0.089 |
| Group 3 4.0 mg/kg AD09699 | 0.526 | 0.091 | 0.589 | 0.095 |
| Group 4 2.0 mg/kg AD09915 | 0.778 | 0.091 | 0.874 | 0.193 |
| Group 5 2.0 mg/kg AD09920 | 0.777 | 0.216 | 0.833 | 0.046 |
| Group 6 2.0 mg/kg AD09697 | 0.650 | 0.197 | 0.679 | 0.174 |
| Group 7 2.0 mg/kg AD09698 | 0.689 | 0.051 | 0.876 | 0.167 |
| Group 8 2.0 mg/kg AD09700 | 0.624 | 0.089 | 0.852 | 0.118 |
| Group 9 2.0 mg/kg AD09702 | 0.511 | 0.038 | 0.616 | 0.113 |
| Group 10 2.0 mg/kg AD09704 | 0.746 | 0.113 | 0.789 | 0.049 |
| Group 11 2.0 mg/kg AD09705 | 0.614 | 0.085 | 0.692 | 0.193 |
| Group 12 2.0 mg/kg AD08910 | 0.806 | 0.126 | 0.896 | 0.223 |

The DMPK RNAi agents in Groups 2-4, and 5-11 showed reduction in DMPK-AAV as compared to the saline control (Group 1) at Day 8, reduction for all groups at Day 15, and reduction for all groups at Day 22. Group 9 showed particularly robust knockdown at day 22 at 2.0 mg/kg. Furthermore, dose response of DMPK RNAi agent AD09699 was observed.

### Example 4. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 1.0 mg/kg (mpk), 2.0 mg/kg (mpk), 4.0 mg/kg (mpk) of a DMPK RNAi agent, or saline without an DMPK RNAi agent to be used as a control, according to the following Table 9.

TABLE 9
DMPK RNAi Agent and Dosing for Example 4

| Group ID | Dosing Regimen |
|---|---|
| Group 1 Saline | Single SQ injection on day 1 |
| Group 2 1.0 mg/kg AD09699 | Single SQ injection on day 1 |
| Group 3 2.0 mg/kg AD09699 | Single SQ injection on day 1 |
| Group 4 4.0 mg/kg AD09699 | Single SQ injection on day 1 |
| Group 5 2.0 mg/kg AD10261 | Single SQ injection on day 1 |
| Group 6 2.0 mg/kg AD10262 | Single SQ injection on day 1 |
| Group 7 2.0 mg/kg AD10263 | Single SQ injection on day 1 |
| Group 8 2.0 mg/kg AD10264 | Single SQ injection on day 1 |
| Group 9 2.0 mg/kg AD10265 | Single SQ injection on day 1 |
| Group 10 1.0 mg/kg AD10302 | Single SQ injection on day 1 |
| Group 11 2.0 mg/kg AD10302 | Single SQ injection on day 1 |
| Group 12 4.0 mg/kg AD10302 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 10.

TABLE 10
Average DMPK Normalized to Pre-Treatment and Control
in AAV-hDMPK Mice from Example 4

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.272 |
| Group 2 1.0 mg/kg AD09699 | 1.000 | 0.000 | 0.979 | 0.082 |
| Group 3 2.0 mg/kg AD09699 | 1.000 | 0.000 | 0.975 | 0.164 |
| Group 4 4.0 mg/kg AD09699 | 1.000 | 0.000 | 0.957 | 0.133 |
| Group 5 2.0 mg/kg AD10261 | 1.000 | 0.000 | 1.087 | 0.212 |
| Group 6 2.0 mg/kg AD10262 | 1.000 | 0.000 | 1.191 | 0.078 |
| Group 7 2.0 mg/kg AD10263 | 1.000 | 0.000 | 1.045 | 0.213 |
| Group 8 2.0 mg/kg AD10264 | 1.000 | 0.000 | 1.186 | 0.172 |
| Group 9 2.0 mg/kg AD10265 | 1.000 | 0.000 | 0.927 | 0.108 |
| Group 10 1.0 mg/kg AD10302 | 1.000 | 0.000 | 0.974 | 0.176 |
| Group 11 2.0 mg/kg AD10302 | 1.000 | 0.000 | 1.140 | 0.080 |
| Group 12 4.0 mg/kg AD10302 | 1.000 | 0.000 | 0.623 | 0.170 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.130 | 1.000 | 0.412 |
| Group 2 1.0 mg/kg AD09699 | 0.898 | 0.110 | 0.838 | 0.283 |
| Group 3 2.0 mg/kg AD09699 | 0.891 | 0.100 | 1.037 | 0.121 |
| Group 4 4.0 mg/kg AD09699 | 1.003 | 0.071 | 1.145 | 0.317 |
| Group 5 2.0 mg/kg AD10261 | 0.839 | 0.137 | 0.978 | 0.271 |
| Group 6 2.0 mg/kg AD10262 | 1.208 | 0.129 | 1.262 | 0.266 |
| Group 7 2.0 mg/kg AD10263 | 1.056 | 0.193 | 0.968 | 0.253 |
| Group 8 2.0 mg/kg AD10264 | 1.120 | 0.288 | 1.126 | 0.359 |
| Group 9 2.0 mg/kg AD10265 | 0.944 | 0.164 | 1.071 | 0.125 |
| Group 10 1.0 mg/kg AD10302 | 0.924 | 0.227 | 0.973 | 0.480 |
| Group 11 2.0 mg/kg AD10302 | 1.094 | 0.227 | 1.345 | 0.360 |
| Group 12 4.0 mg/kg AD10302 | 0.585 | 0.202 | 0.673 | 0.068 |

The DMPK RNAi agents in Groups 2, 3, 4, 9, 10, and 12 showed reduction in DMPK-AAV as compared to the saline control (Group 1) at Day 8, reduction for Groups 2, 3, 5, 9, 10, and 12 at Day 15, and reduction for Groups 2, 5, 7, 10, and 12 at Day 22.

### Example 5. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 1.0 mg/kg (mpk), 3.0 mg/kg (mpk), 5.0 mg/kg (mpk) of a DMPK RNAi agent, or saline without a DMPK RNAi agent to be used as a control, according to the following Table 11.

TABLE 11
DMPK RNAi Agent and Dosing for Example 5

| Group ID | Dosing Regimen |
|---|---|
| Group 1 Saline | Single SQ injection on day 1 |
| Group 2 1.0 mg/kg AD09699 | Single SQ injection on day 1 |
| Group 3 3.0 mg/kg AD09699 | Single SQ injection on day 1 |
| Group 4 5.0 mg/kg AD09699 | Single SQ injection on day 1 |
| Group 5 3.0 mg/kg AD09702 | Single SQ injection on day 1 |
| Group 6 3.0 mg/kg AD10261 | Single SQ injection on day 1 |
| Group 7 3.0 mg/kg AD10262 | Single SQ injection on day 1 |
| Group 8 3.0 mg/kg AD10263 | Single SQ injection on day 1 |
| Group 9 3.0 mg/kg AD10264 | Single SQ injection on day 1 |
| Group 10 3.0 mg/kg AD10265 | Single SQ injection on day 1 |
| Group 11 1.0 mg/kg AD10302 | Single SQ injection on day 1 |
| Group 12 3.0 mg/kg AD10302 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 12.

TABLE 12
Average DMPK Normalized to Pre-Treatment and Control
in AAV-hDMPK Mice from Example 5

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.323 |
| Group 2 1.0 mg/kg AD09699 | 1.000 | 0.000 | 0.604 | 0.058 |
| Group 3 3.0 mg/kg AD09699 | 1.000 | 0.000 | 0.507 | 0.068 |
| Group 4 5.0 mg/kg AD09699 | 1.000 | 0.000 | 0.399 | 0.025 |
| Group 5 3.0 mg/kg AD09702 | 1.000 | 0.000 | 0.581 | 0.047 |
| Group 6 3.0 mg/kg AD10261 | 1.000 | 0.000 | 0.591 | 0.085 |
| Group 7 3.0 mg/kg AD10262 | 1.000 | 0.000 | 0.671 | 0.149 |
| Group 8 3.0 mg/kg AD10263 | 1.000 | 0.000 | 0.669 | 0.161 |
| Group 9 3.0 mg/kg AD10264 | 1.000 | 0.000 | 0.596 | 0.108 |
| Group 10 3.0 mg/kg AD10265 | 1.000 | 0.000 | 0.675 | 0.127 |
| Group 11 1.0 mg/kg AD10302 | 1.000 | 0.000 | 0.722 | 0.394 |
| Group 12 3.0 mg/kg AD10302 | 1.000 | 0.000 | 0.633 | 0.084 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.321 | 1.000 | 0.366 |
| Group 2 1.0 mg/kg AD09699 | 0.651 | 0.044 | 0.568 | 0.138 |
| Group 3 3.0 mg/kg AD09699 | 0.578 | 0.139 | 0.548 | 0.149 |
| Group 4 5.0 mg/kg AD09699 | 0.436 | 0.041 | 0.451 | 0.009 |
| Group 5 3.0 mg/kg AD09702 | 0.571 | 0.025 | 0.622 | 0.045 |
| Group 6 3.0 mg/kg AD10261 | 0.674 | 0.083 | 0.687 | 0.103 |
| Group 7 3.0 mg/kg AD10262 | 0.666 | 0.130 | 0.631 | 0.242 |
| Group 8 3.0 mg/kg AD10263 | 0.630 | 0.068 | 0.587 | 0.133 |
| Group 9 3.0 mg/kg AD10264 | 0.617 | 0.151 | 0.712 | 0.160 |
| Group 10 3.0 mg/kg AD10265 | 0.674 | 0.139 | 0.678 | 0.169 |
| Group 11 1.0 mg/kg AD10302 | 0.683 | 0.236 | 0.745 | 0.202 |
| Group 12 3.0 mg/kg AD10302 | 0.615 | 0.095 | 0.688 | 0.101 |

The DMPK RNAi agents in each of the dosing groups (i.e., Groups 2 through 12) showed reduction in DMPK-AAV as compared to the saline control (Group 1) across all measured time points. AD09699 demonstrated greater knockdown at higher doses (i.e., a dose-response.)

### Example 6. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 3.0 mg/kg (mpk) of a DMPK RNAi agent or saline without a DMPK RNAi agent to be used as a control, according to the following Table 13.

TABLE 13
DMPK RNAi Agent and Dosing for Example 6

| Group ID | Dosing Regimen |
|---|---|
| Group 1 Saline | Single SQ injection on day 1 |
| Group 2 3.0 mg/kg AD09699 | Single SQ injection on day 1 |
| Group 3 3.0 mg/kg AD11130 | Single SQ injection on day 1 |
| Group 4 3.0 mg/kg AD11131 | Single SQ injection on day 1 |
| Group 5 3.0 mg/kg AD11132 | Single SQ injection on day 1 |
| Group 6 3.0 mg/kg AD11133 | Single SQ injection on day 1 |
| Group 7 3.0 mg/kg AD11134 | Single SQ injection on day 1 |
| Group 8 3.0 mg/kg AD11135 | Single SQ injection on day 1 |
| Group 9 3.0 mg/kg AD11136 | Single SQ injection on day 1 |
| Group 10 3.0 mg/kg AD11137 | Single SQ injection on day 1 |
| Group 11 3.0 mg/kg AD11138 | Single SQ injection on day 1 |
| Group 12 3.0 mg/kg AD11139 | Single SQ injection on day 1 |
| Group 13 3.0 mg/kg AD11140 | Single SQ injection on day 1 |
| Group 14 3.0 mg/kg AD11141 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 14.

TABLE 14
Average DMPK Normalized to Pre-Treatment and Control
in AAV-hDMPK Mice from Example 6

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.173 |
| Group 2 3.0 mg/kg AD09699 | 1.000 | 0.000 | 0.792 | 0.187 |
| Group 3 3.0 mg/kg AD11130 | 1.000 | 0.000 | 0.622 | 0.169 |
| Group 4 3.0 mg/kg AD11131 | 1.000 | 0.000 | 0.564 | 0.035 |
| Group 5 3.0 mg/kg AD11132 | 1.000 | 0.000 | 0.713 | 0.120 |
| Group 6 3.0 mg/kg AD11133 | 1.000 | 0.000 | 0.587 | 0.129 |
| Group 7 3.0 mg/kg AD11134 | 1.000 | 0.000 | 0.668 | 0.113 |
| Group 8 3.0 mg/kg AD11135 | 1.000 | 0.000 | 0.614 | 0.079 |
| Group 9 3.0 mg/kg AD11136 | 1.000 | 0.000 | 0.570 | 0.108 |
| Group 10 3.0 mg/kg AD11137 | 1.000 | 0.000 | 0.670 | 0.165 |
| Group 11 3.0 mg/kg AD11138 | 1.000 | 0.000 | 0.755 | 0.237 |
| Group 12 3.0 mg/kg AD11139 | 1.000 | 0.000 | 0.985 | 0.365 |
| Group 13 3.0 mg/kg AD11140 | 1.000 | 0.000 | 0.573 | 0.043 |
| Group 14 3.0 mg/kg AD11141 | 1.000 | 0.000 | 0.709 | 0.167 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.132 | 1.000 | 0.264 |
| Group 2 3.0 mg/kg AD09699 | 0.666 | 0.065 | 0.869 | 0.214 |
| Group 3 3.0 mg/kg AD11130 | 0.535 | 0.099 | 0.818 | 0.366 |
| Group 4 3.0 mg/kg AD11131 | 0.614 | 0.079 | 0.629 | 0.123 |
| Group 5 3.0 mg/kg AD11132 | 0.708 | 0.110 | 0.733 | 0.135 |
| Group 6 3.0 mg/kg AD11133 | 0.610 | 0.075 | 0.569 | 0.072 |
| Group 7 3.0 mg/kg AD11134 | 0.716 | 0.101 | 0.642 | 0.153 |
| Group 8 3.0 mg/kg AD11135 | 0.706 | 0.083 | 0.623 | 0.089 |
| Group 9 3.0 mg/kg AD11136 | 0.573 | 0.097 | 0.572 | 0.122 |
| Group 10 3.0 mg/kg AD11137 | 0.587 | 0.056 | 0.650 | 0.021 |
| Group 11 3.0 mg/kg AD11138 | 0.759 | 0.290 | 0.651 | 0.278 |
| Group 12 3.0 mg/kg AD11139 | 0.806 | 0.064 | 0.765 | 0.159 |
| Group 13 3.0 mg/kg AD11140 | 0.616 | 0.055 | 0.700 | 0.122 |
| Group 14 3.0 mg/kg AD11141 | 0.731 | 0.138 | 0.629 | 0.079 |

The DMPK RNAi agents in each of the dosing groups (i.e., Groups 2 through 14) showed reduction in DMPK-AAV as compared to the saline control (Group 1) across all measured time points. Groups 6 and 9 showed greater than 40% knockdown at day 22.

### Example 7. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 3.0 mg/kg (mpk) of a DMPK RNAi agent or saline without a DMPK RNAi agent to be used as a control, according to the following Table 15.

TABLE 15
DMPK RNAi Agent and Dosing for Example 7

| Group ID | Dosing Regimen |
|---|---|
| Group 1 Saline | Single SQ injection on day 1 |
| Group 2 3.0 mg/kg AD11136 | Single SQ injection on day 1 |
| Group 3 3.0 mg/kg AD11633 | Single SQ injection on day 1 |
| Group 4 3.0 mg/kg AD11634 | Single SQ injection on day 1 |
| Group 5 3.0 mg/kg AD11635 | Single SQ injection on day 1 |
| Group 6 3.0 mg/kg AD11636 | Single SQ injection on day 1 |
| Group 7 3.0 mg/kg AD11637 | Single SQ injection on day 1 |
| Group 8 3.0 mg/kg AD11638 | Single SQ injection on day 1 |
| Group 9 3.0 mg/kg AD11639 | Single SQ injection on day 1 |
| Group 10 3.0 mg/kg AD11640 | Single SQ injection on day 1 |
| Group 11 3.0 mg/kg AD11642 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 16.

TABLE 16
Average DMPK Normalized to Pre-Treatment and Control
in AAV-hDMPK Mice from Example 7

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.086 |
| Group 2 3.0 mg/kg AD11136 | 1.000 | 0.000 | 0.764 | 0.091 |
| Group 3 3.0 mg/kg AD11633 | 1.000 | 0.000 | 0.599 | 0.076 |
| Group 4 3.0 mg/kg AD11634 | 1.000 | 0.000 | 0.570 | 0.113 |
| Group 5 3.0 mg/kg AD11635 | 1.000 | 0.000 | 0.472 | 0.089 |
| Group 6 3.0 mg/kg AD11636 | 1.000 | 0.000 | 0.675 | 0.141 |
| Group 7 3.0 mg/kg AD11637 | 1.000 | 0.000 | 0.737 | 0.095 |
| Group 8 3.0 mg/kg AD11638 | 1.000 | 0.000 | 0.703 | 0.079 |
| Group 9 3.0 mg/kg AD11639 | 1.000 | 0.000 | 0.835 | 0.634 |
| Group 10 3.0 mg/kg AD11640 | 1.000 | 0.000 | 0.645 | 0.046 |
| Group 11 3.0 mg/kg AD11642 | 1.000 | 0.000 | 0.666 | 0.055 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.044 | 1.000 | 0.084 |
| Group 2 3.0 mg/kg AD11136 | 0.717 | 0.096 | 0.643 | 0.154 |
| Group 3 3.0 mg/kg AD11633 | 0.767 | 0.128 | 0.670 | 0.129 |
| Group 4 3.0 mg/kg AD11634 | 0.519 | 0.134 | 0.503 | 0.118 |
| Group 5 3.0 mg/kg AD11635 | 0.450 | 0.143 | 0.425 | 0.051 |
| Group 6 3.0 mg/kg AD11636 | 0.681 | 0.076 | 0.611 | 0.099 |
| Group 7 3.0 mg/kg AD11637 | 0.696 | 0.110 | 0.749 | 0.228 |
| Group 8 3.0 mg/kg AD11638 | 0.581 | 0.072 | 0.683 | 0.058 |
| Group 9 3.0 mg/kg AD11639 | 1.799 | 2.631 | 0.690 | 0.122 |
| Group 10 3.0 mg/kg AD11640 | 0.570 | 0.072 | 0.615 | 0.073 |
| Group 11 3.0 mg/kg AD11642 | 0.756 | 0.086 | 0.690 | 0.123 |

The DMPK RNAi agents in each of the dosing groups (i.e., Groups 2 through 11) showed reduction in DMPK-AAV as compared to the saline control (Group 1) at Day 8 and Day 22. DMPK RNAi agents of Groups 2-8, 10, and 11 showed reduction in DMPK-AAV as compared to the saline control (Group 1) at Day 15. Group 5 showed particularly robust (>50%) knockdown at days 15 and 22.

### Example 8. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 4.0 mg/kg (mpk) of a DMPK RNAi agent or saline without a DMPK RNAi agent to be used as a control, according to the following Table 17.

TABLE 17
DMPK RNAi Agent and Dosing for Example 8

| Group ID | Dosing Regimen |
|---|---|
| Group 1 Saline | Single SQ injection on day 1 |
| Group 2 4.0 mg/kg AD10557 | Single SQ injection on day 1 |
| Group 3 4.0 mg/kg AD11891 | Single SQ injection on day 1 |
| Group 4 4.0 mg/kg AD11892 | Single SQ injection on day 1 |
| Group 5 4.0 mg/kg AD11893 | Single SQ injection on day 1 |
| Group 6 4.0 mg/kg AD11894 | Single SQ injection on day 1 |
| Group 7 4.0 mg/kg AD11895 | Single SQ injection on day 1 |
| Group 8 4.0 mg/kg AD11896 | Single SQ injection on day 1 |
| Group 9 4.0 mg/kg AD11897 | Single SQ injection on day 1 |
| Group 10 4.0 mg/kg AD11898 | Single SQ injection on day 1 |
| Group 11 4.0 mg/kg AD11899 | Single SQ injection on day 1 |
| Group 12 4.0 mg/kg AD11900 | Single SQ injection on day 1 |
| Group 13 4.0 mg/kg AD11953 | Single SQ injection on day 1 |
| Group 14 4.0 mg/kg AD11635 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 18.

TABLE 18
Average DMPK Normalized to Pre-Treatment and Control
in AAV-hDMPK Mice from Example 8

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.208 |
| Group 2 4.0 mg/kg AD10557 | 1.000 | 0.000 | 0.734 | 0.092 |
| Group 3 4.0 mg/kg AD11891 | 1.000 | 0.000 | 0.921 | 0.221 |
| Group 4 4.0 mg/kg AD11892 | 1.000 | 0.000 | 0.936 | 0.184 |
| Group 5 4.0 mg/kg AD11893 | 1.000 | 0.000 | 0.828 | 0.129 |
| Group 6 4.0 mg/kg AD11894 | 1.000 | 0.000 | 0.840 | 0.103 |
| Group 7 4.0 mg/kg AD11895 | 1.000 | 0.000 | 0.893 | 0.035 |
| Group 8 4.0 mg/kg AD11896 | 1.000 | 0.000 | 0.921 | 0.147 |
| Group 9 4.0 mg/kg AD11897 | 1.000 | 0.000 | 0.669 | 0.160 |
| Group 10 4.0 mg/kg AD11898 | 1.000 | 0.000 | 0.738 | 0.163 |
| Group 11 4.0 mg/kg AD11899 | 1.000 | 0.000 | 0.687 | 0.112 |
| Group 12 4.0 mg/kg AD11900 | 1.000 | 0.000 | 0.974 | 0.125 |
| Group 13 4.0 mg/kg AD11953 | 1.000 | 0.000 | 0.544 | 0.214 |
| Group 14 4.0 mg/kg AD11635 | 1.000 | 0.000 | 0.732 | 0.190 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.236 | 1.000 | 0.321 |
| Group 2 4.0 mg/kg AD10557 | 0.460 | 0.089 | 0.679 | 0.159 |
| Group 3 4.0 mg/kg AD11891 | 0.763 | 0.088 | 0.891 | 0.123 |
| Group 4 4.0 mg/kg AD11892 | 0.737 | 0.118 | 0.864 | 0.106 |
| Group 5 4.0 mg/kg AD11893 | 0.698 | 0.132 | 0.962 | 0.162 |
| Group 6 4.0 mg/kg AD11894 | 0.690 | 0.067 | 0.748 | 0.106 |
| Group 7 4.0 mg/kg AD11895 | 0.558 | 0.074 | 0.677 | 0.093 |
| Group 8 4.0 mg/kg AD11896 | 0.662 | 0.067 | 0.831 | 0.113 |
| Group 9 4.0 mg/kg AD11897 | 0.590 | 0.106 | 0.665 | 0.072 |
| Group 10 4.0 mg/kg AD11898 | 0.610 | 0.223 | 0.607 | 0.347 |
| Group 11 4.0 mg/kg AD11899 | 0.552 | 0.158 | 0.824 | 0.168 |
| Group 12 4.0 mg/kg AD11900 | 0.741 | 0.166 | 0.887 | 0.123 |
| Group 13 4.0 mg/kg AD11953 | 0.398 | 0.111 | 0.680 | 0.195 |
| Group 14 4.0 mg/kg AD11635 | 0.544 | 0.307 | 0.847 | 0.277 |

The DMPK RNAi agents in each of the dosing groups (i.e., Groups 2 through 14) showed reduction in DMPK-AAV as compared to the saline control (Group 1) across all measured time points. Groups 2 and 13 averaged greater than 50% knockdown at day 15.

### Example 9. In Vivo Testing of DMPK RNAi Agents in Cynomolgus Monkeys

Cynomolgus monkeys were weighed and dosed with an RNAi agent on Day 1 via intravenous route of injection. Muscle biopsies were collected on Days-14 (pre-treatment), 29, and 57. At each timepoint, the cynomolgus study animals were fasted overnight (at least 12 but no more than 18 hours). The animals were dosed in accordance with Table 19.

TABLE 19
DMPK RNAi Agent and Dosing for Example 9

| | | | Number of |
|---|---|---|---|
| | | Dose | Animals |
| Group ID | Dose | Volume | (n=) |
| Group 1 | Single intravenous (i.v.) | 1.0 ml/kg | 3 |
| 20 mg/kg | injection on day 1 | | |
| AC001731 | | | |
| Group 2 | Single intravenous (i.v.) | 1.0 ml/kg | 3 |
| 20 mg/kg | injection on day 1 | | |
| AC001889 | | | |
| Group 3 | Single intravenous (i.v.) | 1.0 ml/kg | 3 |
| 20 mg/kg | injection on day 1 | | |
| AC001890 | | | |

Individual doses were calculated based on body weights recorded on each day of dosing. Test articles were administered as a single intravenous (i.v.) injection utilizing the cephalic vein. IV injection volume was slowly administered over 1 minute. The cephalic vein was selected and prepped, a 0.5 ml saline flush was administered just prior to IV administration, and a 1.0 ml saline flush administer post-test article administration.

Muscle biopsies were collected while the study animals were sedated. Animals were sedated using Telazol (4-6 mg/kg) and supplemented with Ketamine (~5 mg/kg) if necessary to maintain appropriate plane of sedation. The biopsy collection included at least 100 mg each from quadriceps and triceps on Days-14 (pre-treatment), 29, and 57. The biopsies were collected from alternating limbs. Biopsies collected on Days-14 and 57 were collected from sited separated by 1-2 cm. Table 20 shows mRNA expression sampled from Cynomolgus quadriceps. Table 21 shows mRNA expression sampled from Cynomolgus triceps.

Cynomolgus DMPK mRNA expression was quantified by probe-based quantitative PCR, normalized to Day-14 Pre-Treatment of each test group (geometric mean, +/− geometric SD).

TABLE 20
Average Relative Cynomolgus Monkey DMPK mRNA Expression in Example 9, in Quadriceps.
Quadriceps

| | Day −14 | | | Day 29 | | | Day 57 | | |
|---|---|---|---|---|---|---|---|---|---|
| | | Low | High | | Low | High | | Low | High |
| | Average | (error) | (error) | Average | (error) | (error) | Average | (error) | (error) |
| Group 1 | 1.000 | 0.223 | 0.286 | 0.152 | 0.047 | 0.068 | 0.168 | 0.045 | 0.062 |
| 20 mg/kg | | | | | | | | | |
| AC001731 | | | | | | | | | |
| Group 2 | 1.000 | 0.163 | 0.194 | 0.132 | 0.031 | 0.041 | 0.145 | 0.023 | 0.027 |
| 20 mg/kg | | | | | | | | | |
| AC001889 | | | | | | | | | |
| Group 3 | 1.000 | 0.167 | 0.201 | 0.220 | 0.029 | 0.034 | 0.271 | 0.094 | 0.143 |
| 20 mg/kg | | | | | | | | | |
| AC001890 | | | | | | | | | |
| | Day 85 | | | Day 116 | | | Day 169 | | |
| | | Low | High | | Low | High | | Low | High |
| | Average | (error) | (error) | Average | (error) | (error) | Average | (error) | (error) |
| Group 1 | 0.373 | 0.129 | 0.197 | 0.504 | 0.241 | 0.461 | 0.219 | 0.107 | 0.209 |
| 20 mg/kg | | | | | | | | | |
| AC001731 | | | | | | | | | |
| Group 2 | 0.262 | 0.050 | 0.062 | 0.374 | 0.254 | 0.789 | 0.459 | 0.121 | 0.165 |
| 20 mg/kg | | | | | | | | | |
| AC001889 | | | | | | | | | |
| Group 3 | 0.716 | 0.466 | 1.336 | 0.761 | 0.295 | 0.481 | 1.223 | 0.542 | 0.974 |
| 20 mg/kg | | | | | | | | | |
| AC001890 | | | | | | | | | |

TABLE 21
Average Relative Cynomolgus Monkey DMPK mRNA Expression in Example 9, in Triceps.
Triceps

| | Day −14 | | | Day 29 | | | Day 57 | | |
|---|---|---|---|---|---|---|---|---|---|
| | | Low | High | | Low | High | | Low | High |
| | Average | (error) | (error) | Average | (error) | (error) | Average | (error) | (error) |
| Group 1 | 1.000 | 0.373 | 0.595 | 0.161 | 0.027 | 0.032 | 0.753 | 0.297 | 0.490 |
| 20 mg/kg | | | | | | | | | |
| AC001731 | | | | | | | | | |
| Group 2 | 1.000 | 0.346 | 0.530 | 0.173 | 0.047 | 0.064 | 0.226 | 0.032 | 0.038 |
| 20 mg/kg | | | | | | | | | |
| AC001889 | | | | | | | | | |
| Group 3 | 1.000 | 0.405 | 0.680 | 0.157 | 0.040 | 0.054 | 0.237 | 0.082 | 0.125 |
| 20 mg/kg | | | | | | | | | |
| AC001890 | | | | | | | | | |
| | Day 85 | | | Day 116 | | | Day 169 | | |
| | | Low | High | | Low | High | | Low | High |
| | Average | (error) | (error) | Average | (error) | (error) | Average | (error) | (error) |
| Group 1 | 0.248 | 0.053 | 0.067 | 0.894 | 0.445 | 0.886 | 0.756 | 0.364 | 0.701 |
| 20 mg/kg | | | | | | | | | |
| AC001731 | | | | | | | | | |
| Group 2 | 0.598 | 0.361 | 0.911 | 0.226 | 0.284 | 0.782 | 0.577 | 0.240 | 0.410 |
| 20 mg/kg | | | | | | | | | |
| AC001889 | | | | | | | | | |
| Group 3 | 0.291 | 0.082 | 0.114 | 0.237 | 0.210 | 0.344 | 0.667 | 0.278 | 0.478 |
| 20 mg/kg | | | | | | | | | |
| AC001890 | | | | | | | | | |

As shown above in Tables 20 and 21, the DMPK RNAi agents showed inhibition in both quadriceps and triceps, demonstrating the ability to silence DMPK expression in non-human primates. More specifically, RNAi agent AC001890 achieved ~84% inhibition at 20 mg/kg on Day 29 in the triceps.

### Example 10. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 3.0 mg/kg (mpk) of a DMPK RNAi agent or saline without a DMPK RNAi agent to be used as a control, according to the following Table 22.

TABLE 22
DMPK RNAi Agent and Dosing for Example 10

| Group ID | Dosing Regimen |
|---|---|
| Group 1 Saline | Single SQ injection on day 1 |
| Group 2 3.0 mg/kg AD09699 | Single SQ injection on day 1 |
| Group 3 3.0 mg/kg AD10869 | Single SQ injection on day 1 |
| Group 4 3.0 mg/kg AD10870 | Single SQ injection on day 1 |
| Group 5 3.0 mg/kg AD10871 | Single SQ injection on day 1 |
| Group 6 3.0 mg/kg AD10872 | Single SQ injection on day 1 |
| Group 7 3.0 mg/kg AD10873 | Single SQ injection on day 1 |
| Group 8 3.0 mg/kg AD10874 | Single SQ injection on day 1 |
| Group 9 3.0 mg/kg AD10875 | Single SQ injection on day 1 |
| Group 10 3.0 mg/kg AD10876 | Single SQ injection on day 1 |
| Group 11 3.0 mg/kg AD10877 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 23.

TABLE 23
Average DMPK Normalized to Pre-Treatment and Control
in AAV-hDMPK Mice from Example 10

| | Day 1 | | Day 8 | |
|---|---|---|---|---|
| ID | Avg | Std Dev | Avg | Std Dev |
| Group | DMPK | (+/−) | DMPK | (+/−) |
| Group 1 Saline | 1.000 | 0.000 | 1.000 | 0.194 |
| Group 2 3.0 mg/kg AD09699 | 1.000 | 0.000 | 0.681 | 0.176 |
| Group 3 3.0 mg/kg AD10869 | 1.000 | 0.000 | 0.915 | 0.334 |
| Group 4 3.0 mg/kg AD10870 | 1.000 | 0.000 | 0.679 | 0.105 |
| Group 5 3.0 mg/kg AD10871 | 1.000 | 0.000 | 0.667 | 0.131 |
| Group 6 3.0 mg/kg AD10872 | 1.000 | 0.000 | 0.571 | 0.060 |
| Group 7 3.0 mg/kg AD10873 | 1.000 | 0.000 | 0.618 | 0.076 |
| Group 8 3.0 mg/kg AD10874 | 1.000 | 0.000 | 0.684 | 0.189 |
| Group 9 3.0 mg/kg AD10875 | 1.000 | 0.000 | 0.551 | 0.112 |
| Group 10 3.0 mg/kg AD10876 | 1.000 | 0.000 | 0.644 | 0.039 |
| Group 11 3.0 mg/kg AD10877 | 1.000 | 0.000 | 0.605 | 0.067 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.190 | 1.000 | 0.217 |
| Group 2 3.0 mg/kg AD09699 | 0.681 | 0.077 | 0.603 | 0.049 |
| Group 3 3.0 mg/kg AD10869 | 0.938 | 0.226 | 0.817 | 0.291 |
| Group 4 3.0 mg/kg AD10870 | 0.764 | 0.133 | 0.615 | 0.083 |
| Group 5 3.0 mg/kg AD10871 | 0.840 | 0.111 | 0.633 | 0.127 |
| Group 6 3.0 mg/kg AD10872 | 0.817 | 0.090 | 0.560 | 0.144 |
| Group 7 3.0 mg/kg AD10873 | 0.909 | 0.119 | 0.615 | 0.050 |
| Group 8 3.0 mg/kg AD10874 | 0.702 | 0.201 | 0.574 | 0.186 |
| Group 9 3.0 mg/kg AD10875 | 0.622 | 0.093 | 0.489 | 0.178 |
| Group 10 3.0 mg/kg AD10876 | 0.710 | 0.095 | 0.537 | 0.123 |
| Group 11 3.0 mg/kg AD10877 | 0.763 | 0.277 | 0.726 | 0.152 |

The DMPK RNAi agents in each of the dosing groups (i.e., Groups 2 through 11) showed reduction in DMPK-AAV as compared to the saline control (Group 1) across all measured time points. More specifically, AD10875 achieved ~51% inhibition at 3.0 mg/kg on Day 22.

### Example 11. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 2.0 mg/kg (mpk) of a DMPK RNAi agent or saline without a DMPK RNAi agent to be used as a control, according to the following Table 24.

TABLE 24
DMPK RNAi Agent and Dosing for Example 11

| Group ID | Dosing Regimen |
|---|---|
| Group 1 Saline | Single SQ injection on day 1 |
| Group 2 2.0 mg/kg AD09721 | Single SQ injection on day 1 |
| Group 3 2.0 mg/kg AD10129 | Single SQ injection on day 1 |
| Group 4 2.0 mg/kg AD10130 | Single SQ injection on day 1 |
| Group 5 2.0 mg/kg AD10131 | Single SQ injection on day 1 |
| Group 6 2.0 mg/kg AD10132 | Single SQ injection on day 1 |
| Group 7 2.0 mg/kg AD10133 | Single SQ injection on day 1 |
| Group 8 2.0 mg/kg AD10134 | Single SQ injection on day 1 |
| Group 9 2.0 mg/kg AD10135 | Single SQ injection on day 1 |
| Group 10 2.0 mg/kg AD10136 | Single SQ injection on day 1 |
| Group 11 2.0 mg/kg AD10137 | Single SQ injection on day 1 |
| Group 12 2.0 mg/kg AD10138 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 25.

TABLE 25
Average DMPK Normalized to Pre-Treatment and
Control in AAV-hDMPK Mice from Example 11

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.180 |
| Group 2 2.0 mg/kg AD09721 | 1.000 | 0.000 | 0.730 | 0.084 |
| Group 3 2.0 mg/kg AD10129 | 1.000 | 0.000 | 0.881 | 0.104 |
| Group 4 2.0 mg/kg AD10130 | 1.000 | 0.000 | 0.979 | 0.083 |
| Group 5 2.0 mg/kg AD10131 | 1.000 | 0.000 | 0.804 | 0.182 |
| Group 6 2.0 mg/kg AD10132 | 1.000 | 0.000 | 0.779 | 0.159 |
| Group 7 2.0 mg/kg AD10133 | 1.000 | 0.000 | 0.918 | 0.125 |
| Group 8 2.0 mg/kg AD10134 | 1.000 | 0.000 | 1.027 | 0.125 |
| Group 9 2.0 mg/kg AD10135 | 1.000 | 0.000 | 1.018 | 0.200 |
| Group 10 2.0 mg/kg AD10136 | 1.000 | 0.000 | 1.008 | 0.194 |
| Group 11 2.0 mg/kg AD10137 | 1.000 | 0.000 | 0.961 | 0.060 |
| Group 12 2.0 mg/kg AD10138 | 1.000 | 0.000 | 0.813 | 0.138 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.080 | 1.000 | 0.398 |
| Group 2 2.0 mg/kg AD09721 | 0.790 | 0.109 | 0.867 | 0.140 |
| Group 3 2.0 mg/kg AD10129 | 0.949 | 0.132 | 1.023 | 0.164 |
| Group 4 2.0 mg/kg AD10130 | 1.047 | 0.097 | 0.984 | 0.133 |
| Group 5 2.0 mg/kg AD10131 | 1.070 | 0.197 | 1.137 | 0.355 |
| Group 6 2.0 mg/kg AD10132 | 1.021 | 0.112 | 0.898 | 0.063 |
| Group 7 2.0 mg/kg AD10133 | 1.030 | 0.200 | 1.022 | 0.212 |
| Group 8 2.0 mg/kg AD10134 | 1.283 | 0.351 | 1.060 | 0.291 |
| Group 9 2.0 mg/kg AD10135 | 1.103 | 0.270 | 1.118 | 0.384 |
| Group 10 2.0 mg/kg AD10136 | 1.322 | 0.192 | 1.341 | 0.361 |
| Group 11 2.0 mg/kg AD10137 | 0.991 | 0.172 | 0.864 | 0.226 |
| Group 12 2.0 mg/kg AD10138 | 1.090 | 0.060 | 0.952 | 0.205 |

The DMPK RNAi agents in Groups 2, 3, 4, 5, 6, 7, 11, and 12 showed reduction in DMPK-AAV as compared to the saline control (Group 1) at Day 8, reduction in Groups 2, 3, and 11 at Day 15, and reduction in Groups 2, 4, 6, 11, and 12 at Day 22.

### Example 12. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 1.0 mg/kg (mpk), 2.0 mg/kg (mpk), or 4.0 mg/kg (mpk) of a DMPK RNAi agent, or saline without a DMPK RNAi agent to be used as a control, according to the following Table 26.

TABLE 26
DMPK RNAi Agent and Dosing for Example 12

| Group ID | Dosing Regimen |
|---|---|
| Group 1 Saline | Single SQ injection on day 1 |
| Group 2 1.0 mg/kg AD09721 | Single SQ injection on day 1 |
| Group 3 2.0 mg/kg AD09721 | Single SQ injection on day 1 |
| Group 4 4.0 mg/kg AD09721 | Single SQ injection on day 1 |
| Group 5 2.0 mg/kg AD09722 | Single SQ injection on day 1 |
| Group 6 2.0 mg/kg AD09923 | Single SQ injection on day 1 |
| Group 7 2.0 mg/kg AD09706 | Single SQ injection on day 1 |
| Group 8 2.0 mg/kg AD09707 | Single SQ injection on day 1 |
| Group 9 2.0 mg/kg AD09709 | Single SQ injection on day 1 |
| Group 10 2.0 mg/kg AD09708 | Single SQ injection on day 1 |
| Group 11 2.0 mg/kg AD09922 | Single SQ injection on day 1 |
| Group 12 2.0 mg/kg AD09723 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 27.

TABLE 27
Average DMPK Normalized to Pre-Treatment and
Control in AAV-hDMPK Mice from Example 12

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.101 |
| Group 2 1.0 mg/kg AD09721 | 1.000 | 0.000 | 0.732 | 0.105 |
| Group 3 2.0 mg/kg AD09721 | 1.000 | 0.000 | 0.536 | 0.067 |
| Group 4 4.0 mg/kg AD09721 | 1.000 | 0.000 | 0.440 | 0.062 |
| Group 5 2.0 mg/kg AD09722 | 1.000 | 0.000 | 0.521 | 0.051 |
| Group 6 2.0 mg/kg AD09923 | 1.000 | 0.000 | 0.790 | 0.060 |
| Group 7 2.0 mg/kg AD09706 | 1.000 | 0.000 | 0.740 | 0.115 |
| Group 8 2.0 mg/kg AD09707 | 1.000 | 0.000 | 0.775 | 0.099 |
| Group 9 2.0 mg/kg AD09709 | 1.000 | 0.000 | 0.740 | 0.082 |
| Group 10 2.0 mg/kg AD09708 | 1.000 | 0.000 | 0.815 | 0.058 |
| Group 11 2.0 mg/kg AD09922 | 1.000 | 0.000 | 0.568 | 0.045 |
| Group 12 2.0 mg/kg AD09723 | 1.000 | 0.000 | 0.654 | 0.133 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.084 | 1.000 | 0.218 |
| Group 2 1.0 mg/kg AD09721 | 0.704 | 0.251 | 0.663 | 0.096 |
| Group 3 2.0 mg/kg AD09721 | 0.485 | 0.122 | 0.558 | 0.079 |
| Group 4 4.0 mg/kg AD09721 | 0.485 | 0.126 | 0.577 | 0.029 |
| Group 5 2.0 mg/kg AD09722 | 0.649 | 0.083 | 0.647 | 0.050 |
| Group 6 2.0 mg/kg AD09923 | 0.962 | 0.230 | 0.829 | 0.076 |
| Group 7 2.0 mg/kg AD09706 | 0.879 | 0.171 | 0.849 | 0.177 |
| Group 8 2.0 mg/kg AD09707 | 0.915 | 0.190 | 0.953 | 0.383 |
| Group 9 2.0 mg/kg AD09709 | 0.910 | 0.241 | 0.926 | 0.128 |
| Group 10 2.0 mg/kg AD09708 | 1.007 | 0.220 | 1.042 | 0.239 |
| Group 11 2.0 mg/kg AD09922 | 0.701 | 0.089 | 0.711 | 0.157 |
| Group 12 2.0 mg/kg AD09723 | 0.908 | 0.097 | 0.903 | 0.165 |

The DMPK RNAi agents in each of the dosing groups (i.e., Groups 2 through 12) showed reduction in DMPK-AAV as compared to the saline control (Group 1) across all measured time points, except for Group 10 at Day 15 and Day 22. More specifically, AD09721 achieved ~56% inhibition at 4.0 mg/kg on Day 8.

### Example 13. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 4.0 mg/kg (mpk) of a DMPK RNAi agent or saline without a DMPK RNAi agent to be used as a control, according to the following Table 28.

TABLE 28
DMPK RNAi Agent and Dosing for Example 13

| Group ID | Dosing Regimen |
|---|---|
| Group 1 Saline | Single SQ injection on day 1 |
| Group 2 4.0 mg/kg AD09721 | Single SQ injection on day 1 |
| Group 3 4.0 mg/kg AD08910 | Single SQ injection on day 1 |
| Group 4 4.0 mg/kg AD08911 | Single SQ injection on day 1 |
| Group 5 4.0 mg/kg AD08912 | Single SQ injection on day 1 |
| Group 6 4.0 mg/kg AD08913 | Single SQ injection on day 1 |
| Group 7 4.0 mg/kg AD08914 | Single SQ injection on day 1 |
| Group 8 4.0 mg/kg AD08915 | Single SQ injection on day 1 |
| Group 9 4.0 mg/kg AD08916 | Single SQ injection on day 1 |
| Group 10 4.0 mg/kg AD08917 | Single SQ injection on day 1 |
| Group 11 4.0 mg/kg AD08918 | Single SQ injection on day 1 |
| Group 12 4.0 mg/kg AD08919 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 29.

TABLE 29
Average DMPK Normalized to Pre-Treatment and
Control in AAV-hDMPK Mice from Example 13

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.244 |
| Group 2 4.0 mg/kg AD09721 | 1.000 | 0.000 | 0.633 | 0.210 |
| Group 3 4.0 mg/kg AD08910 | 1.000 | 0.000 | 0.707 | 0.174 |
| Group 4 4.0 mg/kg AD08911 | 1.000 | 0.000 | 0.962 | 0.187 |
| Group 5 4.0 mg/kg AD08912 | 1.000 | 0.000 | 0.681 | 0.085 |
| Group 6 4.0 mg/kg AD08913 | 1.000 | 0.000 | 0.654 | 0.097 |
| Group 7 4.0 mg/kg AD08914 | 1.000 | 0.000 | 1.389 | 0.864 |
| Group 8 4.0 mg/kg AD08915 | 1.000 | 0.000 | 1.190 | 0.253 |
| Group 9 4.0 mg/kg AD08916 | 1.000 | 0.000 | 1.030 | 0.183 |
| Group 10 4.0 mg/kg AD08917 | 1.000 | 0.000 | 1.004 | 0.254 |
| Group 11 4.0 mg/kg AD08918 | 1.000 | 0.000 | 1.461 | 0.961 |
| Group 12 4.0 mg/kg AD08919 | 1.000 | 0.000 | 2.828 | 0.154 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.412 | 1.000 | 0.280 |
| Group 2 4.0 mg/kg AD09721 | 0.509 | 0.121 | 0.675 | 0.122 |
| Group 3 4.0 mg/kg AD08910 | 0.806 | 0.187 | 0.997 | 0.282 |
| Group 4 4.0 mg/kg AD08911 | 1.010 | 0.354 | 1.130 | 0.400 |
| Group 5 4.0 mg/kg AD08912 | 0.721 | 0.179 | 1.049 | 0.201 |
| Group 6 4.0 mg/kg AD08913 | 0.968 | 0.114 | 0.756 | 0.017 |
| Group 7 4.0 mg/kg AD08914 | 1.075 | 0.495 | 1.395 | 0.872 |
| Group 8 4.0 mg/kg AD08915 | 0.776 | 0.144 | 0.819 | 0.197 |
| Group 9 4.0 mg/kg AD08916 | 1.041 | 0.073 | 1.121 | 0.186 |
| Group 10 4.0 mg/kg AD08917 | 1.031 | 0.126 | 1.067 | 0.201 |
| Group 11 4.0 mg/kg AD08918 | 0.900 | 0.133 | 0.984 | 0.161 |
| Group 12 4.0 mg/kg AD08919 | 1.304 | 0.332 | 1.230 | 0.495 |

The DMPK RNAi agents in Groups 2, 3, 4, 5, and 6 showed reduction in DMPK-AAV as compared to the saline control (Group 1) across at Day 8, reduction for Groups 2, 3, 5, 6, 8, and 11 at Day 15, and reduction for Groups 2, 3, 6, 8, and 11 at Day 22.

### Example 14. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 3.0 mg/kg (mpk), 6.0 mg/kg (mpk) of a DMPK RNAi agent, or saline without a DMPK RNAi agent to be used as a control, according to the following Table 30.

TABLE 30
DMPK RNAi Agent and Dosing for Example 14

| Group ID | Dosing Regimen |
|---|---|
| Group 1 Saline | Single SQ injection on day 1 |
| Group 2 3.0 mg/kg AD12246 | Single SQ injection on day 1 |
| Group 3 3.0 mg/kg AD11953 | Single SQ injection on day 1 |
| Group 4 3.0 mg/kg AD11752 | Single SQ injection on day 1 |
| Group 5 3.0 mg/kg AD12247 | Single SQ injection on day 1 |
| Group 6 3.0 mg/kg AD10557 | Single SQ injection on day 1 |
| Group 7 3.0 mg/kg AD12248 | Single SQ injection on day 1 |
| Group 8 6.0 mg/kg AD12246 | Single SQ injection on day 1 |
| Group 9 6.0 mg/kg AD11953 | Single SQ injection on day 1 |
| Group 10 6.0 mg/kg AD11752 | Single SQ injection on day 1 |
| Group 11 6.0 mg/kg AD12247 | Single SQ injection on day 1 |
| Group 12 6.0 mg/kg AD10557 | Single SQ injection on day 1 |
| Group 13 6.0 mg/kg AD12248 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 31.

TABLE 31
Average DMPK Normalized to Pre-Treatment and
Control in AAV-hDMPK Mice from Example 14

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.162 |
| Group 2 3.0 mg/kg AD12246 | 1.000 | 0.000 | 0.721 | 0.095 |
| Group 3 3.0 mg/kg AD11953 | 1.000 | 0.000 | 0.457 | 0.093 |
| Group 4 3.0 mg/kg AD11752 | 1.000 | 0.000 | 0.788 | 0.194 |
| Group 5 3.0 mg/kg AD12247 | 1.000 | 0.000 | 0.939 | 0.130 |
| Group 6 3.0 mg/kg AD10557 | 1.000 | 0.000 | 0.866 | 0.086 |
| Group 7 3.0 mg/kg AD12248 | 1.000 | 0.000 | 0.704 | 0.045 |
| Group 8 6.0 mg/kg AD12246 | 1.000 | 0.000 | 0.604 | 0.111 |
| Group 9 6.0 mg/kg AD11953 | 1.000 | 0.000 | 0.437 | 0.040 |
| Group 10 6.0 mg/kg AD11752 | 1.000 | 0.000 | 0.796 | 0.109 |
| Group 11 6.0 mg/kg AD12247 | 1.000 | 0.000 | 0.870 | 0.214 |
| Group 12 6.0 mg/kg AD10557 | 1.000 | 0.000 | 0.557 | 0.103 |
| Group 13 6.0 mg/kg AD12248 | 1.000 | 0.000 | 0.484 | 0.077 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.179 | 1.000 | 0.073 |
| Group 2 3.0 mg/kg AD12246 | 0.660 | 0.091 | 0.650 | 0.111 |
| Group 3 3.0 mg/kg AD11953 | 0.430 | 0.096 | 0.413 | 0.125 |
| Group 4 3.0 mg/kg AD11752 | 0.700 | 0.068 | 0.662 | 0.088 |
| Group 5 3.0 mg/kg AD12247 | 0.852 | 0.199 | 0.704 | 0.068 |
| Group 6 3.0 mg/kg AD10557 | 0.554 | 0.071 | 0.598 | 0.090 |
| Group 7 3.0 mg/kg AD12248 | 0.508 | 0.070 | 0.448 | 0.101 |
| Group 8 6.0 mg/kg AD12246 | 0.409 | 0.033 | 0.397 | 0.070 |
| Group 9 6.0 mg/kg AD11953 | 0.307 | 0.056 | 0.345 | 0.030 |
| Group 10 6.0 mg/kg AD11752 | 0.734 | 0.105 | 0.773 | 0.090 |
| Group 11 6.0 mg/kg AD12247 | 0.767 | 0.118 | 0.837 | 0.068 |
| Group 12 6.0 mg/kg AD10557 | 0.426 | 0.073 | 0.419 | 0.055 |
| Group 13 6.0 mg/kg AD12248 | 0.358 | 0.044 | 0.308 | 0.076 |

The DMPK RNAi agents in each of the dosing groups (i.e., Groups 2 through 13) showed reduction in DMPK-AAV as compared to the saline control (Group 1) across all measured time points. More specifically, AD11953 achieved ~69% inhibition at 6.0 mg/kg on Day 15.

### Example 15. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 2.0 mg/kg (mpk), 4.0 mg/kg (mpk) of a DMPK RNAi agent, or saline without a DMPK RNAi agent to be used as a control, according to the following Table 32.

TABLE 32
DMPK RNAi Agent and Dosing for Example 15

| Group ID | Dosing Regimen |
|---|---|
| Group 1 Saline | Single SQ injection on day 1 |
| Group 2 2.0 mg/kg AD09702 | Single SQ injection on day 1 |
| Group 3 2.0 mg/kg AD09699 | Single SQ injection on day 1 |
| Group 4 2.0 mg/kg AD10557 | Single SQ injection on day 1 |
| Group 5 2.0 mg/kg AD10560 | Single SQ injection on day 1 |
| Group 6 2.0 mg/kg AD10875 | Single SQ injection on day 1 |
| Group 7 4.0 mg/kg AD09702 | Single SQ injection on day 1 |
| Group 8 4.0 mg/kg AD09699 | Single SQ injection on day 1 |
| Group 9 4.0 mg/kg AD10557 | Single SQ injection on day 1 |
| Group 10 4.0 mg/kg AD10560 | Single SQ injection on day 1 |
| Group 11 4.0 mg/kg AD10875 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 33.

TABLE 33
Average DMPK Normalized to Pre-Treatment and
Control in AAV-hDMPK Mice from Example 15

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.438 |
| Group 2 2.0 mg/kg AD09702 | 1.000 | 0.000 | 0.792 | 0.254 |
| Group 3 2.0 mg/kg AD09699 | 1.000 | 0.000 | 0.864 | 0.362 |
| Group 4 2.0 mg/kg AD10557 | 1.000 | 0.000 | 0.615 | 0.166 |
| Group 5 2.0 mg/kg AD10560 | 1.000 | 0.000 | 0.780 | 0.213 |
| Group 6 2.0 mg/kg AD10875 | 1.000 | 0.000 | 0.805 | 0.140 |
| Group 7 4.0 mg/kg AD09702 | 1.000 | 0.000 | 0.615 | 0.076 |
| Group 8 4.0 mg/kg AD09699 | 1.000 | 0.000 | 0.761 | 0.072 |
| Group 9 4.0 mg/kg AD10557 | 1.000 | 0.000 | 0.337 | 0.078 |
| Group 10 4.0 mg/kg AD10560 | 1.000 | 0.000 | 0.404 | 0.086 |
| Group 11 4.0 mg/kg AD10875 | 1.000 | 0.000 | 0.671 | 0.123 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.280 | 1.000 | 0.212 |
| Group 2 2.0 mg/kg AD09702 | 0.627 | 0.117 | 0.699 | 0.159 |
| Group 3 2.0 mg/kg AD09699 | 0.929 | 0.307 | 0.950 | 0.205 |
| Group 4 2.0 mg/kg AD10557 | 0.513 | 0.169 | 0.434 | 0.120 |
| Group 5 2.0 mg/kg AD10560 | 0.713 | 0.183 | 0.656 | 0.250 |
| Group 6 2.0 mg/kg AD10875 | 0.880 | 0.160 | 0.931 | 0.154 |
| Group 7 4.0 mg/kg AD09702 | 0.592 | 0.104 | 0.710 | 0.143 |
| Group 8 4.0 mg/kg AD09699 | 0.937 | 0.186 | 0.845 | 0.078 |
| Group 9 4.0 mg/kg AD10557 | 0.306 | 0.151 | 0.325 | 0.209 |
| Group 10 4.0 mg/kg AD10560 | 0.286 | 0.082 | 0.286 | 0.066 |
| Group 11 4.0 mg/kg AD10875 | 0.903 | 0.259 | 0.898 | 0.234 |

The DMPK RNAi agents in each of the dosing groups (i.e., Groups 2 through 11) showed reduction in DMPK-AAV as compared to the saline control (Group 1) across all measured time points. More specifically, AD10560 achieved ~71% inhibition at 4.0 mg/kg on Day 22.

### Example 16. In Vivo Testing of DMPK RNAi Agents in DMPK-AAV Mice

The DMPK-AAV mouse model described in Example 2, above, was used. At day 1, each mouse was given a single subcutaneous (SQ) injection of 250 μl per 25 g body weight containing either 3.0 mg/kg (mpk) of a DMPK RNAi agent or saline without a DMPK RNAi agent to be used as a control, according to the following Table 34.

TABLE 34
DMPK RNAi Agent and Dosing for Example 16

| Group ID | Dosing Regimen |
|---|---|
| Group 1 Saline | Single SQ injection on day 1 |
| Group 2 3.0 mg/kg AD09721 | Single SQ injection on day 1 |
| Group 3 3.0 mg/kg AD08913 | Single SQ injection on day 1 |
| Group 4 3.0 mg/kg AD11667 | Single SQ injection on day 1 |
| Group 5 3.0 mg/kg AD11668 | Single SQ injection on day 1 |
| Group 6 3.0 mg/kg AD11669 | Single SQ injection on day 1 |
| Group 7 3.0 mg/kg AD11670 | Single SQ injection on day 1 |
| Group 8 3.0 mg/kg AD09923 | Single SQ injection on day 1 |

At days 1, 8, 15, and 22, serum was collected, from which Gaussia luciferase glow assay was performed to quantify GLuc expression levels. GLuc expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

Four (4) mice in each group were tested (n=4). DMPK expression levels were determined pursuant to the procedure set forth above. Data from the experiment are shown in the following Table 35.

TABLE 3
Average DMPK Normalized to Pre-Treatment and
Control in AAV-hDMPK Mice from Example 16

| | Day 1 | | Day 8 | |
|---|---|---|---|---|
| | Avg | Std Dev | Avg | Std Dev |
| Group ID | DMPK | (+/−) | DMPK | (+/−) |
| Group 1 Saline | 1.000 | 0.000 | 1.000 | 0.217 |
| Group 2 3.0 mg/kg AD09721 | 1.000 | 0.000 | 0.666 | 0.077 |
| Group 3 3.0 mg/kg AD08913 | 1.000 | 0.000 | 0.913 | 0.108 |
| Group 4 3.0 mg/kg AD11667 | 1.000 | 0.000 | 0.695 | 0.110 |
| Group 5 3.0 mg/kg AD11668 | 1.000 | 0.000 | 0.694 | 0.222 |
| Group 6 3.0 mg/kg AD11669 | 1.000 | 0.000 | 0.689 | 0.151 |
| Group 7 3.0 mg/kg AD11670 | 1.000 | 0.000 | 0.569 | 0.107 |
| Group 8 3.0 mg/kg AD09923 | 1.000 | 0.000 | 0.740 | 0.100 |
| | Day 15 | | Day 22 | |
| | Avg | Std Dev | Avg | Std Dev |
| Group ID | DMPK | (+/−) | DMPK | (+/−) |
| Group 1 Saline | 1.000 | 0.129 | 1.000 | 0.110 |
| Group 2 3.0 mg/kg AD09721 | 0.760 | 0.133 | 0.623 | 0.087 |
| Group 3 3.0 mg/kg AD08913 | 1.038 | 0.082 | 0.969 | 0.098 |
| Group 4 3.0 mg/kg AD11667 | 0.909 | 0.160 | 0.856 | 0.072 |
| Group 5 3.0 mg/kg AD11668 | 0.927 | 0.180 | 0.852 | 0.205 |
| Group 6 3.0 mg/kg AD11669 | 0.843 | 0.147 | 0.789 | 0.086 |
| Group 7 3.0 mg/kg AD11670 | 0.725 | 0.083 | 0.751 | 0.090 |
| Group 8 3.0 mg/kg AD09923 | 0.924 | 0.111 | 0.968 | 0.067 |

The DMPK RNAi agents in each of the dosing groups showed reduction in DMPK-AAV as compared to the saline control at Day 8 and Day 22, reduction for all groups except for Group 3 at Day 15. More specifically, AD11670 achieved ~43% inhibition at 3.0 mg/kg on Day 8.

### Example 17. TREDT960I/HSA-rtTA Mouse Model

To assess DMPK RNAi agents in vivo, a transgenic model of myotonic dystrophy 1 was used. TREDT960I (FVB/NJ-Tg(tetO-DMPK*) A2352Coop/J) were commercially acquired and crossed with HSA-rtTA mice (B6;C3-Tg (ACTA1-rtTA,tetO-cre) 102Monk/J) by Jackson Laboratories (JAX) to produce homozygous offspring that express human DMPK exons 11-15, UTR, and 960 interrupted CTG repeats in skeletal muscle upon administration of tetracycline.

TREDT960I Mouse Background: The TREDT960I mice were created by standard techniques in an FVB background. The TREDT960I transgene contains a human genomic segment containing exons 11-15 of DMPK with 960 interrupted CTG repeats in the natural site of the repeats. Each interrupted CTG repeat contains contain 20 CTGs separated by a 5 nucleotide spacer formed by ligation of SalI and XhoI restriction sites. Additionally, the transgene contains 307 bp of the DMPK 3′ flanking region containing the natural polyadenylation signals and two copies of the cHS4 insulator flanking the 5′ and 3′ ends of the expression construct to prevent effects of the chromosomal insertion site. This mutant DMPK gene is under direction of the tetO (tet-responsive element) promoter. The transgenic construct was microinjected into the pronucleus of FVB/NJ fertilized eggs. TREDT960I transgenic founder line A2352 was subsequently established and maintained on the FVB/NJ background for at least 20 generations.

Because this mutant DMPK construct is under direction of the tetO promoter, expression is determined by which tissue(s) express tetracycline transactivator (rtTA). When bred to mice that express rtTA in a particular tissue, the resulting offspring will, in the presence of tetracycline, have the DMPK transgene expressed in that tissue.

HSA-rtTA/TRE-Cre Mouse Background: HSA-rtTA/TRE-Cre mice These transgenic mice have a tetracycline (doxycycline) inducible Cre-mediated recombination system that is specific for skeletal muscle myocytes. To achieve this, a transgenic construct containing cre, cre recombinase under the control of the tetO, tetracycline-responsive regulatory element and a second transgenic construct containing rtTA, reverse tetracycline-controlled transactivator under the control of the human ACTA1, actin, alpha 1 were coinjected into fertilized (C57BL/6×C3H)F2 mouse eggs then backcrossed to C57BL/6J for 3 generations.

Progeny from the cross of TREDT960I and HSA-rtTA/TRE-Cre mice used for studies supporting this application were either bi-transgenic homozygous for the TREDT960I transgene and hemizygous HSA-rtTA/TRE-Cre or homozygous TREDT960I and noncarriers for HSA-rtTA/TRE-Cre. Cre-recombinase expression had no effect on transgene expression in the TREDT960I animals and was considered an inert byproduct of the model.

Doxycycline induction of DMPK expression: Doxycycline, a stable antibiotic of the tetracycline class, was provided to animals via laced rodent diet. In some studies, administration of doxycycline beginning at birth (via the nursing mothers fed doxycycline-laced rodent diet) through weaning and was then provided ad libitum until termination unless the doxycycline-laced rodent diet was replaced by standard rodent diet as part of study procedures. Doxycycline-laced chow contained 2 grams doxycycline hyclate per kilogram of rodent diet. In some studies, administration of doxycycline did not begin until 4 weeks prior to study start when standard rodent diet was replaced by doxycycline-laced rodent diet as part of study procedures. Doxycycline-laced chow contained 2 grams doxycycline hyclate per kilogram of rodent diet.

Bodyweight assessment: Bodyweight was recorded on all days of RNAi agent administration and on day of tissue harvest. Bodyweights were normalized to the first day of tamoxifen administration and average bodyweight of either the homozygous TREDT960I/HSA-rtTA/TRE-Cre noncarrier control group or the homozygous TREDT960I transgene/hemizygous HSA-rtTA/TRE-Cre control group fed standard rodent diet (no doxycycline) administered saline (containing no RNAi agent).

Tissue collection: Mice were anesthetized with 3-4% isoflurane and euthanized via exsanguination. Tissues of interest intended for gene expression analysis were harvested and snap frozen in liquid nitrogen and then later stored at −80° C. Tissues of interest intended for histology were fixed in formalin then embedded in paraffin wax and stained via histochemical or immunohistochemical protocols.

Gene expression analysis: Whole frozen tissues were homogenized using a Tissue Homogenization System (Precellys) and RNA was isolated via acid guanidinium thiocyanate-phenol-chloroform extraction. Extracted RNA was used to synthesize complimentary DNA using a SuperScript™ VILO™ CDNA Synthesis Kit (Thermo) and gene expression was measured using either QX200 droplet digital PCR (Bio-Rad) or QuantFlex7 qRT-PCR (Applied Biosystems) systems employing Taqman primer/probe sets (Thermo-Fisher) designed to detect genes of interest. Gene expression was normalized to the average of the doxycycline induced control group which was administered doxycycline-laced rodent diet and saline (containing no RNAi agent).

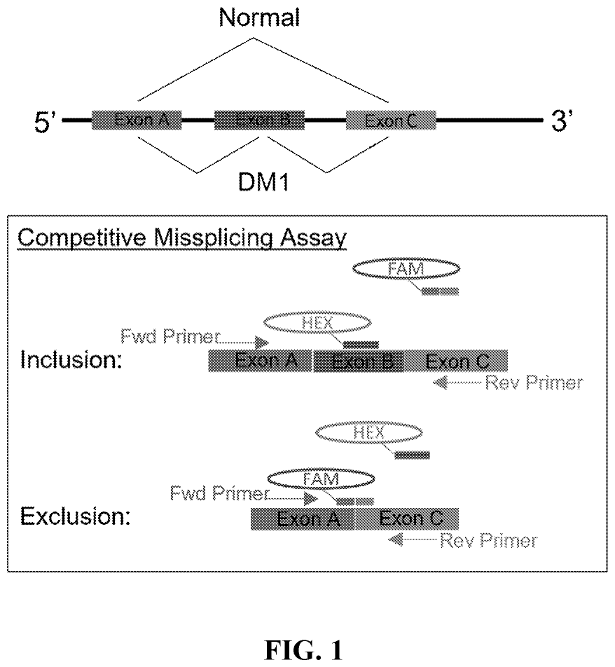

Competitive Missplicing Analysis: Primer sets were designed against transcripts known to be misspliced when mutant DMPK-CUG accumulates in myonuclei. Forward and reverse primers were designed against exons flanking exons known to be excluded or included under normal transcript splicing conditions and that are misspliced and erroneously included or excluded, respectively, in the accumulated presence of the mutant DMPK-CUG transcript in the nucleus. FAM-labeled probes were designed against the interface between flanking exons which would only interface in the case that the excluded exon is not present. HEX-labeled probes were designed against the excluded exon. FIG. 1 shows a graphical representation of the primer design.

Complimentary DNA was created from RNA isolated from collected tissues. For each transcript of interest, a mixture of primer set and FAM- and HEX-labeled probes were used to assay the percentage of misspliced transcript isolated from tissues administered or not administered doxycycline and/or RNAi agent. Examples of transcripts of interest may include Atp2a1 exon 22, Cacna1 exon 28, Ldb3 exon 8, and Mbnl1 exon 7.

### Example 18. In Vivo Testing of DMPK RNAi Agents in TREDT960I/HSA-rtTA Mouse Model

The DMPK TREDT960I/HAS-rtTA mouse model described in Example 17, above, was used. At days 1, 8, 15, 22, and 29, each mouse was given a single intravenous (i.v.) injection of 200 μl per 20 g body weight containing either 20.0 mg/kg (mpk), 40.0 mg/kg (mpk) of a DMPK RNAi agent or saline without a DMPK RNAi agent to be used as a control. The dosing groups are shown below in Table 36. Eight (n=8) mice were injected for each group, with each group containing four male and four female mice.

The test groups were also given either regular or doxycycline-laced rodent diet chow. All animals were maintained on doxycycline chow from receipt until study initiation. At study initiation, Group 1 was switched to normal chow, while all other Groups remained on doxycycline chow.

At Day 36, tissues were harvested. Tibialis anterior (TA) muscle, tricep (Tri) muscle, and gastrocnemius (Gas) muscle tissues were collected in transverse and longitudinal/oblique sections. The tissue samples were processed for CUG-(ISH)/mouse MBNL1 (Ab) and DMPK RNAscope staining. The tissue samples were processed for RNA isolation, cDNA generation. Quantitative polymerase chain reaction (qPCR) analysis was then performed for human DMPK and mouse Arl1 and missplicing analysis of mSerca1 (Atp2A1), mMbnl1, mCac1.1 (Cacna1), and mLdb3.

TABLE 36
DMPK RNAi Agent and Dosing for Example 18

| | | | | | # Animals |
|---|---|---|---|---|---|
| Group ID | Dose | Mouse Strain | Chow | Dosing Regimen | per Group |
| Group 1 | Saline | DM1-like (HOM TREDT960I/HEMI rtTA) | Regular | Single i.v. injection on | 4M/4F |
| | | | | day 1, 8, 15, 22, 29 | |
| Group 2 | Saline | DM1-like (HOM TREDT960I/HEMI rtTA) | Doxycycline | Single i.v. injection on | 4M/4F |
| | | | | day 1, 8, 15, 22, 29 | |
| Group 3 | 20.0 mg/kg | DM1-like (HOM TREDT960I/HEMI rtTA) | Doxycycline | Single i.v. injection on | 4M/4F |
| | AC002324 | | | day 1, 8, 15, 22, 29 | |
| Group 4 | 40.0 mg/kg | DM1-like (HOM TREDT960I/HEMI rtTA) | Doxycycline | Single i.v. injection on | 4M/4F |
| | AC002324 | | | day 1, 8, 15, 22, 29 | |
| Group 5 | 40.0 mg/kg | C57BL/6J | Doxycycline | Single i.v. injection on | 4M/4F |
| | AC002324 | | | day 1, 8, 15, 22, 29 | |
| Group 6 | 40.0 mg/kg | NCAR (Non-Carrier) | Doxycycline | Single i.v. injection on | 4M/4F |
| | AC002324 | | | day 1, 8, 15, 22, 29 | |
| Group 7 | Saline | NCAR (Non-Carrier) | Doxycycline | Single i.v. injection on | 4M/4F |
| | | | | day 1, 8, 15, 22, 29 | |

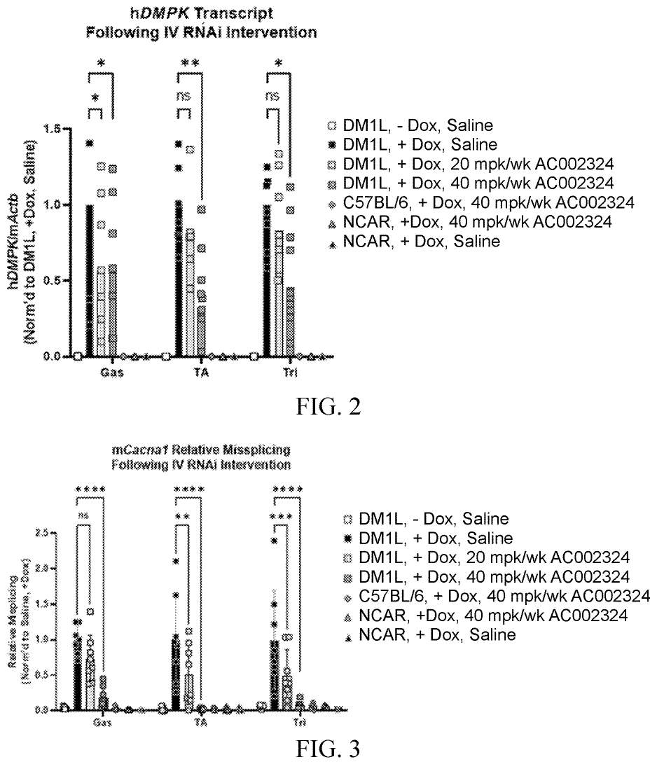

FIG. 2 shows the relative hDMPK transcript levels of the test groups. The hDMPK transcript levels are shown normalized to Group 2. FIG. 2 shows introducing AC002324 inhibits hDMPK transcript levels.

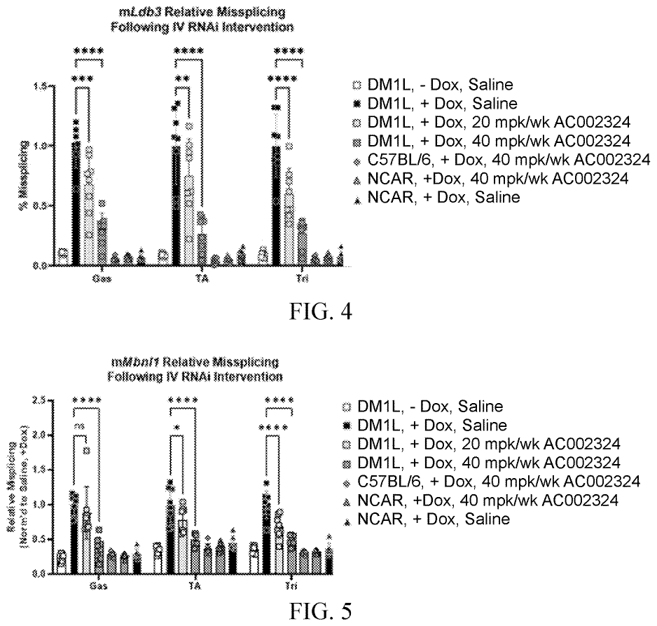

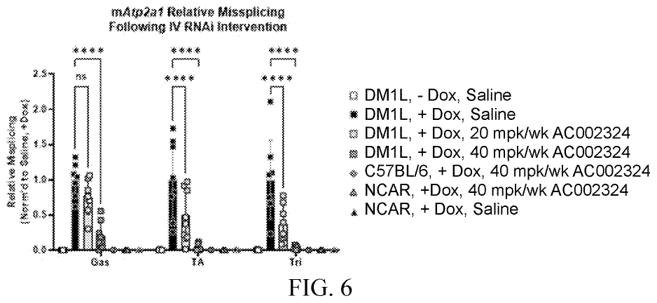

FIGS. 3 through 7 show relative missplicing of mCacna1, mLdb3, mMbnl1, and mAtp2a1 of the test groups. The relative missplicing levels are normalized to Group 2. FIGS. 3 through 6 show introducing DMPK RNAi agent AC002324 prevents splicing of these measured genes.

For FIGS. 2 through 6, the statistical significance is represented by: * is p<0.05, ** is p<0.01, *** is p<0.001, and **** is p<0.0001.

### Example 19. In Vivo Testing of DMPK RNAi Agents in Cynomolgus Monkeys

Cynomolgus study animals were weighed and dosed on Day 1 via intravenous route of injection. Muscle biopsies were collected on Days-14 (pre-treatment), 29, and 57. At each timepoint, the cynomolgus study animals were fasted overnight (at least 12 but no more than 18 hours). The animals were dosed in accordance with Table 37.

TABLE 37
DMPK RNAi Agent and Dosing for Example 19

| | | | Number of |
|---|---|---|---|
| | | Dose | Animals |
| Group ID | Dose | Volume | (n=) |
| Group 1 | Single intravenous (i.v.) | 1.0 ml/kg | 3 |
| 10 mg/kg | injection on day 1 | | |
| AC002324 | | | |
| Group 2 | Single intravenous (i.v.) | 1.0 ml/kg | 3 |
| 10 mg/kg | injection on day 1 | | |
| AC002690 | | | |
| Group 3 | Single intravenous (i.v.) | 1.0 ml/kg | 3 |
| 10 mg/kg | injection on day 1 | | |
| AC002691 | | | |

Individual doses were calculated based on body weights recorded on each day of dosing. Test articles were administered as a single intravenous (i.v.) injection utilizing the cephalic vein. For Groups 1-3, IV injection volume was slowly administered over 1 minute. The cephalic vein was selected and prepped, a 0.5 ml saline flush was administered just prior to IV administration, and a 1.0 ml saline flush administer post-test article administration.

Muscle biopsies were collected as a sedated procedure. Animals were sedated using Telazol (4-6 mg/kg) and supplemented with Ketamine (~5 mg/kg) if necessary to maintain appropriate plane of sedation. The biopsy collection included at least 100 mg each from quadriceps and triceps on Days-14 (pre-treatment), 29, 57, and 85. The biopsies were collected from alternating limbs. Biopsies collected on Days-14/57 and Days 29/85 were collected from sited separated by 1-2 cm. Table 38 shows mRNA expression sampled from Cynomolgus quadriceps. Table 39 shows mRNA expression sampled from Cynomolgus triceps.

Cynomolgus DMPK mRNA expression was quantified by probe-based quantitative PCR, normalized to Day-14 Pre-Treatment of each test group (geometric mean, +/− geometric SD).

TABLE 38
Average Relative Cynomolgus Monkey DMPK mRNA Expression in Example 19, in Quadriceps.
Quadriceps

| | Day −14 | | | Day 29 | | | Day 57 | | |
|---|---|---|---|---|---|---|---|---|---|
| | | Low | High | | Low | High | | Low | High |
| | Average | (error) | (error) | Average | (error) | (error) | Average | (error) | (error) |
| Group 1 | 1.000 | 0.351 | 0.542 | 0.330 | 0.073 | 0.094 | 0.217 | 0.071 | 0.105 |
| 10 mg/kg | | | | | | | | | |
| AC002324 | | | | | | | | | |
| Group 2 | 1.000 | 0.349 | 0.535 | 0.468 | 0.289 | 0.753 | 0.304 | 0.192 | 0.524 |
| 10 mg/kg | | | | | | | | | |
| AC002690 | | | | | | | | | |
| Group 3 | 1.000 | 0.294 | 0.416 | 0.352 | 0.213 | 0.538 | 0.600 | 0.233 | 0.381 |
| 10 mg/kg | | | | | | | | | |
| AC002691 | | | | | | | | | |

| | Day 85 | | |
|---|---|---|---|
| | | Low | High |
| | Average | (error) | (error) |
| Group 1 | 0.316 | 0.187 | 0.460 |
| 10 mg/kg | | | |
| AC002324 | | | |
| Group 2 | 0.139 | 0.023 | 0.028 |
| 10 mg/kg | | | |
| AC002690 | | | |
| Group 3 | 0.263 | 0.164 | 0.439 |
| 10 mg/kg | | | |
| AC002691 | | | |

TABLE 39
Average Relative Cynomolgus Monkey DMPK mRNA Expression in Example 19, in Triceps.
Triceps

| | Day −14 | | | Day 29 | | | Day 57 | | |
|---|---|---|---|---|---|---|---|---|---|
| | | Low | High | | Low | High | | Low | High |
| | Average | (error) | (error) | Average | (error) | (error) | Average | (error) | (error) |
| Group 1 | 1.000 | 0.310 | 0.449 | 0.298 | 0.096 | 0.143 | 0.531 | 0.151 | 0.211 |
| 10 mg/kg | | | | | | | | | |
| AC002324 | | | | | | | | | |
| Group 2 | 1.000 | 0.380 | 0.613 | 0.150 | 0.047 | 0.069 | 0.301 | 0.080 | 0.108 |
| 10 mg/kg | | | | | | | | | |
| AC002690 | | | | | | | | | |
| Group 3 | 1.000 | 0.295 | 0.419 | 0.107 | 0.015 | 0.017 | 0.200 | 0.109 | 0.241 |
| 10 mg/kg | | | | | | | | | |
| AC002691 | | | | | | | | | |

| | Day 85 | | |
|---|---|---|---|
| | | Low | High |
| | Average | (error) | (error) |
| Group 1 | 0.465 | 0.080 | 0.097 |
| 10 mg/kg | | | |
| AC002324 | | | |
| Group 2 | 0.525 | 0.101 | 0.125 |
| 10 mg/kg | | | |
| AC002690 | | | |
| Group 3 | 0.112 | 0.019 | 0.023 |
| 10 mg/kg | | | |
| AC002691 | | | |

As shown above in Tables 38 and 39, the DMPK RNAi agents showed inhibition in both quadriceps and triceps, demonstrating the ability to silence DMPK expression in non-human primates. More specifically, RNAi agent AC002691 achieved ~89% inhibition at 10 mg/kg on Day 29 in triceps, and ~88% inhibition at 10 mg/kg on Day 85 in triceps.

### Example 20. In Vivo Testing of DMPK RNAi Agents in Cynomolgus Monkeys

Cynomolgus study animals were weighed and dosed on Day 1 (for Group 1), and Day 1 and Day 29 (for Group 2) via subcutaneous (SQ) route of injection. Muscle biopsies were collected on Days-14 (pre-treatment), 29, 57, and 85. The animals were dosed in accordance with Table 40.

TABLE 40
DMPK RNAi Agent and Dosing for Example 20.

| | | | | Number of |
|---|---|---|---|---|
| | | | Dose | Animals |
| Group ID | Dosing Route | Dose | Volume | (n=) |
| Group 1 | Single SQ | 10 mg/kg | 0.5 mL/kg | 3 |
| 10 mg/kg | injection on day 1 | | | |
| AC002691 | | | | |
| Group 2 | Single SQ | 5 mg/kg | 0.5 mL/kg | 3 |
| 5 mg/kg | injection on day 1 | | | |
| AC002691 | and day 29 | | | |

Individual doses were calculated based on body weights recorded on each day of dosing. Test articles were administered as a single subcutaneous (SQ) injection in the mid-scapular region on Day 1 for Group 1 and Days 1 and 29 for Group 2. Injection sites were shaven and prepped per standard safety procedures prior to injection.

Muscle biopsies were collected as a sedated procedure. Animals were sedated using Telazol (5-8 mg/kg) and supplemented with Ketamine (~5 mg/kg) if necessary to maintain appropriate plane of sedation. The biopsy collection included at least 100 mg each from quadriceps and triceps on Days-14 (pre-treatment), 29, 57, and 85. The biopsies were collected from alternating limbs. Biopsies collected on Days-14/57 and Days 29/85 were collected from sites separated by 1-2 cm. Table 41 shows mRNA expression sampled from Cynomolgus quadriceps. Table 42 shows mRNA expression sampled from Cynomolgus triceps. Cynomolgus DMPK mRNA expression was quantified by probe-based quantitative PCR, normalized to Day-14 Pre-Treatment of each test group (geometric mean, +/− geometric SD).

TABLE 41
Average Relative Cynomolgus Monkey DMPK mRNA Expression in Example 20, in Quadriceps.
Quadriceps

| | Day −14 | | | Day 29 | | | Day 57 | | |
|---|---|---|---|---|---|---|---|---|---|
| | | Low | High | | Low | High | | Low | High |
| | Average | (error) | (error) | Average | (error) | (error) | Average | (error) | (error) |
| Group 1 | 1.000 | 0.183 | 0.224 | 0.168 | 0.024 | 0.029 | 0.131 | 0.028 | 0.035 |
| 10 mg/kg | | | | | | | | | |
| AC002691 | | | | | | | | | |
| Group 2 | 1.000 | 0.217 | 0.277 | 0.159 | 0.055 | 0.083 | 0.224 | 0.116 | 0.239 |
| 5 mg/kg | | | | | | | | | |
| AC002691 | | | | | | | | | |

| | Day 85 | | |
|---|---|---|---|
| | | Low | High |
| | Average | (error) | (error) |
| Group 1 | 0.125 | 0.063 | 0.128 |
| 10 mg/kg | | | |
| AC002691 | | | |
| Group 2 | 0.183 | 0.068 | 0.110 |
| 5 mg/kg | | | |
| AC002691 | | | |

TABLE 42
Average Relative Cynomolgus Monkey DMPK mRNA Expression in Example 20, in Triceps.
Triceps

| | Day −14 | | | Day 29 | | | Day 57 | | |
|---|---|---|---|---|---|---|---|---|---|
| | | Low | High | | Low | High | | Low | High |
| | Average | (error) | (error) | Average | (error) | (error) | Average | (error) | (error) |
| Group 1 | 1.000 | 0.317 | 0.465 | 0.099 | 0.020 | 0.025 | 0.161 | 0.096 | 0.237 |
| 10 mg/kg | | | | | | | | | |
| AC002691 | | | | | | | | | |
| Group 2 | 1.000 | 0.168 | 0.202 | 0.171 | 0.034 | 0.043 | 0.116 | 0.044 | 0.070 |
| 5 mg/kg | | | | | | | | | |
| AC002691 | | | | | | | | | |

| | Day 85 | | |
|---|---|---|---|
| | | Low | High |
| | Average | (error) | (error) |
| Group 1 | 0.182 | 0.098 | 0.210 |
| 10 mg/kg | | | |
| AC002691 | | | |
| Group 2 | 0.146 | 0.044 | 0.064 |
| 5 mg/kg | | | |
| AC002691 | | | |

As shown above in Tables 41 and 42, AC002691 showed significant inhibition in both quadriceps and triceps lasting until at least day 85, demonstrating the ability to silence DMPK expression in non-human primates. More specifically, RNAi agent AC002691 achieved ~90% inhibition at 10 mg/kg on Day 29 in triceps, and ~87% inhibition at 10 mg/kg on Day 85 in quadriceps.

### Example 21. In Vivo Testing of DMPK RNAi Agents in Cynomolgus Monkeys

Cynomolgus study animals were weighed and dosed on Day 1 and Day 29 (for Groups 1-3) via intravenous (IV) injection. For Groups 1-3, muscle biopsies and blood were collected on Days-14 (pre-treatment), 29, 57, and 85. The animals were dosed in accordance with Table 43.

TABLE 43
DMPK RNAi Agent and Dosing for Example 21.

| | | | | Number of |
|---|---|---|---|---|
| | | | Dose | Animals |
| Group ID | Dosing Route | Dose | Volume | (n=) |
| Group 1 | Single IV | 2.5 mg/kg | 1.0 mL/kg | 3 |
| 2.5 mg/kg | injection on day 1 | | | |
| AC002691 | and day 29 | | | |
| Group 2 | Single IV | 5.0 mg/kg | 1.0 mL/kg | 3 |
| 5.0 mg/kg | injection on day 1 | | | |
| AC002691 | and day 29 | | | |
| Group 3 | Single IV | 10.0 mg/kg | 1.0 mL/kg | 3 |
| 10.0 mg/kg | injection on day 1 | | | |
| AC002691 | and day 29 | | | |

Individual doses were calculated based on body weights recorded on each day of dosing. Test articles were administered as and single intravenous (IV) injection Days 1 and 29 for Groups 1-3. Injection sites were shaven and prepped per standard safety procedures prior to injection.

Muscle biopsies were collected as a sedated procedure. Animals were sedated using Telazol (5-8 mg/kg) and supplemented with Ketamine (~5 mg/kg) if necessary to maintain appropriate plane of sedation.

Groups 1-3 muscle biopsy collection included at least 100 mg each from quadriceps and triceps on Days-14 (pre-treatment), 29, 57, and 85. The biopsies were collected from alternating limbs. Biopsies collected on Days-14/57 and Days 29/85 were collected from sites separated by 1-2 cm. Table 44 shows mRNA expression sampled from Cynomolgus quadriceps. Table 45 shows mRNA expression sampled from Cynomolgus triceps. Cynomolgus DMPK mRNA expression was quantified by probe-based quantitative PCR, normalized to Day −14 Pre-Treatment of each test group (geometric mean, +/− geometric SD).

TABLE 44
Average Relative Cynomolgus Monkey DMPK mRNA
Expression in Example 21, in Quadriceps.
Quadriceps

| | Day −14 | | Day 29 | | Day 57 | |
|---|---|---|---|---|---|---|
| | Average | SD | Average | SD | Average | SD |
| Group 1 | 1.000 | 0.000 | 0.322 | 0.239 | 0.151 | 0.201 |
| 2.5 mg/kg AC002691 | | | | | | |
| Group 2 | 1.000 | 0.000 | 0.091 | 0.053 | 0.461 | 0.707 |
| 5.0 mg/kg AC002691 | | | | | | |
| Group 3 | 1.000 | 0.000 | 0.062 | 0.034 | 0.130 | 0.130 |
| 10.0 mg/kg AC002691 | | | | | | |

Day 85

| | Average | SD |
|---|---|---|
| Group 1 | 1.719 | 2.492 |
| 2.5 mg/kg AC002691 | | |
| Group 2 | 0.458 | 0.250 |
| 5.0 mg/kg AC002691 | | |
| Group 3 | 0.299 | 0.299 |
| 10.0 mg/kg AC002691 | | |

TABLE 45
Average Relative Cynomolgus Monkey DMPK mRNA
Expression in Example 21, in Triceps.
Triceps

| | Day −14 | | Day 29 | | Day 57 | |
|---|---|---|---|---|---|---|
| | Average | SD | Average | SD | Average | SD |
| Group 2 | 1.000 | 0.000 | 0.247 | 0.067 | 0.655 | 0.268 |
| 2.5 mg/kg AC002691 | | | | | | |
| Group 3 | 1.000 | 0.000 | 0.107 | 0.066 | 0.205 | 0.219 |
| 5.0 mg/kg AC002691 | | | | | | |
| Group 4 | 1.000 | 0.000 | 0.070 | 0.015 | 0.141 | 0.016 |
| 10.0 mg/kg AC002691 | | | | | | |

Day 85

| | Average | SD |
|---|---|---|
| Group 2 | 0.235 | 0.191 |
| 2.5 mg/kg AC002691 | | |
| Group 3 | 0.168 | 0.132 |
| 5.0 mg/kg AC002691 | | |
| Group 4 | 0.140 | 0.037 |
| 10.0 mg/kg AC002691 | | |

As shown above in Tables 44 and 45, AC002691 showed significant inhibition in both quadriceps and triceps lasting until at least day 85, demonstrating the ability to silence DMPK expression in non-human primates. More specifically, RNAi agent AC002691 achieved ~93% inhibition at 10.0 mg/kg on Day 29 in triceps, and ~90% inhibition at 5.0 mg/kg on Day 29 in quadriceps.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-58. (canceled)

59. An RNAi agent, or a pharmaceutically acceptable salt thereof, comprising:
i. an antisense strand comprising a sequence (5′ to 3′) cPrpusCfsGfuAfauacUfcCfaUfgAfccagsg (SEQ ID NO: 757); and
ii. a sense strand comprising a sequence (5′ to 3′) (invAb)sccuggucaUfgGfAfguauuacgas(invAb) (SEQ ID NO: 1054);
wherein a, as, c, g, gs, and u represent 2′-O-methyladenosine-3′-phosphate, 2′-O-methyladenosine-3′-phosphorothioate, 2′-O-methylcytidine-3′-phosphate, 2′-O-methylguanosine-3′-phosphate, 2′-O-methylguanosine-3′-phosphorothioate, and 2′-O-methyluridine-3′-phosphate, respectively;
Af, Cf, Cfs, Gf, and Uf represent 2′-fluoroadenosine-3′-phosphate, 2′-fluorocytidine-3′-phosphate, 2′-fluorocytidine-3′-phosphorothioate, 2′-fluoroguanosine-3′-phosphate, and 2′-fluorouridine-3′-phosphate, respectively;
cPrpus represents 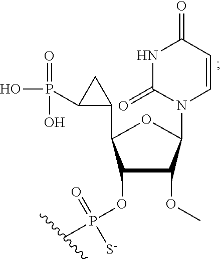
(invAb)s represents 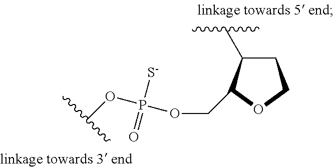
and
(invAb) represents

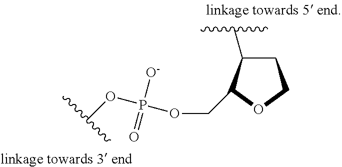

60. The RNAi agent, or a pharmaceutically acceptable salt thereof, of claim 59, wherein the RNAi agent is linked to a targeting ligand at the 5′ end of the sense strand via a

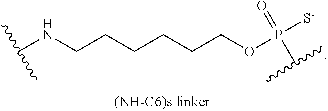 [SMILES: *NCCCCCCOP(*)(=O)[S-]]

61. The RNAi agent, or pharmaceutically acceptable salt thereof, of claim 60, wherein the targeting ligand is avb6-pep1:

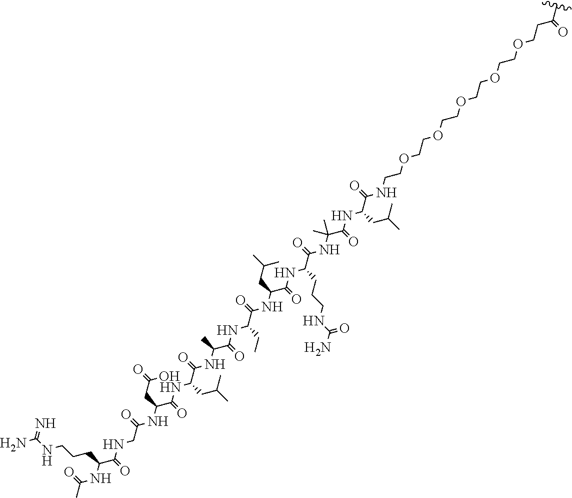 [SMILES: *C(=O)CCOCCOCCOCCOCCOCCNC(=O)[C@H](CC(C)C)NC(=O)C(C)(C)NC(=O)[C@H](CCCNC(N)=O)NC(=O)[C@H](CC(C)C)NC(=O)[C@H](CC)NC(=O)[C@H](C)NC(=O)[C@H](CC(C)C)NC(=O)[C@H](CC(=O)O)NC(=O)CNC(=O)[C@H](CCCNC(=N)N)NC(C)=O]

62. The RNAi agent, or a pharmaceutically acceptable salt thereof, of claim 61, wherein the RNAi agent is linked to a PK/PD modulator at the 3′ end of the sense strand via a linker, and wherein the linker is (C6-S):

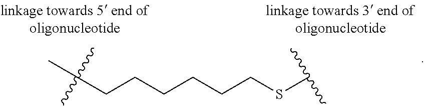 [SMILES: *CCCCCCS*]

63. The RNAi agent, or pharmaceutically acceptable salt thereof, of claim 62, wherein the PK/PD modulator is LP238b: 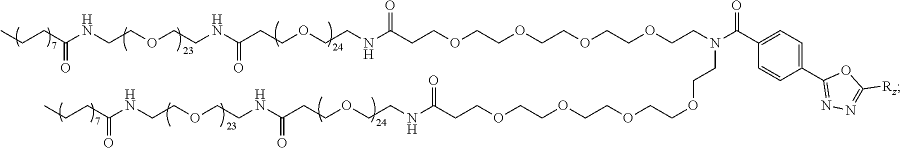
wherein R_{z} is the remainder of the RNAi agent.

64. An RNAi agent, or a pharmaceutically acceptable salt thereof, comprising:
i. an antisense strand comprising a sequence (5′ to 3′) cPrpusCfsGfuAfauacUfcCfaUfgAfccagsg (SEQ ID NO: 757); and
ii. a sense strand comprising a sequence (5′ to 3′) avb6-pep1-(NH-C6)s(invAb) sccuggucaUfgGfAfguauuacgas(invAb)(C6-S)-LP238b (SEQ ID NO: 1435); wherein:
(a) a, as, c, g, gs, and u represent 2′-O-methyladenosine-3′-phosphate, 2′-O-methyladenosine-3′-phosphorothioate, 2′-O-methylcytidine-3′-phosphate, 2′-O-methylguanosine-3′-phosphate, 2′-O-methylguanosine-3′-phosphorothioate, and 2′-O-methyluridine-3′-phosphate, respectively;
(b) Af, Cf, Cfs, Gf, and Uf represent 2′-fluoroadenosine-3′-phosphate, 2′-fluorocytidine-3′-phosphate, 2′-fluorocytidine-3′-phosphorothioate, 2′-fluoroguanosine-3′-phosphate, and 2′-fluorouridine-3′-phosphate, respectively;
(c) cPrpus represents 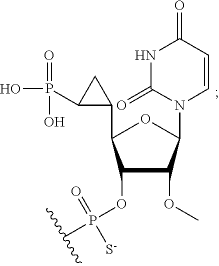
(d) avb6-pep1 represents 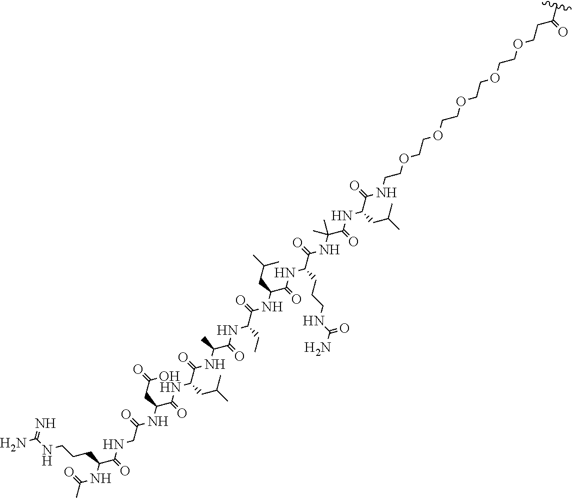 [SMILES: *C(=O)CCOCCOCCOCCOCCOCCNC(=O)[C@H](CC(C)C)NC(=O)C(C)(C)NC(=O)[C@H](CCCNC(N)=O)NC(=O)[C@H](CC(C)C)NC(=O)[C@H](CC)NC(=O)[C@H](C)NC(=O)[C@H](CC(C)C)NC(=O)[C@H](CC(=O)O)NC(=O)CNC(=O)[C@H](CCCNC(=N)N)NC(C)=O]
(e) (NH—C6) s represents 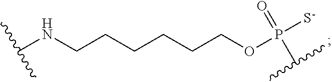 [SMILES: *NCCCCCCOP(*)(=O)[S-]]
(f) (invAb)s represents 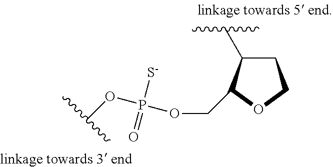
(g) (invAb) represents 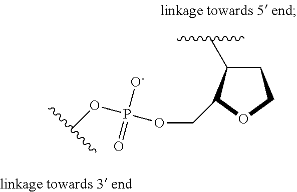
(h) (C6-S) represents 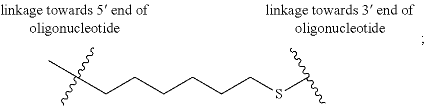 [SMILES: *CCCCCCS*]
and
(i) LP238b represents 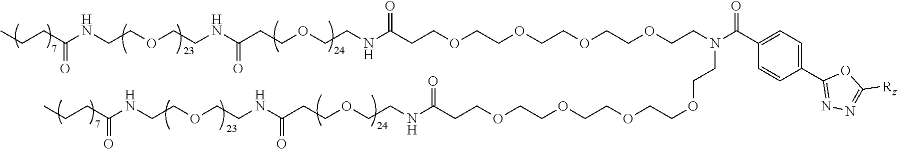
wherein
R_{z} is the remainder of the RNAi agent,
and wherein [symbol] indicates a point of connection.

65. A pharmaceutically acceptable salt of the RNAi agent of claim 64.

66. The pharmaceutically acceptable salt of claim 65, wherein the pharmaceutically acceptable salt is a sodium salt.

67. A pharmaceutical composition comprising the RNAi agent of claim 64, wherein the pharmaceutical composition comprises a pharmaceutically acceptable excipient.

68. A method of treating one or more symptoms or diseases that can be ameliorated at least in part by a reduction in DMPK protein levels, and/or a reduction in DMPK mRNA levels, the method comprising administering to a human subject in need thereof a therapeutically effective amount of the pharmaceutical composition of claim 67, wherein the disease is myotonic dystrophy type I.

69. The method of claim 68, wherein the RNAi agent is administered intravenously.

70. A method for inhibiting expression of a DMPK gene in a cell, the method comprising introducing into a cell an effective amount of an RNAi agent of claim 64.

71. The method of claim 70, wherein the cell is within a human subject.

72. The method of claim 71, wherein the DMPK gene expression is reduced in one or more of paraspinal, facial, torso, abdominal, and limb muscle tissues of the subject.

73. The method of claim 72, wherein the DMPK gene expression is reduced in one or more of the triceps, biceps, quadriceps, gastrocnemius, soleus, extensor digitorum longus (EDL), tibialis anterior (TA), or diaphragm of the subject.

74. An RNAi agent, or a pharmaceutically acceptable salt thereof, comprising:
i. an antisense strand consisting of a sequence (5′ to 3′) cPrpusCfsGfuAfauacUfcCfaUfgAfccagsg (SEQ ID NO: 757); and
ii. a sense strand comprising a sequence (5′ to 3′) (invAb) sccuggucaUfgGfAfguauuacgas (invAb) (SEQ ID NO: 1054);
wherein a, as, c, g, gs, and u represent 2′-O-methyladenosine-3′-phosphate, 2′-O-methyladenosine-3′-phosphorothioate, 2′-O-methylcytidine-3′-phosphate, 2′-O-methylguanosine-3′-phosphate, 2′-O-methylguanosine-3′-phosphorothioate, and 2′-O-methyluridine-3′-phosphate, respectively;
Af, Cf, Cfs, Gf, and Uf represent 2′-fluoroadenosine-3′-phosphate, 2′-fluorocytidine-3′-phosphate, 2′-fluorocytidine-3′-phosphorothioate, 2′-fluoroguanosine-3′-phosphate, and 2′-fluorouridine-3′-phosphate, respectively;
cPrpus represents 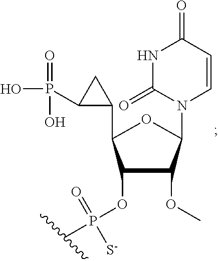
(invAb)s represents 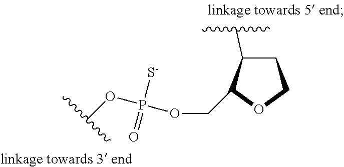
and
(invAb) represents

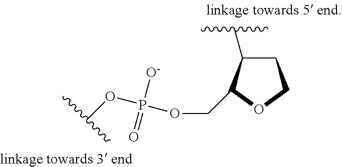

75. The RNAi agent, or a pharmaceutically acceptable salt thereof, of claim 74, wherein the RNAi agent is linked to a targeting ligand at the 5′ end of the sense strand via a

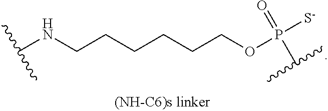 [SMILES: *NCCCCCCOP(*)(=O)[S-]]

76. The RNAi agent, or pharmaceutically acceptable salt thereof, of claim 75, wherein the targeting ligand is avb6-pep1:

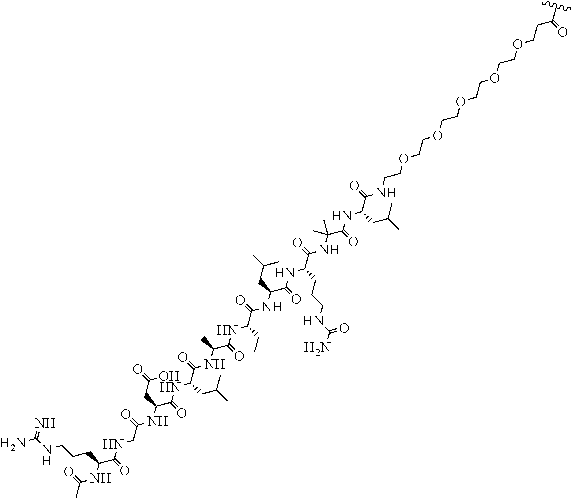 [SMILES: *C(=O)CCOCCOCCOCCOCCOCCNC(=O)[C@H](CC(C)C)NC(=O)C(C)(C)NC(=O)[C@H](CCCNC(N)=O)NC(=O)[C@H](CC(C)C)NC(=O)[C@H](CC)NC(=O)[C@H](C)NC(=O)[C@H](CC(C)C)NC(=O)[C@H](CC(=O)O)NC(=O)CNC(=O)[C@H](CCCNC(=N)N)NC(C)=O]

77. The RNAi agent, or a pharmaceutically acceptable salt thereof, of claim 76, wherein the RNAi agent is linked to a PK/PD modulator at the 3′ end of the sense strand via a linker, and wherein the linker is (C6-S):

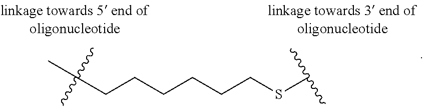 [SMILES: *CCCCCCS*]

78. The RNAi agent, or pharmaceutically acceptable salt thereof, of claim 77, wherein the PK/PD modulator is LP238b: 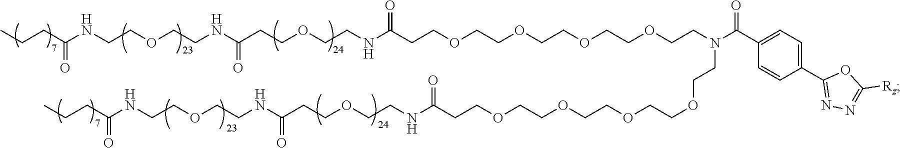
wherein R_{z} is the remainder of the RNAi agent.

79. An RNAi agent, or a pharmaceutically acceptable salt thereof, comprising:
i. an antisense strand consisting of a sequence (5′ to 3′) cPrpusCfsGfuAfauacUfcCfaUfgAfccagsg (SEQ ID NO: 757); and
ii. a sense strand comprising a sequence (5′ to 3′) avb6-pep1-(NH-C6)s(invAb)sccuggucaUfgGfAfguauuacgas(invAb)(C6-S)-LP238b (SEQ ID NO: 1435); wherein:
(a) a, as, c, g, gs, and u represent 2′-O-methyladenosine-3′-phosphate, 2′-O-methyladenosine-3′-phosphorothioate, 2′-O-methylcytidine-3′-phosphate, 2′-O-methylguanosine-3′-phosphate, 2′-O-methylguanosine-3′-phosphorothioate, and 2′-O-methyluridine-3′-phosphate, respectively;
(b) Af, Cf, Cfs, Gf, and Uf represent 2′-fluoroadenosine-3′-phosphate, 2′-fluorocytidine-3′-phosphate, 2′-fluorocytidine-3′-phosphorothioate, 2′-fluoroguanosine-3′-phosphate, and 2′-fluorouridine-3′-phosphate, respectively;
(c) cPrpus represents 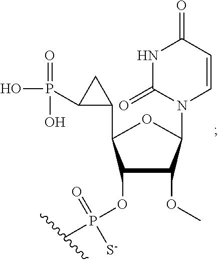
(d) avb6-pep1 represents 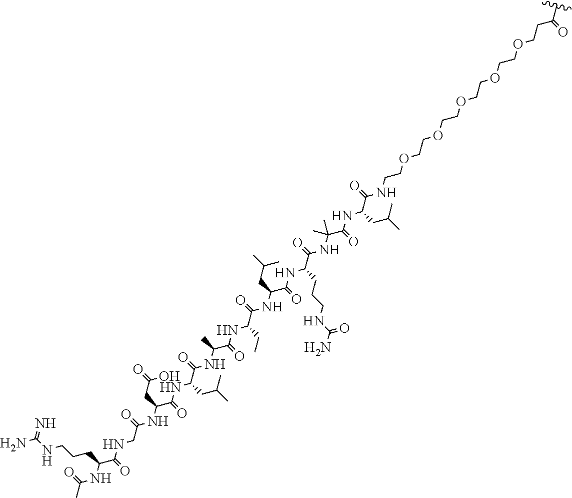 [SMILES: *C(=O)CCOCCOCCOCCOCCOCCNC(=O)[C@H](CC(C)C)NC(=O)C(C)(C)NC(=O)[C@H](CCCNC(N)=O)NC(=O)[C@H](CC(C)C)NC(=O)[C@H](CC)NC(=O)[C@H](C)NC(=O)[C@H](CC(C)C)NC(=O)[C@H](CC(=O)O)NC(=O)CNC(=O)[C@H](CCCNC(=N)N)NC(C)=O]
(e) (NH—C6) s represents 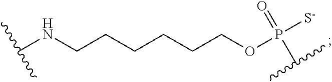 [SMILES: *NCCCCCCOP(*)(=O)[S-]]
(f) (invAb)s represents 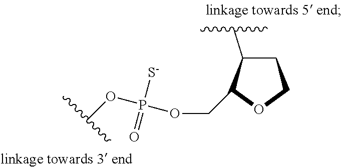
(g) (invAb) represents 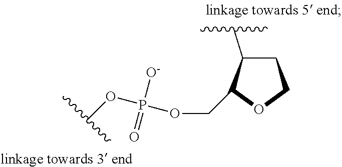
(h) (C6-S) represents 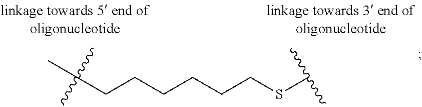 [SMILES: *CCCCCCS*]
and
(i) LP238b represents 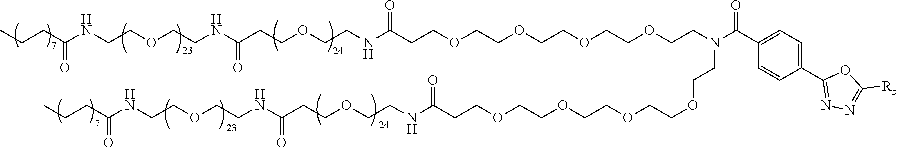
wherein
R_{z} is the remainder of the RNAi agent,
and wherein [symbol] indicates a point of connection.

80. A pharmaceutically acceptable salt of the RNAi agent of claim 79.

81. The pharmaceutically acceptable salt of claim 80, wherein the pharmaceutically acceptable salt is a sodium salt.

82. A pharmaceutical composition comprising the RNAi agent of claim 79, wherein the pharmaceutical composition comprises a pharmaceutically acceptable excipient.

83. A method of treating one or more symptoms or diseases that can be ameliorated at least in part by a reduction in DMPK protein levels, and/or a reduction in DMPK mRNA levels, the method comprising administering to a human subject in need thereof a therapeutically effective amount of the pharmaceutical composition of claim 82, wherein the disease is myotonic dystrophy type I.

84. The method of claim 83, wherein the RNAi agent is administered intravenously.

85. A method for inhibiting expression of a DMPK gene in a cell, the method comprising introducing into a cell an effective amount of an RNAi agent of claim 79.

86. The method of claim 85, wherein the cell is within a human subject.

87. The method of claim 86, wherein the DMPK gene expression is reduced in one or more of paraspinal, facial, torso, abdominal, and limb muscle tissues of the subject.

88. The method of claim 87, wherein the DMPK gene expression is reduced in one or more of the triceps, biceps, quadriceps, gastrocnemius, soleus, extensor digitorum longus (EDL), tibialis anterior (TA), or diaphragm of the subject.
